SEQUENCE LISTING (793 sequences)

SEQ ID NO: 1-8 - skipped (8 entries)

SEQ ID NO: 9 - RNA, 154 nt, synthetic construct
  source 1..154: mol_type = other RNA
atacttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgc

SEQ ID NO: 10 - RNA, 154 nt, synthetic construct
  source 1..154: mol_type = other RNA
tacttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgct

SEQ ID NO: 11 - RNA, 154 nt, synthetic construct
  source 1..154: mol_type = other RNA
acttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctt

SEQ ID NO: 12 - RNA, 154 nt, synthetic construct
  source 1..154: mol_type = other RNA
cttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgcttt

SEQ ID NO: 13 - RNA, 154 nt, synthetic construct
  source 1..154: mol_type = other RNA
ttacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttc

SEQ ID NO: 14 - RNA, 154 nt, synthetic construct
  source 1..154: mol_type = other RNA
tacgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttct

SEQ ID NO: 15 - RNA, 154 nt, synthetic construct
  source 1..154: mol_type = other RNA
acgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttctt

SEQ ID NO: 16 - RNA, 154 nt, synthetic construct
  source 1..154: mol_type = other RNA
cgtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttc

SEQ ID NO: 17 - RNA, 154 nt, synthetic construct
  source 1..154: mol_type = other RNA
gtaacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttct

SEQ ID NO: 18 - RNA, 153 nt, synthetic construct
  source 1..153: mol_type = other RNA
taacaggagaaaatacggccatgaagttggtgtttctcgggggcgatttctccattgtactcagtatgtgctgactgactcctgttacttccacatgtggggaaactggactgtaatttgtggtggtggggaattgcgttcgcgctttcttct

SEQ ID NO: 19 - RNA, 154 nt, synthetic construct
  source 1..154: mol_type = other RNA
atatttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgt

SEQ ID NO: 20 - RNA, 154 nt, synthetic construct
  source 1..154: mol_type = other RNA
tatttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtg

SEQ ID NO: 21 - RNA, 154 nt, synthetic construct
  source 1..154: mol_type = other RNA
atttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgc

SEQ ID NO: 22 - RNA, 154 nt, synthetic construct
  source 1..154: mol_type = other RNA
tttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgct

SEQ ID NO: 23 - RNA, 154 nt, synthetic construct
  source 1..154: mol_type = other RNA
ttacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgctt

SEQ ID NO: 24 - RNA, 154 nt, synthetic construct
  source 1..154: mol_type = other RNA
tacttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttt

SEQ ID NO: 25 - RNA, 154 nt, synthetic construct
  source 1..154: mol_type = other RNA
acttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgctttt

SEQ ID NO: 26 - RNA, 154 nt, synthetic construct
  source 1..154: mol_type = other RNA
cttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttc

SEQ ID NO: 27 - RNA, 154 nt, synthetic construct
  source 1..154: mol_type = other RNA
ttggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttcc

SEQ ID NO: 28 - RNA, 154 nt, synthetic construct
  source 1..154: mol_type = other RNA
tggcaggggagataacgtgaccacgaaggtggttttcccagggctgaggcttattcattgtactccggatgtgctgacccctgcgatttccccaaatgtgggaaactcgactgcataatttgtggtagtggggggctgtgtccgtgcttttccc

SEQ ID NO: 29 - RNA, 154 nt, synthetic construct
  source 1..154: mol_type = other RNA
atacttacctggcagggcagataccatgatcttaaaggcagttttcccagggcaaggcttatccattccactctggatccattataggggcatgctgatccctggaattgccccaaatgtgggaagctctactgcaaaatttttggtagtgagc

SEQ ID NO: 30 - RNA, 154 nt, synthetic construct
  source 1..154: mol_type = other RNA
tacttacctggcagggcagataccatgatcttaaaggcagttttcccagggcaaggcttatccattccactctggatccattataggggcatgctgatccctggaattgccccaaatgtgggaagctctactgcaaaatttttggtagtgagcg

SEQ ID NO: 31 - RNA, 154 nt, synthetic construct
  source 1..154: mol_type = other RNA
acttacctggcagggcagataccatgatcttaaaggcagttttcccagggcaaggcttatccattccactctggatccattataggggcatgctgatccctggaattgccccaaatgtgggaagctctactgcaaaatttttggtagtgagcga

SEQ ID NO: 32 - RNA, 154 nt, synthetic construct
  source 1..154: mol_type = other RNA
cttacctggcagggcagataccatgatcttaaaggcagttttcccagggcaaggcttatccattccactctggatccattataggggcatgctgatccctggaattgccccaaatgtgggaagctctactgcaaaatttttggtagtgagcgat

SEQ ID NO: 33 - RNA, 154 nt, synthetic construct
  source 1..154: mol_type = other RNA
ttacctggcagggcagataccatgatcttaaaggcagttttcccagggcaaggcttatccattccactctggatccattataggggcatgctgatccctggaattgccccaaatgtgggaagctctactgcaaaatttttggtagtgagcgatg

SEQ ID NO: 34 - RNA, 154 nt, synthetic construct
  source 1..154: mol_type = other RNA
tacctggcagggcagataccatgatcttaaaggcagttttcccagggcaaggcttatccattccactctggatccattataggggcatgctgatccctggaattgccccaaatgtgggaagctctactgcaaaatttttggtagtgagcgatgg

SEQ ID NO: 35 - RNA, 154 nt, synthetic construct
  source 1..154: mol_type = other RNA
acctggcagggcagataccatgatcttaaaggcagttttcccagggcaaggcttatccattccactctggatccattataggggcatgctgatccctggaattgccccaaatgtgggaagctctactgcaaaatttttggtagtgagcgatggc

SEQ ID NO: 36 - RNA, 154 nt, synthetic construct
  source 1..154: mol_type = other RNA
cctggcagggcagataccatgatcttaaaggcagttttcccagggcaaggcttatccattccactctggatccattataggggcatgctgatccctggaattgccccaaatgtgggaagctctactgcaaaatttttggtagtgagcgatggca

SEQ ID NO: 37 - RNA, 154 nt, synthetic construct
  source 1..154: mol_type = other RNA
ctggcagggcagataccatgatcttaaaggcagttttcccagggcaaggcttatccattccactctggatccattataggggcatgctgatccctggaattgccccaaatgtgggaagctctactgcaaaatttttggtagtgagcgatggcat

SEQ ID NO: 38 - RNA, 154 nt, synthetic construct
  source 1..154: mol_type = other RNA
tggcagggcagataccatgatcttaaaggcagttttcccagggcaaggcttatccattccactctggatccattataggggcatgctgatccctggaattgccccaaatgtgggaagctctactgcaaaatttttggtagtgagcgatggcatt

SEQ ID NO: 39 - RNA, 154 nt, synthetic construct
  source 1..154: mol_type = other RNA
atacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcg

SEQ ID NO: 40 - RNA, 154 nt, synthetic construct
  source 1..154: mol_type = other RNA
tacttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgc

SEQ ID NO: 41 - RNA, 154 nt, synthetic construct
  source 1..154: mol_type = other RNA
acttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgct

SEQ ID NO: 42 - RNA, 154 nt, synthetic construct
  source 1..154: mol_type = other RNA
cttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctt

SEQ ID NO: 43 - RNA, 154 nt, synthetic construct
  source 1..154: mol_type = other RNA
ttacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgcttt

SEQ ID NO: 44 - RNA, 154 nt, synthetic construct
  source 1..154: mol_type = other RNA
tacctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttc

SEQ ID NO: 45 - RNA, 154 nt, synthetic construct
  source 1..154: mol_type = other RNA
acctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcc

SEQ ID NO: 46 - RNA, 154 nt, synthetic construct
  source 1..154: mol_type = other RNA
cctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttccc

SEQ ID NO: 47 - RNA, 154 nt, synthetic construct
  source 1..154: mol_type = other RNA
ctggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccc

SEQ ID NO: 48 - RNA, 154 nt, synthetic construct
  source 1..154: mol_type = other RNA
tggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccct

SEQ ID NO: 49 - RNA, 154 nt, synthetic construct
  source 1..154: mol_type = other RNA
atacttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 50 - RNA, 153 nt, synthetic construct
  source 1..153: mol_type = other RNA
tacttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 51 - RNA, 152 nt, synthetic construct
  source 1..152: mol_type = other RNA
acttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 52 - RNA, 151 nt, synthetic construct
  source 1..151: mol_type = other RNA
cttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 53 - RNA, 150 nt, synthetic construct
  source 1..150: mol_type = other RNA
ttccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 54 - RNA, 149 nt, synthetic construct
  source 1..149: mol_type = other RNA
tccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 55 - RNA, 148 nt, synthetic construct
  source 1..148: mol_type = other RNA
ccctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 56 - RNA, 147 nt, synthetic construct
  source 1..147: mol_type = other RNA
cctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 57 - RNA, 146 nt, synthetic construct
  source 1..146: mol_type = other RNA
ctgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 58 - RNA, 145 nt, synthetic construct
  source 1..145: mol_type = other RNA
tgacaggggagatacctaggaatccaacttcccagggcaagactgatctattgcactatggatgtgccgacccctgagatttacaaaattgtgggaaactcaactgcataatttatggaaatgaaggactgtgtttgcgctttca

SEQ ID NO: 59 - RNA, 154 nt, synthetic construct
  source 1..154: mol_type = other RNA
aaacttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtg

SEQ ID NO: 60 - RNA, 154 nt, synthetic construct
  source 1..154: mol_type = other RNA
aacttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgc

SEQ ID NO: 61 - RNA, 154 nt, synthetic construct
  source 1..154: mol_type = other RNA
acttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgct

SEQ ID NO: 62 - RNA, 154 nt, synthetic construct
  source 1..154: mol_type = other RNA
cttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctt

SEQ ID NO: 63 - RNA, 154 nt, synthetic construct
  source 1..154: mol_type = other RNA
ttctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgcttt

SEQ ID NO: 64 - RNA, 154 nt, synthetic construct
  source 1..154: mol_type = other RNA
tctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttc

SEQ ID NO: 65 - RNA, 154 nt, synthetic construct
  source 1..154: mol_type = other RNA
ctctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttca

SEQ ID NO: 66 - RNA, 154 nt, synthetic construct
  source 1..154: mol_type = other RNA
tctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttcat

SEQ ID NO: 67 - RNA, 154 nt, synthetic construct
  source 1..154: mol_type = other RNA
ctgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttcatg

SEQ ID NO: 68 - RNA, 154 nt, synthetic construct
  source 1..154: mol_type = other RNA
tgccaggggagataccatgatcacaaaggtggctttctcagggaaaggctgatttattgcactccagatgtgctgacccctgagatttctccaaatgtgggaaactcagctgcataatttgtggaagcgaaggactgtgtttgtgctttcatgt

SEQ ID NO: 69 - RNA, 154 nt, synthetic construct
  source 1..154: mol_type = other RNA
atacttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcac

SEQ ID NO: 70 - RNA, 154 nt, synthetic construct
  source 1..154: mol_type = other RNA
tacttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcact

SEQ ID NO: 71 - RNA, 154 nt, synthetic construct
  source 1..154: mol_type = other RNA
acttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactt

SEQ ID NO: 72 - RNA, 154 nt, synthetic construct
  source 1..154: mol_type = other RNA
cttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcacttt

SEQ ID NO: 73 - RNA, 154 nt, synthetic construct
  source 1..154: mol_type = other RNA
ttctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttc

SEQ ID NO: 74 - RNA, 154 nt, synthetic construct
  source 1..154: mol_type = other RNA
tctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttca

SEQ ID NO: 75 - RNA, 154 nt, synthetic construct
  source 1..154: mol_type = other RNA
ctctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcac

SEQ ID NO: 76 - RNA, 154 nt, synthetic construct
  source 1..154: mol_type = other RNA
tctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcacg

SEQ ID NO: 77 - RNA, 154 nt, synthetic construct
  source 1..154: mol_type = other RNA
ctgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcacgt

SEQ ID NO: 78 - RNA, 153 nt, synthetic construct
  source 1..153: mol_type = other RNA
tgccaggggagatactgtgatcatgaaggtggctttcccagggcaaggctgatctattgcattctggatgtgctgactcctacgatttccccaagtgtgggaaactcaactgcataatttgtggaagtaatgactgtgtttgcactttcacgt

SEQ ID NO: 79 - RNA, 154 nt, synthetic construct
  source 1..154: mol_type = other RNA
atacttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtg

SEQ ID NO: 80 - RNA, 154 nt, synthetic construct
  source 1..154: mol_type = other RNA
tacttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgc

SEQ ID NO: 81 - RNA, 154 nt, synthetic construct
  source 1..154: mol_type = other RNA
acttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgct

SEQ ID NO: 82 - RNA, 154 nt, synthetic construct
  source 1..154: mol_type = other RNA
cttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctt

SEQ ID NO: 83 - RNA, 154 nt, synthetic construct
  source 1..154: mol_type = other RNA
ttccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgcttt

SEQ ID NO: 84 - RNA, 154 nt, synthetic construct
  source 1..154: mol_type = other RNA
tccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttc

SEQ ID NO: 85 - RNA, 154 nt, synthetic construct
  source 1..154: mol_type = other RNA
ccctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 86 - RNA, 153 nt, synthetic construct
  source 1..153: mol_type = other RNA
cctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 87 - RNA, 152 nt, synthetic construct
  source 1..152: mol_type = other RNA
ctgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 88 - RNA, 151 nt, synthetic construct
  source 1..151: mol_type = other RNA
tgccaggggagataccgtgatcatgaaggtggctttcccagggaaaggccgatatgttgcactctagatgtgctgacccgtgagatttccccaaatgtgggacactcaaatgcataattggtggaagtgaaggactgtgtttgtgctttca

SEQ ID NO: 89 - RNA, 154 nt, synthetic construct
  source 1..154: mol_type = other RNA
ttaactacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatg

SEQ ID NO: 90 - RNA, 154 nt, synthetic construct
  source 1..154: mol_type = other RNA
taactacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgt

SEQ ID NO: 91 - RNA, 154 nt, synthetic construct
  source 1..154: mol_type = other RNA
aactacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgtt

SEQ ID NO: 92 - RNA, 154 nt, synthetic construct
  source 1..154: mol_type = other RNA
actacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttc

SEQ ID NO: 93 - RNA, 154 nt, synthetic construct
  source 1..154: mol_type = other RNA
ctacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttct

SEQ ID NO: 94 - RNA, 154 nt, synthetic construct
  source 1..154: mol_type = other RNA
tacctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctc

SEQ ID NO: 95 - RNA, 154 nt, synthetic construct
  source 1..154: mol_type = other RNA
acctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctcc

SEQ ID NO: 96 - RNA, 154 nt, synthetic construct
  source 1..154: mol_type = other RNA
cctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctccc

SEQ ID NO: 97 - RNA, 154 nt, synthetic construct
  source 1..154: mol_type = other RNA
ctgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctcccc

SEQ ID NO: 98 - RNA, 154 nt, synthetic construct
  source 1..154: mol_type = other RNA
tgacagaggagatactgtgatcatgaaagtggttttcctagggcaagacttatccgttgcactccagatgtgctgactcatgcaatttccccaaatgtgggaaactcgactacataatttctggtggtaggggactgcgttcatgttctcccct

SEQ ID NO: 99 - RNA, 147 nt, synthetic construct
  source 1..147: mol_type = other RNA
agacttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 100 - RNA, 146 nt, synthetic construct
  source 1..146: mol_type = other RNA
gacttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 101 - RNA, 145 nt, synthetic construct
  source 1..145: mol_type = other RNA
acttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 102 - RNA, 144 nt, synthetic construct
  source 1..144: mol_type = other RNA
cttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 103 - RNA, 143 nt, synthetic construct
  source 1..143: mol_type = other RNA
ttatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 104 - RNA, 142 nt, synthetic construct
  source 1..142: mol_type = other RNA
tatctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 105 - RNA, 141 nt, synthetic construct
  source 1..141: mol_type = other RNA
atctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 106 - RNA, 140 nt, synthetic construct
  source 1..140: mol_type = other RNA
tctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 107 - RNA, 139 nt, synthetic construct
  source 1..139: mol_type = other RNA
ctggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 108 - RNA, 138 nt, synthetic construct
  source 1..138: mol_type = other RNA
tggcaggggagacaccatgaacatgaggatagttttcccaaggcaagtttcaacccttgcactctagatgatgagattacttaatgggtacaatgcatgtgatttgggtaatggatactgtagaaacactgacgtcac

SEQ ID NO: 109 - RNA, 149 nt, synthetic construct
  source 1..149: mol_type = other RNA
agactcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 110 - RNA, 148 nt, synthetic construct
  source 1..148: mol_type = other RNA
gactcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 111 - RNA, 147 nt, synthetic construct
  source 1..147: mol_type = other RNA
actcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 112 - RNA, 146 nt, synthetic construct
  source 1..146: mol_type = other RNA
ctcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 113 - RNA, 145 nt, synthetic construct
  source 1..145: mol_type = other RNA
tcatctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 114 - RNA, 144 nt, synthetic construct
  source 1..144: mol_type = other RNA
catctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 115 - RNA, 143 nt, synthetic construct
  source 1..143: mol_type = other RNA
atctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 116 - RNA, 142 nt, synthetic construct
  source 1..142: mol_type = other RNA
tctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 117 - RNA, 141 nt, synthetic construct
  source 1..141: mol_type = other RNA
ctggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 118 - RNA, 140 nt, synthetic construct
  source 1..140: mol_type = other RNA
tggcagcggagataccatgaacatcacggtagttttcccaaagcaagtttccacccttgcactccggatgatgagacattacttaatggatacaatgcatgtgatttgagtgatggatactgtggaaacaccgacttcac

SEQ ID NO: 119 - RNA, 154 nt, synthetic construct
  source 1..154: mol_type = other RNA
agacttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttt

SEQ ID NO: 120 - RNA, 154 nt, synthetic construct
  source 1..154: mol_type = other RNA
gacttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacactttt

SEQ ID NO: 121 - RNA, 154 nt, synthetic construct
  source 1..154: mol_type = other RNA
acttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttc

SEQ ID NO: 122 - RNA, 154 nt, synthetic construct
  source 1..154: mol_type = other RNA
cttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcc

SEQ ID NO: 123 - RNA, 154 nt, synthetic construct
  source 1..154: mol_type = other RNA
ttggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttccc

SEQ ID NO: 124 - RNA, 154 nt, synthetic construct
  source 1..154: mol_type = other RNA
tggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccg

SEQ ID NO: 125 - RNA, 154 nt, synthetic construct
  source 1..154: mol_type = other RNA
ggcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 126 - RNA, 153 nt, synthetic construct
  source 1..153: mol_type = other RNA
gcaggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 127 - RNA, 152 nt, synthetic construct
  source 1..152: mol_type = other RNA
caggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 128 - RNA, 151 nt, synthetic construct
  source 1..151: mol_type = other RNA
aggggagatagcatgatcacgaaggtggttttcccaaggcaagatttattcactgcactctggatgtgctgacccctacgatttccgcccaatggggaagcttgactgcttagtttgttgtggcaggggactgtgttcacacttttcccgg

SEQ ID NO: 129 - RNA, 154 nt, synthetic construct
  source 1..154: mol_type = other RNA
atacttaacatggcagaggagatatcataatcacaaaggtagttttcccagggcaagccttatccactgcattccagatgtgctcacctctgtggtttccccaaatgtggaaaactggactgcataatttgtggtagcgggggactgcattaat

SEQ ID NO: 130 - RNA, 154 nt, synthetic construct
  source 1..154: mol_type = other RNA
tacttaacatggcagaggagatatcataatcacaaaggtagttttcccagggcaagccttatccactgcattccagatgtgctcacctctgtggtttccccaaatgtggaaaactggactgcataatttgtggtagcgggggactgcattaata

SEQ ID NO: 131 - RNA, 154 nt, synthetic construct
  source 1..154: mol_type = other RNA
acttaacatggcagaggagatatcataatcacaaaggtagttttcccagggcaagccttatccactgcattccagatgtgctcacctctgtggtttccccaaatgtggaaaactggactgcataatttgtggtagcgggggactgcattaatac

SEQ ID NO: 132 - RNA, 154 nt, synthetic construct
  source 1..154: mol_type = other RNA
cttaacatggcagaggagatatcataatcacaaaggtagttttcccagggcaagccttatccactgcattccagatgtgctcacctctgtggtttccccaaatgtggaaaactggactgcataatttgtggtagcgggggactgcattaatacc

SEQ ID NO: 133 - RNA, 154 nt, synthetic construct
  source 1..154: mol_type = other RNA
ttaacatggcagaggagatatcataatcacaaaggtagttttcccagggcaagccttatccactgcattccagatgtgctcacctctgtggtttccccaaatgtggaaaactggactgcataatttgtggtagcgggggactgcattaatacct

SEQ ID NO: 134 - RNA, 154 nt, synthetic construct
  source 1..154: mol_type = other RNA
taacatggcagaggagatatcataatcacaaaggtagttttcccagggcaagccttatccactgcattccagatgtgctcacctctgtggtttccccaaatgtggaaaactggactgcataatttgtggtagcgggggactgcattaatacctt

SEQ ID NO: 135 - RNA, 154 nt, synthetic construct
  source 1..154: mol_type = other RNA
aacatggcagaggagatatcataatcacaaaggtagttttcccagggcaagccttatccactgcattccagatgtgctcacctctgtggtttccccaaatgtggaaaactggactgcataatttgtggtagcgggggactgcattaataccttc

SEQ ID NO: 136 - RNA, 154 nt, synthetic construct
  source 1..154: mol_type = other RNA
acatggcagaggagatatcataatcacaaaggtagttttcccagggcaagccttatccactgcattccagatgtgctcacctctgtggtttccccaaatgtggaaaactggactgcataatttgtggtagcgggggactgcattaataccttcc

SEQ ID NO: 137 - RNA, 154 nt, synthetic construct
  source 1..154: mol_type = other RNA
catggcagaggagatatcataatcacaaaggtagttttcccagggcaagccttatccactgcattccagatgtgctcacctctgtggtttccccaaatgtggaaaactggactgcataatttgtggtagcgggggactgcattaataccttcct

SEQ ID NO: 138 - RNA, 154 nt, synthetic construct
  source 1..154: mol_type = other RNA
atggcagaggagatatcataatcacaaaggtagttttcccagggcaagccttatccactgcattccagatgtgctcacctctgtggtttccccaaatgtggaaaactggactgcataatttgtggtagcgggggactgcattaataccttcctc

SEQ ID NO: 139 - RNA, 133 nt, synthetic construct
  source 1..133: mol_type = other RNA
aaaaagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 140 - RNA, 132 nt, synthetic construct
  source 1..132: mol_type = other RNA
aaaagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 141 - RNA, 131 nt, synthetic construct
  source 1..131: mol_type = other RNA
aaagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 142 - RNA, 130 nt, synthetic construct
  source 1..130: mol_type = other RNA
aagggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 143 - RNA, 129 nt, synthetic construct
  source 1..129: mol_type = other RNA
agggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 144 - RNA, 128 nt, synthetic construct
  source 1..128: mol_type = other RNA
gggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 145 - RNA, 127 nt, synthetic construct
  source 1..127: mol_type = other RNA
ggcttctgctgtgagtggcacacatagggcatcgtttgctcttggtgccagaatcaacatcaagagatttcagaagcataattttttggtacttgggcagctggtgatcattggtcctgtagccctt

SEQ ID NO: 146 - RNA, 126 nt, synthetic construct
  source 1..126: mol_type = other RNA
gcttctgctgtgagtggcacacatagggcatcgtttgctcttggtgccagaatcaacatcaagagatttcagaagcataattttttggtacttgggcagctggtgatcattggtcctgtagccctt

SEQ ID NO: 147 - RNA, 125 nt, synthetic construct
  source 1..125: mol_type = other RNA
cttctgctgtgagtggcacacatagggcatcgtttgctcttggtgccagaatcaacatcaagagatttcagaagcataattttttggtacttgggcagctggtgatcattggtcctgtagccctt

SEQ ID NO: 148 - RNA, 124 nt, synthetic construct
  source 1..124: mol_type = other RNA
ttctgctgtgagtggcacacatagggcatcgtttgctcttggtgccagaatcaacatcaagagatttcagaagcataattttttggtacttgggcagctggtgatcattggtcctgtagccctt

SEQ ID NO: 149 - RNA, 129 nt, synthetic construct
  source 1..129: mol_type = other RNA
aaaaaaagctgctgttgtgagtgatacatgcagggcaacttgattgctcttagtgcagaattgacatcaaggaattttggaagtataattttttggcaggtggatagctggttgtattagtccattctc

SEQ ID NO: 150 - RNA, 128 nt, synthetic construct
  source 1..128: mol_type = other RNA
aaaaaagctgctgttgtgagtgatacatgcagggcaacttgattgctcttagtgcagaattgacatcaaggaattttggaagtataattttttggcaggtggatagctggttgtattagtccattctc

SEQ ID NO: 151 - RNA, 127 nt, synthetic construct
  source 1..127: mol_type = other RNA
aaaaagctgctgttgtgagtgatacatgcagggcaacttgattgctcttagtgcagaattgacatcaaggaattttggaagtataattttttggcaggtggatagctggttgtattagtccattctc

SEQ ID NO: 152 - RNA, 126 nt, synthetic construct
  source 1..126: mol_type = other RNA
aaaagctgctgttgtgagtgatacatgcagggcaacttgattgctcttagtgcagaattgacatcaaggaattttggaagtataattttttggcaggtggatagctggttgtattagtccattctc

SEQ ID NO: 153 - RNA, 125 nt, synthetic construct
  source 1..125: mol_type = other RNA
aaagctgctgttgtgagtgatacatgcagggcaacttgattgctcttagtgcagaattgacatcaaggaattttggaagtataattttttggcaggtggatagctggttgtattagtccattctc

SEQ ID NO: 154 - RNA, 124 nt, synthetic construct
  source 1..124: mol_type = other RNA
aagctgctgttgtgagtgatacatgcagggcaacttgattgctcttagtgcagaattgacatcaaggaattttggaagtataattttttggcaggtggatagctggttgtattagtccattctc

SEQ ID NO: 155 - RNA, 123 nt, synthetic construct
  source 1..123: mol_type = other RNA
agctgctgttgtgagtgatacatgcagggcaacttgattgctcttagtgcagaattgacatcaaggaattttggaagtataattttttggcaggtggatagctggttgtattagtccattctc

SEQ ID NO: 156 - RNA, 122 nt, synthetic construct
  source 1..122: mol_type = other RNA
gctgctgttgtgagtgatacatgcagggcaacttgattgctcttagtgcagaattgacatcaaggaattttggaagtataattttttggcaggtggatagctggttgtattagtccattctc

SEQ ID NO: 157 - RNA, 121 nt, synthetic construct
  source 1..121: mol_type = other RNA
ctgctgttgtgagtgatacatgcagggcaacttgattgctcttagtgcagaattgacatcaaggaattttggaagtataattttttggcaggtggatagctggttgtattagtccattctc

SEQ ID NO: 158 - RNA, 120 nt, synthetic construct
  source 1..120: mol_type = other RNA
tgctgttgtgagtgatacatgcagggcaacttgattgctcttagtgcagaattgacatcaaggaattttggaagtataattttttggcaggtggatagctggttgtattagtccattctc

SEQ ID NO: 159 - RNA, 133 nt, synthetic construct
  source 1..133: mol_type = other RNA
aaaaagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 160 - RNA, 132 nt, synthetic construct
  source 1..132: mol_type = other RNA
aaaagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 161 - RNA, 131 nt, synthetic construct
  source 1..131: mol_type = other RNA
aaagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 162 - RNA, 130 nt, synthetic construct
  source 1..130: mol_type = other RNA
aagggcttctgtcatgagtggcacacataggacaactcaatttctcttcatgcagaataaacatcaagagattttggaagcgtaatttttggtagttgggcagctggtgatcactggtgccagcaccctt

SEQ ID NO: 163 - RNA, 129 nt, synthetic construct
  source 1..129: mol_type = other RNA
agggcttctgtcatgagtggcacacataggacaactcaatttctcttcatgcagaataaacatcaagagattttggaagcgtaatttttggtagttgggcagctggtgatcactggtgccagcaccctt

SEQ ID NO: 164 - RNA, 128 nt, synthetic construct
  source 1..128: mol_type = other RNA
gggcttctgtcatgagtggcacacataggacaactcaatttctcttcatgcagaataaacatcaagagattttggaagcgtaatttttggtagttgggcagctggtgatcactggtgccagcaccctt

SEQ ID NO: 165 - RNA, 127 nt, synthetic construct
  source 1..127: mol_type = other RNA
ggcttctgtcatgagtggcacacataggacaactcaatttctcttcatgcagaataaacatcaagagattttggaagcgtaatttttggtagttgggcagctggtgatcactggtgccagcaccctt

SEQ ID NO: 166 - RNA, 126 nt, synthetic construct
  source 1..126: mol_type = other RNA
gcttctgtcatgagtggcacacataggacaactcaatttctcttcatgcagaataaacatcaagagattttggaagcgtaatttttggtagttgggcagctggtgatcactggtgccagcaccctt

SEQ ID NO: 167 - RNA, 125 nt, synthetic construct
  source 1..125: mol_type = other RNA
cttctgtcatgagtggcacacataggacaactcaatttctcttcatgcagaataaacatcaagagattttggaagcgtaatttttggtagttgggcagctggtgatcactggtgccagcaccctt

SEQ ID NO: 168 - RNA, 124 nt, synthetic construct
  source 1..124: mol_type = other RNA
ttctgtcatgagtggcacacataggacaactcaatttctcttcatgcagaataaacatcaagagattttggaagcgtaatttttggtagttgggcagctggtgatcactggtgccagcaccctt

SEQ ID NO: 169 - RNA, 133 nt, synthetic construct
  source 1..133: mol_type = other RNA
aaaaagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 170 - RNA, 132 nt, synthetic construct
  source 1..132: mol_type = other RNA
aaaagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 171 - RNA, 131 nt, synthetic construct
  source 1..131: mol_type = other RNA
aaagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 172 - RNA, 130 nt, synthetic construct
  source 1..130: mol_type = other RNA
aagggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 173 - RNA, 129 nt, synthetic construct
  source 1..129: mol_type = other RNA
agggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 174 - RNA, 128 nt, synthetic construct
  source 1..128: mol_type = other RNA
gggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 175 - RNA, 127 nt, synthetic construct
  source 1..127: mol_type = other RNA
ggcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 176 - RNA, 126 nt, synthetic construct
  source 1..126: mol_type = other RNA
gcttctgtcatgagtggcctatgtagggcaaccagattgctcttcatgtggaattgacatcaagagctttcagaagtgtattttttggaagttgggcagctggtaatcattggtcttggcatcctt

SEQ ID NO: 177 - RNA, 125 nt, synthetic construct
  source 1..125: mol_type = other RNA
cttctgtcatgagtggcctatgtagggcaaccagattgctcttcatgtggaattgacatcaagagctttcagaagtgtattttttggaagttgggcagctggtaatcattggtcttggcatcctt

SEQ ID NO: 178 - RNA, 124 nt, synthetic construct
  source 1..124: mol_type = other RNA
ttctgtcatgagtggcctatgtagggcaaccagattgctcttcatgtggaattgacatcaagagctttcagaagtgtattttttggaagttgggcagctggtaatcattggtcttggcatcctt

SEQ ID NO: 179 - RNA, 142 nt, synthetic construct
  source 1..142: mol_type = other RNA
aaagggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 180 - RNA, 141 nt, synthetic construct
  source 1..141: mol_type = other RNA
aagggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 181 - RNA, 140 nt, synthetic construct
  source 1..140: mol_type = other RNA
agggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 182 - RNA, 139 nt, synthetic construct
  source 1..139: mol_type = other RNA
gggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 183 - RNA, 138 nt, synthetic construct
  source 1..138: mol_type = other RNA
ggggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 184 - RNA, 137 nt, synthetic construct
  source 1..137: mol_type = other RNA
gggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 185 - RNA, 136 nt, synthetic construct
  source 1..136: mol_type = other RNA
ggcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 186 - RNA, 135 nt, synthetic construct
  source 1..135: mol_type = other RNA
gcttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 187 - RNA, 134 nt, synthetic construct
  source 1..134: mol_type = other RNA
cttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 188 - RNA, 133 nt, synthetic construct
  source 1..133: mol_type = other RNA
ttctgtcatgagtggcacacattgggcaactcaactgctcttcatgaggaatcaacatcaggaggttttggaagaatgatttttttggtagttgggcagcttgtgagaaaaaaatgttttcaggcaaatcctc

SEQ ID NO: 189 - RNA, 134 nt, synthetic construct
  source 1..134: mol_type = other RNA
aaaaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 190 - RNA, 133 nt, synthetic construct
  source 1..133: mol_type = other RNA
aaaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 191 - RNA, 132 nt, synthetic construct
  source 1..132: mol_type = other RNA
aaagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 192 - RNA, 131 nt, synthetic construct
  source 1..131: mol_type = other RNA
aagggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 193 - RNA, 130 nt, synthetic construct
  source 1..130: mol_type = other RNA
agggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 194 - RNA, 129 nt, synthetic construct
  source 1..129: mol_type = other RNA
gggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 195 - RNA, 128 nt, synthetic construct
  source 1..128: mol_type = other RNA
ggcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 196 - RNA, 127 nt, synthetic construct
  source 1..127: mol_type = other RNA
gcttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 197 - RNA, 126 nt, synthetic construct
  source 1..126: mol_type = other RNA
cttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 198 - RNA, 125 nt, synthetic construct
  source 1..125: mol_type = other RNA
ttctgtcgtgagtggcacacgtagggcaactcgattgctctgcgtgcggaatcgacatcaagagatttcggaagcataattttttggtatttgggcagctggtgatcgttggtcccggcgccctt

SEQ ID NO: 199 - RNA, 131 nt, synthetic construct
  source 1..131: mol_type = other RNA
aaaaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 200 - RNA, 130 nt, synthetic construct
  source 1..130: mol_type = other RNA
aaaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 201 - RNA, 129 nt, synthetic construct
  source 1..129: mol_type = other RNA
aaagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 202 - RNA, 128 nt, synthetic construct
  source 1..128: mol_type = other RNA
aagggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 203 - RNA, 127 nt, synthetic construct
  source 1..127: mol_type = other RNA
agggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 204 - RNA, 126 nt, synthetic construct
  source 1..126: mol_type = other RNA
gggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 205 - RNA, 125 nt, synthetic construct
  source 1..125: mol_type = other RNA
ggcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 206 - RNA, 124 nt, synthetic construct
  source 1..124: mol_type = other RNA
gcttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 207 - RNA, 123 nt, synthetic construct
  source 1..123: mol_type = other RNA
cttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 208 - RNA, 122 nt, synthetic construct
  source 1..122: mol_type = other RNA
ttctgtcgtgagtggcacacgtagggcaactcgattgctctgcgtgcggaatcgacatcaagagatttcggaagcataattttttggtatttgggcagctggtgatcgttggtcccggcgcc

SEQ ID NO: 209 - RNA, 11 nt, synthetic construct
  source 1..11: mol_type = other RNA
aatttttggag

SEQ ID NO: 210 - RNA, 11 nt, synthetic construct
  source 1..11: mol_type = other RNA
aatttttggag

SEQ ID NO: 211 - RNA, 11 nt, synthetic construct
  source 1..11: mol_type = other RNA
tatttttggag

SEQ ID NO: 212 - RNA, 11 nt, synthetic construct
  source 1..11: mol_type = other RNA
gatttttggag

SEQ ID NO: 213 - RNA, 11 nt, synthetic construct
  source 1..11: mol_type = other RNA
catttttggag

SEQ ID NO: 214 - RNA, 11 nt, synthetic construct
  source 1..11: mol_type = other RNA
attttttggag

SEQ ID NO: 215 - RNA, 11 nt, synthetic construct
  source 1..11: mol_type = other RNA
agtttttggag

SEQ ID NO: 216 - RNA, 11 nt, synthetic construct
  source 1..11: mol_type = other RNA
actttttggag

SEQ ID NO: 217 - RNA, 11 nt, synthetic construct
  source 1..11: mol_type = other RNA
aaattttggag

SEQ ID NO: 218 - RNA, 11 nt, synthetic construct
  source 1..11: mol_type = other RNA
aagttttggag

SEQ ID NO: 219 - RNA, 11 nt, synthetic construct
  source 1..11: mol_type = other RNA
aacttttggag

SEQ ID NO: 220 - RNA, 11 nt, synthetic construct
  source 1..11: mol_type = other RNA
aatatttggag

SEQ ID NO: 221 - RNA, 11 nt, synthetic construct
  source 1..11: mol_type = other RNA
aatgtttggag

SEQ ID NO: 222 - RNA, 11 nt, synthetic construct
  source 1..11: mol_type = other RNA
aatctttggag

SEQ ID NO: 223 - RNA, 11 nt, synthetic construct
  source 1..11: mol_type = other RNA
aattattggag

SEQ ID NO: 224 - RNA, 11 nt, synthetic construct
  source 1..11: mol_type = other RNA
aattgttggag

SEQ ID NO: 225 - RNA, 11 nt, synthetic construct
  source 1..11: mol_type = other RNA
aattcttggag

SEQ ID NO: 226 - RNA, 11 nt, synthetic construct
  source 1..11: mol_type = other RNA
aatttatggag

SEQ ID NO: 227 - RNA, 11 nt, synthetic construct
  source 1..11: mol_type = other RNA
aatttgtggag

SEQ ID NO: 228 - RNA, 11 nt, synthetic construct
  source 1..11: mol_type = other RNA
aatttctggag

SEQ ID NO: 229 - RNA, 11 nt, synthetic construct
  source 1..11: mol_type = other RNA
aattttaggag

SEQ ID NO: 230 - RNA, 11 nt, synthetic construct
  source 1..11: mol_type = other RNA
aattttgggag

SEQ ID NO: 231 - RNA, 11 nt, synthetic construct
  source 1..11: mol_type = other RNA
aattttcggag

SEQ ID NO: 232 - RNA, 11 nt, synthetic construct
  source 1..11: mol_type = other RNA
aatttttagag

SEQ ID NO: 233 - RNA, 11 nt, synthetic construct
  source 1..11: mol_type = other RNA
aattttttgag

SEQ ID NO: 234 - RNA, 11 nt, synthetic construct
  source 1..11: mol_type = other RNA
aatttttcgag

SEQ ID NO: 235 - RNA, 11 nt, synthetic construct
  source 1..11: mol_type = other RNA
aatttttgaag

SEQ ID NO: 236 - RNA, 11 nt, synthetic construct
  source 1..11: mol_type = other RNA
aatttttgtag

SEQ ID NO: 237 - RNA, 11 nt, synthetic construct
  source 1..11: mol_type = other RNA
aatttttgcag

SEQ ID NO: 238 - RNA, 11 nt, synthetic construct
  source 1..11: mol_type = other RNA
aatttttggtg

SEQ ID NO: 239 - RNA, 11 nt, synthetic construct
  source 1..11: mol_type = other RNA
aatttttgggg

SEQ ID NO: 240 - RNA, 11 nt, synthetic construct
  source 1..11: mol_type = other RNA
aatttttggcg

SEQ ID NO: 241 - RNA, 11 nt, synthetic construct
  source 1..11: mol_type = other RNA
aatttttggaa

SEQ ID NO: 242 - RNA, 11 nt, synthetic construct
  source 1..11: mol_type = other RNA
aatttttggat

SEQ ID NO: 243 - RNA, 11 nt, synthetic construct
  source 1..11: mol_type = other RNA
aatttttggac

SEQ ID NO: 244 - RNA, 11 nt, synthetic construct
  source 1..11: mol_type = other RNA
aatgttttgag

SEQ ID NO: 245 - RNA, 11 nt, synthetic construct
  source 1..11: mol_type = other RNA
aatttgtgtag

SEQ ID NO: 246 - RNA, 11 nt, synthetic construct
  source 1..11: mol_type = other RNA
agtttttggaa

SEQ ID NO: 247 - RNA, 11 nt, synthetic construct
  source 1..11: mol_type = other RNA
attttgtggag

SEQ ID NO: 248 - RNA, 11 nt, synthetic construct
  source 1..11: mol_type = other RNA
aatgtttgcag

SEQ ID NO: 249 - RNA, 11 nt, synthetic construct
  source 1..11: mol_type = other RNA
aatgttgggag

SEQ ID NO: 250 - RNA, 11 nt, synthetic construct
  source 1..11: mol_type = other RNA
aattgctggag

SEQ ID NO: 251 - RNA, 11 nt, synthetic construct
  source 1..11: mol_type = other RNA
aattttagcag

SEQ ID NO: 252 - RNA, 11 nt, synthetic construct
  source 1..11: mol_type = other RNA
aaggtttggag

SEQ ID NO: 253 - RNA, 11 nt, synthetic construct
  source 1..11: mol_type = other RNA
gatttgtggag

SEQ ID NO: 254 - RNA, 11 nt, synthetic construct
  source 1..11: mol_type = other RNA
actttttggaa

SEQ ID NO: 255 - RNA, 11 nt, synthetic construct
  source 1..11: mol_type = other RNA
aaattatggag

SEQ ID NO: 256 - RNA, 11 nt, synthetic construct
  source 1..11: mol_type = other RNA
aattttgcgag

SEQ ID NO: 257 - RNA, 11 nt, synthetic construct
  source 1..11: mol_type = other RNA
aatttctggaa

SEQ ID NO: 258 - RNA, 11 nt, synthetic construct
  source 1..11: mol_type = other RNA
tatttttggtg

SEQ ID NO: 259 - RNA, 11 nt, synthetic construct
  source 1..11: mol_type = other RNA
gattttaggag

SEQ ID NO: 260 - RNA, 11 nt, synthetic construct
  source 1..11: mol_type = other RNA
aatctgtggag

SEQ ID NO: 261 - RNA, 11 nt, synthetic construct
  source 1..11: mol_type = other RNA
aaattttagag

SEQ ID NO: 262 - RNA, 11 nt, synthetic construct
  source 1..11: mol_type = other RNA
aatttttcgac

SEQ ID NO: 263 - RNA, 11 nt, synthetic construct
  source 1..11: mol_type = other RNA
aatttttgggt

SEQ ID NO: 264 - RNA, 11 nt, synthetic construct
  source 1..11: mol_type = other RNA
aatctttggaa

SEQ ID NO: 265 - RNA, 11 nt, synthetic construct
  source 1..11: mol_type = other RNA
aattattggtg

SEQ ID NO: 266 - RNA, 11 nt, synthetic construct
  source 1..11: mol_type = other RNA
aattttaagag

SEQ ID NO: 267 - RNA, 11 nt, synthetic construct
  source 1..11: mol_type = other RNA
aatgttaggag

SEQ ID NO: 268 - RNA, 11 nt, synthetic construct
  source 1..11: mol_type = other RNA
aattttagaag

SEQ ID NO: 269 - RNA, 11 nt, synthetic construct
  source 1..11: mol_type = other RNA
attttttggcg

SEQ ID NO: 270 - RNA, 11 nt, synthetic construct
  source 1..11: mol_type = other RNA
aacttttggtg

SEQ ID NO: 271 - RNA, 11 nt, synthetic construct
  source 1..11: mol_type = other RNA
aatgtttggat

SEQ ID NO: 272 - RNA, 11 nt, synthetic construct
  source 1..11: mol_type = other RNA
aattttaggat

SEQ ID NO: 273 - RNA, 11 nt, synthetic construct
  source 1..11: mol_type = other RNA
aatttttgatg

SEQ ID NO: 274 - RNA, 11 nt, synthetic construct
  source 1..11: mol_type = other RNA
aattcttggtg

SEQ ID NO: 275 - RNA, 11 nt, synthetic construct
  source 1..11: mol_type = other RNA
aatccttggag

SEQ ID NO: 276 - RNA, 11 nt, synthetic construct
  source 1..11: mol_type = other RNA
aatttttcaag

SEQ ID NO: 277 - RNA, 11 nt, synthetic construct
  source 1..11: mol_type = other RNA
catttttgcag

SEQ ID NO: 278 - RNA, 11 nt, synthetic construct
  source 1..11: mol_type = other RNA
catttttcgag

SEQ ID NO: 279 - RNA, 11 nt, synthetic construct
  source 1..11: mol_type = other RNA
aattttgggcg

SEQ ID NO: 280 - RNA, 11 nt, synthetic construct
  source 1..11: mol_type = other RNA
agtttttgcag

SEQ ID NO: 281 - RNA, 11 nt, synthetic construct
  source 1..11: mol_type = other RNA
aaaatttggag

SEQ ID NO: 282 - RNA, 11 nt, synthetic construct
  source 1..11: mol_type = other RNA
aactcttggag

SEQ ID NO: 283 - RNA, 11 nt, synthetic construct
  source 1..11: mol_type = other RNA
aatttgtggac

SEQ ID NO: 284 - RNA, 11 nt, synthetic construct
  source 1..11: mol_type = other RNA
actttttggtg

SEQ ID NO: 285 - RNA, 11 nt, synthetic construct
  source 1..11: mol_type = other RNA
attttttgaag

SEQ ID NO: 286 - RNA, 11 nt, synthetic construct
  source 1..11: mol_type = other RNA
attttttggaa

SEQ ID NO: 287 - RNA, 11 nt, synthetic construct
  source 1..11: mol_type = other RNA
aagttatggag

SEQ ID NO: 288 - RNA, 11 nt, synthetic construct
  source 1..11: mol_type = other RNA
catttctggag

SEQ ID NO: 289 - RNA, 11 nt, synthetic construct
  source 1..11: mol_type = other RNA
agttcttggag

SEQ ID NO: 290 - RNA, 11 nt, synthetic construct
  source 1..11: mol_type = other RNA
aattttggaag

SEQ ID NO: 291 - RNA, 11 nt, synthetic construct
  source 1..11: mol_type = other RNA
agtatttggag

SEQ ID NO: 292 - RNA, 11 nt, synthetic construct
  source 1..11: mol_type = other RNA
aatttttgggc

SEQ ID NO: 293 - RNA, 11 nt, synthetic construct
  source 1..11: mol_type = other RNA
aattcttgtag

SEQ ID NO: 294 - RNA, 11 nt, synthetic construct
  source 1..11: mol_type = other RNA
aattttgggaa

SEQ ID NO: 295 - RNA, 11 nt, synthetic construct
  source 1..11: mol_type = other RNA
tattttttgag

SEQ ID NO: 296 - RNA, 11 nt, synthetic construct
  source 1..11: mol_type = other RNA
aacttttgtag

SEQ ID NO: 297 - RNA, 11 nt, synthetic construct
  source 1..11: mol_type = other RNA
aattattgcag

SEQ ID NO: 298 - RNA, 11 nt, synthetic construct
  source 1..11: mol_type = other RNA
aatttttggta

SEQ ID NO: 299 - RNA, 11 nt, synthetic construct
  source 1..11: mol_type = other RNA
aattttcgaag

SEQ ID NO: 300 - RNA, 11 nt, synthetic construct
  source 1..11: mol_type = other RNA
tatctttggag

SEQ ID NO: 301 - RNA, 11 nt, synthetic construct
  source 1..11: mol_type = other RNA
aatttttcgtg

SEQ ID NO: 302 - RNA, 11 nt, synthetic construct
  source 1..11: mol_type = other RNA
aatctttggcg

SEQ ID NO: 303 - RNA, 11 nt, synthetic construct
  source 1..11: mol_type = other RNA
gatttatggag

SEQ ID NO: 304 - RNA, 11 nt, synthetic construct
  source 1..11: mol_type = other RNA
aatttttcgaa

SEQ ID NO: 305 - RNA, 11 nt, synthetic construct
  source 1..11: mol_type = other RNA
cctttttggag

SEQ ID NO: 306 - RNA, 11 nt, synthetic construct
  source 1..11: mol_type = other RNA
tatttttgaag

SEQ ID NO: 307 - RNA, 11 nt, synthetic construct
  source 1..11: mol_type = other RNA
aatttttgtaa

SEQ ID NO: 308 - RNA, 11 nt, synthetic construct
  source 1..11: mol_type = other RNA
aattttgagag

SEQ ID NO: 309 - RNA, 11 nt, synthetic construct
  source 1..11: mol_type = other RNA
aagtattggag

SEQ ID NO: 310 - RNA, 11 nt, synthetic construct
  source 1..11: mol_type = other RNA
aattttaggac

SEQ ID NO: 311 - RNA, 11 nt, synthetic construct
  source 1..11: mol_type = other RNA
aatttttaaag

SEQ ID NO: 312 - RNA, 11 nt, synthetic construct
  source 1..11: mol_type = other RNA
aattcttggat

SEQ ID NO: 313 - RNA, 11 nt, synthetic construct
  source 1..11: mol_type = other RNA
aatttgtggtg

SEQ ID NO: 314 - RNA, 11 nt, synthetic construct
  source 1..11: mol_type = other RNA
aattttcgggg

SEQ ID NO: 315 - RNA, 11 nt, synthetic construct
  source 1..11: mol_type = other RNA
acttttttgag

SEQ ID NO: 316 - RNA, 11 nt, synthetic construct
  source 1..11: mol_type = other RNA
aagtttcggag

SEQ ID NO: 317 - RNA, 11 nt, synthetic construct
  source 1..11: mol_type = other RNA
acttttgggag

SEQ ID NO: 318 - RNA, 11 nt, synthetic construct
  source 1..11: mol_type = other RNA
gacttttggag

SEQ ID NO: 319 - RNA, 11 nt, synthetic construct
  source 1..11: mol_type = other RNA
cttttttggag

SEQ ID NO: 320 - RNA, 11 nt, synthetic construct
  source 1..11: mol_type = other RNA
cattgttggag

SEQ ID NO: 321 - RNA, 11 nt, synthetic construct
  source 1..11: mol_type = other RNA
aatattttgag

SEQ ID NO: 322 - RNA, 11 nt, synthetic construct
  source 1..11: mol_type = other RNA
aagttttggcg

SEQ ID NO: 323 - RNA, 11 nt, synthetic construct
  source 1..11: mol_type = other RNA
aattttaggaa

SEQ ID NO: 324 - RNA, 11 nt, synthetic construct
  source 1..11: mol_type = other RNA
aacttgtggag

SEQ ID NO: 325 - RNA, 11 nt, synthetic construct
  source 1..11: mol_type = other RNA
attttttgggg

SEQ ID NO: 326 - RNA, 11 nt, synthetic construct
  source 1..11: mol_type = other RNA
aatttttagat

SEQ ID NO: 327 - RNA, 11 nt, synthetic construct
  source 1..11: mol_type = other RNA
tattttgggag

SEQ ID NO: 328 - RNA, 11 nt, synthetic construct
  source 1..11: mol_type = other RNA
aatttgtgggg

SEQ ID NO: 329 - RNA, 11 nt, synthetic construct
  source 1..11: mol_type = other RNA
aatttttaggg

SEQ ID NO: 330 - RNA, 11 nt, synthetic construct
  source 1..11: mol_type = other RNA
aattttcgtag

SEQ ID NO: 331 - RNA, 11 nt, synthetic construct
  source 1..11: mol_type = other RNA
aattttctgag

SEQ ID NO: 332 - RNA, 11 nt, synthetic construct
  source 1..11: mol_type = other RNA
gattcttggag

SEQ ID NO: 333 - RNA, 11 nt, synthetic construct
  source 1..11: mol_type = other RNA
aatctttggat

SEQ ID NO: 334 - RNA, 11 nt, synthetic construct
  source 1..11: mol_type = other RNA
aaattttggcg

SEQ ID NO: 335 - RNA, 11 nt, synthetic construct
  source 1..11: mol_type = other RNA
aatcattggag

SEQ ID NO: 336 - RNA, 11 nt, synthetic construct
  source 1..11: mol_type = other RNA
aacttttggcg

SEQ ID NO: 337 - RNA, 11 nt, synthetic construct
  source 1..11: mol_type = other RNA
aatttttggga

SEQ ID NO: 338 - RNA, 11 nt, synthetic construct
  source 1..11: mol_type = other RNA
aatgtttggtg

SEQ ID NO: 339 - RNA, 11 nt, synthetic construct
  source 1..11: mol_type = other RNA
aatttatgcag

SEQ ID NO: 340 - RNA, 11 nt, synthetic construct
  source 1..11: mol_type = other RNA
aatctctggag

SEQ ID NO: 341 - RNA, 11 nt, synthetic construct
  source 1..11: mol_type = other RNA
agtttgtggag

SEQ ID NO: 342 - RNA, 11 nt, synthetic construct
  source 1..11: mol_type = other RNA
aagttttggat

SEQ ID NO: 343 - RNA, 11 nt, synthetic construct
  source 1..11: mol_type = other RNA
tatttttggcg

SEQ ID NO: 344 - RNA, 11 nt, synthetic construct
  source 1..11: mol_type = other RNA
aatttctgaag

SEQ ID NO: 345 - RNA, 11 nt, synthetic construct
  source 1..11: mol_type = other RNA
tatttctggag

SEQ ID NO: 346 - RNA, 11 nt, synthetic construct
  source 1..11: mol_type = other RNA
aatttttgctg

SEQ ID NO: 347 - RNA, 11 nt, synthetic construct
  source 1..11: mol_type = other RNA
aaatcttggag

SEQ ID NO: 348 - RNA, 11 nt, synthetic construct
  source 1..11: mol_type = other RNA
aaatttttgag

SEQ ID NO: 349 - RNA, 11 nt, synthetic construct
  source 1..11: mol_type = other RNA
tatttttgtag

SEQ ID NO: 350 - RNA, 11 nt, synthetic construct
  source 1..11: mol_type = other RNA
gattattggag

SEQ ID NO: 351 - RNA, 11 nt, synthetic construct
  source 1..11: mol_type = other RNA
aagttctggag

SEQ ID NO: 352 - RNA, 11 nt, synthetic construct
  source 1..11: mol_type = other RNA
aatctttgaag

SEQ ID NO: 353 - RNA, 11 nt, synthetic construct
  source 1..11: mol_type = other RNA
aaatattggag

SEQ ID NO: 354 - RNA, 11 nt, synthetic construct
  source 1..11: mol_type = other RNA
aactattggag

SEQ ID NO: 355 - RNA, 11 nt, synthetic construct
  source 1..11: mol_type = other RNA
aatatatggag

SEQ ID NO: 356 - RNA, 11 nt, synthetic construct
  source 1..11: mol_type = other RNA
attctttggag

SEQ ID NO: 357 - RNA, 11 nt, synthetic construct
  source 1..11: mol_type = other RNA
gatttttgaag

SEQ ID NO: 358 - RNA, 11 nt, synthetic construct
  source 1..11: mol_type = other RNA
aagttttggtg

SEQ ID NO: 359 - RNA, 11 nt, synthetic construct
  source 1..11: mol_type = other RNA
aatctttgcag

SEQ ID NO: 360 - RNA, 11 nt, synthetic construct
  source 1..11: mol_type = other RNA
actttttggcg

SEQ ID NO: 361 - RNA, 11 nt, synthetic construct
  source 1..11: mol_type = other RNA
gattttgggag

SEQ ID NO: 362 - RNA, 11 nt, synthetic construct
  source 1..11: mol_type = other RNA
catttttggaa

SEQ ID NO: 363 - RNA, 11 nt, synthetic construct
  source 1..11: mol_type = other RNA
aattttttgac

SEQ ID NO: 364 - RNA, 11 nt, synthetic construct
  source 1..11: mol_type = other RNA
aaattttgcag

SEQ ID NO: 365 - RNA, 11 nt, synthetic construct
  source 1..11: mol_type = other RNA
actttttgaag

SEQ ID NO: 366 - RNA, 11 nt, synthetic construct
  source 1..11: mol_type = other RNA
agtctttggag

SEQ ID NO: 367 - RNA, 11 nt, synthetic construct
  source 1..11: mol_type = other RNA
agtttatggag

SEQ ID NO: 368 - RNA, 11 nt, synthetic construct
  source 1..11: mol_type = other RNA
acttcttggag

SEQ ID NO: 369 - RNA, 11 nt, synthetic construct
  source 1..11: mol_type = other RNA
aattttttcag

SEQ ID NO: 370 - RNA, 11 nt, synthetic construct
  source 1..11: mol_type = other RNA
aattttcggat

SEQ ID NO: 371 - RNA, 11 nt, synthetic construct
  source 1..11: mol_type = other RNA
aatttttgtat

SEQ ID NO: 372 - RNA, 11 nt, synthetic construct
  source 1..11: mol_type = other RNA
aatgtctggag

SEQ ID NO: 373 - RNA, 11 nt, synthetic construct
  source 1..11: mol_type = other RNA
aattgttgggg

SEQ ID NO: 374 - RNA, 11 nt, synthetic construct
  source 1..11: mol_type = other RNA
aatttatggac

SEQ ID NO: 375 - RNA, 11 nt, synthetic construct
  source 1..11: mol_type = other RNA
aatttatgaag

SEQ ID NO: 376 - RNA, 11 nt, synthetic construct
  source 1..11: mol_type = other RNA
aatttatgggg

SEQ ID NO: 377 - RNA, 11 nt, synthetic construct
  source 1..11: mol_type = other RNA
aatttgtcgag

SEQ ID NO: 378 - RNA, 11 nt, synthetic construct
  source 1..11: mol_type = other RNA
aattgttggaa

SEQ ID NO: 379 - RNA, 11 nt, synthetic construct
  source 1..11: mol_type = other RNA
tgtttttggag

SEQ ID NO: 380 - RNA, 11 nt, synthetic construct
  source 1..11: mol_type = other RNA
aaatttaggag

SEQ ID NO: 381 - RNA, 11 nt, synthetic construct
  source 1..11: mol_type = other RNA
aattttttgtg

SEQ ID NO: 382 - RNA, 11 nt, synthetic construct
  source 1..11: mol_type = other RNA
cattcttggag

SEQ ID NO: 383 - RNA, 11 nt, synthetic construct
  source 1..11: mol_type = other RNA
aattctcggag

SEQ ID NO: 384 - RNA, 11 nt, synthetic construct
  source 1..11: mol_type = other RNA
aatttttatag

SEQ ID NO: 385 - RNA, 11 nt, synthetic construct
  source 1..11: mol_type = other RNA
actttttagag

SEQ ID NO: 386 - RNA, 11 nt, synthetic construct
  source 1..11: mol_type = other RNA
aattgttagag

SEQ ID NO: 387 - RNA, 11 nt, synthetic construct
  source 1..11: mol_type = other RNA
cgtttttggag

SEQ ID NO: 388 - RNA, 11 nt, synthetic construct
  source 1..11: mol_type = other RNA
aatttttgcgg

SEQ ID NO: 389 - RNA, 11 nt, synthetic construct
  source 1..11: mol_type = other RNA
aagttttgaag

SEQ ID NO: 390 - RNA, 11 nt, synthetic construct
  source 1..11: mol_type = other RNA
aattttcggaa

SEQ ID NO: 391 - RNA, 11 nt, synthetic construct
  source 1..11: mol_type = other RNA
aatttttccag

SEQ ID NO: 392 - RNA, 11 nt, synthetic construct
  source 1..11: mol_type = other RNA
attttttgtag

SEQ ID NO: 393 - RNA, 11 nt, synthetic construct
  source 1..11: mol_type = other RNA
aatctttagag

SEQ ID NO: 394 - RNA, 11 nt, synthetic construct
  source 1..11: mol_type = other RNA
aattttacgag

SEQ ID NO: 395 - RNA, 11 nt, synthetic construct
  source 1..11: mol_type = other RNA
catttttggcg

SEQ ID NO: 396 - RNA, 11 nt, synthetic construct
  source 1..11: mol_type = other RNA
aatttgcggag

SEQ ID NO: 397 - RNA, 11 nt, synthetic construct
  source 1..11: mol_type = other RNA
catctttggag

SEQ ID NO: 398 - RNA, 11 nt, synthetic construct
  source 1..11: mol_type = other RNA
aacttttgaag

SEQ ID NO: 399 - RNA, 11 nt, synthetic construct
  source 1..11: mol_type = other RNA
aatttttctag

SEQ ID NO: 400 - RNA, 33 nt, synthetic construct
  source 1..33: mol_type = other RNA
caggttttccagtgttcactgaaatttgtctct

SEQ ID NO: 401 - RNA, 31 nt, synthetic construct
  source 1..31: mol_type = other RNA
aaggttttctggtctttatcagaaagcctcc

SEQ ID NO: 402 - RNA, 31 nt, synthetic construct
  source 1..31: mol_type = other RNA
caggttttctggttttcactgcaaaacccca

SEQ ID NO: 403 - RNA, 31 nt, synthetic construct
  source 1..31: mol_type = other RNA
cagactttacggtgcttaccagaaagctccc

SEQ ID NO: 404 - RNA, 45 nt, synthetic construct
  source 1..45: mol_type = other RNA
cacgttttccggttgtcactcccaggtaggctggggaagaggcat

SEQ ID NO: 405 - RNA, 31 nt, synthetic construct
  source 1..31: mol_type = other RNA
cagtctttccagtttttgccggaaagcccct

SEQ ID NO: 406 - RNA, 31 nt, synthetic construct
  source 1..31: mol_type = other RNA
caggctttctggtttttgccagaaagccccc

SEQ ID NO: 407 - RNA, 31 nt, synthetic construct
  source 1..31: mol_type = other RNA
caggtttttcagttttcaccagaatgcccac

SEQ ID NO: 408 - RNA, 31 nt, synthetic construct
  source 1..31: mol_type = other RNA
caggttttctggtctttactggaaagcccaa

SEQ ID NO: 409 - RNA, 31 nt, synthetic construct
  source 1..31: mol_type = other RNA
caggctttccggtttttaccagaaagccccc

SEQ ID NO: 410 - RNA, 31 nt, synthetic construct
  source 1..31: mol_type = other RNA
caggtttttcggtttttactggaaagcccca

SEQ ID NO: 411 - RNA, 31 nt, synthetic construct
  source 1..31: mol_type = other RNA
caggctttccagtaaatacaggaaagccctc

SEQ ID NO: 412 - RNA, 31 nt, synthetic construct
  source 1..31: mol_type = other RNA
caggctttccggtttttgctggaaagactcc

SEQ ID NO: 413 - RNA, 31 nt, synthetic construct
  source 1..31: mol_type = other RNA
aaggttttctggtctttactgaaaagcccca

SEQ ID NO: 414 - RNA, 31 nt, synthetic construct
  source 1..31: mol_type = other RNA
tgggctttctgttttttacgagaaagtcctc

SEQ ID NO: 415 - RNA, 30 nt, synthetic construct
  source 1..30: mol_type = other RNA
cagatttctcagttttcactggaaaccctt

SEQ ID NO: 416 - RNA, 31 nt, synthetic construct
  source 1..31: mol_type = other RNA
caggttttcctgtcttcaccggaactcccca

SEQ ID NO: 417 - RNA, 32 nt, synthetic construct
  source 1..32: mol_type = other RNA
caggttttctggtttgcactagaaaaaccata

SEQ ID NO: 418 - RNA, 31 nt, synthetic construct
  source 1..31: mol_type = other RNA
cagattttctggtttttaccagaaaatttaa

SEQ ID NO: 419 - RNA, 31 nt, synthetic construct
  source 1..31: mol_type = other RNA
caggtcttcttgtttttactggaaaatcctc

SEQ ID NO: 420 - RNA, 31 nt, synthetic construct
  source 1..31: mol_type = other RNA
caggttttccggtcttcaccagaaaacccct

SEQ ID NO: 421 - RNA, 31 nt, synthetic construct
  source 1..31: mol_type = other RNA
caggttttccgatttttactggaaagccctt

SEQ ID NO: 422 - RNA, 29 nt, synthetic construct
  source 1..29: mol_type = other RNA
caggttttctggttttccagaaaacctcc

SEQ ID NO: 423 - RNA, 29 nt, synthetic construct
  source 1..29: mol_type = other RNA
caggttttccagttttcactggaacctct

SEQ ID NO: 424 - RNA, 30 nt, synthetic construct
  source 1..30: mol_type = other RNA
ccggctttccagttttgccggaaagccccc

SEQ ID NO: 425 - RNA, 29 nt, synthetic construct
  source 1..29: mol_type = other RNA
caggttttctggtttttaccagaacttaa

SEQ ID NO: 426 - RNA, 45 nt, synthetic construct
  source 1..45: mol_type = other RNA
cacgttttccggttgtcactcccaggtgggctcgggaagaggcat

SEQ ID NO: 427 - RNA, 31 nt, synthetic construct
  source 1..31: mol_type = other RNA
caggctttccggtctttaccggaaagcctat

SEQ ID NO: 428 - RNA, 31 nt, synthetic construct
  source 1..31: mol_type = other RNA
caggctttctggtttttgccggaaagccctc

SEQ ID NO: 429 - RNA, 31 nt, synthetic construct
  source 1..31: mol_type = other RNA
caggctttcaggtctttgccagaaagcccca

SEQ ID NO: 430 - RNA, 30 nt, synthetic construct
  source 1..30: mol_type = other RNA
caggctttcttgttttcactggaagcctcc

SEQ ID NO: 431 - RNA, 31 nt, synthetic construct
  source 1..31: mol_type = other RNA
caggtttttcagtcttcaccggaaagctccc

SEQ ID NO: 432 - RNA, 37 nt, synthetic construct
  source 1..37: mol_type = other RNA
cagattttctggttttcaccacgaaagaaatgtcatt

SEQ ID NO: 433 - RNA, 30 nt, synthetic construct
  source 1..30: mol_type = other RNA
cagttctcctggattttacaggaaaacccc

SEQ ID NO: 434 - RNA, 31 nt, synthetic construct
  source 1..31: mol_type = other RNA
caggctttctggcatttgccagaaagccctg

SEQ ID NO: 435 - RNA, 31 nt, synthetic construct
  source 1..31: mol_type = other RNA
cagactttcctgtttttaacagaaagccccc

SEQ ID NO: 436 - RNA, 31 nt, synthetic construct
  source 1..31: mol_type = other RNA
caggctttccggtttttgctggaaagctccc

SEQ ID NO: 437 - RNA, 28 nt, synthetic construct
  source 1..28: mol_type = other RNA
cagcttttccagttttcactgaggtaaa

SEQ ID NO: 438 - RNA, 31 nt, synthetic construct
  source 1..31: mol_type = other RNA
taggttttctggtttttattgggaaatcaca

SEQ ID NO: 439 - RNA, 32 nt, synthetic construct
  source 1..32: mol_type = other RNA
cagggtttctggtggtaccagcagactccaca

SEQ ID NO: 440 - RNA, 30 nt, synthetic construct
  source 1..30: mol_type = other RNA
caagtttcccagttttcactggaacctccg

SEQ ID NO: 441 - RNA, 31 nt, synthetic construct
  source 1..31: mol_type = other RNA
cagattttccagtttttactagaaacccccc

SEQ ID NO: 442 - RNA, 31 nt, synthetic construct
  source 1..31: mol_type = other RNA
cagggtttccagttttcactgtataccatcc

SEQ ID NO: 443 - RNA, 31 nt, synthetic construct
  source 1..31: mol_type = other RNA
caagttctctggtcttcactggaaaacccat

SEQ ID NO: 444 - RNA, 31 nt, synthetic construct
  source 1..31: mol_type = other RNA
tagattttctggtctttaccagaaagactcc

SEQ ID NO: 445 - RNA, 32 nt, synthetic construct
  source 1..32: mol_type = other RNA
gcaggttttctggtttttattggaaaaccttc

SEQ ID NO: 446 - RNA, 33 nt, synthetic construct
  source 1..33: mol_type = other RNA
caggttttccacttttcaccagaaactgcctct

SEQ ID NO: 447 - RNA, 31 nt, synthetic construct
  source 1..31: mol_type = other RNA
taggctttccggtttttgcaggaaagccccc

SEQ ID NO: 448 - RNA, 31 nt, synthetic construct
  source 1..31: mol_type = other RNA
caggttttccagtctttaccagaaagccact

SEQ ID NO: 449 - RNA, 33 nt, synthetic construct
  source 1..33: mol_type = other RNA
caggttttttggtcttcacaggaaacttcccct

SEQ ID NO: 450 - RNA, 31 nt, synthetic construct
  source 1..31: mol_type = other RNA
caggttttctgcttttcactggaagactccc

SEQ ID NO: 451 - RNA, 31 nt, synthetic construct
  source 1..31: mol_type = other RNA
cagactttccagtttttgccagaaagcccac

SEQ ID NO: 452 - RNA, 31 nt, synthetic construct
  source 1..31: mol_type = other RNA
caggctttctggtttttgcccaaaagcctct

SEQ ID NO: 453 - RNA, 31 nt, synthetic construct
  source 1..31: mol_type = other RNA
caggttttccaatcttcactgaaaagcttta

SEQ ID NO: 454 - RNA, 31 nt, synthetic construct
  source 1..31: mol_type = other RNA
ctggttcctagtcttcactggaagcaccctc

SEQ ID NO: 455 - RNA, 29 nt, synthetic construct
  source 1..29: mol_type = other RNA
ggctttccgctcttcattggaaagcccat

SEQ ID NO: 456 - RNA, 31 nt, synthetic construct
  source 1..31: mol_type = other RNA
caggtttttcagtttttagcagaaaacctcc

SEQ ID NO: 457 - RNA, 29 nt, synthetic construct
  source 1..29: mol_type = other RNA
caggttctctggtttttactgaaaccaaa

SEQ ID NO: 458 - RNA, 31 nt, synthetic construct
  source 1..31: mol_type = other RNA
caggttttccagtcttcactggaaagccctt

SEQ ID NO: 459 - RNA, 31 nt, synthetic construct
  source 1..31: mol_type = other RNA
caggctttctggtttttgccggaaggcctcc

SEQ ID NO: 460 - RNA, 32 nt, synthetic construct
  source 1..32: mol_type = other RNA
caggttttctgttttttaactagaaaactccc

SEQ ID NO: 461 - RNA, 31 nt, synthetic construct
  source 1..31: mol_type = other RNA
taggctttctggtttttgctggaaagccccc

SEQ ID NO: 462 - RNA, 32 nt, synthetic construct
  source 1..32: mol_type = other RNA
caggtttcccggtttttaccagaaaaatctaa

SEQ ID NO: 463 - RNA, 32 nt, synthetic construct
  source 1..32: mol_type = other RNA
caggttttccagtctttaccagaaatgccccc

SEQ ID NO: 464 - RNA, 30 nt, synthetic construct
  source 1..30: mol_type = other RNA
ggttttctggtcatcgctggaaacaccctc

SEQ ID NO: 465 - RNA, 31 nt, synthetic construct
  source 1..31: mol_type = other RNA
caacctttctggtttttgctagaaagttctc

SEQ ID NO: 466 - RNA, 31 nt, synthetic construct
  source 1..31: mol_type = other RNA
caggcctttgggtctttactggaaaacccct

SEQ ID NO: 467 - RNA, 35 nt, synthetic construct
  source 1..35: mol_type = other RNA
tatgttttccggttttcactcccaggtaggctcgg

SEQ ID NO: 468 - RNA, 31 nt, synthetic construct
  source 1..31: mol_type = other RNA
caaattttctggcttttccaggaaaatcccc

SEQ ID NO: 469 - RNA, 28 nt, synthetic construct
  source 1..28: mol_type = other RNA
taggttttccagtgaccagaaaatcctc

SEQ ID NO: 470 - RNA, 31 nt, synthetic construct
  source 1..31: mol_type = other RNA
caggtcttcgggtttttaactggaaaccttc

SEQ ID NO: 471 - RNA, 32 nt, synthetic construct
  source 1..32: mol_type = other RNA
gcaggctttccagttttttctggaaagcctca

SEQ ID NO: 472 - RNA, 29 nt, synthetic construct
  source 1..29: mol_type = other RNA
ggctttctggtttttactggaaagccccc

SEQ ID NO: 473 - RNA, 31 nt, synthetic construct
  source 1..31: mol_type = other RNA
caggtttcccagtattcaccggaaagctcca

SEQ ID NO: 474 - RNA, 43 nt, synthetic construct
  source 1..43: mol_type = other RNA
caggctttctagttttcccacactaagaaacaaaaaaacctgt

SEQ ID NO: 475 - RNA, 32 nt, synthetic construct
  source 1..32: mol_type = other RNA
gcaagctttctggttttggccaaaaagccacc

SEQ ID NO: 476 - RNA, 24 nt, synthetic construct
  source 1..24: mol_type = other RNA
ttctggttttcaccagaaaccact

SEQ ID NO: 477 - RNA, 32 nt, synthetic construct
  source 1..32: mol_type = other RNA
gcaagttttctagcctgtacctgaaagcctca

SEQ ID NO: 478 - RNA, 30 nt, synthetic construct
  source 1..30: mol_type = other RNA
aagttttcttgtttttaccagaaaactcct

SEQ ID NO: 479 - RNA, 29 nt, synthetic construct
  source 1..29: mol_type = other RNA
ggctttccaatttttgccagaaagccccc

SEQ ID NO: 480 - RNA, 33 nt, synthetic construct
  source 1..33: mol_type = other RNA
agctggttttctggttttcaccggaagacccat

SEQ ID NO: 481 - RNA, 32 nt, synthetic construct
  source 1..32: mol_type = other RNA
caggccttctggtttttgctgggaagtcccca

SEQ ID NO: 482 - RNA, 31 nt, synthetic construct
  source 1..31: mol_type = other RNA
caggttttccagtttgtaccagaaaacccct

SEQ ID NO: 483 - RNA, 22 nt, synthetic construct
  source 1..22: mol_type = other RNA
caggtttcacccaaaaacccac

SEQ ID NO: 484 - RNA, 32 nt, synthetic construct
  source 1..32: mol_type = other RNA
taggctttctggctttttaccggaaagcccct

SEQ ID NO: 485 - RNA, 31 nt, synthetic construct
  source 1..31: mol_type = other RNA
cagggtttctggttttcaccaggaaacaaaa

SEQ ID NO: 486 - RNA, 31 nt, synthetic construct
  source 1..31: mol_type = other RNA
caggttttcctgtctttactagaaacccctc

SEQ ID NO: 487 - RNA, 31 nt, synthetic construct
  source 1..31: mol_type = other RNA
caggttttataggttttaccagaaaacttcc

SEQ ID NO: 488 - RNA, 31 nt, synthetic construct
  source 1..31: mol_type = other RNA
caggcttcccaatttttgctggaaagcccct

SEQ ID NO: 489 - RNA, 31 nt, synthetic construct
  source 1..31: mol_type = other RNA
caggctttttgggttaggtcagaaagtcccc

SEQ ID NO: 490 - RNA, 29 nt, synthetic construct
  source 1..29: mol_type = other RNA
caggtttttggttttttagcgaaaccctc

SEQ ID NO: 491 - RNA, 33 nt, synthetic construct
  source 1..33: mol_type = other RNA
caggctttctggttttcaccataaaatgcccca

SEQ ID NO: 492 - RNA, 43 nt, synthetic construct
  source 1..43: mol_type = other RNA
caggtcttcggggttttacaaaaagaagaagaaatccaccccc

SEQ ID NO: 493 - RNA, 32 nt, synthetic construct
  source 1..32: mol_type = other RNA
caagttctctggtctcccaacaagaaaccccc

SEQ ID NO: 494 - RNA, 33 nt, synthetic construct
  source 1..33: mol_type = other RNA
caggatttctggtttctggtggaaagctcccat

SEQ ID NO: 495 - RNA, 30 nt, synthetic construct
  source 1..30: mol_type = other RNA
aggctttccagtttttgctggaaagcccct

SEQ ID NO: 496 - RNA, 30 nt, synthetic construct
  source 1..30: mol_type = other RNA
caggttttccagtctttgtcaaatgccttc

SEQ ID NO: 497 - RNA, 34 nt, synthetic construct
  source 1..34: mol_type = other RNA
caggctttccagtttttgccagtgtgaacccaaa

SEQ ID NO: 498 - RNA, 31 nt, synthetic construct
  source 1..31: mol_type = other RNA
caggctttctggtttttatcagaaagcctcc

SEQ ID NO: 499 - RNA, 33 nt, synthetic construct
  source 1..33: mol_type = other RNA
gaggttttctggttttcaccagaaccaaccctt

SEQ ID NO: 500 - RNA, 31 nt, synthetic construct
  source 1..31: mol_type = other RNA
cagattttccagcttttactggaagccccct

SEQ ID NO: 501 - RNA, 33 nt, synthetic construct
  source 1..33: mol_type = other RNA
caggttttctggttttcactggaaaatacctca

SEQ ID NO: 502 - RNA, 33 nt, synthetic construct
  source 1..33: mol_type = other RNA
catgttttttggtgttaataaaaacccgcacac

SEQ ID NO: 503 - RNA, 31 nt, synthetic construct
  source 1..31: mol_type = other RNA
aaggctttccggtttttgcaggaaagcaacc

SEQ ID NO: 504 - RNA, 30 nt, synthetic construct
  source 1..30: mol_type = other RNA
agtctttccgggttttgtcagaaaggccct

SEQ ID NO: 505 - RNA, 31 nt, synthetic construct
  source 1..31: mol_type = other RNA
gcaggttttctggtttctactggaagcttct

SEQ ID NO: 506 - RNA, 31 nt, synthetic construct
  source 1..31: mol_type = other RNA
caggctttctggtttttactggaaagcccct

SEQ ID NO: 507 - RNA, 31 nt, synthetic construct
  source 1..31: mol_type = other RNA
caggctttccaatttttgccagaaagctccc

SEQ ID NO: 508 - RNA, 31 nt, synthetic construct
  source 1..31: mol_type = other RNA
caggttttctggttctcaccagaaaacgcca

SEQ ID NO: 509 - RNA, 31 nt, synthetic construct
  source 1..31: mol_type = other RNA
caggttttctggtcttcactggaagaccact

SEQ ID NO: 510 - RNA, 34 nt, synthetic construct
  source 1..34: mol_type = other RNA
caggtggttttccggtctttaccagtaagccccc

SEQ ID NO: 511 - RNA, 31 nt, synthetic construct
  source 1..31: mol_type = other RNA
caggttttccagtttctaccagaaaccccta

SEQ ID NO: 512 - RNA, 32 nt, synthetic construct
  source 1..32: mol_type = other RNA
caggttttctggtctttaccagaaaagtctcc

SEQ ID NO: 513 - RNA, 32 nt, synthetic construct
  source 1..32: mol_type = other RNA
ccaggctttctggtttttaccggaaagccccg

SEQ ID NO: 514 - RNA, 32 nt, synthetic construct
  source 1..32: mol_type = other RNA
caggtttttcactttttactagaaaaaccagt

SEQ ID NO: 515 - RNA, 35 nt, synthetic construct
  source 1..35: mol_type = other RNA
taggctttctgggaggtctttaccagaaagcctcc

SEQ ID NO: 516 - RNA, 32 nt, synthetic construct
  source 1..32: mol_type = other RNA
tacattttccagttttcaccaggaatctgcct

SEQ ID NO: 517 - RNA, 31 nt, synthetic construct
  source 1..31: mol_type = other RNA
taggttttccagttttcaccagaaaatcccg

SEQ ID NO: 518 - RNA, 31 nt, synthetic construct
  source 1..31: mol_type = other RNA
caggttttctggtttttacgagactccccca

SEQ ID NO: 519 - RNA, 30 nt, synthetic construct
  source 1..30: mol_type = other RNA
caggctttctgttttttgctaaaatcctcc

SEQ ID NO: 520 - RNA, 32 nt, synthetic construct
  source 1..32: mol_type = other RNA
ctgggcgtactggctcacgcctataatcccaa

SEQ ID NO: 521 - RNA, 29 nt, synthetic construct
  source 1..29: mol_type = other RNA
ggctctccggtttttgccagaatgcccac

SEQ ID NO: 522 - RNA, 31 nt, synthetic construct
  source 1..31: mol_type = other RNA
tgggttttctggtgaaaactagactgccccc

SEQ ID NO: 523 - RNA, 31 nt, synthetic construct
  source 1..31: mol_type = other RNA
caggctttctggtttttacaggaaggcccct

SEQ ID NO: 524 - RNA, 29 nt, synthetic construct
  source 1..29: mol_type = other RNA
ggttttctggttctcactggaaaaccccc

SEQ ID NO: 525 - RNA, 31 nt, synthetic construct
  source 1..31: mol_type = other RNA
caggctttccggtaaagactggaaagcccct

SEQ ID NO: 526 - RNA, 31 nt, synthetic construct
  source 1..31: mol_type = other RNA
caggttttccagtttttaccagaaaactctc

SEQ ID NO: 527 - RNA, 31 nt, synthetic construct
  source 1..31: mol_type = other RNA
aaggttttctggtcttcactggaagaccact

SEQ ID NO: 528 - RNA, 31 nt, synthetic construct
  source 1..31: mol_type = other RNA
gaggttttctggtcttcactggaagaccact

SEQ ID NO: 529 - RNA, 31 nt, synthetic construct
  source 1..31: mol_type = other RNA
taggttttctggtcttcactggaagaccact

SEQ ID NO: 530 - RNA, 31 nt, synthetic construct
  source 1..31: mol_type = other RNA
ccggttttctggtcttcactggaagaccact

SEQ ID NO: 531 - RNA, 31 nt, synthetic construct
  source 1..31: mol_type = other RNA
cgggttttctggtcttcactggaagaccact

SEQ ID NO: 532 - RNA, 31 nt, synthetic construct
  source 1..31: mol_type = other RNA
ctggttttctggtcttcactggaagaccact

SEQ ID NO: 533 - RNA, 31 nt, synthetic construct
  source 1..31: mol_type = other RNA
catgttttctggtcttcactggaagacaact

SEQ ID NO: 534 - RNA, 31 nt, synthetic construct
  source 1..31: mol_type = other RNA
cacgttttctggtcttcactggaagacgact

SEQ ID NO: 535 - RNA, 31 nt, synthetic construct
  source 1..31: mol_type = other RNA
caagttttctggtcttcactggaagactact

SEQ ID NO: 536 - RNA, 31 nt, synthetic construct
  source 1..31: mol_type = other RNA
cagcttttctggtcttcactggaagagcact

SEQ ID NO: 537 - RNA, 31 nt, synthetic construct
  source 1..31: mol_type = other RNA
cagattttctggtcttcactggaagatcact

SEQ ID NO: 538 - RNA, 31 nt, synthetic construct
  source 1..31: mol_type = other RNA
cagtttttctggtcttcactggaagaacact

SEQ ID NO: 539 - RNA, 31 nt, synthetic construct
  source 1..31: mol_type = other RNA
cagggtttctggtcttcactggaagcccact

SEQ ID NO: 540 - RNA, 31 nt, synthetic construct
  source 1..31: mol_type = other RNA
caggatttctggtcttcactggaagtccact

SEQ ID NO: 541 - RNA, 31 nt, synthetic construct
  source 1..31: mol_type = other RNA
caggctttctggtcttcactggaaggccact

SEQ ID NO: 542 - RNA, 31 nt, synthetic construct
  source 1..31: mol_type = other RNA
caggtgttctggtcttcactggaacaccact

SEQ ID NO: 543 - RNA, 31 nt, synthetic construct
  source 1..31: mol_type = other RNA
caggtattctggtcttcactggaataccact

SEQ ID NO: 544 - RNA, 31 nt, synthetic construct
  source 1..31: mol_type = other RNA
caggtcttctggtcttcactggaagaccact

SEQ ID NO: 545 - RNA, 31 nt, synthetic construct
  source 1..31: mol_type = other RNA
caggttttctggtcttcactggaaaaccact

SEQ ID NO: 546 - RNA, 31 nt, synthetic construct
  source 1..31: mol_type = other RNA
caggttctctggtcttcactggaggaccact

SEQ ID NO: 547 - RNA, 31 nt, synthetic construct
  source 1..31: mol_type = other RNA
caggttgtctggtcttcactggacgaccact

SEQ ID NO: 548 - RNA, 31 nt, synthetic construct
  source 1..31: mol_type = other RNA
caggttatctggtcttcactggatgaccact

SEQ ID NO: 549 - RNA, 31 nt, synthetic construct
  source 1..31: mol_type = other RNA
caggtttactggtcttcactggtagaccact

SEQ ID NO: 550 - RNA, 31 nt, synthetic construct
  source 1..31: mol_type = other RNA
caggtttgctggtcttcactggcagaccact

SEQ ID NO: 551 - RNA, 31 nt, synthetic construct
  source 1..31: mol_type = other RNA
caggtttcctggtcttcactgggagaccact

SEQ ID NO: 552 - RNA, 31 nt, synthetic construct
  source 1..31: mol_type = other RNA
caggttttttggtcttcactgaaagaccact

SEQ ID NO: 553 - RNA, 31 nt, synthetic construct
  source 1..31: mol_type = other RNA
caggttttgtggtcttcactgcaagaccact

SEQ ID NO: 554 - RNA, 31 nt, synthetic construct
  source 1..31: mol_type = other RNA
caggttttatggtcttcactgtaagaccact

SEQ ID NO: 555 - RNA, 31 nt, synthetic construct
  source 1..31: mol_type = other RNA
caggttttccggtcttcactggaagaccact

SEQ ID NO: 556 - RNA, 31 nt, synthetic construct
  source 1..31: mol_type = other RNA
caggttttcaggtcttcacttgaagaccact

SEQ ID NO: 557 - RNA, 31 nt, synthetic construct
  source 1..31: mol_type = other RNA
caggttttctggtcttcactagaagaccact

SEQ ID NO: 558 - RNA, 31 nt, synthetic construct
  source 1..31: mol_type = other RNA
caggttttcgggtcttcactcgaagaccact

SEQ ID NO: 559 - RNA, 31 nt, synthetic construct
  source 1..31: mol_type = other RNA
caggttttcttgtcttcacaggaagaccact

SEQ ID NO: 560 - RNA, 31 nt, synthetic construct
  source 1..31: mol_type = other RNA
caggttttctagtcttcactggaagaccact

SEQ ID NO: 561 - RNA, 31 nt, synthetic construct
  source 1..31: mol_type = other RNA
caggttttctggtcttcaccggaagaccact

SEQ ID NO: 562 - RNA, 31 nt, synthetic construct
  source 1..31: mol_type = other RNA
caggttttctcgtcttcacgggaagaccact

SEQ ID NO: 563 - RNA, 31 nt, synthetic construct
  source 1..31: mol_type = other RNA
caggttttctgttcttcaatggaagaccact

SEQ ID NO: 564 - RNA, 31 nt, synthetic construct
  source 1..31: mol_type = other RNA
caggttttctgatcttcattggaagaccact

SEQ ID NO: 565 - RNA, 31 nt, synthetic construct
  source 1..31: mol_type = other RNA
caggttttctgctcttcagtggaagaccact

SEQ ID NO: 566 - RNA, 31 nt, synthetic construct
  source 1..31: mol_type = other RNA
caggttttctggccttcgctggaagaccact

SEQ ID NO: 567 - RNA, 31 nt, synthetic construct
  source 1..31: mol_type = other RNA
caggttttctgggcttccctggaagaccact

SEQ ID NO: 568 - RNA, 31 nt, synthetic construct
  source 1..31: mol_type = other RNA
caggttttctggacttctctggaagaccact

SEQ ID NO: 569 - RNA, 31 nt, synthetic construct
  source 1..31: mol_type = other RNA
caggttttctggttttcactggaagaccact

SEQ ID NO: 570 - RNA, 31 nt, synthetic construct
  source 1..31: mol_type = other RNA
caggttttctggtattcactggaagaccact

SEQ ID NO: 571 - RNA, 31 nt, synthetic construct
  source 1..31: mol_type = other RNA
caggttttctggtgttcactggaagaccact

SEQ ID NO: 572 - RNA, 31 nt, synthetic construct
  source 1..31: mol_type = other RNA
caggttttctggtcatcactggaagaccact

SEQ ID NO: 573 - RNA, 31 nt, synthetic construct
  source 1..31: mol_type = other RNA
caggttttctggtcgtcactggaagaccact

SEQ ID NO: 574 - RNA, 31 nt, synthetic construct
  source 1..31: mol_type = other RNA
caggttttctggtcctcactggaagaccact

SEQ ID NO: 575 - RNA, 31 nt, synthetic construct
  source 1..31: mol_type = other RNA
caggttttctggtctacactggaagaccact

SEQ ID NO: 576 - RNA, 31 nt, synthetic construct
  source 1..31: mol_type = other RNA
caggttttctggtctgcactggaagaccact

SEQ ID NO: 577 - RNA, 31 nt, synthetic construct
  source 1..31: mol_type = other RNA
caggttttctggtctccactggaagaccact

SEQ ID NO: 578 - RNA, 31 nt, synthetic construct
  source 1..31: mol_type = other RNA
caggttttctggtcttaactggaagaccact

SEQ ID NO: 579 - RNA, 31 nt, synthetic construct
  source 1..31: mol_type = other RNA
caggttttctggtcttgactggaagaccact

SEQ ID NO: 580 - RNA, 31 nt, synthetic construct
  source 1..31: mol_type = other RNA
caggttttctggtctttactggaagaccact

SEQ ID NO: 581 - RNA, 31 nt, synthetic construct
  source 1..31: mol_type = other RNA
caggttttctggtcttcactggaagaccgct

SEQ ID NO: 582 - RNA, 31 nt, synthetic construct
  source 1..31: mol_type = other RNA
caggttttctggtcttcactggaagacccct

SEQ ID NO: 583 - RNA, 31 nt, synthetic construct
  source 1..31: mol_type = other RNA
caggttttctggtcttcactggaagacctct

SEQ ID NO: 584 - RNA, 31 nt, synthetic construct
  source 1..31: mol_type = other RNA
caggttttctggtcttcactggaagaccagt

SEQ ID NO: 585 - RNA, 31 nt, synthetic construct
  source 1..31: mol_type = other RNA
caggttttctggtcttcactggaagaccatt

SEQ ID NO: 586 - RNA, 31 nt, synthetic construct
  source 1..31: mol_type = other RNA
caggttttctggtcttcactggaagaccaat

SEQ ID NO: 587 - RNA, 31 nt, synthetic construct
  source 1..31: mol_type = other RNA
caggttttctggtcttcactggaagaccaca

SEQ ID NO: 588 - RNA, 31 nt, synthetic construct
  source 1..31: mol_type = other RNA
caggttttctggtcttcactggaagaccacg

SEQ ID NO: 589 - RNA, 31 nt, synthetic construct
  source 1..31: mol_type = other RNA
caggttttctggtcttcactggaagaccacc

SEQ ID NO: 590 - RNA, 132 nt, synthetic construct
  source 1..132: mol_type = other RNA
tcctcctacaaaggcgtgtctgtggttccctgtctttggacacgtaagaattggaggaaaataaatgtggatttgggaaactttgaggccagcttgcttcttgcaggctcatgatcaaccaatctcacataa

SEQ ID NO: 591 - RNA, 131 nt, synthetic construct
  source 1..131: mol_type = other RNA
ctccatgtatctttgggacctgtcagccgtggcagtctcccttcctagccatggaagagcatatccttgtttattggcaaagctgtcaccatttaattggtatcagattctgacttgcacaagtaacattc

SEQ ID NO: 592 - RNA, 132 nt, synthetic construct
  source 1..132: mol_type = other RNA
ctgcgaatattctcgctgttctgattttgtaatagtcaggacaggctaaacattcgctatattaagaccatgcatgtgtccccaaacctagttctttccctaggtctggtttcataaatgctggtgataaac

SEQ ID NO: 593 - RNA, 133 nt, synthetic construct
  source 1..133: mol_type = other RNA
gcatggccgaatactgtgtttttatcagtagtttacacagccagacaccatgcaaaagcagtcttccctttagaatgactgatggtatgctaaggtttttcatagcatatcattattaaaggtgaatacaaat

SEQ ID NO: 594 - RNA, 139 nt, synthetic construct
  source 1..139: mol_type = other RNA
tgcactgcatggtatctgcactcagcagtttacacctgctagggtgttcaaaggtcagtgctatagaaattcagtatctggcatcgttggttttcttggctttgtgcttgttaaacctggtatttctactgatacagta

SEQ ID NO: 595 - RNA, 134 nt, synthetic construct
  source 1..134: mol_type = other RNA
tggtccatcctaatccctgccggtccatctgtggcctgccaggtttcgcttgtggaccagagcaccctagaagcctcacccgaggagtgagcagggctccagtgggctcacgtcatgggcacttctagacactc

SEQ ID NO: 596 - RNA, 136 nt, synthetic construct
  source 1..136: mol_type = other RNA
cacctgcattcaaaaatgatcacgggctgcctgtgctctggtcatcaataacgcagggagaggaattgctgaaagccgtttcccgtgtttggagggttcacacctgtccctttcaaatgctggcgctttcacacac

SEQ ID NO: 597 - RNA, 134 nt, synthetic construct
  source 1..134: mol_type = other RNA
tgcattcttaaaccctcttggtggcttccctgtaaatgcttccaagatatgagcgaatgctatagaaattgcaggaaagtccaaagggctgcgcgtctcctgtggctcagtcttatttcatacctgcaacatct

SEQ ID NO: 598 - RNA, 133 nt, synthetic construct
  source 1..133: mol_type = other RNA
attgcacctaaacccaagaatcactgtttcttatagcggtggtttaaacagaggtgcaaacagcaagcggatcttgtcgcctttggggggctgtggccgtgcccctcaaagtgaatttggaggttccacaact

SEQ ID NO: 599 - RNA, 132 nt, synthetic construct
  source 1..132: mol_type = other RNA
cttcccatttatttgctgcttgtagtctcacagtgatacgagcagttatacgcatgggataaaataacattgggccactgtaaattgagatgaagtaaccattttcatctcttctgcagggactagacattg

SEQ ID NO: 600 - RNA, 135 nt, synthetic construct
  source 1..135: mol_type = other RNA
ctgcattcttaaaccctcttggtagcttcgttctaagtgcttccaagatatgagtgaatgctatagaaattgcaggggagtccaaagggctgcgcttctcccgtggctcagtcttatttcatacctgcgacatct

SEQ ID NO: 601 - RNA, 131 nt, synthetic construct
  source 1..131: mol_type = other RNA
aggcaggatctagttacattgtagctgtgaagtgctgcattgtctttgccccctgctcaaaataaaactgttacctttcaagccctgtctgccatggtgctgtagcagcagggatgtttggtctcatacat

SEQ ID NO: 602 - RNA, 123 nt, synthetic construct
  source 1..123: mol_type = other RNA
actggaggactaaggaggctgggtctgatgaggcaagattttgctgatacattgctcctagaaaaaagggttggcaagagcagccctggagactcacacggctgactgttctacccaacactc

SEQ ID NO: 603 - RNA, 129 nt, synthetic construct
  source 1..129: mol_type = other RNA
aagcaggattcagactacaatatagctgttaagtgctgtattgtcattccccctgctcaaattaaagttgtttcttaactatacccatctgctattctgtagcagccagggatgcttggtcacatacat

SEQ ID NO: 604 - RNA, 132 nt, synthetic construct
  source 1..132: mol_type = other RNA
ggcttcctagtacttaccatggtctgtgttcttacgctgactgtatagaaacaggaggcagagtaaaccgaccccacatatacctcagcccaggccctgtgctgcgtctgtattgtgaatcaggagacatgg

SEQ ID NO: 605 - RNA, 139 nt, synthetic construct
  source 1..139: mol_type = other RNA
tggctcgatttcctggggggtggtctcagcccactccacctcccctcagccgagcctagagtagaggggccaggcatcctccccaggggaggggcgttgaagcaaggagcctctcctgggctgtcctagcctcacattt

SEQ ID NO: 606 - RNA, 132 nt, synthetic construct
  source 1..132: mol_type = other RNA
gttgaggtctatcccgataggtcttttcctgtagcctgcacgttgttggaaatgcctcatagagtaactctgtgattttactttacttacaggactattgttacatctgtgggaaggaaccacaagacagtt

SEQ ID NO: 607 - RNA, 133 nt, synthetic construct
  source 1..133: mol_type = other RNA
ttctcacctaaacccaagaatcactgtttcttatagcggtggtttaaacagaggtgcaaacagcaagtgaatctcgtcgcctttgcggggctgtggccatgcccctcaaaggaaatttggaggctctacagcc

SEQ ID NO: 608 - RNA, 128 nt, synthetic construct
  source 1..128: mol_type = other RNA
aggtcatttcaaagaggtcttgtgaggctgtgaaaccaagagctcttaacactgcgaccaaagatggaagttctctataggatgccatggcatttgatggtgctatgttttcttgaggagatataaga

SEQ ID NO: 609 - RNA, 131 nt, synthetic construct
  source 1..131: mol_type = other RNA
ccctcctacaaaggcatgtctatagttccttgtctttggacatgtaagaattggaggcaaagaaatgtggacttggagaaatctggggccagcttgctctccgcaggctcaagatcaaccatcccacatag

SEQ ID NO: 610 - RNA, 125 nt, synthetic construct
  source 1..125: mol_type = other RNA
gcagactcactatgcacctgactgtacttccaggcaggtgctttttctgtctgccagagaaacattccagggtgctgtggctgcctcacctatccagggcgatgcagctccctggggacacaggt

SEQ ID NO: 611 - RNA, 125 nt, synthetic construct
  source 1..125: mol_type = other RNA
gcagactcactctgcatctgactatacttccaggcgggtgctttttctgtctgccagataaacattccagggtgctgtggccgcctcacgtatccagagtgatgcagctccctggggacacaggt

SEQ ID NO: 612 - RNA, 148 nt, synthetic construct
  source 1..148: mol_type = other RNA
tgatggctgttcctctcactgcttgaagccttaggcagtgggattttgatccatcatatatcaaaaatggcttatcttcactcagggcaccatgaggatgggctggctgtccgttagtgccttctgatttttgcggagtcaaacaatt

SEQ ID NO: 613 - RNA, 127 nt, synthetic construct
  source 1..127: mol_type = other RNA
ctggggaattcaaacttgtgttaagaaaatgtgtcccagtgtgcaatggctgcaaacagcagcttccttggtagtgtatgcagcctgtttgttgtacgggttgctctaaagggccttggagacagtc

SEQ ID NO: 614 - RNA, 126 nt, synthetic construct
  source 1..126: mol_type = other RNA
aggtcacttcaaagagggcttgtggggctgtgaaaccaagaggtcttaacagtatgaccaaaaactgaagttctctataggatgctgtagcactcaatggtgctatgttttcctcaggagatatga

SEQ ID NO: 615 - RNA, 139 nt, synthetic construct
  source 1..139: mol_type = other RNA
ctgtccgttgctggcttcacaagtactagtataatttttaaaatgttttattattttgaaaataatgttgtaattcatgccagggactgacaaaagacttgagacaggatggttattcttgtcagctaaggtcacattg

SEQ ID NO: 616 - RNA, 113 nt, synthetic construct
  source 1..113: mol_type = other RNA
tccaaacagacactgatggcaccttctgccatttaggaatttgttttaaaacagacatttgtctagatatttcctttgtggcctcctccccatcaaaagtcaatcaaacatcg

SEQ ID NO: 617 - RNA, 143 nt, synthetic construct
  source 1..143: mol_type = other RNA
gccctatgttaaaattttaattctgcacttactaactatcttgggaaccttgggcaagtaaccaacctcttgtgctttggtttcctcattggtaaaatggggataacagtacttacctcacagagttgttgagaggaacaaat

SEQ ID NO: 618 - RNA, 125 nt, synthetic construct
  source 1..125: mol_type = other RNA
atgcaggtactgttacaatacaactgatgtgttttgttgtcgttccccctgcttaaagcacttgatgcataactctgtctaccttcattccgtagtaagacagagacgcttggcttcagacattt

SEQ ID NO: 619 - RNA, 147 nt, synthetic construct
  source 1..147: mol_type = other RNA
tcactgccctgctcacccttcctgagtccggcggcaagggtaactctgggagcatcgtagagggcagagaagaagaaaccctgaggtcccattatgtcagccccttctatcacacgggaggagactgaggacagaaagggaacagag

SEQ ID NO: 620 - RNA, 147 nt, synthetic construct
  source 1..147: mol_type = other RNA
cttttctcagtggtgcaagaagattaagccacattctggctttagagaggcatttctgagagagatgaaggacacttcgttccccagccccaacctaagcatgtgactgtactcaccttgtcagatgctgttggaacctggctgaca

SEQ ID NO: 621 - RNA, 122 nt, synthetic construct
  source 1..122: mol_type = other RNA
ctggaggactaaggaggctgggtctgatgaggcaagattttgctgatacattgctcctagaaaaaagggttggcaagagcagccctggagactcacacggctgactgttctacccaacactc

SEQ ID NO: 622 - RNA, 118 nt, synthetic construct
  source 1..118: mol_type = other RNA
tagtgtggaactgtctactcctcattcctgtggaagcaggaatacattcataacatgctccattaaaaaaggagttctaggccaggcagcgtgcctcatgcctggaatcccagcactt

SEQ ID NO: 623 - RNA, 126 nt, synthetic construct
  source 1..126: mol_type = other RNA
atacctaaacccaagaatcactttcttatagtgatgatttaaacagatgcaaacagcgagcacatcttgtcacctttgcgggactgtggctgtgcccctcgcagtaaatttggaggttctacatcc

SEQ ID NO: 624 - RNA, 133 nt, synthetic construct
  source 1..133: mol_type = other RNA
aggcaggatctagttacattgtagctgtgaagtgctgcattgtctttgccccctgctcaaaataaaactgttacctttcaagccctgtctgccatggtgctgtagcagcagggatgtttggtctcatacatgt

SEQ ID NO: 625 - RNA, 81 nt, synthetic construct
  source 1..81: mol_type = other RNA
cactagaagacagaattcacagaagtagcatttcaccttttgcctttacagaagtatatttggctgttttgtgagacattc

SEQ ID NO: 626 - RNA, 112 nt, synthetic construct
  source 1..112: mol_type = other RNA
acttttacaggtagaatagtaaagcacagtgttgattgcccaagatttattttactttgaaaaaattagaaatttattactatagcaaatgtctagaactttggaaacaagt

SEQ ID NO: 627 - RNA, 134 nt, synthetic construct
  source 1..134: mol_type = other RNA
atgcatctatttgacagacctggagcagttgctatctgctgctatggtttccaccacagatgcaagaagaacatgtccttgcgctttccgtctgtctaattgtggcagctgagattgaatagaggaatacagga

SEQ ID NO: 628 - RNA, 136 nt, synthetic construct
  source 1..136: mol_type = other RNA
aagcactgcctttgaacctgatgtgtcttgtttgtagcttcacgggccaagcaacagtgctagagcataacgacttgttataactggggctcttcagctctcaactgaactgctcttttaaaaacaaggtacattt

SEQ ID NO: 629 - skipped

SEQ ID NO: 630 - RNA, 133 nt, synthetic construct
  source 1..133: mol_type = other RNA
agcctttgtgttgcccattcactttggaaactagtgaatgtggtgtcaaaaaaggcgtaaattaaacgctttgcagccttttcctgcccttaaatttgatacctttggtgtaggagctgcataagtaacagtt

SEQ ID NO: 631 - RNA, 132 nt, synthetic construct
  source 1..132: mol_type = other RNA
ttccaaagtgttgagttcagtccagggcagcttccctgttctgttaattaaactttgggacattaaaatgggctaagggagatgattgggtagaaagtattattctattcatttgcctcccagcctacaaaa

SEQ ID NO: 632 - RNA, 126 nt, synthetic construct
  source 1..126: mol_type = other RNA
aagcaacactctgtggcagatgatcaaaactgtctgacacaatttgagcttgctatagcaagaaagtctaacctattccggtgttctctctcccatgagacaagccgttatatagacttaaacagt

SEQ ID NO: 633 - skipped

SEQ ID NO: 634 - RNA, 132 nt, synthetic construct
  source 1..132: mol_type = other RNA
ttccacagctactggtctgcagctgttcttatggtagcagttgtggcattcctctgtgggaaagaaactgttaacacaaacacctctttcttagcaaaacagaaagtgggtatatatgtgtgacagacacaa

SEQ ID NO: 635 - RNA, 134 nt, synthetic construct
  source 1..134: mol_type = other RNA
tgcagccgtgtcaaattcagtacctgtcctatgcatggtaggcactggcccagaaggctgccacagaaacactgtgactcatgggccctgttcctgtgtcccaggctcagggataaatttggttacagacatca

SEQ ID NO: 636-637 - skipped (2 entries)

SEQ ID NO: 638 - RNA, 133 nt, synthetic construct
  source 1..133: mol_type = other RNA
tgcagccgtgtcaattcagtacctgtcctatgcatggtaggcactggcccagaaggctgccacagaaacactgtgactcatgggccctgttcctgtgtcccaggctcagggataaatttggttacagacatca

SEQ ID NO: 639 - skipped

SEQ ID NO: 640 - RNA, 134 nt, synthetic construct
  source 1..134: mol_type = other RNA
tggtaatggatttatggtgggtccttctctgtgggcctctcatagtgtacccatgccatagcaaatggcagcctcgaaccattgcccagtccccttacctgtgggctgtgagcactgaagggggttgcacagtg

SEQ ID NO: 641 - skipped

SEQ ID NO: 642 - RNA, 132 nt, synthetic construct
  source 1..132: mol_type = other RNA
cagcatgtttccaagggctgtggctggtcatagccatgggatctccaactgcatgcaagagcaacctggaaagactttgacagcgcaggtcagtacaatacctgcaagctgccactcagctttcctataatg

SEQ ID NO: 643 - skipped

SEQ ID NO: 644 - RNA, 133 nt, synthetic construct
  source 1..133: mol_type = other RNA
ggtctctcagctccgcttaaccacacgggtccagtgtgtgcttggcgtgttttcagggaggcagagaaaggctctcctaatgcacgacagacccgcccagaatggcctctctgttcctaggagtgcgacaatt

SEQ ID NO: 645 - RNA, 135 nt, synthetic construct
  source 1..135: mol_type = other RNA
agcactatatttaaacctgtggatgggaatattccccattcttggttacgctgtagtgcaaaagaattcctggctctctgttgcacagctgacttgtgccattctgctgttgctgtatagagttaaggaacatgg

SEQ ID NO: 646 - skipped

SEQ ID NO: 647 - RNA, 130 nt, synthetic construct
  source 1..130: mol_type = other RNA
tgcctcattctagagaatgggcactgttgatcatggtgtccaaaaatagttaatgtggctaaattgagacaggttatgcttccatcacagtatgcatattgcagtggtgacaatgagacctgtaacattt

SEQ ID NO: 648 - RNA, 135 nt, synthetic construct
  source 1..135: mol_type = other RNA
taggccctgaatcaagaccaatggtttgctgtagctgttggtttcaaacaggagctaagagtgatgtcttccttgtggtctgttggctattcagtattccagtgcgaattgccaattcagttggaagaaacatag

SEQ ID NO: 649 - skipped

SEQ ID NO: 650 - RNA, 130 nt, synthetic construct
  source 1..130: mol_type = other RNA
atcgaggctagagtcacgcttgggtatcggctattgcctgagtgtgctagagtcctcgaagagtaactgctgaccttattcactggctgtgggccttatggcacagtcagtcaccaggttagagacatgc

SEQ ID NO: 651 - skipped

SEQ ID NO: 652 - RNA, 149 nt, synthetic construct
  source 1..149: mol_type = other RNA
tgcacactattaaagctcagggtggaggccagtcttggctcatgaacttctgagtgtcggaagtgtgctatatcaatggcaggattttcgctaacaccagtagagcttgcctctatgactggagtttggtagtactcgctgccacatag

SEQ ID NO: 653 - RNA, 139 nt, synthetic construct
  source 1..139: mol_type = other RNA
gacctcctgggatcgcatctggagagtgcctagtattctgccagcttcggaaagggagggaaagcaagcctggcagaggcacccattccattcccagcttgctccgtagctggcgattggaagacactctgcgacagtg

SEQ ID NO: 654 - skipped

SEQ ID NO: 655 - RNA, 133 nt, synthetic construct
  source 1..133: mol_type = other RNA
tagcaagcctccagcgtgcttgggtctgcggtgaccctatgcattccttcagtgcttgctagaacagttttgaaacggtttgaggccttgccctgctccatccagagcaaggttatagaaatttcagacaatg

SEQ ID NO: 656 - skipped

SEQ ID NO: 657 - RNA, 134 nt, synthetic construct
  source 1..134: mol_type = other RNA
ttggcccttatcgaagctgcagctgcttccgcatagctgctgtggtcaaaaaggagcccagagtgacagttttccttgacggtcgccgttctgtttgttgtaactgatctgcaacattttgggaaaatacagtt

SEQ ID NO: 658 - DNA, 31 bp, synthetic construct
  source 1..31: mol_type = other DNA
acacaggcactggccactgaaatttttggag

SEQ ID NO: 659 - DNA, 47 bp, synthetic construct
  source 1..47: mol_type = other DNA
taggctttctggcttttcaccggaaagcccctaaatgattaaattaa

SEQ ID NO: 660 - DNA, 31 bp, synthetic construct
  source 1..31: mol_type = other DNA
gattaggcacacacaggcacaatttttggag

SEQ ID NO: 661 - DNA, 47 bp, synthetic construct; identical to SEQ ID NO: 659
  source 1..47: mol_type = other DNA

SEQ ID NO: 662 - DNA, 31 bp, synthetic construct
  source 1..31: mol_type = other DNA
cttccttgacgattaggcacaatttttggag

SEQ ID NO: 663 - DNA, 47 bp, synthetic construct; identical to SEQ ID NO: 659
  source 1..47: mol_type = other DNA

SEQ ID NO: 664 - DNA, 31 bp, synthetic construct
  source 1..31: mol_type = other DNA
accttcttccttgacgattaaatttttggag

SEQ ID NO: 665 - DNA, 47 bp, synthetic construct; identical to SEQ ID NO: 659
  source 1..47: mol_type = other DNA

SEQ ID NO: 666 - DNA, 31 bp, synthetic construct
  source 1..31: mol_type = other DNA
acacaggcactggccactgatgcgtgtcatt

SEQ ID NO: 667 - DNA, 47 bp, synthetic construct; identical to SEQ ID NO: 659
  source 1..47: mol_type = other DNA

SEQ ID NO: 668 - DNA, 31 bp, synthetic construct
  source 1..31: mol_type = other DNA
ggcacacacaggcactggcctgcgtgtcatt

SEQ ID NO: 669 - DNA, 47 bp, synthetic construct; identical to SEQ ID NO: 659
  source 1..47: mol_type = other DNA

SEQ ID NO: 670 - DNA, 31 bp, synthetic construct
  source 1..31: mol_type = other DNA
acgagctcagcctatgcgagaatttttggag

SEQ ID NO: 671 - DNA, 47 bp, synthetic construct; identical to SEQ ID NO: 659
  source 1..47: mol_type = other DNA

SEQ ID NO: 672 - DNA, 31 bp, synthetic construct
  source 1..31: mol_type = other DNA
ccacctaccctatcgtgcggaatttttggag

SEQ ID NO: 673 - protein, 47 aa, synthetic construct
TAGGCTTTCTGGCTTTTCACCGGAAAGCCCCTAAATGATTAAATTAA

SEQ ID NO: 674 - DNA, 83 bp, synthetic construct
  source 1..83: mol_type = other DNA
ggtcaatgatgtgttggcatgtatcaggtattcctgtggctgatgtgtaataacactggatgaagggacacacactgagacct

SEQ ID NO: 675 - DNA, 103 bp, synthetic construct
  source 1..103: mol_type = other DNA
tatgatagggacttagggtgggtcaatgatgtgttggcatgtatcaggtattcctgtggctgatgtgtaataacactggatgaagggacacacactgagacct

SEQ ID NO: 676 - DNA, 103 bp, synthetic construct
  source 1..103: mol_type = other DNA
ggtcaatgatgtgttggcatgtatcaggtattcctgtggctgatgtgtaataacactggatgaagggacacacactgagaccttatgatagggacttagggtg

SEQ ID NO: 677 - DNA, 83 bp, synthetic construct
  source 1..83: mol_type = other DNA
ggtcaatgatgtgttggcatgtatggtacaggtattcctctgatgtgtaataacactctgtggatgaagggacactgagacct

SEQ ID NO: 678 - DNA, 103 bp, synthetic construct
  source 1..103: mol_type = other DNA
tatgatagggacttagggtgggtcaatgatgtgttggcatgtatggtacaggtattcctctgatgtgtaataacactctgtggatgaagggacactgagacct

SEQ ID NO: 679 - DNA, 103 bp, synthetic construct
  source 1..103: mol_type = other DNA
ggtcaatgatgtgttggcatgtatggtacaggtattcctctgatgtgtaataacactctgtggatgaagggacactgagaccttatgatagggacttagggtg

SEQ ID NO: 680 - DNA, 135 bp, synthetic construct
  source 1..135: mol_type = other DNA
agcactataaagggacctgtggatgggaatattccccattcttggtacacacatagtgcaaaagaattcctggctctctgttgcacagctgacttgtgccattctgctgttgctgtatagagttaaggaacatgg

SEQ ID NO: 681 - DNA, 155 bp, synthetic construct
  source 1..155: mol_type = other DNA
tatgatagggacttagggtgagcactataaagggacctgtggatgggaatattccccattcttggtacacacatagtgcaaaagaattcctggctctctgttgcacagctgacttgtgccattctgctgttgctgtatagagttaaggaacatgg

SEQ ID NO: 682 - DNA, 155 bp, synthetic construct
  source 1..155: mol_type = other DNA
agcactataaagggacctgtggatgggaatattccccattcttggtacacacatagtgcaaaagaattcctggctctctgttgcacagctgacttgtgccattctgctgttgctgtatagagttaaggaacatggtatgatagggacttagggtg

SEQ ID NO: 683 - DNA, 132 bp, synthetic construct
  source 1..132: mol_type = other DNA
cagcatgtttccaagggctgtggctggtcatagccatgggatctccaactgcatgcaagagcaacctggaaagacacacacagcgcaggtcagtacaatacctgcaagctgcatgccagctttcctataatg

SEQ ID NO: 684 - DNA, 152 bp, synthetic construct
  source 1..152: mol_type = other DNA
tatgatagggacttagggtgcagcatgtttccaagggctgtggctggtcatagccatgggatctccaactgcatgcaagagcaacctggaaagacacacacagcgcaggtcagtacaatacctgcaagctgcatgccagctttcctataatg

SEQ ID NO: 685 - DNA, 152 bp, synthetic construct
  source 1..152: mol_type = other DNA
cagcatgtttccaagggctgtggctggtcatagccatgggatctccaactgcatgcaagagcaacctggaaagacacacacagcgcaggtcagtacaatacctgcaagctgcatgccagctttcctataatgtatgatagggacttagggtg

SEQ ID NO: 686-700 - skipped (15 entries)

SEQ ID NO: 701 - DNA, 11 bp, synthetic construct
  source 1..11: mol_type = other DNA
aatttttggag

SEQ ID NO: 702 - DNA, 20 bp, synthetic construct
  source 1..20: mol_type = other DNA
taaaatatatttaaaaggtg

SEQ ID NO: 703 - DNA, 11 bp, synthetic construct
  source 1..11: mol_type = other DNA
aacgcgtcatg

SEQ ID NO: 704-705 - skipped (2 entries)

SEQ ID NO: 706 - DNA, 20 bp, synthetic construct
  source 1..20: mol_type = other DNA
tcacacctaagccctaaaga

SEQ ID NO: 707 - DNA, 20 bp, synthetic construct
  source 1..20: mol_type = other DNA
gttccgcgttacataactta

SEQ ID NO: 708 - DNA, 579 bp, synthetic construct
  source 1..579: mol_type = other DNA
tcacacctaagccctaaagagcctgtacttgttaacaccgaatgtgatgccctgtaggccttcctgactatggcggcctacttatcctgtcccttttttttccacaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatggcttgtttattgcagcttataatggttacaaataa

SEQ ID NO: 709 - DNA, 622 bp, synthetic construct
  source 1..622: mol_type = other DNA
tcacacctaagccctaaagaaatttttggataggctttctggctttttaccggaaagcccctgcctgtacttgttaacaccgaatgtgatgccctgtaggccttcctgactatggcggcctacttatcctgtcccttttttttccacaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatggcttgtttattgcagcttataatggttacaaataaa

SEQ ID NO: 710 - DNA, 621 bp, synthetic construct
  source 1..621: mol_type = other DNA
gttccgcgttacataacttaaatttttggataggctttctggctttttaccggaaagcccctgcctgtacttgttaacaccgaatgtgatgccctgtaggccttcctgactatggcggcctacttatcctgtcccttttttttccacaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatggcttgtttattgcagcttataatggttacaaataa

SEQ ID NO: 711 - DNA, 610 bp, synthetic construct
  source 1..610: mol_type = other DNA
gtcggtctcggatctcacacctaagccctaaagaaacgcgtcatgtaggctttctggctttttaccggaaagcccctgcctgtacttgttaacaccgaatgtgatgccctgtaggccttcctgactatggcggcctacttatcctgtcccttttttttccacaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatggccgagacc

SEQ ID NO: 712 - DNA, 250 bp, synthetic construct
  source 1..250: mol_type = other DNA
tccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggag

SEQ ID NO: 713 - DNA, 4216 bp, synthetic construct
  source 1..4216: mol_type = other DNA
ccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaaggccggcatggtcccagcctcctcgctggcgccggctgggcaacatgcttcggcatggcgaatgggactggcgatccagacatgataagatacattgatgagtttggacaaaccacaactagaatgcagtgaaaaaaatgctttatttgtgaaatttgtgatgctattgctttatttgtaaccattataagctgcaataaacgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtacagcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagagact

SEQ ID NO: 714 - DNA, 4381 bp, synthetic construct
  source 1..4381: mol_type = other DNA
gcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcaggtgtccctgacctgggtagagtggcatctggttggtgatgcccatctcatatcagccagggactgggcaactccttgttctctccagcttggcttttgatccgtgcccatgcctggcctggttcttgggcgcgtggggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtaca

SEQ ID NO: 715 - DNA, 4381 bp, synthetic construct
  source 1..4381: mol_type = other DNA
gcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcaggtgtccctgacctgggtagagtggcatctggttggtgatgcccatctcatatcagccagggacaaagcaactccttgttctctccagcttggcttttgatccgtgcccatgcctggcctggttcttggacacatagggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtaca

SEQ ID NO: 716 - DNA, 4531 bp, synthetic construct
  source 1..4531: mol_type = other DNA
gcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcaggtgtccctgacctgggtagagtggcatctggttggtgatgcccatctcatatcagccagggactgggcaactccttgttcatcccagcttggcttttgatccgtgcccatgcctggttcatgccttgggcgcgtgggtttcctttaaagaggtggtattgtagccagcttatatttgcatctacagccatgtttctagtccagcttggtgtgcaatactagatgagttaataactggtccttgtttctgatctggttcccattgtgtaactgtgttgattgggaagggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtaca

SEQ ID NO: 717 - DNA, 4381 bp, synthetic construct
  source 1..4381: mol_type = other DNA
gcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcaggtgtccctgacctgggtagagtggcatctggttggtgatgcccatctcatatcagccagggactgggcaactccttgttcatcccagcttggcttttgatccgtgcccatgcctggttcatgccttgggcgcgtggggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtaca

SEQ ID NO: 718 - DNA, 4531 bp, synthetic construct
  source 1..4531: mol_type = other DNA
gcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcaggtgtccctgacctgggtagagtggcatctggttggtgatgcccatctcatatcagccagggacaaagcaactccttgttcatcccagcttggcttttgatccgtgcccatgcctggttcatgccttggacacataggtttcctttaaagaggtggtattgtagccagcttatatttgcatctacagccatgtttctagtccagcttggtgtgcaatactagatgagttaataactggtccttgtttctgatctggttcccattgtgtaactgtgttgattgggaagggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtaca

SEQ ID NO: 719 - DNA, 4381 bp, synthetic construct
  source 1..4381: mol_type = other DNA
gcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcaggtgtccctgacctgggtagagtggcatctggttggtgatgcccatctcatatcagccagggacaaagcaactccttgttcatcccagcttggcttttgatccgtgcccatgcctggttcatgccttggacacatagggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtaca

SEQ ID NO: 720 - DNA, 5312 bp, synthetic construct
  source 1..5312: mol_type = other DNA
actggtctctgcaggctccttgttctctccagcttggcttttgatccgtgcccatgcctggcctggttcttggggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaagagaccgtcgcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctggagaccgagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggaaaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgaggtctcatacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtacaactggtctctccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcagtgagaccagt

SEQ ID NO: 721 - DNA, 5280 bp, synthetic construct
  source 1..5280: mol_type = other DNA
actggtctctgcaggctccttgttctctacctggttcttggggccggcatggtcccagcctcctcgctggcgccggctgggcaacatgcttcggcatggcgaatgggactggcttgtttattgcagcttataatggttacaaataaagcaatagcatcacaaatttcacaaataaagcatttttttcactgcattctagttgtggtttgtccaaactcatcaatgtatcttatgctgtttatgagacgggaatgtacggaacctcgatgccattgcatctgaacgcctggcgatcagacgacactagagtcacgacgataccaggtctgtcatatgagcggaacccatgccagattgacccaactccatgaaactacgatgcagagactggactagtgcactgcagtacaagagaccgtcgcggccgcgattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgggacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtgcggccgcccctgaattcgcatctagaggaatgtacggaacctcgatgccattgcatctgaacgcctggcgatcagacgacactagagtcacgacgataccaggtctgtcatatgagcggaacccatgccagattgacccaactccatgaaactacgatgcagaccctggagaccgagtcgaccgacagccttccaaatgttcttcgggtgatgctgccaacttagtcgaccgacagccttccaaatgttcttctcaaacggaatcgtcgtatccagcctactcgctattgtcctcaatgccgtattaaatcataaaaagaaataagaaaaagaggtgcgagcctcttttttgtgtgacaaaataaaaacatctacctattcatatacgctagtgtcatagtcctgaaaatcatctgcatcaagaacaatttcacaactcttatacttttctcttacaagtcgttcggcttcatctggattttcagcctctatacttactaaacgtgataaagtttctgtaatttctactgtatcgacctgcagactggctgtgtataagggagcctgacatttatattccccagaacatcaggttaatggcgtttttgatgtcattttcgcggtggctgagatcagccacttcttccccgataacggaaaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgaggtctcatacaaatctgctcgtcagtggtgctcacactgacgaatcatgtacagatcataccgatgactgcctggcgactcacaactaagcaagacagccggaaccagcgccggcgaacaccactgcatatatggcatatcacaacagtccaactagtgcactgcagtacaactggtctctccctgaattcgcatctagaccagacagcacgtaattgaccgccagaacgcgcaatttgacgaccagcggatatgagattctgtgcactcacgagaacgttaccgacgatttcccagtatttggcgctgccataatccaaatatcaagcgaatcccatgtgaccgtctcagcgggcgacgatctcagaacagttaaacgtacttctgcctgcctagagatccagtttatcgattagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcggatcttccatttcgggtgtcgtgacggccactatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtaagtgtaacgcaaaagtttacagtcacatctctattaccatctggaataaaattctctatttgatttcttaactgaagagtaaagcaaatttctttgtcacggacagcccgggagcacatgaggatcacccatgtgccacgagcgacatgaggatcacccatgtcgctttcactagtctgtggtgtgatatccatgggagcacatgaggatcacccatgtgccacgagcgacatgaggatcacccatgtccaatttggatgaaattaactgtttaggagaagttacctaagttaacaaaaggaatgtcattgtgcactgaaaatgtaatacatttaaatgattaaattaagcagtgagaccagt

SEQ ID NO: 722 - RNA, 11 nt, synthetic construct
  source 1..11: mol_type = other RNA
gaagtagtgat

SEQ ID NO: 723 - RNA, 11 nt, synthetic construct
  source 1..11: mol_type = other RNA
tttacagcaag

SEQ ID NO: 724 - RNA, 11 nt, synthetic construct
  source 1..11: mol_type = other RNA
aattcactact

SEQ ID NO: 725 - RNA, 11 nt, synthetic construct
  source 1..11: mol_type = other RNA
tgaacagaacc

SEQ ID NO: 726 - RNA, 11 nt, synthetic construct
  source 1..11: mol_type = other RNA
gaataggtttg

SEQ ID NO: 727 - RNA, 11 nt, synthetic construct
  source 1..11: mol_type = other RNA
atgaaagttca

SEQ ID NO: 728 - RNA, 11 nt, synthetic construct
  source 1..11: mol_type = other RNA
gagtctgcgtg

SEQ ID NO: 729 - RNA, 11 nt, synthetic construct
  source 1..11: mol_type = other RNA
tccgagttcca

SEQ ID NO: 730 - RNA, 11 nt, synthetic construct
  source 1..11: mol_type = other RNA
tagccggtact

SEQ ID NO: 731 - RNA, 11 nt, synthetic construct
  source 1..11: mol_type = other RNA
tcatgaatgtg

SEQ ID NO: 732 - RNA, 137 nt, synthetic construct
  source 1..137: mol_type = other RNA
atccaaggtgattccctctccaaggggacatcagtgcctctcaggaaagtagcagcttggaatagaatctggcatgcctaaggcctttggggaactgggatgcttatttcctctgccttccttggctgcccacatgg

SEQ ID NO: 733 - RNA, 134 nt, synthetic construct
  source 1..134: mol_type = other RNA
ttgcacagtgaacacccaagtgtgctttatagttcccttggctttgaccctgtgctagagcattgcctgctcttctcctctgcattaaaaggaatatttatccttttaaatgtattcagaaagccagcacatta

SEQ ID NO: 734 - RNA, 139 nt, synthetic construct
  source 1..139: mol_type = other RNA
tacctcccaggatggcatctggaagtggatagtattctgccagctttggaaactggatgaaaagcaaatctggcagaggtacccatttcattcccagcttgctcagtagctggtgattggaagaaactctgcaacagtg

SEQ ID NO: 735 - RNA, 134 nt, synthetic construct
  source 1..134: mol_type = other RNA
gggtcatttcaaagagggcttatgaggctgtgaaacccagagctcttaacgctgtgaccaaagatggaagttctctataggaagccatagcactcctaatgtttggtgctatgttttcctgaggagatataaaa

SEQ ID NO: 736 - RNA, 135 nt, synthetic construct
  source 1..135: mol_type = other RNA
ctgcagccaattaagccgactgagttcctttcctcatgggggcccagtgtgcaatggctgtaaatagcagcttccttggtagtgtatgcagcctgtttgttgtatgggttgctctaagggaccttggagacagtc

SEQ ID NO: 737 - RNA, 131 nt, synthetic construct
  source 1..131: mol_type = other RNA
ctccatgtatctttgggacctgtcaagtgtggcagtctcccttccttgccatggaagagcatattcttgtttaccagcaaagctgtcaccatttaattggtatcagattctgacttgcacaagtaacattc

SEQ ID NO: 738 - RNA, 131 nt, synthetic construct; identical to SEQ ID NO: 591
  source 1..131: mol_type = other RNA

SEQ ID NO: 739 - RNA, 131 nt, synthetic construct; identical to SEQ ID NO: 601
  source 1..131: mol_type = other RNA

SEQ ID NO: 740 - RNA, 132 nt, synthetic construct; identical to SEQ ID NO: 634
  source 1..132: mol_type = other RNA

SEQ ID NO: 741 - RNA, 132 nt, synthetic construct
  source 1..132: mol_type = other RNA
gcctgcattcgtaagtgatcacgggctgcccgtgtcctggtcattggtagtgcaggcagaggaaatgcgggaaaggttgctgtgtttggagggtccacatcttcaccctcctgtcccaggagctttcctaca

SEQ ID NO: 742 - RNA, 133 nt, synthetic construct
  source 1..133: mol_type = other RNA
gtgcaaactcgatcactagctctgcgtgatgtggcagaagcgaagggaaccaggtttgcaaaagtaactgtggtgatggaaatgtgttagcctcagacactactgaggtggttctttctatcctagtacagtc

SEQ ID NO: 743 - RNA, 132 nt, synthetic construct
  source 1..132: mol_type = other RNA
ttccaaagtgttgagttcagtccagggcagcttccctgttctgttaattaaactttgggacattgaaatgggctaggggagatgattgggtagaaagcattattttattcatttgcctcccagcctacaaaa

SEQ ID NO: 744 - RNA, 137 nt, synthetic construct
  source 1..137: mol_type = other RNA
cccttggcccttatcgaagctgcagctgcttccgcatagctgctgtggtcaaaaaggagcccagagtgacagttttccttgacggtcgccgttctgtttgttgtaactgatctgcaacattttgggaaaatacagtt

SEQ ID NO: 745 - RNA, 138 nt, synthetic construct
  source 1..138: mol_type = other RNA
tggaggactgagaaggtgaggcagttttgccccgtgctgccttccaccggttaagacctccaaaatcgaagggctgcccaggcagaggatgtccccttgccacccttggaggggcagcgctgtgctggccgacatttg

SEQ ID NO: 746 - RNA, 132 nt, synthetic construct
  source 1..132: mol_type = other RNA
gttgaggtctatcccgatggggcttttcctgtagcctgcacatcgttggaaacgcctcatagagtaactctgtggttttactttactcacaggactattgttagatctgtgggaaggaattacaagacagtt

SEQ ID NO: 747 - RNA, 139 nt, synthetic construct; identical to SEQ ID NO: 594
  source 1..139: mol_type = other RNA

SEQ ID NO: 748 - RNA, 141 nt, synthetic construct
  source 1..141: mol_type = other RNA
tccagctgtaggcagctgcctaggttgtcttgtacctaggcaagtgttacactgctgggagaacagcagccaatagctggttggcattctggccctggttcatgccaactcttgtgttgactaccccaggatgccagcata

SEQ ID NO: 749 - RNA, 136 nt, synthetic construct
  source 1..136: mol_type = other RNA
cttcctcagccttactccagggactttttgttgcctgtaaagtgctctggcattgcctgaggatagatgagaaagcacatatccctccccagtaagacgctgttttcttttggggcctacaagttgagctgacagt

SEQ ID NO: 750 - RNA, 133 nt, synthetic construct; identical to SEQ ID NO: 644
  source 1..133: mol_type = other RNA

SEQ ID NO: 751 - RNA, 137 nt, synthetic construct
  source 1..137: mol_type = other RNA
gcatggtaatggatttatggtgggtccttctctgtgggcctctcatagtgtacccatgccatagcaaatggcagcctcgaaccattgcccagtccccttacctgtgggctgtgagcactgaagggggttgcacagtg

SEQ ID NO: 752 - RNA, 133 nt, synthetic construct
  source 1..133: mol_type = other RNA
gcgctgtctttgagcccccgccgagcttcctcgtggcgccgggggtcaatctgcagcgctagagcatgtgcttgcgcataactggggccgcctggcctcccgcgggcggcctttttaaccgcgagcgacaaga

SEQ ID NO: 753 - RNA, 135 nt, synthetic construct
  source 1..135: mol_type = other RNA
gcatgggtttggatttatgatgggctggattccctaggcctctcatagtaccccatgccagagcaaactgtagccccaaccattgccgggcctctatgcctgtaggctgctggcactgaagtgggttgcacagta

SEQ ID NO: 754 - RNA, 138 nt, synthetic construct
  source 1..138: mol_type = other RNA
tgcactgcatggtatctgcactcagcagtttatacctgctagggtgttcaaaggtcagtgctatagaaattcagtatctggcatcgttggttttcttggctttgtgcttgttaaacctggtatttctactgatacagt

SEQ ID NO: 755 - RNA, 133 nt, synthetic construct
  source 1..133: mol_type = other RNA
gccctgtggttgctggatgctgttgtgcatggacagctctccagtggattcgatgggccatagcaatcctgtgatttatgcatggaggctgcttctcctcagcagctgccatagcccggtcgctggtacatga

SEQ ID NO: 756 - RNA, 134 nt, synthetic construct
  source 1..134: mol_type = other RNA
acctgcattcaaaaatgatcacgggctgcctgtgctctggtcatcaataacgcagggagaggaattgctgaaagccgtttcccgtgtttggagggttcacacctgtccctttcaaatgctggcgctttcacaca

SEQ ID NO: 757 - RNA, 131 nt, synthetic construct
  source 1..131: mol_type = other RNA
gccgagactagagtcacatcctgacacaactcttgtcctggtgtgctagagtactcgaagagaatctactggtcttgattcactggtgggggcagtcggtgcccccgttagtgcccagatcagaaacatac

SEQ ID NO: 758 - RNA, 135 nt, synthetic construct
  source 1..135: mol_type = other RNA
gcatgggtttggattatgacaggcccatccccctggacctctcatagtgccccatgccagagcaaactgtggccccgaaccattgcctggcttctgtacccgtgggccactggcactgaagagggttacacagtg

SEQ ID NO: 759 - RNA, 136 nt, synthetic construct; identical to SEQ ID NO: 628
  source 1..136: mol_type = other RNA

SEQ ID NO: 760 - RNA, 135 nt, synthetic construct
  source 1..135: mol_type = other RNA
ctgcagccaattaagccgactgagttcctttcctcatgggggcccagtgtgcaatcgctgcaaacagcagcttccttggtagtatatgcagcctgtttattgtacgggttgctctacgggaccttggagacaggc

SEQ ID NO: 761 - RNA, 132 nt, synthetic construct
  source 1..132: mol_type = other RNA
aagcaggattcagactacaatatagctgctaagtgctgtgttgtcgttccccctgcttaaaataaagttgtttcttaactatacctgtctgctattctcctgtagcagccagggacgcttggtctcatacat

SEQ ID NO: 762 - RNA, 132 nt, synthetic construct; identical to SEQ ID NO: 590
  source 1..132: mol_type = other RNA

SEQ ID NO: 763 - RNA, 123 nt, synthetic construct
  source 1..123: mol_type = other RNA
gagcactgttcgtaacccgttagcctggctgtagctaatgggttccattccggtgcaatagcatttccagcgacacatgactgactgactggtggctttcagtttcaggtcttggagacaaat

SEQ ID NO: 764 - RNA, 135 nt, synthetic construct
  source 1..135: mol_type = other RNA
tttccctgacctgggtagagtggcatccagttggtggtgcccatctcatatcagccagggacaaagcaaccccttgttcctcccagcttggcttttcatctgtgcctatgcctggttcatgccttggacacattt

SEQ ID NO: 765 - RNA, 137 nt, synthetic construct
  source 1..137: mol_type = other RNA
ttggccctgaatcaaggccagcagtttgctgaagctgttggtttcaagcaggagcctaaagaattgtctttctatggtctgttggccatttcataactttggaaatgtaatggtcaattcattagaaagaaacatga

SEQ ID NO: 766 - RNA, 134 nt, synthetic construct; identical to SEQ ID NO: 635
  source 1..134: mol_type = other RNA

SEQ ID NO: 767 - RNA, 136 nt, synthetic construct
  source 1..136: mol_type = other RNA
gcatgggtttggatttatgatgggcccgtccccctggacctctcatagtaccccatgccagagcaaactgtagccctgaaccattgcctggcctctgttcccgtaggctgctggcactgaagtgggttgcacaata

SEQ ID NO: 768 - RNA, 132 nt, synthetic construct; identical to SEQ ID NO: 642
  source 1..132: mol_type = other RNA

SEQ ID NO: 769 - RNA, 133 nt, synthetic construct; identical to SEQ ID NO: 630
  source 1..133: mol_type = other RNA

SEQ ID NO: 770 - RNA, 132 nt, synthetic construct; identical to SEQ ID NO: 599
  source 1..132: mol_type = other RNA

SEQ ID NO: 771 - RNA, 132 nt, synthetic construct
  source 1..132: mol_type = other RNA
aaagcaggttgcaattacagtgcttcattttgtggaagtactgccattatcctgctgaaagaaaagccgtgttaatcatttttgattttgcctttatgagggtaaaatcatgacagattgacatggacaatt

SEQ ID NO: 772 - RNA, 135 nt, synthetic construct
  source 1..135: mol_type = other RNA
gcactgctttagagcctctgcgtgtctggctgtggcctcaggggtctagcagcagtgctagagcagacccagcttgtcagatccggggctgcttagaggccacctaagtgattcctttggcagcaagcaacattc

SEQ ID NO: 773 - RNA, 136 nt, synthetic construct
  source 1..136: mol_type = other RNA
gctgtcctggacctgttggcaccacagacagttgctctgctgtgcctgtggcctcggggcaaagagaaagtggcgatttctacactcagtgctcgggaaccagtgggcactgagaatggtttatggcctgacatta

SEQ ID NO: 774 - RNA, 131 nt, synthetic construct
  source 1..131: mol_type = other RNA
ttccaaagtgttaagttcagttcagggtagcttccctgctctgttaattaaactttggaacattgaaactggctagggaaaatgattggatagaaactattattctattcatttatccccagcctacaaaa

SEQ ID NO: 775 - RNA, 133 nt, synthetic construct; identical to SEQ ID NO: 598
  source 1..133: mol_type = other RNA

SEQ ID NO: 776 - RNA, 137 nt, synthetic construct
  source 1..137: mol_type = other RNA
tgcagtcaagtcaaattcagtgcccgtttctgtcatagcgggggctggcccagatggctgccacagcaagctccacagctcatgggccctgggtcacctaccctgggacctggggataagtttggctgtggacagtg

SEQ ID NO: 777 - RNA, 135 nt, synthetic construct
  source 1..135: mol_type = other RNA
tgtccctgacctgggtagagtggcatctggttggtgatgcccatctcatatcagccagggacaaagcaactccttgttcatcccagcttggcttttgatccgtgcccatgcctggttcatgccttggacacatag

SEQ ID NO: 778 - RNA, 135 nt, synthetic construct
  source 1..135: mol_type = other RNA
ccgcagccaattaagccgactgagttcctttcctcatggggacccagtgtgcgatggctgcacacagcagcttccttggtagtgtacgcagcctgttggttgtatgggttgctctaagggaccttggagacaggc

SEQ ID NO: 779 - RNA, 131 nt, synthetic construct; identical to SEQ ID NO: 609
  source 1..131: mol_type = other RNA

SEQ ID NO: 780 - RNA, 136 nt, synthetic construct
  source 1..136: mol_type = other RNA
gggcatacccgtagaccttgtctgactgtgctcatggccaggcaggggggacagtgtatgcaagagtaatgtggagtttgtgctaactctagccagcttaattagtgactggataaattgcacaactctcacattc

SEQ ID NO: 781 - RNA, 133 nt, synthetic construct
  source 1..133: mol_type = other RNA
actgcccctagaggcgttgcagctgtggctgccgtgtcacatctgtgtcattaggtggcagagattagagaggctatgtctacgctcagcgttctgccccgtgaacgtttgaatgtttgatagtctcacactc

SEQ ID NO: 782 - RNA, 135 nt, synthetic construct
  source 1..135: mol_type = other RNA
gcacctgaatctttcccattccttgctgcctcgtgccggtgtggggacagatggtgctacagaatgagcagaggaaatccagacaggttgttttccatttgtcttggggcctgtctctacagctctgccacattt

SEQ ID NO: 783 - RNA, 138 nt, synthetic construct
  source 1..138: mol_type = other RNA
ctaccaaaagttagctttttggggggcaggtttttaagtaacctttgccaacttgggctatttggaagagtaaaagaccacactccacagtgggctataccacttagtatagttcgctactattttgtggcctacatg

SEQ ID NO: 784 - RNA, 136 nt, synthetic construct
  source 1..136: mol_type = other RNA
gcatgggtttggatttatgacaggcccgtcaccctgggcctgtcatagtaccccatgccagagcaaactgtgtccccgaaccattgcctggcctctgtgcccgtaggctgctggcactgaagtgggttgcacagtg

SEQ ID NO: 785 - RNA, 134 nt, synthetic construct
  source 1..134: mol_type = other RNA
actctctcggctctgcatagttgcacttggcttcacccgtgtgactttcgtaacggggagagagagaaaagatctcctcaggacctcggatgggccttactgtggcctctctttccttgaggggtgcaacaggc

SEQ ID NO: 786 - RNA, 132 nt, synthetic construct
  source 1..132: mol_type = other RNA
ccccttttaaaagcactcaatgggcctgtggctaatgacctattgagccgtcaagaaaggggagagtgaaaacatcgcttttgggtgaagtggcaacatgtgttgtttgcttcaatcggtggtgtgacaagg

SEQ ID NO: 787 - RNA, 132 nt, synthetic construct
  source 1..132: mol_type = other RNA
ggcctcctggtgcttaccacaggctgtgttcttacactgactgtatagaaagaggaggtagagtaaacctaccccatatacacctcagctcaggccctgtgcctggtctgtattgtgaatgggggaacatag

SEQ ID NO: 788 - RNA, 137 nt, synthetic construct
  source 1..137: mol_type = other RNA
gggcatactcgtagaccttgcctgactgtgctcatgtccaggcaggggggacagtgtatgcaagaataatttggagttcctgccagctctaaccagcttcatcagtggctggataaattgcaggactctaaacattt

SEQ ID NO: 789 - RNA, 132 nt, synthetic construct
  source 1..132: mol_type = other RNA
ctgcgaatattctcgctgttctgattttgtaatagtcaggacaggctaaacattcgctatattaagaccatgcatgtgtccccaaacctagttctttccctaggtctggttttataaatgctggtgataaac

SEQ ID NO: 790 - RNA, 133 nt, synthetic construct; identical to SEQ ID NO: 655
  source 1..133: mol_type = other RNA

SEQ ID NO: 791 - RNA, 132 nt, synthetic construct; identical to SEQ ID NO: 631
  source 1..132: mol_type = other RNA

SEQ ID NO: 792 - RNA, 135 nt, synthetic construct; identical to SEQ ID NO: 645
  source 1..135: mol_type = other RNA

SEQ ID NO: 793 - RNA, 135 nt, synthetic construct; identical to SEQ ID NO: 600
  source 1..135: mol_type = other RNA

(19) United States
(12) Patent Application Publication
(10) Pub. No.: US 2025/0250563 A1
(43) Pub. Date: Aug. 7, 2025

(54) METHODS AND COMPOSITIONS FOR TRANS-SPLICING UTILIZING SMALL NUCLEAR RNAS AND SMALL NUCLEOLAR RNAS

(71) Applicant: Amber Bio Inc., San Francisco, CA (US)

(72) Inventors: Jacob BORRAJO, San Francisco, CA (US); Sam DEMARIO, San Francisco, CA (US); Kamyab JAVANMARDI, San Francisco, CA (US); David Yao, San Francisco, CA (US); Brigit Riley, San Francisco, CA (US)

(21) Appl. No.: 18/938,180

(22) Filed: Nov. 5, 2024

Related U.S. Application Data
(60) Provisional application No. 63/656,569, filed on Jun. 5, 2024.
(60) Provisional application No. 63/554,139, filed on Feb. 15, 2024.
(60) Provisional application No. 63/552,646, filed on Feb. 12, 2024.
(60) Provisional application No. 63/550,019, filed on Feb. 5, 2024.

(51) Int. Cl. C12N 15/11; C12N 15/88

(52) U.S. Cl. CPC C12N 15/111; C12N 15/88; C12N 2310/12; C12N 2830/50

(57) ABSTRACT

The present disclosure provides compositions and methods of use thereof for targeting trans-splicing of a pre-mRNA in a cell utilizing small nuclear RNAs (snRNAs) and small nucleolar RNAs (snoRNAs), and related methods.

### CROSS-REFERENCE TO RELATED APPLICATIONS

This application claims the benefit of and priority to U.S. Provisional Application No. 63/656,569, filed on Jun. 5, 2024, U.S. Provisional Application No. 63/554,139, filed on Feb. 15, 2024, U.S. Provisional Application No. 63/552,646, filed on Feb. 12, 2024, and U.S. Provisional Application No. 63/550,019, filed on Feb. 5, 2024, the entire contents of each of which are incorporated herein.

### FIELD

The disclosure relates to a nucleic acid composition for targeting trans-splicing of a pre-mRNA in a cell utilizing small nuclear RNAs (snRNAs) and small nucleolar RNAs (snoRNAs), and related methods.

### SEQUENCE LISTING

The instant application contains a sequence listing, which has been submitted in XML format via PatentCenter. The contents of the XML copy named “AMR-016_Sequence Listing,” which was created on Nov. 5, 2024, and is approximately 759,558 bytes in size, the contents of which are incorporated herein by reference in their entirety.

### BACKGROUND

Trans-splicing techniques for gene editing have been used to target a wide range of diseases in both in vitro and in vivo models, resulting in RNA, protein and functional correction. Trans-splicing occurs between two splice sites located on two different pre-mRNAs. While trans-splicing has been shown to demonstrate in vitro and in vivo activity, it is a relatively inefficient process that has not yet progressed to the clinic. Thus, there is a need to develop novel compositions and methods that enable specific and efficient trans-splicing to introduce desired genetic information to a cell.

### SUMMARY OF THE DISCLOSURE

Therefore, the present disclosure provides, in aspects, a composition or system suitable for targeting trans-splicing of a pre-mRNA in a cell comprising one or more nucleic acids comprising one or more nucleotide sequences comprising at least one intronic sequence comprising a small nuclear RNA (snRNA), or a small nucleolar RNA (snoRNA) sequence, a splice acceptor and/or splice donor sequence.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA. In embodiments, the snoRNA comprises an H/ACA box or C/D box. In embodiments, the snRNA or snoRNA sequence assembles into an RNP. In embodiments, the snRNA or snoRNA sequence comprises a sequence motif that assembles into an RNP. In embodiments, the snRNA or snoRNA sequence comprises a secondary structure that assembles into an RNP. In embodiments, the snRNA or snoRNA sequence comprises a sequence motif and a secondary structure that assembles into an RNP. In embodiments, the secondary structure comprises one or more stem loops. In embodiments, the secondary structure is or comprises a stem, internal loop, multibranch loop, or a pseudoknot. In embodiments, the RNP is selected from a small nuclear RNP (snRNP), a small nucleolar RNP (snoRNP), a small cajal body RNP (scaRNP), and a combination thereof. In embodiments, the RNP is selected from U1, U2, U4, U4atac, U5, U6, U6atac, U7, U11, and U12. In embodiments, the RNP is selected from a C/D box snoRNP and a H/ACA box snoRNP.

In embodiments, the snRNA or snoRNA target one or more exonic splicing enhancers (ESEs), one or more intronic splicing enhancers (ISEs), one or more exonic splicing silencers (ESSs), and/or one or more intronic splicing silencers (ISSs). In embodiments, the snRNA or snoRNA comprise a modification to include at least one or more exonic splicing enhancers (ESEs), at least one or more intronic splicing enhancers (ISEs), at least one or more exonic splicing silencers (ESSs), and/or at least one or more intronic splicing silencers (ISSs).

In embodiments, the composition or system comprises a repair RNA (repRNA) and a small RNA that induces cleavage in an RNA. In embodiments, the small RNA that induces cleavage in an RNA is one or more of an siRNA, small hairpin RNA (shRNA), U7 snRNA, a U1 snRNA, a U2 snRNA, a U4 snRNA, a U4atac snRNA, a U5 snRNA, a U6 snRNA, a U6atac snRNA, a U11 snRNA, a U12 snRNA, and an antisense oligonucleotide (ASO). In embodiments, the composition or system comprises a repair repRNA and the small RNA comprises a modification or mutation that attenuates, weakens, reduces, decreases, or ablates activity. In embodiments, the composition or system comprises a repair repRNA and the small RNA comprises a modification or mutation that increases, stimulates, or enhances activity as compared to an unmodified form. In embodiments, the composition or system comprises a repair repRNA and a small RNA that induces cleavage in an RNA when the present methods are undertaken in cis or trans, as described herein. In embodiments, the snRNA or snoRNA comprises a N°-Methyladenosine (M6A) modification. In embodiments, the snRNA or snoRNA comprises a M6A modification when the present methods are undertaken in cis or trans, as described herein. In embodiments, the snRNA or snoRNA is modified to comprise at least one or more M6A sites. In embodiments, the snRNA or snoRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the snRNA or snoRNA or (ii) an exonic sequence. In embodiments, the snRNA or snoRNA is modified to not comprise M6A sites. In embodiments, the repRNA comprises at least one or more M6A sites. In embodiments, the repRNA is modified to comprise at least 1, 2, 34, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the repRNA or (ii) an exonic sequence. In embodiments, the repRNA comprises no M6A sites.

In embodiments, the repRNA comprises at least one intronic spacer sequence comprising at least one ISE and ESS sequences. In embodiments, the at least one intronic spacer sequence comprising at least one ISE and ESS sequences increases trans-splicing efficiency of a target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences decreases trans-splicing efficiency of a target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences increases trans-splicing efficiency of a off-target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences decreases trans-splicing efficiency of a off-target RNA as compared to an unmodified form.

In embodiments, the repRNA comprises a ESS, ESE, ISS, and/or ISE sequence. In embodiments, the repRNA targets one or more of ESS, ESE, ISS, and/or ISE. In embodiments, an interaction, modulation and/or binding to one or more of ESS, ESE, ISS, and/or ISE reduces or ablates interaction, modulation and/or binding of the one or more of the ESS, ESE, ISS, and/or ISE with a target. In embodiments, the repRNA comprises exon sequences with ESE and ESS sequences.

In embodiments, the exon sequences with ESE and ESS sequences increase or decrease trans-splicing efficiency to an RNA target as compared to an unmodified form. In embodiments, the repRNA comprises exon sequences with ESE and ESS sequences. In embodiments, the repRNA comprises exon sequences with ESE and ESS sequences increase or decrease trans-splicing efficiency to an RNA off-target as compared to an unmodified form. In embodiments, the repRNA comprises at least one or more G4 structures. In embodiments, the repRNA comprises at least one or more G4 structures sequester SD/SA motifs. In embodiments, the G4 structure is unwound, such as by DHX36 or CNBP, and remains trapped in the unwound state in the presence of a complementary sequence (e.g., endogenous target or exogenously delivered trigger RNA). In embodiments, the G4 structure decreases off-targets as compared to an unmodified form.

In embodiments, the repRNA comprises a modification comprising at least one or more scaffolding sequences. In embodiments, the at least one or more scaffolding sequences mediates (e.g., recruits) phase condensate-like formation and/or improves local concentrations of repRNAs and other targeted proteins and/or RNA. In embodiments, the repRNA comprises a modification comprising at least one or more sequences to target the repRNA to the promoter of the target gene of interest, or to proximal condensates that may contain the promoter. In embodiments, the one or more sequences comprises an enhancer RNA, snRNA and/or snoRNA sequences.

In embodiments, the repRNA comprises a modification. In embodiments, the repRNA modification improves interaction and localization to a DNA sequence of the non-template strand of the target gene. In embodiments, the DNA sequence of the non-template strand of the target gene is the promoter, intron, exon, or enhancer. In embodiments, the modification improves interaction and localization to the DNA sequence of the non-template strand of the target gene through protein-directed (e.g. transcription factor, dCas, ZNF, or other RBP) or nucleotide-directed (e.g., R-loop) methods.

In embodiments, the repRNA comprises a modification comprising additional RNA elements. In embodiments, the repRNA modification comprising additional RNA elements improves subnuclear localization to nuclear speckles for enhanced trans-splicing efficiency. In embodiments, the additional RNA element comprise NEAT1 and/or MALAT1, or a fragment thereof. In embodiments, the additional RNA element comprises a nucleotide sequence of SEQ ID NO: 712, or a fragment or variant thereof, optionally having at least about 90%, or at least about 95%, or at least about 97%, or at least about 98%, or at least about 99% identity thereto and/or or having about 1 to about 20 (e.g. about 1, or about 2, or about 3, or about 4, or about 5) nucleic acid modifications, optionally selected from substitutions, additions, or deletions. In embodiments, the repRNA modification enables targeting to site of transcription of target RNAs. In embodiments, the repRNA comprises a modification comprising 5′ UTR or 3′ UTR modifications. In embodiments, the modification alters intracellular or intranuclear localization based on interactions with endogenous or exogenously supplied molecules (e.g., RNA G4 interactions with transcription factors or other proteins that are localized to specific cellular compartments). In embodiments, the repRNA comprises a modification in the 5′ UTR of the repRNA. In embodiments, the modification in the 5′ UTR of the repRNA increases stability as compared to an unmodified form. In embodiments, the modification in the 5′ UTR of the repRNA decreases stability as compared to an unmodified form. In embodiments, the modification in the 5′ UTR of the repRNA increases or decreases translation efficiency as compared to an unmodified form. In embodiments, the repRNA comprises a modification in the 3′ UTR of the repRNA. In embodiments, the modification in the 3′ UTR of the repRNA increases stability as compared to an unmodified form. In embodiments, the modification in the 3′ UTR of the repRNA decreases stability as compared to an unmodified form. In embodiments, the modification in the 3′ UTR of the repRNA increases or decreases translation efficiency as compared to an unmodified form.

In embodiments, the repRNA comprises a modification comprising modifying the repRNA to comprise a G4 structure that mediates recruitment of splicing-associated RNA binding protein (RBPs).

In embodiments, the repRNA comprises a modification comprising at least one or more toehold switches in the repRNA. In embodiments, the at least one or more toehold switches in the repRNA conditionally activate or deactivate (e.g., SD/SA occlusion, binding motif occlusion, or RBP occlusion) upon detection of an endogenous or exogenously supplied target RNA.

In embodiments, the repRNA comprises a modification comprising at least one or more complementary riboregulators in repRNAs (in cis). In embodiments, the at least one or more complementary riboregulators in repRNAs (in cis) occlude splice donor (SD) site and reduce off-target trans-splicing.

In embodiments, the repRNA comprises a modification comprising at least one or more self-complementary riboregulators in repRNAs (in cis). In embodiments, the at least one or more self-complementary riboregulators in repRNAs (in cis) occlude splice acceptor (SA) site and reduce off-target trans-splicing.

In embodiments, the repRNA comprises a modification comprising at least one or more self-complementary riboregulators in repRNAs (in trans). In embodiments, the at least one or more self-complementary riboregulators in repRNAs (in trans) occlude splice donor (SD) site and reduce off-target trans-splicing.

In embodiments, the repRNA comprises a modification comprising at least one or more self-complementary riboregulators in repRNAs (in trans). In embodiments, the at least one or more self-complementary riboregulators in repRNAs (in trans) occlude splice acceptor (SA) site and reduce off-target trans-splicing.

In embodiments, the repRNA comprises a modification comprising at least one or more binding motifs. In embodiments, the at least one or more binding motifs increase trans-splicing efficiency, target specificity, and target site occlusion (SA, SD, ISS, ISE, ESE, and ESS) as compared to an unmodified form.

In embodiments, the repRNA comprises a modification. In embodiments, the repRNA modification enables induction of trans-splicing in response to a stimulus as compared to an unmodified form. In embodiments, the repRNA comprises a modification to turn off or decrease trans-splicing in response to a stimulus as compared to an unmodified form.

In embodiments, the repRNA comprises a modification. In embodiments, the repRNA modification enables small molecule induction of trans-splicing as compared to an unmodified form. In embodiments, the repRNA comprises a modification. In embodiments, the repRNA modification represses small molecule induction of trans-splicing as compared to an unmodified form.

In embodiments, the repRNA comprises a modification. In embodiments, the repRNA modification enables light induction of trans-splicing.

In embodiments, the repRNA comprises a modification comprising at least one or more motifs that are bound and regulated by light-sensitive proteins.

In embodiments, the snRNA or snoRNA comprises a sequence at the 3′ untranslated region (3′UTR). In embodiments, the sequence at the 3′ untranslated region (3′UTR) of the snRNA or snoRNA increases trans-splicing efficiency as compared to an unmodified form. In embodiments, the sequence is from the MALAT1 gene. In embodiments, the sequence is a nucleotide sequence of SEQ ID NO: 712, or a fragment or variant thereof, optionally having at least about 90%, or at least about 95%, or at least about 97%, or at least about 98%, or at least about 99% identity thereto and/or or having about 1 to about 20 (e.g. about 1, or about 2, or about 3, or about 4, or about 5) nucleic acid modifications, optionally selected from substitutions, additions, or deletions.

In embodiments, the RNP assembles on the repRNA and/or the target. In embodiments, the RNP assembles on the repRNA. In embodiments, the RNP assembles on the target. In embodiments, the RNP sterically occludes and inhibits cis-splicing.

In embodiments, the repRNA comprises a minimal intron. In embodiments, the minimal intron is less than about 50 nucleotides, less than about 60 nucleotides, less than about 70 nucleotides, less than about 80 nucleotides, less than about 90 nucleotides, less than about 100 nucleotides, less than about 110 nucleotides, less than about 120 nucleotides, less than about 130 nucleotides, less than about 140 nucleotides, or less than about 150 nucleotides, or about 50 to about 150 nucleotides, or about 50 to about 100 nucleotides, or about 50 to about 75 nucleotides, or about 75 to about 150 nucleotides, or about 100 to about 150 nucleotides, or about 120 to about 150 nucleotides.

In embodiments, the repRNA further comprises a ribozyme site. In embodiments, the ribozyme site is a hairpin, hammerhead, hepatitis delta virus (HDV), Varkud satellite (VS), or glmS ribozyme site, or a variant thereof. In embodiments, the ribozyme site is a HDV ribozyme site. In embodiments, the ribozyme site is a twister ribozyme site. In embodiments, the ribozyme site is upstream of the one or more exons and/or introns of the repRNA. In embodiments, the ribozyme cleaves the target. In embodiments, the ribozyme is a trans-cleaving ribozyme. In embodiments, the repRNA comprises a M6A modification. In embodiments, the repRNA comprises a ribozyme site that cleaves at the 5′ end of the repRNA. In embodiments, the repRNA comprises a ribozyme site that cleaves at the 3′ end of the repRNA. In embodiments, the repRNA comprises a ribozyme site that cleaves the snRNA or snoRNA at the 5′ end of the repRNA. In embodiments, the repRNA comprises a ribozyme site that cleaves the snRNA or snoRNA at the 3′ end of the repRNA.

In embodiments, the repRNA comprises a M6A modification when the present methods are undertaken in cis or trans, as described herein. In embodiments, the snRNA or snoRNA is modified to comprise at least one or more M6A sites. In embodiments, the snRNA or snoRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the snRNA or snoRNA or (ii) an exonic sequence. In embodiments, the snRNA or snoRNA is modified to not comprise M6A sites. In embodiments, the repRNA comprises at least one or more M6A sites. In embodiments, the repRNA is modified to comprise at least 1, 2, 34, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the repRNA or (ii) an exonic sequence. In embodiments, the repRNA comprises no M6A sites.

In embodiments, the composition or system further comprises at least one pre-rRNA stemloop. In embodiments, the at least one pre-rRNA stemloop removes either the 5′cap or 3′ polyA tail.

In embodiments, the repRNA comprises at least one or more snRNA or snoRNA sequences. In embodiments, the at least one or more snRNA or snoRNA sequences stabilize the repRNA. In embodiments, the repRNA comprises an artificial smU7 system. In embodiments, the artificial smU7 system stabilizes the repRNA. In embodiments, the least one or more snRNA or snoRNA sequences comprise a pseudoknot at the 5′ end of the snRNA or snoRNA. In embodiments, the pseudoknot at the 5′ end of the snRNA or snoRNA stabilizes the repRNA. In embodiments, the least one or more snRNA or snoRNA sequences comprise a pseudoknot at the 3′ end of the snRNA or snoRNA. In embodiments, the pseudoknot at the 3′ end of the snRNA or snoRNA stabilizes the repRNA.

In embodiments, there are a plurality of repRNAs under the control of the same, different, or a plurality of promoters. In embodiments, the repRNA and one or more other components of the present system are under the control of the same or different promoters.

In embodiments, the repRNA comprises alternative promoters. In embodiments, the repRNA comprises at least one or more alternative Pol II promoters. In embodiments, the one or more alternative Pol II promoters cap the 5′ end of the repRNA with 7 mG (7-methylguanosine) or TMG (tri-methylguanosine). In embodiments, the one or more alternative Pol II promoters cap the 5′ end of the repRNA with 7 mG (7-methylguanosine) or TMG (tri-methylguanosine) stabilize the repRNA.

In embodiments, the repRNA comprises at least one or more circularized 5′ replacement splice donor (SD) repRNAs. In embodiments, the one or more circularized 5′ replacement splice donor (SD) repRNAs stabilize the repRNA. In embodiments, the repRNA comprising one or more circularized 5′ replacement (SD) repRNAs improves stability as compared to an unmodified form and is resistant to exonucleases. In embodiments, the repRNA comprises at least one or more circularized 3′ replacement splice acceptor (SA) repRNAs. In embodiments, the repRNA comprises at least one or more circularized 3′ replacement splice acceptor (SA) repRNAs stabilize the repRNA. In embodiments, the repRNA comprising one or more circularized 3′ replacement (SA) repRNAs. In embodiments, the repRNA comprising one or more circularized 3′ replacement (SA) repRNAs improves stability as compared to an unmodified form and is resistant to exonucleases. In embodiments, the repRNA comprises at least one or more circularized internal replacement (SD+SA) repRNAs. In embodiments, the repRNA comprises at least one or more circularized internal replacement (SD+SA) repRNAs stabilize the repRNA. In embodiments, the repRNA comprising one or more circularized internal replacement (SD+SA) repRNAs improves stability as compared to an unmodified form and is resistant to exonucleases.

In embodiments, the composition or system further comprises a repair RNA (repRNA), a small RNA that induces cleavage in an RNA, and a CRISPR/Cas system. In embodiments, the CRISPR/Cas system comprises a guide RNA (gRNA) and a repRNA in cis or trans capable of Cas protein binding and trans-splicing. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system. In embodiments, the, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA), a small RNA that induces cleavage in an RNA, and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx 10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms or is within an RNP, a protein within the RNP, and/or a nucleic acid encoding the protein that forms or is within the RNP comprises a modification or mutation that attenuates, weakens, reduces, decreases, or ablates RNP activity, and/or leads to attenuation of RNA modifying activity as compared to an unmodified form, the RNP activity optionally being selected from cleavage, nucleic acid processing, pseudouridylation, and/or methylation. In embodiments, the snRNA, snoRNA, protein that forms or is within an RNP, and/or a nucleic acid encoding the protein that forms or is within the RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the snRNA, snoRNA, protein that forms or is within an RNP, and/or a nucleic acid encoding the protein that forms or is within the RNP comprises at least one or more pseudouridylation sites. In embodiments, the snRNA, snoRNA, protein that forms or is within an RNP, and/or a nucleic acid encoding the protein that forms or is within the RNP comprises no pseudouridylation sites. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In embodiments, the composition or system comprises a repair RNA (repRNA) and/or a protein that forms or is within an RNP, with the snRNA or snoRNA, or a nucleic acid encoding the protein that forms, or is within, the RNP and/or a small RNA that induces cleavage in an RNA and/or a CRISPR/Cas system. In embodiments, the composition or system comprises a repair RNA (repRNA) and/or a protein that forms or is within an RNP, with the snRNA or snoRNA, or a nucleic acid encoding the protein that forms, or is within, the RNP and/or a small RNA that induces cleavage in an RNA and/or a CRISPR/Cas system when the present methods are undertaken in cis or trans, as described herein. In embodiments, cleavage is initiated from RNPs that are formed on the repRNA, or from RNPs that are formed in cis or trans.

In embodiments, the composition or system comprises a chimeric CRISPR-Cas gRNA and a repRNA in cis or trans capable of Cas protein binding and trans-splicing.

In embodiments, the snRNA or snoRNA comprises an Sm sequence motif, and/or wherein the snRNA or snoRNA comprises an antisense region sequence (ASR). In embodiments, the Sm sequence motif assembles with an Sm or Lsm protein into an RNP. In embodiments, the Sm or Lsm proteins are selected from a B/B′, D3, D2, D1, E, F, G, LSm5, LSm7, LSm4, LSm8, LSm2, LSm3, LSm6 and LSm10 proteins.

In embodiments, the snRNA or snoRNA is at least about 10 to about 80 nucleotides, about 10 to about 70 nucleotides, about 10 to about 60 nucleotides, about 10 to about 50 nucleotides, about 10 to about 40 nucleotides, about 10 to about 30 nucleotides, about 10 to about 20 nucleotides, or about 80, 70, 60, 50, 40, 30, 20, or 10 nucleotides in length. In embodiments, wherein the snRNA or snoRNA comprises an antisense region sequence (ASR) selected from SEQ ID NOs: 700-707.

In embodiments, the Sm sequence motif comprises a nucleotide sequence selected from SEQ ID NOs: 1-8.

In embodiments, the snRNA or snoRNA comprises a guide repair RNA (grepRNA) sequence. In embodiments, wherein the grepRNA sequence is selected from SEQ ID NOs: 708-711.

In embodiments, composition or system comprises a splice acceptor. In embodiments, composition or system comprises a splice donor.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans. In embodiments, these elements are under the control of one or more promoters. In embodiments, these elements are under the control of different promoters. In embodiments, these elements are operably linked, but separated by a cleavable sequence (e.g., a self-cleaving ribozyme). In embodiments, (i) multiple populations of repRNA are under the control of different promoters or (ii) the repRNA and another system member under control of different promoters.

In embodiments, the at least one intronic sequence comprises one or more splicing signals. In embodiments, the one or more splicing signals are selected from an exonic splicing enhancer (ESE), an intronic splicing enhancer (ISE), an exonic splicing silencer (ESS), intronic splicing silencer (ISS), a U1 binding motif (e.g., among other snRNA binding motifs), a polypyrimidine tract, a branch point, and a combination thereof.

In embodiments, the at least one intronic sequence comprises a branch point and a polypyrimidine tract.

In embodiments, the composition or system comprises 1, 2, 3, 4, 5, 6, 7, 8, 9, or 10 exons.

In embodiments, the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length.

In embodiments, wherein the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length.

In embodiments, the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences.

In embodiments, when the composition or system is introduced to the cell an exon in the pre-mRNA is targeted for trans-splicing. In embodiments, the target sequence is positioned in a region of the pre-mRNA comprising the exon targeted for trans-splicing. In embodiments, the target sequence is positioned proximal to a splice site. In embodiments, the target sequence is positioned proximal to a splice donor or a splice acceptor.

In embodiments, further comprising one or more binding domain sequences of about 4 to about 300 nucleotides each with complementarity to a pre-mRNA target sequence. In embodiments the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length. In embodiments the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length. In embodiments the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences. In embodiments, the small nuclear RNA (snRNA) or a small nucleolar RNA (snoRNA) sequence is greater than about 5 nucleotides in length which forms a secondary structure, and optionally comprises a sequence motif to direct the one or more binding domains to the pre-mRNA target sequence. In embodiments, small nuclear RNA (snRNA) or a small nucleolar RNA (snoRNA) sequence is about 5 to about 500, or 5 to about 400, or 5 to about 300, or 5 to about 200, or 5 to about 100, or 5 to about 90, or 5 to about 80, or 5 to about 70, or 5 to about 60, or 5 to about 50, or 5 to about 40, or 5 to about 30, or 5 to about 20, or 5 to about 10, or 7 to about 500, or 7 to about 400, or 7 to about 300, or 7 to about 200, or 7 to about 100, or 7 to about 90, or 7 to about 80, or 7 to about 70, or 7 to about 60, or 7 to about 50, or 7 to about 40, or 7 to about 30, or 7 to about 20, or 7 to about 10 nucleotides in length.

In embodiments, the composition comprises at least one exonic sequence.

In embodiments, the composition or system comprises a CRISPR/Cas system, e.g., a protein and/or nucleic acid thereof. In embodiments, the composition or system further comprises a repair RNA (repRNA) and a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA) and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type

V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In aspects, the present disclosure provides a composition or system for targeting trans-splicing of a pre-mRNA in a cell, comprising one or more nucleic acids comprising one or more nucleotide sequences comprising from 5′ to 3′ at least one intronic sequence comprising a small nuclear RNA (snRNA), or a small nucleolar RNA (snoRNA) sequence, a splice acceptor and/or splice donor sequence.

In embodiments, when the composition or system is introduced to the cell an exon in the pre-mRNA is targeted for trans-splicing. In embodiments, the target sequence is positioned upstream the exon in the pre-mRNA targeted for trans-splicing. In embodiments, the target sequence is positioned proximal to a splice site. In embodiments, the target sequence is positioned proximal to a splice acceptor or a splice donor. In embodiments, trans-splicing occurs between a splice donor upstream the exon in the pre-mRNA and the splice acceptor of the composition or system. In embodiments, trans-splicing results in ligation of the 3′ end of an exon upstream the splice donor in the pre-mRNA with the 5′ end of the at least one exonic sequence of the composition or system. In embodiments, the one or more splicing signals comprises a branch point and a polypyrimidine tract.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA. In embodiments, the snoRNA comprises an H/ACA box or C/D box. In embodiments, the snRNA or snoRNA sequence assembles into an RNP. In embodiments, the snRNA or snoRNA sequence comprises a sequence motif that assembles into an RNP. In embodiments, the composition or system further comprises a protein that forms or is within an RNP, with the snRNA or snoRNA, or a nucleic acid encoding the protein that forms, or is within, the RNP. In embodiments, the snRNA, snoRNA, protein that forms or is within an RNP, and/or a nucleic acid encoding the protein that forms or is within the RNP comprises a modification or mutation that attenuates, weakens, reduces, decreases, or ablates RNP activity, and/or leads to attenuation of RNA modifying activity as compared to an unmodified form, the RNP activity optionally being selected from cleavage, nucleic acid processing, pseudouridylation, and/or methylation. In embodiments, the snRNA, snoRNA, protein that forms or is within an RNP, and/or a nucleic acid encoding the protein that forms or is within the RNP comprises a modification or mutation that increases, stimulates, or enhances RNP activity, or enhances RNA modifying activity, the RNP activity optionally being selected from cleavage, nucleic acid processing, pseudouridylation, and/or methylation. In embodiments, the snRNA, snoRNA, protein that forms or is within an RNP, and/or a nucleic acid encoding the protein that forms or is within the RNP comprises at least one or more pseudouridylation sites. In embodiments, the snRNA, snoRNA, protein that forms or is within an RNP, and/or a nucleic acid encoding the protein that forms or is within the RNP comprises no pseudouridylation sites. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In embodiments, further comprising one or more binding domain sequences of about 4 to about 300 nucleotides each with complementarity to a pre-mRNA target sequence. In embodiments the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length. In embodiments the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length. In embodiments the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences. In embodiments, the small nuclear RNA (snRNA) or a small nucleolar RNA (snoRNA) sequence is greater than about 5 nucleotides in length which forms a secondary structure, and optionally comprises a sequence motif to direct the one or more binding domains to the pre-mRNA target sequence. In embodiments, small nuclear RNA (snRNA) or a small nucleolar RNA (snoRNA) sequence is about 5 to about 500, or 5 to about 400, or 5 to about 300, or 5 to about 200, or 5 to about 100, or 5 to about 90, or 5 to about 80, or 5 to about 70, or 5 to about 60, or 5 to about 50, or 5 to about 40, or 5 to about 30, or 5 to about 20, or 5 to about 10, or 7 to about 500, or 7 to about 400, or 7 to about 300, or 7 to about 200, or 7 to about 100, or 7 to about 90, or 7 to about 80, or 7 to about 70, or 7 to about 60, or 7 to about 50, or 7 to about 40, or 7 to about 30, or 7 to about 20, or 7 to about 10 nucleotides in length.

In embodiments, the composition comprises at least one exonic sequence.

In embodiments, the composition or system comprises a CRISPR/Cas system, e.g., a protein and/or nucleic acid thereof. In embodiments, the composition or system further comprises a repair RNA (repRNA) and a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12c (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA) and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx 10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI. In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In aspects, the present disclosure provides a composition or system for targeting trans-splicing of a pre-mRNA in a cell, comprising one or more nucleic acids comprising one or more nucleotide sequences comprising from 5′ to 3′ comprising at least one intronic sequence comprising a small nuclear RNA (snRNA), or a small nucleolar RNA (snoRNA) sequence, and/or splice donor sequence, wherein the snRNA or snoRNA forms a secondary structure and/or comprises a sequence motif to direct the one or more binding domains to the pre-mRNA target sequence.

In embodiments, when the composition or system is introduced to the cell an exon in the pre-mRNA is targeted for trans-splicing. In embodiments, the target sequence is positioned downstream the exon in the pre-mRNA. In embodiments, the target sequence is positioned proximal to a splice site. In embodiments, the target sequence is positioned proximal to a splice donor or a splice acceptor.

In embodiments, trans-splicing occurs between the splice donor of the nucleic acid and a splice acceptor downstream the exon in the pre-mRNA.

In embodiments, trans-splicing results in ligation of the 3′ end of the at least one exonic sequence of the nucleic acid with the 5′ end of an exon downstream the splice acceptor in the pre-mRNA.

In embodiments, the intronic sequence is an snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA assembles into an snRNP. In embodiments, the snRNA is a U7 snRNA. In embodiments, the U7 snRNA assembles into a U7 RNP. In embodiments, the snRNA is a U1 snRNA. In embodiments, the U1 snRNA assembles into a U1 RNP. In embodiments, the snRNA is a U11 snRNA. In embodiments, the U11 snRNA assembles into a U11 RNP. The composition or system of any one of the embodiments described herein, wherein the snRNA or snoRNA sequence comprises an Sm sequence motif, and/or the snRNA or snoRNA comprises an antisense region sequence (ASR).

In embodiments, the snRNA or snoRNA is at least about 10 to about 80 nucleotides, about 10 to about 70 nucleotides, about 10 to about 60 nucleotides, about 10 to about 50 nucleotides, about 10 to about 40 nucleotides, about 10 to about 30 nucleotides, about 10 to about 20 nucleotides, or about 80, 70, 60, 50, 40, 30, 20, or 10 nucleotides in length.

In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity as compared to an unmodified form, and/or leads to attenuation of RNA modifying activity, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the snRNA, snoRNA, protein that forms or is within an RNP, and/or a nucleic acid encoding the protein that forms or is within the RNP comprises at least one or more pseudouridylation sites. In embodiments, the snRNA, snoRNA, protein that forms or is within an RNP, and/or a nucleic acid encoding the protein that forms or is within the RNP comprises no pseudouridylation sites. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In embodiments, the snRNA or snoRNA comprises an antisense region sequence (ASR) selected from SEQ ID NOs: 700-707. In embodiments, the Sm sequence motif comprises a nucleotide sequence selected from SEQ ID NOs: 1-8.

In embodiments, the snRNA or snoRNA comprises a grepRNA sequence. In embodiments, the grepRNA sequence is selected from SEQ ID NOs: 708-711.

In embodiments, the snRNA or snoRNA sequence comprises an Sm sequence motif, and/or the snRNA or snoRNA comprises an antisense region sequence (ASR), and a U7 snRNA. In embodiments, the Sm sequence motif comprises a sequence set forth in SEQ ID NOs: 3 and 4.

In embodiments, the Sm sequence motif assembles with an Sm protein into an RNP. In embodiments, the Sm protein is selected from a B/B′, D3, D2, D1, E, F, and G Sm protein.

In embodiments, the snRNA or snoRNA sequence comprises a sequence having at least 80% sequence identity to a sequence selected from SEQ ID NOs: 9-589 or a portion thereof. In embodiments, the snRNA or snoRNA sequence comprises a region of about 7 to about 40 nucleotides in length, wherein the region comprises an Sm sequence motif, and/or wherein the snRNA or snoRNA comprises an antisense region sequence (ASR).

In embodiments, the snRNA or snoRNA comprises an antisense region sequence (ASR) selected from SEQ ID NOs: 700-707. In embodiments, the snRNA or snoRNA is at least about 10 to about 80 nucleotides, about 10 to about 70 nucleotides, about 10 to about 60 nucleotides, about 10 to about 50 nucleotides, about 10 to about 40 nucleotides, about 10 to about 30 nucleotides, about 10 to about 20 nucleotides, or about 80, 70, 60, 50, 40, 30, 20, or 10 nucleotides in length. In embodiments, the snRNA or snoRNA sequence comprises a region of about 40 to about 300 nucleotides in length, wherein the region comprises a secondary structure and/or an Sm sequence motif. In embodiments, the composition or system comprises one binding domain sequence. In embodiments, the composition or system comprises more than one binding domain sequence. In embodiments, the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In embodiments, further comprising one or more binding domain sequences of about 4 to about 300 nucleotides each with complementarity to a pre-mRNA target sequence. In embodiments the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length. In embodiments the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length. In embodiments the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences. In embodiments, the small nuclear RNA (snRNA) or a small nucleolar RNA (snoRNA) sequence is greater than about 5 nucleotides in length which forms a secondary structure, and optionally comprises a sequence motif to direct the one or more binding domains to the pre-mRNA target sequence. In embodiments, small nuclear RNA (snRNA) or a small nucleolar RNA (snoRNA) sequence is about 5 to about 500, or 5 to about 400, or 5 to about 300, or 5 to about 200, or 5 to about 100, or 5 to about 90, or 5 to about 80, or 5 to about 70, or 5 to about 60, or 5 to about 50, or 5 to about 40, or 5 to about 30, or 5 to about 20, or 5 to about 10, or 7 to about 500, or 7 to about 400, or 7 to about 300, or 7 to about 200, or 7 to about 100, or 7 to about 90, or 7 to about 80, or 7 to about 70, or 7 to about 60, or 7 to about 50, or 7 to about 40, or 7 to about 30, or 7 to about 20, or 7 to about 10 nucleotides in length.

In embodiments, the composition comprises at least one exonic sequence.

In embodiments, the composition or system comprises a CRISPR/Cas system, e.g., a protein and/or nucleic acid thereof. In embodiments, the composition or system further comprises a repair RNA (repRNA) and a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA) and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In aspects, the present disclosure provides a composition or system for targeting trans-splicing of a pre-mRNA in a cell, comprising one or more nucleic acids comprising one or more nucleotide sequences comprising from 5′ to 3′ at least one intronic sequence comprising a small nuclear RNA (snRNA), or a small nucleolar RNA (snoRNA) sequence, and/or a splice acceptor. In embodiments, described herein is a composition or system for targeting trans-splicing of a pre-mRNA in a cell, comprising one or more nucleic acids comprising one or more nucleotide sequences comprising from 5′ to 3′: at least one intronic sequence comprising: (i) a snRNA or snoRNA sequence comprising an H/ACA box or a C/D box and one or more binding domain sequences of about 4 to about 30 nucleotides each with complementarity to a pre-mRNA target sequence; and (ii) one or more splicing signals; a splice acceptor; and at least one exonic sequence.

In embodiments, when the composition or system is introduced to the cell an exon in the pre-mRNA is targeted for trans-splicing. In embodiments, the target sequence is positioned upstream the exon in the pre-mRNA. In embodiments, the target sequence is positioned proximal to a splice site. In embodiments, the target sequence is positioned proximal to a splice donor or splice acceptor. In embodiments, trans-splicing occurs between a splice donor upstream the exon in the pre-mRNA and the splice acceptor of the composition or system. In embodiments, trans-splicing results in ligation of the 3′ end of an exon upstream the splice donor in the pre-mRNA with the 5′ end of the at least one exonic sequence of the composition or system.

In embodiments, the one or more splicing signals comprises a branch point and a polypyrimidine tract.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA. In embodiments, the snoRNA comprises an H/ACA box or C/D box. In embodiments, the snRNA or snoRNA sequence assembles into an RNP. In embodiments, the snRNA or snoRNA sequence comprises a sequence motif that assembles into an RNP. In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity as compared to an unmodified form, and/or leads to attenuation of RNA modifying activity, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In embodiments, further comprising one or more binding domain sequences of about 4 to about 300 nucleotides each with complementarity to a pre-mRNA target sequence. In embodiments the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length. In embodiments the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length. In embodiments the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences. In embodiments, the small nuclear RNA (snRNA) or a small nucleolar RNA (snoRNA) sequence is greater than about 5 nucleotides in length which forms a secondary structure, and optionally comprises a sequence motif to direct the one or more binding domains to the pre-mRNA target sequence. In embodiments, small nuclear RNA (snRNA) or a small nucleolar RNA (snoRNA) sequence is about 5 to about 500, or 5 to about 400, or 5 to about 300, or 5 to about 200, or 5 to about 100, or 5 to about 90, or 5 to about 80, or 5 to about 70, or 5 to about 60, or 5 to about 50, or 5 to about 40, or 5 to about 30, or 5 to about 20, or 5 to about 10, or 7 to about 500, or 7 to about 400, or 7 to about 300, or 7 to about 200, or 7 to about 100, or 7 to about 90, or 7 to about 80, or 7 to about 70, or 7 to about 60, or 7 to about 50, or 7 to about 40, or 7 to about 30, or 7 to about 20, or 7 to about 10 nucleotides in length.

In embodiments, the composition comprises at least one exonic sequence.

In embodiments, the composition or system comprises a CRISPR/Cas system, e.g., a protein and/or nucleic acid thereof. In embodiments, the composition or system further comprises a repair RNA (repRNA) and a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA) and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In aspects, the present disclosure provides a composition or system for targeting trans-splicing of a pre-mRNA in a cell, comprising one or more nucleic acids comprising one or more nucleotide sequences comprising from 5′ to 3′: (a) at least one exonic sequence; (b) a splice donor; and (c) at least one intronic sequence comprising a snRNA or snoRNA sequence comprising an H/ACA box or a C/D box and one or more binding domain sequences of about 4 to about 30 nucleotides each with complementarity to a pre-mRNA target sequence.

In embodiments, when the composition or system is introduced to the cell an exon in the pre-mRNA is targeted for trans-splicing. In embodiments, the target sequence is positioned downstream the exon in the pre-mRNA. In embodiments, the target sequence is positioned proximal to a splice site. In embodiments, the target sequence is positioned proximal to a splice donor or a splice acceptor.

In embodiments, trans-splicing occurs between the splice donor of the composition or system and a splice acceptor downstream the exon in the pre-mRNA. In embodiments, trans-splicing results in ligation of the 3′ end of the at least one exonic sequence of the composition or system with the 5′ end of an exon downstream the splice acceptor in the pre-mRNA.

In embodiments, the snRNA or snoRNA sequence comprises an H/ACA box comprising 5′ to 3′ an H consensus sequence and an ACA consensus sequence. In embodiments, the composition or system comprises at least one binding domain sequence positioned: (i) upstream the H consensus sequence; (ii) downstream the ACA consensus sequence; (iii) between the H consensus sequence and the ACA consensus sequence; or (iv) a combination of (i)-(iii).

In embodiments, the snRNA or snoRNA sequence comprises C/D box comprising 5′ to 3′ a C consensus sequence, a D′ consensus sequence, a C′ consensus sequence, and a D consensus sequence.

In embodiments, the composition or system comprises at least one binding domain positioned (i) upstream the C consensus sequence; (ii) between the C consensus sequence and the D′ consensus sequence; (iii) between the D′ consensus sequence and the C′ consensus sequence; (iv) between the C′ consensus sequence and the D consensus sequence; (v) downstream the D consensus sequence; or (vi) a combination of (i)-(v).

In embodiments, the snRNA or snoRNA sequence comprises a sequence having at least 80% sequence identity to a sequence selected from SEQ ID NOs: 590-657 and 732-793, or a portion thereof. In embodiments, the snRNA or snoRNA sequence comprises a region of about 40 to about 300 nucleotides in length and comprising an H consensus sequence and an ACA consensus sequence.

In embodiments, the snRNA or snoRNA sequence comprises one binding domain sequence. In embodiments, the snRNA or snoRNA sequence comprises more than one binding domain sequence. In embodiments, the composition or system comprises at least one binding domain sequence with full complementarity to the pre-mRNA target sequence. In embodiments, the composition or system comprises at least one binding domain sequence with partial complementarity to the pre-mRNA target sequence. In embodiments, the at least one binding domain sequence comprises one or more mismatches relative to the pre-mRNA target sequence.

In embodiments, the at least one binding domain sequence has at least 95% complementarity to the pre-mRNA target sequence.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In embodiments, the composition or system comprises a sequence up to about 20,000 nucleotides in length.

In embodiments, the composition or system comprises a sequence of about 50 to about 500, about 50 to about 1000, about 100 to about 500, about 100 to about 1000, about 500 to about 1000, about 500 to about 2000, about 500 to about 3,000, about 500 to about 4,000, about 500 to about 5,000, about 1,000 to about 5,000, about 1,000 to about 10,000, about 5,000 to about 15,000, or about 5,000 to about 20,000 nucleotides in length.

In embodiments, the composition or system is introduced to the cell as an RNA. In embodiments, the composition or system is introduced to the cell as a DNA.

In embodiments, the composition or system is introduced to the cell by a single viral vector. In embodiments, the viral vector is an AAV.

In embodiments, the composition or system is introduced to the cell by a non-viral vector.

In embodiments, the introduction of the composition or system to the cell results in an efficiency of trans-splicing that is greater than a composition or system lacking the snRNA or snoRNA sequence. In embodiments, the efficiency of trans-splicing is greater than about 10%, about 20%, about 30%, about 40%, about 50%, about 60%, about 70%, about 80%, about 90%, or about 99%.

In embodiments, introduction of the composition or system to the cell is without any additional protein to guide activity.

In embodiments, introduction of the composition or system to the cell results in an immunogenicity that is less than a composition or system lacking the snRNA or snoRNA sequence.

In embodiments, the composition or system is formulated as a lipid nanoparticle.

In aspects, the present disclosure provides a viral vector comprising the composition or system of any one of the embodiments described herein.

In aspects, the present disclosure provides a lipid nanoparticle comprising the composition or system of any one of the embodiments described herein.

In aspects, the present disclosure provides a cell comprising the composition or system of any one of the embodiments described herein, the viral vector of any one of the embodiments described herein, or the lipid nanoparticle of any one of the embodiments described herein.

In aspects, the present disclosure provides a pharmaceutical composition comprising the composition or system of any one of the embodiments described herein, the viral vector of any one of the embodiments described herein, or the lipid nanoparticle of any one of the embodiments described herein, and a pharmaceutically acceptable carrier.

In aspects, the present disclosure provides a pharmaceutical composition comprising the cell of any one of the embodiments described herein, and a pharmaceutically acceptable carrier.

In aspects, the present disclosure provides a method of targeting trans-splicing of a pre-mRNA in a cell, the method comprising contacting the cell with the composition or system of any one of the embodiments described herein, the viral vector of any one of the embodiments described herein, or the lipid nanoparticle of any one of the embodiments described herein, or the pharmaceutical composition of any one of the embodiments described herein, wherein when the composition or system, the viral vector, the lipid nanoparticle, or the pharmaceutical composition contacts the cell, the one or more binding domain sequences bind to the pre-mRNA, thereby targeting the pre-mRNA for trans-splicing.

In aspects, the present disclosure provides a method of correcting a mutation in a pre-mRNA in a cell, the method comprising contacting the cell with the composition or system of any one of the embodiments described herein, the viral vector of any one of the embodiments described herein, or the lipid nanoparticle of any one of the embodiments described herein, or the pharmaceutical composition of any one of the embodiments described herein, wherein when the composition or system, the viral vector, the lipid nanoparticle, or the pharmaceutical composition contacts the cell, the one or more binding domain sequences bind to the pre-mRNA at a location proximal to the mutation, and wherein trans-splicing replaces one or more exons in the pre-mRNA comprising the mutation, thereby correcting the mutation.

In aspects, the present disclosure provides a method of treating a patient with a disease or disorder associated with a mutation in a pre-mRNA, the method comprising administering to the patient an effective amount of the composition or system of any one of the embodiments described herein, the viral vector of any one of the embodiments described herein, or the lipid nanoparticle of any one of the embodiments described herein, or the pharmaceutical composition of any one of the embodiments described herein, wherein when the composition or system, the viral vector, the lipid nanoparticle, or the pharmaceutical composition is administered, the one or more binding domain sequences bind to the pre-mRNA at a location proximal to the mutation, and wherein trans-splicing replaces one or more exons in the pre-mRNA comprising the mutation, thereby correcting the mutation.

In embodiments, the present methods are conducted as essentially in FIG. 7A, FIG. 8A, and FIG. 10.

In embodiments, trans-splicing results in an mRNA that alleviates the disease or does not cause or contribute to the disease.

In embodiments, the composition or system of any one of the embodiments described herein, the viral vector of any one of the embodiments described herein, or the lipid nanoparticle of any one of the embodiments described herein, or the pharmaceutical composition of any one of the embodiments described herein for use in treating a patient with a disease or disorder associated with a mutation in a pre-mRNA, the treatment comprising administering to the patient the composition or system, the viral vector, the lipid nanoparticle, or the pharmaceutical composition, wherein when the composition or system, the viral vector, the lipid nanoparticle, or the pharmaceutical composition is administered, the one or more binding domain sequences bind to the pre-mRNA at a location proximal to the mutation, and wherein trans-splicing replaces one or more exons in the pre-mRNA comprising the mutation, thereby correcting the mutation.

In embodiments, the composition or system of any one of the embodiments described herein, the viral vector of any one of the embodiments described herein, or the lipid nanoparticle of any one of the embodiments described herein, or the pharmaceutical composition of any one of the embodiments described herein for the manufacture of a medicament for use in treating a patient with a disease or disorder associated with a mutation in a pre-mRNA, the treatment comprising administering to the patient the medicament, wherein when the medicament is administered, the one or more binding domain sequences of the composition or system binds to the pre-mRNA at a location proximal to the mutation, and wherein trans-splicing replaces one or more exons in the pre-mRNA comprising the mutation, thereby correcting the mutation.

In embodiments, the present methods are conducted as essentially in FIG. 7A, FIG. 8A, and FIG. 10.

In aspects, the present disclosure provides a kit comprising a container comprising the composition or system of any one of the embodiments described herein, the viral vector of any one of the embodiments described herein, or the lipid nanoparticle of any one of the embodiments described herein, or the pharmaceutical composition of any one of the embodiments described herein, with instructions for use in correcting a mutation in a pre-mRNA.

In embodiment, the present disclosure provides a composition or system of any one of the embodiments described herein, the viral vector of any one of the embodiments described herein, the lipid nanoparticle of any one of the embodiments described herein, the pharmaceutical composition of any one of the embodiments described herein, the method of any one of any one of the embodiments described herein, or the kit of any one of the embodiments described herein, wherein the trans-splicing system comprises an active cas nuclease. In embodiments, wherein the active cas nuclease is a Type III CRISPR/Cas system, or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, Type IIIF CRISPR/Cas system, optionally wherein the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6.

In aspects, the present disclosure provides a method of slowing, reducing, or ablating transcription of a target gene, and/or stimulating, enhancing, or increasing the Pol II stalling/release. In embodiments, this method increases trans-splicing, without wishing to be bound by theory, by forcing G4 or similarly bulky RNA-RNA motif formation downstream of the repRNA's binding motif as compared to an unmodified form.

In aspects, the present disclosure provides compositions, systems, and/or methods for separate delivery of at least one repRNA capable of trans-splicing at least one other repRNA (e.g., daisychain) and/or an endogenous RNA target, e.g., for multi-kilobase edits larger than the cargo capacity of a single AAV. In embodiments, this permits single AAV delivery, e.g., by effectively reducing the need for a cargo size that is greater than AAV loading capacity.

In aspects, the present disclosure provides a method for modifying and converting an endogenous RNA, such as and without limitation a pre-mRNA, mRNA, lncRNA, into a repRNA for trans-splicing.

In aspects, disclosed herein is a composition or system for targeting trans-splicing of a pre-mRNA in a cell, comprising one or more nucleic acids comprising one or more nucleotide sequences comprising from 5′ to 3′: (a) a splice donor and/or a splice acceptor; and (b) at least one intronic sequence comprising a snRNA or snoRNA sequence comprising an H/ACA box or a C/D box and one or more binding domain sequences of about 4 to about 300 nucleotides each with complementarity to a pre-mRNA target sequence.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the binding domain sequence is between about 1 nucleotide, or about 5 nucleotides, or about 10 nucleotides, or about 50 nucleotides, or about 100 nucleotides, or about 1000 nucleotides near a H/ACA box or C/D box sequence.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is a H/ACA snoRNA (SNORA), C/D snoRNA (SNORD), or a small cajal RNA (scaRNA).

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is SNORA101B, SNORA48, SNORA54, SNORA66, SNORA73A, or SNORA8, or the snoRNA is SNORA101B, SNORA48, SNORA54, SNORA66, SNORA73A, or SNORA8 with at least one or more mutations.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is in cis with respect to the repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is in trans with respect to the repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the repRNA comprises a splice donor and/or a splice acceptor.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the repRNA comprises a splice donor and/or a splice acceptor, and wherein the snoRNA and snRNA are in trans with respect to the repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein a mutation in H and/or ACA motifs substantially decreases the trans-splicing efficiency.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 3′ end of a 5′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the addition of a SNORA modification on a transcript stabilizes the 3′ end of the transcript after ribozyme cleavage and polyA removal.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 5′ end of a 3′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 3′ end of a 3′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 5′ end of a 5′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the SNORA sequence has at least about 80%, or at least about 85%, or at least about 90%, or at least about 95%, or at least about 96%, or at least about 97%, or at least about 98%, or at least about 99% identity to any one of SEQ ID NOs: 590-657 or 732-793.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE FIGURES

FIG. 1A and FIG. 1B provide schematics, without wishing to be bound by theory, depicting cis-splicing of a pre-mRNA (FIG. 1A) and trans-splicing between two pre-mRNA molecules (FIG. 1B). “SD” refers to a splice donor. “SA” refers to a splice acceptor.

FIG. 1C provides a schematic, without wishing to be bound by theory, depicting an exemplary composition, system, and/or the one or more nucleic acids comprising one or more nucleotide sequences described herein for targeted trans-splicing of a pre-mRNA. Labeling of the composition, system, and/or the one or more nucleic acids comprising one or more nucleotide sequences described herein indicates the segments corresponding to an RNA binding domain, a non-coding RNA (ncRNA), SA, and exon. Labeling of the pre-mRNA indicates segments of the pre-mRNA corresponding to the 5′ exon, SD, SA, and 3′ exon.

FIG. 1D provides a schematic, without wishing to be bound by theory, depicting a predicted secondary structure of an exemplary composition, system, and/or the one or more nucleic acids comprising one or more nucleotide sequences described herein and pre-mRNA and the interactions between the composition, system, and/or the one or more nucleic acids comprising one or more nucleotide sequences described herein and pre-mRNA that yield a trans-splicing mRNA product.

FIG. 1E provides a graph showing the proportion of ncRNAs identified as containing a sequence motif (Sm sequence motif, H/ACA box, and/or C/D box).

FIG. 1F provides a graph showing the length in number of nucleotides for exemplary ncRNAs of the disclosure.

FIG. 2A, FIG. 2B, and FIG. 2C provide a schematic, without wishing to be bound by theory, depicting the extended and folded secondary structure of an exemplary composition, system, and/or the one or more nucleic acids comprising one or more nucleotide sequences described herein having 5′ to 3′ an RNA binding domain, an ncRNA having an Sm sequence motif and a U7 snRNA, intron, SA, and exon (FIG. 2A) and the interaction of the exemplary composition, system, and/or the one or more nucleic acids comprising one or more nucleotide sequences described herein with a target pre-mRNA that initiates a trans-splicing event between the SD of the pre-mRNA and the SA of the exemplary composition, system, and/or the one or more nucleic acids comprising one or more nucleotide sequences described herein (FIG. 2A, FIG. 2B, and FIG. 2C).

FIG. 3A, FIG. 3B, and FIG. 3C provide a schematic, without wishing to be bound by theory, depicting the extended and folded secondary structure of an exemplary composition, system, and/or the one or more nucleic acids comprising one or more nucleotide sequences described herein having 5′ to 3′ an RNA binding domain, U1 snRNA, intron, SA, and exon (FIG. 3A) and the interaction of the exemplary composition, system, and/or the one or more nucleic acids comprising one or more nucleotide sequences described herein with a target pre-mRNA that initiates a trans-splicing event between the SD of the pre-mRNA and the SA of the exemplary composition, system, and/or the one or more nucleic acids comprising one or more nucleotide sequences described herein (FIG. 3A, FIG. 3B, and FIG. 3C).

FIG. 4A, FIG. 4B, and FIG. 4C provide a schematic, without wishing to be bound by theory, depicting the extended and folded secondary structure of a composition, system, and/or the one or more nucleic acids comprising one or more nucleotide sequences described herein having 5′ to 3′ an RNA binding domain, U11 snRNA, intron, SA, and exon (FIG. 4A) and the interaction of the exemplary composition, system, and/or the one or more nucleic acids comprising one or more nucleotide sequences described herein with a target pre-mRNA that initiates a trans-splicing event between the SD of the pre-mRNA and the SA of the exemplary composition, system, and/or the one or more nucleic acids comprising one or more nucleotide sequences described herein (FIG. 4A, FIG. 4B, and FIG. 4C).

FIG. 5A, FIG. 5B, and FIG. 5C provides a schematic, without wishing to be bound by theory, depicting the extended and folded secondary structure of an exemplary composition, system, and/or the one or more nucleic acids comprising one or more nucleotide sequences described herein having 5′ to 3′ an RNA binding domain, an ncRNA having an Sm sequence motif, intron, SA, and exon (FIG. 5A) and the interaction of the exemplary composition, system, and/or the one or more nucleic acids comprising one or more nucleotide sequences described herein with a target pre-mRNA that initiates a trans-splicing event between the SD of the pre-mRNA and the SA of the exemplary composition, system, and/or the one or more nucleic acids comprising one or more nucleotide sequences described herein (FIG. 5B, and FIG. 5C).

FIG. 6A, FIG. 6B, and FIG. 6C provides a schematic, without wishing to be bound by theory, depicting the extended and folded secondary structure of an exemplary composition, system, and/or the one or more nucleic acids comprising one or more nucleotide sequences described herein having 5′ to 3′ a snoRNA having an insertion of two RNA binding domains, intron, SA, and exon (FIG. 6A) and the interaction of the exemplary composition, system, and/or the one or more nucleic acids comprising one or more nucleotide sequences described herein with a target pre-mRNA that initiates a trans-splicing event between the SD of the pre-mRNA and the SA of the exemplary composition, system, and/or the one or more nucleic acids comprising one or more nucleotide sequences described herein (FIG. 6B, and FIG. 6C).

FIG. 7A and FIG. 7B show a schematic (FIG. 7A) and experimental data (FIG. 7B) demonstrating U7 guide RNAs (“gRNAs”) enhance Cas13-based trans-splicing. FIG. 7A provides a schematic, without wishing to be bound by theory, depicting a U7 gRNA enhancing Cas13-based trans-splicing. FIG. 7B is a graph showing data of the USH2A trans-splicing system and the combination U7 gRNAs with an RNA-targeting CRISPR-Cas (protein and CRISPR gRNA) and a repair RNA enables, compared to conditions where the U7 gRNA are not provided (RNA-targeting CRISPR-Cas system and repair RNA alone), or conditions where the U7 gRNA sequence is mutated, or conditions where the U7 gRNA is targeting a different sequence (non-targeting guide).

FIG. 8A and FIG. 8B show a schematic (FIG. 8A) and experimental data (FIG. 8B) demonstrating the combination of U7 gRNAs with a repRNA in trans, which enables exon replacement. FIG. 8A provides a schematic, without wishing to be bound by theory, depicting a U7 gRNA enhancing Cas13-based trans-splicing. FIG. 8B is a graph showing data of the USH2A trans-splicing system and the combination U7 gRNAs with a repair RNA in trans, which enables exon replacement, compared to conditions where the U7 gRNA is not provided (repRNA alone), or conditions where the gRNA sequence is mutated, or conditions where the U7 gRNA is targeting a different sequence (non-targeting guide).

FIG. 9 provides a schematic, without wishing to be bound by theory, depicting U7 grepRNA design for 3′ replacement.

FIG. 10A and FIG. 10B show experimental data demonstrating 3′ trans-splicing using a U7 grepRNA. FIG. 10A provides a schematic, without wishing to be bound by theory, depicting an experimental design to test 3′ trans-splicing and exon replacement using a U7 grepRNA, compared to a non-targeting ASR or a grepRNA lacking the U7 hairpin. FIG. 10B is a graph showing U7-guided trans-splicing on an USH2A target (3′ RNA replacement) with transient transfection leading to 45% trans-splicing efficiency.

FIG. 11 is a graph showing how the addition of SNORA48-like sequences to the 3′end of 5′ repRNAs significantly increased trans-splicing efficiency. FIG. 11 shows pATK0388 (SEQ ID NO: 713), pATK0945 (SEQ ID NO: 714), pATK0946 (SEQ ID NO: 715), pATK0947 (SEQ ID NO: 716), pATK0948 (SEQ ID NO: 717), pATK0949 (SEQ ID NO: 718), pATK0950 (SEQ ID NO: 719), pATK0973 (SEQ ID NO: 720), and pATK0974 (SEQ ID NO: 721).

FIG. 12A and FIG. 12B show experimental data showing snoRNAs broadly increase trans-splicing efficiency.

FIG. 13A is an image, and FIG. 13B and FIG. 13C are graphs showing experimental data illustrating how SNORA modifications on transcripts stabilize the 3′ ends of transcripts after ribozyme cleavage and polyA removal.

FIG. 14A is a graphical representation of a snoRNA-based trans-splice molecule. FIG. 14B is a graph showing how SNORA8 is permissive of extensions and deletions in the targeting regions.

FIG. 15A is an image, and FIG. 15B is a graph showing how repRNAs with SNORA modifications are delivered to cells via AAV to edit endogenous reporter RNAs.

FIG. 16A and FIG. 16B are graphs showing how repRNAs with SNORA modifications are delivered to cells via AAV to edit endogenous reporter RNAs.

FIG. 17 is a non-limiting pictorial representation of an AAV-deliverable snoRNA-based trans-splicing molecule.

FIG. 18 is a non-limiting graphical representation of various trans-splicing molecules that were modified to include snoRNA sequences (e.g., SEQ ID NOs: 732-793, Table 6). All snoRNA sequences containing trans-splicing repair RNAs demonstrated higher trans-splicing activity than trans-splicing repair RNAs which did not form human RNPs.

FIG. 19 is a non-limiting graphical representation of a repair RNA (repRNA) modified with snoRNAs (e.g., SEQ ID NOs: 732-793, Table 6). snoRNA trans-splicing molecules showed significantly higher trans-splicing activity compared to repRNA with a mutated snoRNA sequence that does not form human RNPs.

FIG. 20 is a non-limiting graphical representation of repRNAs with SNORA54 (as an illustrative example) being able to efficiently trans-splice independent of the size of the edit. Larger edits did not result in lower efficiency.

FIG. 21 is a non-limiting graphical representation of GFP correction using repRNAs with C/D-box snoRNA sequences enabling high trans-splicing rates.

FIG. 22 is a non-limiting graphical representation of GFP correction using repRNAs with C/D-box snoRNA sequences enabling high trans-splicing rates, showing RNP formation increases efficiency.

### DETAILED DESCRIPTION

The present disclosure provides a composition or system suitable for targeting trans-splicing of a pre-mRNA in a cell comprising one or more nucleic acids comprising one or more nucleotide sequences comprising at least one intronic sequence comprising a small nuclear RNA (snRNA), or a small nucleolar RNA (snoRNA) sequence, a splice acceptor and/or splice donor sequence. Without being bound by theory, the binding event brings the composition, system, and/or the one or more nucleic acids comprising one or more nucleotide sequences described herein into proximity of a region of the target RNA (e.g., pre-mRNA) selected for trans-splicing and recruits the spliceosome to the target RNA (e.g., pre-mRNA) such that efficient trans-splicing occurs. In some embodiments, the target RNA is a pre-mRNA. In some embodiments, the pre-mRNA comprises a nucleotide sequence comprising a disease-causing mutation. In some embodiments, the trans-splicing generates a mRNA comprising a desired alteration compared to an mRNA generated by cis-splicing of the pre-mRNA. For example, in some embodiments, the desired alteration is correction of a disease-causing mutation in the pre-mRNA.

Small nucleolar RNAs (snoRNAs) are small non-coding RNAs widely present in the nucleoli of eukaryotic cells and typically have a length of about 60-300 nucleotides. snoRNAs are mainly encoded by intronic regions of both protein coding and non-protein coding genes. Normally, snoRNAs can be mainly classified into three groups: H/ACA box snoRNAs, C/D box snoRNAs, and small cajal RNAs (scaRNAs).

Small nuclear RNAs (snRNAs) are recognized as key components of the spliceosome and are involved in the splicing of pre-mRNA. snRNAs can be complexed with many proteins to form RNA-protein complexes, which are known as small nuclear ribonucleoproteins (snRNPs), in the cell nucleus.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA.

In embodiments, the disclosure provides methods of targeting trans-splicing of a target RNA (e.g., pre-mRNA) in a cell, comprising introducing to the cell a composition, system, and/or the one or more nucleic acids comprising one or more nucleotide sequences described herein. In embodiments, the disclosure provides methods of correcting a mutation in at target RNA (e.g., a pre-mRNA) in a cell, comprising introducing to the cell a composition, system, and/or the one or more nucleic acids comprising one or more nucleotide sequences described herein. In some aspects, the introducing is performed in vivo. In some embodiments, the introducing is performed ex vivo. In some embodiments, the methods described herein are used to introduce a desired edit to a target nucleic acid edit in a manner that avoids certain disadvantages of gene-editing, e.g., gene-editing performed using a CRISPR/Cas system. Whereas gene-editing is associated with a risk of introducing a permanent and disease-causing off-target edit to the genome, the present disclosure provides methods of trans-splicing that avoid altering genomic DNA and enable transient editing. Thus, and without being bound by theory, the methods of the disclosure are used to introduce edits to nucleic acids in a cell in a manner that is safer than gene-editing. Additionally, in some embodiments, the methods of the disclosure are used to inactivate an undesirable off-target gene edit introduced to the genome, thereby preventing or ameliorating deleterious phenotypes associated with gene editing approaches.

In embodiments, introduction of the composition or system to the cell results in an immunogenicity that is less than a composition or system lacking the snRNA or snoRNA sequence. In embodiments, the composition or system is introduced to a cell by a single viral vector (e.g., AAV). In embodiments, introduction of the composition or system to the cell is without any additional protein to guide activity.

In embodiments, the disclosure provides methods for treating a disease or disorder in a subject in need thereof, the disease or disorder associated with (i) one or more genetic mutations, and/or (ii) an aberrant expression level and/or activity of a gene, or a transcriptional or translational product thereof, comprising administering to a subject composition, system, and/or the one or more nucleic acids comprising one or more nucleotide sequences described herein.

In embodiments, the disclosure provides methods and compositions for delivery of the composition, system, and/or the one or more nucleic acids comprising one or more nucleotide sequences described herein to a cell or a subject. In some embodiments, the composition, system, and/or the one or more nucleic acids comprising one or more nucleotide sequences described herein is delivered as a DNA. In some embodiments, the composition, system, and/or the one or more nucleic acids comprising one or more nucleotide sequences described herein is delivered as an RNA. In some aspects, the delivery comprises administering a recombinant expression vector (e.g., a viral vector, e.g., an AAV) comprising the splice editor nucleic acid molecule. In some aspects, the delivery comprises administering a non-viral vector (e.g., a lipid particle) comprising the splice editor nucleic acid molecule.

In embodiments, further comprising one or more binding domain sequences of about 4 to about 300 nucleotides each with complementarity to a pre-mRNA target sequence. In embodiments the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length. In embodiments the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length. In embodiments the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences. In embodiments, the small nuclear RNA (snRNA) or a small nucleolar RNA (snoRNA) sequence is greater than about 5 nucleotides in length which forms a secondary structure, and optionally comprises a sequence motif to direct the one or more binding domains to the pre-mRNA target sequence. In embodiments, small nuclear RNA (snRNA) or a small nucleolar RNA (snoRNA) sequence is about 5 to about 500, or 5 to about 400, or 5 to about 300, or 5 to about 200, or 5 to about 100, or 5 to about 90, or 5 to about 80, or 5 to about 70, or 5 to about 60, or 5 to about 50, or 5 to about 40, or 5 to about 30, or 5 to about 20, or 5 to about 10, or 7 to about 500, or 7 to about 400, or 7 to about 300, or 7 to about 200, or 7 to about 100, or 7 to about 90, or 7 to about 80, or 7 to about 70, or 7 to about 60, or 7 to about 50, or 7 to about 40, or 7 to about 30, or 7 to about 20, or 7 to about 10 nucleotides in length.

In embodiments, the composition comprises at least one exonic sequence.

In embodiments, the composition or system comprises a CRISPR/Cas system, e.g., a protein and/or nucleic acid thereof. In embodiments, the composition or system further comprises a repair RNA (repRNA) that induces cleavage in a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b

(C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA) and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

Exemplary Compositions, System, and/or Nucleic Acids

In embodiments, the disclosure provides a composition or system suitable for targeting trans-splicing of a pre-mRNA in a cell comprising one or more nucleic acids comprising one or more nucleotide sequences comprising at least one intronic sequence comprising a small nuclear RNA (snRNA), or a small nucleolar RNA (snoRNA) sequence, a splice acceptor and/or splice donor sequence.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA.

In embodiments, the snoRNA comprises an H/ACA box or C/D box. In embodiments, the snRNA or snoRNA sequence assembles into an RNP. In embodiments, the snRNA or snoRNA sequence comprises a sequence motif that assembles into an RNP. In embodiments, the snRNA or snoRNA sequence comprises a secondary structure that assembles into an RNP. In embodiments, the snRNA or snoRNA sequence comprises a sequence motif and a secondary structure that assembles into an RNP. In embodiments, the secondary structure comprises one or more stem loops. In embodiments, the secondary structure is or comprises a stem, internal loop, multibranch loop, or a pseudoknot. In embodiments, the RNP is selected from a small nuclear RNP (snRNP), a small nucleolar RNP (snoRNP), a small cajal body RNP (scaRNP), and a combination thereof. In embodiments, the RNP is selected from U1, U2, U4, U4atac, U5, U6, U6atac, U7, U11, and U12. In embodiments, the RNP is selected from a C/D box snoRNP and a H/ACA box snoRNP.

In embodiments, the snRNA or snoRNA target one or more exonic splicing enhancers (ESEs), one or more intronic splicing enhancers (ISEs), one or more exonic splicing silencers (ESSs), and/or one or more intronic splicing silencers (ISSs). In embodiments, the snRNA or snoRNA comprise a modification to include at least one or more exonic splicing enhancers (ESEs), at least one or more intronic splicing enhancers (ISEs), at least one or more exonic splicing silencers (ESSs), and/or at least one or more intronic splicing silencers (ISSs).

In embodiments, the composition or system comprises a repair RNA (repRNA) and a small RNA that induces cleavage in an RNA. In embodiments, the small RNA that induces cleavage in an RNA is one or more of an siRNA, small hairpin RNA (shRNA), U7 snRNA, a U1 snRNA, a U2 snRNA, a U4 snRNA, a U4atac snRNA, a U5 snRNA, a U6 snRNA, a U6atac snRNA, a U11 snRNA, a U12 snRNA, and an antisense oligonucleotide (ASO). In embodiments, the composition or system comprises a repair RNA (repRNA) and the small RNA comprises a modification or mutation that attenuates, weakens, reduces, decreases, or ablates activity as compared to an unmodified form. In embodiments, the composition or system comprises a repair RNA (repRNA) and the small RNA comprises a modification or mutation that increases, stimulates, or enhances activity as compared to an unmodified form. In embodiments, the composition or system comprises a repair RNA (repRNA) and a small RNA that induces cleavage in an RNA when the present methods are undertaken in cis or trans, as described herein.

In embodiments, the snRNA comprises a M6A modification. In embodiments, the snRNA comprises a M6A modification when the present methods are undertaken in cis or trans, as described herein. In embodiments, the snRNA or snoRNA is modified to comprise at least one or more M6A sites. In embodiments, the snRNA or snoRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the snRNA or snoRNA or (ii) an exonic sequence. In embodiments, the snRNA or snoRNA is modified to not comprise M6A sites. In embodiments, the repRNA comprises at least one or more M6A sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the repRNA or (ii) an exonic sequence. In embodiments, the repRNA comprises no M6A sites.

In embodiments, the repRNA comprises at least one intronic spacer sequence comprising at least one ISE and ESS sequences. In embodiments, the at least one intronic spacer sequence comprising at least one ISE and ESS sequences increases trans-splicing efficiency of a target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences decreases trans-splicing efficiency of a target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences increases trans-splicing efficiency of a off-target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences decreases trans-splicing efficiency of a off-target RNA as compared to an unmodified form.

In embodiments, the repRNA comprises a ESS, ESE, ISS, and/or ISE sequence. In embodiments, the repRNA targets one or more of ESS, ESE, ISS, and/or ISE. In embodiments, an interaction, modulation and/or binding to one or more of ESS, ESE, ISS, and/or ISE reduces or ablates interaction, modulation and/or binding of the one or more of the ESS, ESE, ISS, and/or ISE with a target. In embodiments, the repRNA comprises exon sequences with ESE and ESS sequences. In embodiments, the exon sequences with ESE and ESS sequences increase or decrease trans-splicing efficiency to an RNA target as compared to an unmodified form. In embodiments, the repRNA comprises exon sequences with ESE and ESS sequences. In embodiments, the repRNA comprises exon sequences with ESE and ESS sequences increase or decrease trans-splicing efficiency to an RNA off-target as compared to an unmodified form. In embodiments, the repRNA comprises at least one or more G4 structures. In embodiments, the repRNA comprises at least one or more G4 structures sequester SD/SA motifs. In embodiments, the G4 structure is unwound, such as by DHX36 or CNBP, and remains trapped in the unwound state in the presence of a complementary sequence (e.g., endogenous target or exogenously delivered trigger RNA). In embodiments, the G4 structure decreases off-targets as compared to an unmodified form.

In embodiments, the repRNA comprises a modification comprising at least one or more scaffolding sequences. In embodiments, the at least one or more scaffolding sequences mediates (e.g., recruits) phase condensate-like formation and/or improves local concentrations of repRNAs compared to an unmodified form, and other targeted proteins and/or RNA. In embodiments, the repRNA comprises a modification comprising at least one or more sequences to target the repRNA to the promoter of the target gene of interest, or to proximal condensates that may contain the promoter. In embodiments, the one or more sequences comprises an enhancer RNA, snRNA and/or snoRNA sequences.

In embodiments, the repRNA comprises a modification. In embodiments, the repRNA modification improves interaction and localization to a DNA sequence of the non-template strand of the target gene as compared to an unmodified form. In embodiments, the DNA sequence of the non-template strand of the target gene is the promoter, intron, exon, or enhancer. In embodiments, the modification improves interaction and localization to the DNA sequence of the non-template strand of the target gene through protein-directed (e.g. transcription factor, dCas, ZNF, or other RBP) or nucleotide-directed (e.g., R-loop) methods as compared to an unmodified form.

In embodiments, the repRNA comprises a modification comprising additional RNA elements. In embodiments, the repRNA modification comprising additional RNA elements improves subnuclear localization to nuclear speckles for enhanced trans-splicing efficiency as compared to an unmodified form. In embodiments, the additional RNA element comprise NEAT1 and/or MALAT1, or a fragment thereof. In embodiments, the additional RNA element comprises a nucleotide sequence of SEQ ID NO: 712, or a fragment or variant thereof, optionally having at least about 90%, or at least about 95%, or at least about 97%, or at least about 98%, or at least about 99% identity thereto and/or or having about 1 to about 20 (e.g. about 1, or about 2, or about 3, or about 4, or about 5) nucleic acid modifications, optionally selected from substitutions, additions, or deletions. In embodiments, the repRNA comprises a modification. In embodiments, the repRNA modification enables targeting to site of transcription of target RNAs. In embodiments, the repRNA comprises a modification comprising 5′ UTR or 3′ UTR modifications.

In embodiments, the modification alters intracellular or intranuclear localization based on interactions with endogenous or exogenously supplied molecules (e.g., RNA G4 interactions with transcription factors or other proteins that are localized to specific cellular compartments).

In embodiments, the repRNA comprises a modification in the 5′ UTR of the repRNA. In embodiments, the modification in the 5′ UTR of the repRNA increases stability as compared to an unmodified form. In embodiments, the modification in the 5′ UTR of the repRNA decreases stability as compared to an unmodified form. In embodiments, the modification in the 5′ UTR of the repRNA increases or decreases translation efficiency as compared to an unmodified form. In embodiments, the repRNA comprises a modification in the 3′ UTR of the repRNA. In embodiments, the modification in the 3′ UTR of the repRNA increases stability as compared to an unmodified form. In embodiments, the modification in the 3′ UTR of the repRNA decreases stability as compared to an unmodified form. In embodiments, the modification in the 3′ UTR of the repRNA increases or decreases translation efficiency as compared to an unmodified form.

In embodiments, the repRNA comprises a modification comprising modifying the repRNA to comprise a G4 structure that mediates recruitment of splicing-associated RBPs.

In embodiments, the repRNA comprises a modification comprising at least one or more toehold switches in the repRNA. In embodiments, the at least one or more toehold switches in the repRNA conditionally activate or deactivate (e.g., SD/SA occlusion, binding motif occlusion, or RBP occlusion) upon detection of an endogenous or exogenously supplied target RNA.

In embodiments, the repRNA comprises a modification comprising at least one or more complementary riboregulators in repRNAs (in cis). In embodiments, the at least one or more complementary riboregulators in repRNAs (in cis) occlude splice donor (SD) site and reduce off-target trans-splicing.

In embodiments, the repRNA comprises a modification comprising at least one or more self-complementary riboregulators in repRNAs (in cis). In embodiments, the at least one or more self-complementary riboregulators in repRNAs (in cis) occlude splice acceptor (SA) site and reduce off-target trans-splicing.

In embodiments, the repRNA comprises a modification comprising at least one or more self-complementary riboregulators in repRNAs (in trans). In embodiments, the at least one or more self-complementary riboregulators in repRNAs (in trans) occlude splice donor (SD) site and reduce off-target trans-splicing.

In embodiments, the repRNA comprises a modification comprising at least one or more self-complementary riboregulators in repRNAs (in trans). In embodiments, the at least one or more self-complementary riboregulators in repRNAs (in trans) occlude splice acceptor (SA) site and reduce off-target trans-splicing.

In embodiments, the repRNA comprises a modification comprising at least one or more binding motifs. In embodiments, the at least one or more binding motifs increase trans-splicing efficiency, target specificity, and target site occlusion (SA, SD, ISS, ISE, ESE, and ESS) as compared to an unmodified form.

In embodiments, the repRNA comprises a modification. In embodiments, the repRNA modification enables induction of trans-splicing in response to a stimulus as compared to an unmodified form. In embodiments, the repRNA comprises a modification to turn off or decrease trans-splicing in response to a stimulus as compared to an unmodified form.

In embodiments, the repRNA comprises a modification. In embodiments, the repRNA modification enables small molecule induction of trans-splicing as compared to an unmodified form. In embodiments, the repRNA comprises a modification. In embodiments, the repRNA modification represses small molecule induction of trans-splicing as compared to an unmodified form.

In embodiments, the repRNA comprises a modification. In embodiments, the repRNA modification enables light induction of trans-splicing.

In embodiments, the repRNA comprises a modification comprising at least one or more motifs that are bound and regulated by light-sensitive proteins.

In embodiments, the snRNA or snoRNA comprises a sequence at the 3′ untranslated region (3′UTR). In embodiments, the sequence at the 3′ untranslated region (3′UTR) of the snRNA or snoRNA increases trans-splicing efficiency as compared to an unmodified form. In embodiments, the sequence is from the MALAT1 gene. In embodiments, the sequence is a nucleotide sequence of SEQ ID NO: 712, or a fragment or variant thereof, optionally having at least about 90%, or at least about 95%, or at least about 97%, or at least about 98%, or at least about 99% identity thereto and/or or having about 1 to about 20 (e.g. about 1, or about 2, or about 3, or about 4, or about 5) nucleic acid modifications, optionally selected from substitutions, additions, or deletions.

In embodiments, the RNP assembles on the repRNA and/or the target. In embodiments, the RNP assembles on the repRNA. In embodiments, the RNP assembles on the target. In embodiments, the RNP sterically occludes and inhibits cis-splicing.

In embodiments, the repRNA comprises a minimal intron. In embodiments, the minimal intron is less than about 50 nucleotides, less than about 60 nucleotides, less than about 70 nucleotides, less than about 80 nucleotides, less than about 90 nucleotides, less than about 100 nucleotides, less than about 110 nucleotides, less than about 120 nucleotides, less than about 130 nucleotides, less than about 140 nucleotides, or less than about 150 nucleotides, or about 50 to about 150 nucleotides, or about 50 to about 100 nucleotides, or about 50 to about 75 nucleotides, or about 75 to about 150 nucleotides, or about 100 to about 150 nucleotides, or about 120 to about 150 nucleotides.

In embodiments, the repRNA further comprises a ribozyme site. In embodiments, the ribozyme site is a hairpin, hammerhead, hepatitis delta virus (HDV), Varkud satellite (VS), or glmS ribozyme site, or a variant thereof. In embodiments, the ribozyme site is a HDV ribozyme site. In embodiments, the ribozyme site is a twister ribozyme site. In embodiments, the ribozyme site is upstream of the one or more exons and/or introns of the repRNA. In embodiments, the ribozyme cleaves the target. In embodiments, the ribozyme is a trans-cleaving ribozyme.

In embodiments, the repRNA comprises a ribozyme site that cleaves at the 5′ end of the repRNA. In embodiments, the repRNA comprises a ribozyme site that cleaves at the 3′ end of the repRNA. In embodiments, the repRNA comprises a ribozyme site that cleaves the snRNA or snoRNA at the 5′ end of the repRNA. In embodiments, the repRNA comprises a ribozyme site that cleaves the snRNA or snoRNA at the 3′ end of the repRNA.

In embodiments, the repRNA comprises a M6A modification. In embodiments, the repRNA comprises a M6A modification when the present methods are undertaken in cis or trans, as described herein. In embodiments, the snRNA or snoRNA is modified to comprise at least one or more M6A sites. In embodiments, the snRNA or snoRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A modifications in (i) an unmodified state of the snRNA or snoRNA or (ii) an exonic sequence. In embodiments, the snRNA or snoRNA is modified to not comprise M6A sites. In embodiments, the repRNA comprises at least one or more M6A sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A modifications in (i) an unmodified state of the repRNA or (ii) an exonic sequence. In embodiments, the repRNA comprises no M6A sites.

In embodiments, the composition or system further comprises at least one pre-rRNA stemloop to remove either the 5′cap or 3′ polyA tail. In embodiments, stemloop intramolecular base pairing is a pattern in single-stranded RNA.

In embodiments, there are a plurality of repRNAs under the control of the same, different, or a plurality of promoters. In embodiments, the repRNA and one or more other components of the present system are under the control of the same or different promoters.

In embodiments, the repRNA comprises alternative promoters. In embodiments, the repRNA comprises at least one or more alternative Pol II promoters. In embodiments, the one or more alternative Pol II promoters cap the 5′ end of the repRNA with 7 mG (7-methylguanosine) or TMG (tri-methylguanosine). In embodiments, the one or more alternative Pol II promoters cap the 5′ end of the repRNA with 7 mG (7-methylguanosine) or TMG (tri-methylguanosine) stabilize the repRNA.

In embodiments, the repRNA comprises at least one or more circularized repRNAs. In embodiments, the repRNA comprises at least one or more circularized repRNAs stabilize the repRNA. In embodiments, the circular RNA (or circRNA, or circularized RNA) is a type of single-stranded RNA which, unlike linear RNA, forms a covalently closed continuous loop. Circularized RNAs can be categorized into several types, e.g., depending on processing: exonic circRNAs, intronic circRNAs, exon-intron circRNAs, readthrough circRNAs, fusion circRNAs, and tRNA-derived circRNAs. In embodiments, the 3′ and 5′ ends of the circular RNA are joined together. In embodiments, the repRNA comprises at least one or more circularized 5′ replacement (SD) repRNAs. In embodiments, the repRNA comprises at least one or more circularized 5′ replacement (SD) repRNAs stabilize the repRNA. In embodiments, the repRNA comprising one or more circularized 5′ replacement (SD) repRNAs improves stability and is resistant to exonucleases as compared to an unmodified form. In embodiments, the repRNA comprises at least one or more circularized 3′ replacement (SA) repRNAs. In embodiments, the repRNA comprises at least one or more circularized 3′ replacement (SA) repRNAs stabilize the repRNA. In embodiments, the repRNA comprising one or more circularized 3′ replacement (SA) repRNAs improves stability and is resistant to exonucleases as compared to an unmodified form. In embodiments, the repRNA comprises at least one or more circularized internal replacement (SD+SA) repRNAs. In embodiments, the repRNA comprises at least one or more circularized internal replacement (SD+SA) repRNAs stabilize the repRNA. In embodiments, the repRNA comprising one or more circularized internal replacement (SD+SA) repRNAs improves stability and is resistant to exonucleases as compared to an unmodified form.

In embodiments, the composition or system further comprises a repair RNA (repRNA), a small RNA that induces cleavage in an RNA, and a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system. In embodiments, the, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmcCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA), a small RNA that induces cleavage in an RNA, and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type @-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity as compared to an unmodified form, and/or leads to attenuation of RNA modifying activity, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In embodiments, the composition or system comprises a repair RNA (repRNA) and/or a protein that forms an RNP or is within an RNP, with the snRNA or snoRNA, or a nucleic acid encoding the protein that forms, or is within, the RNP and/or a small RNA that induces cleavage in an RNA and/or a CRISPR/Cas system. In embodiments, the composition or system comprises a repair RNA (repRNA) and/or a protein that forms an RNP or is within an RNP, with the snRNA or snoRNA, or a nucleic acid encoding the protein that forms, or is within, the RNP and/or a small RNA that induces cleavage in an RNA and/or a CRISPR/Cas system when the present methods are undertaken in cis or trans, as described herein. In embodiments, cleavage is initiated from RNPs that are formed on the repRNA, or from RNPs that are formed in cis or trans.

In embodiments, the snRNA or snoRNA comprises an Sm sequence motif, and/or wherein the snRNA or snoRNA comprises an antisense region sequence (ASR). In embodiments, the Sm sequence motif assembles with an Sm or Lsm protein into an RNP. In embodiments, the Sm or Lsm proteins are selected from a B/B′, D3, D2, D1, E, F, G, LSm5, LSm7, LSm4, LSm8, LSm2, LSm3, LSm6 and LSm10 proteins.

In embodiments, the snRNA or snoRNA is at least about 10 to about 80 nucleotides, about 10 to about 70 nucleotides, about 10 to about 60 nucleotides, about 10 to about 50 nucleotides, about 10 to about 40 nucleotides, about 10 to about 30 nucleotides, about 10 to about 20 nucleotides, or about 80, 70, 60, 50, 40, 30, 20, or 10 nucleotides in length. In embodiments, wherein the snRNA or snoRNA comprises an antisense region sequence (ASR) selected from SEQ ID NOs: 700-707.

In embodiments, the Sm sequence motif comprises a nucleotide sequence selected from SEQ ID NOs:: 1-8.

In embodiments, the snRNA or snoRNA comprises a guide repair RNA (grepRNA) sequence. In embodiments, wherein the grepRNA sequence is selected from SEQ ID NOs: 708-711.

In embodiments, composition or system comprises a splice acceptor. In embodiments, composition or system comprises a splice donor.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In embodiments, the at least one intronic sequence comprises one or more splicing signals. In embodiments, the one or more splicing signals are selected from an exonic splicing enhancer (ESE), an intronic splicing enhancer (ISE), an exonic splicing silencer (ESS), intronic splicing silencer (ISS), a U1 binding motif (e.g., among other snRNA binding motifs), a polypyrimidine tract, a branch point, and a combination thereof.

In embodiments, the at least one intronic sequence comprises a branch point and a polypyrimidine tract.

In embodiments, the composition or system comprises 1, 2, 3, 4, 5, 6, 7, 8, 9, or 10 exons.

In embodiments, the one or more binding domain sequences is at least about 5 to about 10, about 5 to about 15, about 5 to about 20, about 10 to about 15, about 10 to about 20, about 15 to about 20, or about 20, or about 19, or about 18, or about 17, or about 16, or about 15, or about 14, or about 13, or about 12, or about 11, or about 10, or about 9, or about 8, or about 7, or about 6, or about or 5 nucleotides in length.

In embodiments, wherein the one or more binding domain sequences is less than about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, or about 250, or about 200, or about 150, or about 100, or about 50 nucleotides in length.

In embodiments, the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the composition or system comprises one binding domain sequence. In embodiments, the composition or system comprises at least two binding domain sequences. In embodiments, the composition or system comprises 3, 4, 5, 6, 7, 8, 9, or 10 binding domain sequences.

In embodiments, when the composition or system is introduced to the cell an exon in the pre-mRNA is targeted for trans-splicing. In embodiments, the target sequence is positioned in a region of the pre-mRNA comprising the exon targeted for trans-splicing. In embodiments, the target sequence is positioned proximal to a splice site. In embodiments, the target sequence is positioned proximal to a splice donor or a splice acceptor.

In aspects, the present disclosure provides a composition or system for targeting trans-splicing of a pre-mRNA in a cell, comprising one or more nucleic acids comprising one or more nucleotide sequences comprising from 5′ to 3′: (a) at least one intronic sequence comprising: (i) one or more binding domain sequences of about 4 to about 300 nucleotides each with complementarity to a pre-mRNA target sequence; (ii) a small nuclear RNA (snRNA) or a small nucleolar RNA (snoRNA) sequence of about 7 to about 300 nucleotides in length which forms a secondary structure and/or comprises a sequence motif to direct the one or more binding domains to the pre-mRNA target sequence; and (iii) one or more splicing signals; (b) a splice acceptor; and (c) at least one exonic sequences.

In embodiments, when the composition or system is introduced to the cell an exon in the pre-mRNA is targeted for trans-splicing. In embodiments, the target sequence is positioned upstream the exon in the pre-mRNA targeted for trans-splicing. In embodiments, the target sequence is positioned proximal to a splice site. In embodiments, the target sequence is positioned proximal to a splice acceptor or a splice donor. In embodiments, trans-splicing occurs between a splice donor upstream the exon in the pre-mRNA and the splice acceptor of the composition or system. In embodiments, trans-splicing results in ligation of the 3′ end of an exon upstream the splice donor in the pre-mRNA with the 5′ end of the at least one exonic sequence of the composition or system. In embodiments, the one or more splicing signals comprises a branch point and a polypyrimidine tract.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA. In embodiments, the snoRNA comprises an H/ACA box or C/D box. In embodiments, the snRNA or snoRNA sequence assembles into an RNP. In embodiments, the snRNA or snoRNA sequence comprises a sequence motif that assembles into an RNP.

In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity as compared to an unmodified form, and/or leads to attenuation of RNA modifying activity, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In aspects, the present disclosure provides a composition or system for targeting trans-splicing of a pre-mRNA in a cell, comprising one or more nucleic acids comprising one or more nucleotide sequences comprising from 5′ to 3′: at least one exonic sequence; a splice donor; at least one intronic sequence comprising: a small nuclear RNA (snRNA) or a small nucleolar RNA (snoRNA) sequence of about 7 to about 300 nucleotides in length, and (ii) one or more binding domain sequences of about 4 to about 300 nucleotides each with complementarity to a pre-mRNA target sequence, wherein the snRNA or snoRNA forms a secondary structure and/or comprises a sequence motif to direct the one or more binding domains to the pre-mRNA target sequence.

In embodiments, when the composition or system is introduced to the cell an exon in the pre-mRNA is targeted for trans-splicing. In embodiments, the target sequence is positioned downstream the exon in the pre-mRNA. In embodiments, the target sequence is positioned proximal to a splice site. In embodiments, the target sequence is positioned proximal to a splice donor or a splice acceptor.

In embodiments, trans-splicing occurs between the splice donor of the nucleic acid and a splice acceptor downstream the exon in the pre-mRNA.

In embodiments, trans-splicing results in ligation of the 3′ end of the at least one exonic sequence of the nucleic acid with the 5′ end of an exon downstream the splice acceptor in the pre-mRNA.

In embodiments, the intronic sequence is an snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA assembles into an snRNP. In embodiments, the snRNA is a U7 snRNA. In embodiments, the U7 snRNA assembles into a U7 RNP. In embodiments, the snRNA is a U1 snRNA. In embodiments, the U1 snRNA assembles into a UI RNP. In embodiments, the snRNA is a U11 snRNA. In embodiments, the U11 snRNA assembles into a U11 RNP. The composition or system of any one of the embodiments described herein, wherein the snRNA or snoRNA sequence comprises an Sm sequence motif, and/or the snRNA or snoRNA comprises an antisense region sequence (ASR).

In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity as compared to an unmodified form, and/or leads to attenuation of RNA modifying activity, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In embodiments, the snRNA or snoRNA is at least about 10 to about 80 nucleotides, about 10 to about 70 nucleotides, about 10 to about 60 nucleotides, about 10 to about 50 nucleotides, about 10 to about 40 nucleotides, about 10 to about 30 nucleotides, about 10 to about 20 nucleotides, or about 80, 70, 60, 50, 40, 30, 20, or 10 nucleotides in length.

In embodiments, the snRNA or snoRNA comprises an antisense region sequence (ASR) selected from SEQ ID NOs: 700-707. In embodiments, the Sm sequence motif comprises a nucleotide sequence selected from SEQ ID NOs: 1-8.

In embodiments, the snRNA or snoRNA comprises a grepRNA sequence. In embodiments, the grepRNA sequence is selected from SEQ ID NOs: 708-711.

In embodiments, the snRNA or snoRNA sequence comprises an Sm sequence motif, and/or the snRNA or snoRNA comprises an antisense region sequence (ASR), and a U7 snRNA. In embodiments, the Sm sequence motif comprises a sequence set forth in SEQ ID NOs: 3 and 4.

In embodiments, the Sm sequence motif assembles with an Sm protein into an RNP. In embodiments, the Sm protein is selected from a B/B′, D3, D2, D1, E, F, and G Sm protein.

In embodiments, the snRNA or snoRNA sequence comprises a sequence having at least 80% sequence identity to a sequence selected from SEQ ID NOs: 9-589 or a portion thereof. In embodiments, the snRNA or snoRNA sequence comprises a region of about 7 to about 40 nucleotides in length, wherein the region comprises an Sm sequence motif, and/or the snRNA or snoRNA comprises an antisense region sequence (ASR).

In embodiments, the snRNA or snoRNA comprises an antisense region sequence (ASR) selected from SEQ ID NOs: 700-707. In embodiments, the snRNA or snoRNA is at least about 10 to about 80 nucleotides, about 10 to about 70 nucleotides, about 10 to about 60 nucleotides, about 10 to about 50 nucleotides, about 10 to about 40 nucleotides, about 10 to about 30 nucleotides, about 10 to about 20 nucleotides, or about 80, 70, 60, 50, 40, 30, 20, or 10 nucleotides in length. In embodiments, the snRNA or snoRNA sequence comprises a region of about 40 to about 300 nucleotides in length, wherein the region comprises a secondary structure and/or an Sm sequence motif. In embodiments, the composition or system comprises one binding domain sequence. In embodiments, the composition or system comprises more than one binding domain sequence. In embodiments, the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In aspects, the present disclosure provides a composition or system for targeting trans-splicing of a pre-mRNA in a cell, comprising one or more nucleic acids comprising one or more nucleotide sequences comprising from 5′ to 3′: at least one intronic sequence comprising: (i) a snRNA or snoRNA sequence comprising an H/ACA box or a C/D box and one or more binding domain sequences of about 4 to about 30 nucleotides each with complementarity to a pre-mRNA target sequence; and (ii) one or more splicing signals; a splice acceptor; and at least one exonic sequence.

In embodiments, the composition or system comprises a CRISPR/Cas system, e.g., a protein and/or nucleic acid thereof. In embodiments, the composition or system further comprises a repair RNA (repRNA) and a CRISPR/Cas system. In embodiments, the CRISPR/Cas system is active, e.g., catalytically active. In embodiments, the CRISPR/Cas system is inactive, e.g., catalytically inactive, e.g., “dead”. In embodiments, the CRISPR/Cas system is a Type III CRISPR/Cas system. In embodiments, the Type III CRISPR/Cas system is or comprises a Type IIIA, Type IIIB, Type IIIC, Type IIID, Type IIIE, or Type IIIF CRISPR/Cas system, optionally wherein the Type IIIA, Type IIIB, Type IIIC, Type IIID, Type IIIE, or Type IIIF CRISPR/Cas system is active, catalytically inactive or has reduced activity relative to wild type. In embodiments, the Type III CRISPR/Cas system is or comprises a Cas10 (Csm1), Csm2, Cas7 (Csm3), Csm4, Csm5, Cas6, and, Cas7-11, or a fragment or variant thereof, optionally wherein the Type III CRISPR/Cas system is or comprises a Cas10 (Csm1), Csm2, Cas7 (Csm3), Csm4, Csm5, Cas6, and, Cas7-11 is active, catalytically inactive or has reduced activity relative to wild type. In embodiments, the Type III CRISPR/Cas system comprises a domain from a different endonuclease, optionally wherein the different endonuclease is a Cas endonuclease, optionally wherein the domain is one or more of a PAM-interacting domain, optionally wherein the domain is derived from one or more of Cas9, Cas12a (Cpf1), Cas12e (CasX), Cas12d (CasY), Cas12b (C2c1), Cas13a (C2c2), Cas13b, Cas13c, Cas13d, Cas13X/Cas13bt, Cas13Y, Cas12c (C2c3), GeoCas9, CjCas9, NmeCas9, Cas12J (CasPhi), Cas12L (CasLambda), Cas12f (Cas14), Cas12g, Cas12h, Cas12i, Cas12k, NmeCas9, Nme2Cas9, CjCas9, GeoCas9, BlatCas9, PpCas9, and Cas14. In embodiments, the composition or system comprises an intronic sequence comprising a sequence which interacts with or is suitable for interacting with the CRISPR/Cas system. In embodiments, the composition or system further comprises a repair RNA (repRNA) and a CRISPR/Cas system when the present methods are undertaken in cis or trans, as described herein.

In embodiments, the Cas is a type I. In embodiments, the Cas is a type I-A, e.g., without limitation Cas8a or Cas5. In embodiments, the Cas is a type I. In embodiments, the Cas is a type I-B, e.g., without limitation Cas8b. In embodiments, the Cas is a type I. In embodiments, the Cas is a type I-C, e.g., without limitation Cas8c. In embodiments, the Cas is a type I. In embodiments, the Cas is a type I-D, e.g., without limitation Cas10d. In embodiments, the Cas is a type I. In embodiments, the Cas is a type I-E, e.g., without limitation Cse1 or Cse2. In embodiments, the Cas is a type I. In embodiments, the Cas is a type I-F, e.g., without limitation Csyl, Csy2, or Csy3. In embodiments, the Cas is a type I. In embodiments, the Cas is a type I-G, e.g., without limitation GSU0054. In embodiments, the Cas is a type I. In embodiments, the Cas type I is without limitation, Cas3.

In embodiments, the Cas is a type II. In embodiments, the Cas is a type II-A, e.g., without limitation Csn2. In embodiments, the Cas is a type II. In embodiments, the Cas is a type II-B, e.g., without limitation Cas4. In embodiments, the Cas is a type II. In embodiments, the Cas is a type II-C. In embodiments, the Cas is a type II. In embodiments, the Cas type II is without limitation Cas9.

In embodiments, the Cas is a type III. In embodiments, the Cas is a type III-A, e.g., without limitation Csm2. In embodiments, the Cas is a type III. In embodiments, the Cas is a type III-B, e.g., without limitation Cmr5. In embodiments, the Cas is a type III. In embodiments, the Cas is a type III-C, e.g., without limitation Cas10 or Csx11. In embodiments, the Cas is a type III. In embodiments, the Cas is a type III-D, e.g., without limitation Csx 10. In embodiments, the Cas is a type III. In embodiments, the Cas is a type III-E. In embodiments, the Cas is a type III. In embodiments, the Cas is a type III-F. In embodiments, the Cas is a type III. In embodiments, the Cas type III is without limitation Cas10.

In embodiments, the Cas is a type IV. In embodiments, the Cas is a type IV-A. In embodiments, the Cas is a type IV. In embodiments, the Cas is a type IV-B. In embodiments, the Cas is a type IV. In embodiments, the Cas is a type IV-C.

In embodiments, the Cas is a type V. In embodiments, the Cas is a type V-A, e.g., without limitation Cas12a (Cpf1). In embodiments, the Cas is a type V. In embodiments, the Cas is a type V-B, e.g., without limitation Cas12b (C2c1). In embodiments, the Cas is a type V. In embodiments, the Cas is a type V-C, e.g., without limitation Cas12c (C2c3). In embodiments, the Cas is a type V. In embodiments, the Cas is a type V-D, e.g., without limitation Cas12d (CasY). In embodiments, the Cas is a type V. In embodiments, the Cas is a type V-E, e.g., without limitation Cas12e (CasX). In embodiments, the Cas is a type V. In embodiments, the Cas is a type V-F, e.g., without limitation Cas12f (Cas14, or C2c10). In embodiments, the Cas is a type V. In embodiments, the Cas is a type V-G, e.g., without limitation Cas12g. In embodiments, the Cas is a type V. In embodiments, the Cas is a type V-H, e.g., without limitation Cas12h. In embodiments, the Cas is a type V. In embodiments, the Cas is a type V-I, e.g., without limitation Cas12i. In embodiments, the Cas is a type V. In embodiments, the Cas is a type V-K, e.g., without limitation Cas12k (C2c5). In embodiments, the Cas is a type V. In embodiments, the Cas is a type V-U, e.g., without limitation C2c4, C2c8, or C2c9. In embodiments, the Cas is a type V. In embodiments, the Cas type V is without limitation Cas12. In embodiments, the Cas is a type VI.

In embodiments, the Cas is a type VI-A, e.g., without limitation Cas13a (C2c2). In embodiments, the Cas is a type VI. In embodiments, the Cas is a type VI-B, e.g., without limitation Cas13b. In embodiments, the Cas is a type VI. In embodiments, the Cas is a type VI-C, e.g., without limitation Cas13c. In embodiments, the Cas is a type VI. In embodiments, the Cas is a type VI-D, e.g., without limitation Cas13d. In embodiments, the Cas is a type VI. In embodiments, the Cas is a type VI-X, e.g., without limitation Cas13x.1. In embodiments, the Cas is a type VI. In embodiments, the Cas is a type VI-Y. In embodiments, the Cas is a type VI. In embodiments, the Cas type VI is without limitation Cas13.

In embodiments the Cas is Cas3, Cas8a, Cas5, Cas8b, Cas8c, Cas10d, Cse1, Cse2, Csy1, Csy2, Csy3, GSU0054, Cas10, Csm2, Cmr5, Cas10 or Csx11, Csx10, Csf1, Cas9, Csn2, Cas4, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, or Cas13x.1.

In embodiments, when the composition or system is introduced to the cell an exon in the pre-mRNA is targeted for trans-splicing. In embodiments, the target sequence is positioned upstream the exon in the pre-mRNA. In embodiments, the target sequence is positioned proximal to a splice site. In embodiments, the target sequence is positioned proximal to a splice donor or splice acceptor. In embodiments, trans-splicing occurs between a splice donor upstream the exon in the pre-mRNA and the splice acceptor of the composition or system. In embodiments, trans-splicing results in ligation of the 3′ end of an exon upstream the splice donor in the pre-mRNA with the 5′ end of the at least one exonic sequence of the composition or system.

In embodiments, the one or more splicing signals comprises a branch point and a polypyrimidine tract.

In embodiments, the intronic sequence comprises a snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the composition or system comprises a snoRNA. In embodiments, the snoRNA comprises an H/ACA box or C/D box. In embodiments, the snRNA or snoRNA sequence assembles into an RNP. In embodiments, the snRNA or snoRNA sequence comprises a sequence motif that assembles into an RNP. In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity, and/or leads to attenuation of RNA modifying activity as compared to an unmodified form, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence. In embodiments, the (a) at least one intronic sequence, (b) splice acceptor and/or splice donor sequence, and (c) at least one exonic sequence are provided in cis or trans, or are suitable for being provided in cis or trans. In embodiments, the (a) at least one intronic sequence, (b) splice acceptor and/or splice donor sequence, and (c) at least one exonic sequence are provided in trans, or are suitable for being provided in trans.

In aspects, the present disclosure provides a composition or system for targeting trans-splicing of a pre-mRNA in a cell, comprising one or more nucleic acids comprising one or more nucleotide sequences comprising from 5′ to 3′: (a) at least one exonic sequence; (b) a splice donor; and (c) at least one intronic sequence comprising a snRNA or snoRNA sequence comprising an H/ACA box or a C/D box and one or more binding domain sequences of about 4 to about 30 nucleotides each with complementarity to a pre-mRNA target sequence.

In embodiments, when the composition or system is introduced to the cell an exon in the pre-mRNA is targeted for trans-splicing. In embodiments, the target sequence is positioned downstream the exon in the pre-mRNA. In embodiments, the target sequence is positioned proximal to a splice site. In embodiments, the target sequence is positioned proximal to a splice donor or a splice acceptor.

In embodiments, trans-splicing occurs between the splice donor of the composition or system and a splice acceptor downstream the exon in the pre-mRNA. In embodiments, trans-splicing results in ligation of the 3′ end of the at least one exonic sequence of the composition or system with the 5′ end of an exon downstream the splice acceptor in the pre-mRNA.

In embodiments, the snRNA or snoRNA sequence comprises an H/ACA box comprising 5′ to 3′ an H consensus sequence and an ACA consensus sequence. In embodiments, the composition or system comprises at least one binding domain sequence positioned: (i) upstream the H consensus sequence; (ii) downstream the ACA consensus sequence; (iii) between the H consensus sequence and the ACA consensus sequence; or (iv) a combination of (i)-(iii).

In embodiments, the snRNA or snoRNA sequence comprises C/D box comprising 5′ to 3′ a C consensus sequence, a D′ consensus sequence, a C′ consensus sequence, and a D consensus sequence.

In embodiments, the composition or system comprises at least one binding domain positioned (i) upstream the C consensus sequence; (ii) between the C consensus sequence and the D′ consensus sequence; (iii) between the D′ consensus sequence and the C′ consensus sequence; (iv) between the C′ consensus sequence and the D consensus sequence; (v) downstream the D consensus sequence; or (vi) a combination of (i)-(v).

In embodiments, the snRNA or snoRNA sequence comprises a sequence having at least 80% sequence identity to a sequence selected from any one of SEQ ID NOs: 590-657 and 732-793, or a portion thereof. In embodiments, the snRNA or snoRNA sequence comprises a region of about 40 to about 300 nucleotides in length and comprising an H consensus sequence and an ACA consensus sequence.

In embodiments, the snRNA or snoRNA sequence comprises one binding domain sequence. In embodiments, the snRNA or snoRNA sequence comprises more than one binding domain sequence. In embodiments, the composition or system comprises at least one binding domain sequence with full complementarity to the pre-mRNA target sequence. In embodiments, the composition or system comprises at least one binding domain sequence with partial complementarity to the pre-mRNA target sequence. In embodiments, the at least one binding domain sequence comprises one or more mismatches relative to the pre-mRNA target sequence. In embodiments, the at least one binding domain sequence has at least 95% complementarity to the pre-mRNA target sequence.

In embodiments, the (a) at least one intronic sequence, (b) splice acceptor and/or splice donor sequence, and (c) at least one exonic sequence are provided in cis or trans, or are suitable for being provided in cis or trans.

In embodiments, the (a) at least one intronic sequence, (b) splice acceptor and/or splice donor sequence, and (c) at least one exonic sequence are provided in trans, or are suitable for being provided in trans. In embodiments, these elements are under the control of one or more promoters. In embodiments, these elements are under the control of different promoters. In embodiments, these elements are operably linked, but separated by a cleavable sequence (e.g., a self-cleaving ribozyme). In embodiments, (i) multiple populations of repRNA are under the control of different promoters or (ii) the repRNA and another system member under control of different promoters.

In some embodiments, a nucleic acid of the disclosure comprises at least one binding domain sequence with full complementarity to the target sequence. In some embodiments, the nucleic acid comprises at least one binding domain sequence with partial complementarity to the target sequence (e.g., comprising at least 95% complementarity to the target sequence). In some embodiments, the nucleic acid comprises at least one binding domain sequence with full complementarity to the target sequence and at least one binding domain sequence with partial complementarity to the target sequence (e.g., comprising at least 95% complementarity to the target sequence).

In embodiments, the composition or system comprises a sequence up to about 20,000 nucleotides in length.

In embodiments, the composition or system comprises a sequence of about 50 to about 500, about 50 to about 1000, about 100 to about 500, about 100 to about 1000, about 500 to about 1000, about 500 to about 2000, about 500 to about 3,000, about 500 to about 4,000, about 500 to about 5,000, about 1,000 to about 5,000, about 1,000 to about 10,000, about 5,000 to about 15,000, or about 5,000 to about 20,000 nucleotides in length.

In embodiments, the nucleic acid of the disclosure a sequence of up to about 20,000 nucleotides in length. In some embodiments, the sequence is up to about 10,000 nucleotides in length. In some embodiments, the sequence is up to about 9,000 nucleotides in length. In some embodiments, the sequence is up to about 8,000 nucleotides in length. In some embodiments, the sequence is up to about 7,000 nucleotides in length. In some embodiments, the sequence is up to about 6,000 nucleotides in length. In some embodiments, the sequence is up to about 5,000 nucleotides in length. In some embodiments, the sequence is about 50 to about 500 nucleotides in length. In some embodiments, the sequence about 50 to about 1000 nucleotides in length. In some embodiments, the sequence about 100 to about 500 nucleotides in length. In some embodiments, the sequence about 100 to about 1000 nucleotides in length. In some embodiments, the sequence about 500 to about 1000 nucleotides in length. In some embodiments, the sequence about 500 to about 2000 nucleotides in length. In some embodiments, the sequence about 500 to about 3,000 nucleotides in length. In some embodiments, the sequence about 500 to about 4,000 nucleotides in length. In some embodiments, the sequence about 500 to about 5,000 nucleotides in length. In some embodiments, the sequence about 1,000 to about 5,000 nucleotides in length. In some embodiments, the sequence about 1,000 to about 10,000 nucleotides in length. In some embodiments, the sequence about 5,000 to about 15,000 nucleotides in length. In some embodiments, the sequence about 5,000 to about 20,000 nucleotides in length.

In embodiments, a composition, system, and/or the one or more nucleic acids comprising one or more nucleotide sequences described herein comprises a sequence selected from Table 6 or a portion thereof. Table 6 provides exemplary nucleotide sequences of composition, system, and/or the one or more nucleic acids comprising one or more nucleotide sequences described herein. As presented in Table 6, the regions of the sequence are demarcated by hyphens (-) and identification of the regions from 5′ to 3′ are provided as Region 1, Region 2, Region 3, and optionally Region 4. In some embodiments, a composition, system, and/or the one or more nucleic acids comprising one or more nucleotide sequences described herein comprises a sequence having the formula 5′-[A]-[B]-3′, wherein [A] is a nucleotide sequence selected from Table 6 and [B] is a sequence comprising from 5′ to 3′ a splice acceptor and one or more exonic sequences. In some embodiments, [A] comprises a nucleotide sequence selected from Table 6, wherein the RNA-binding domain is exchanged with an RNA-binding domain described herein.

In embodiments, a composition, system, and/or the one or more nucleic acids comprising one or more nucleotide sequences described herein comprises from 5′ to 3′ one or more RNA binding domains described herein, a snRNA and/or snoRNA, an intronic sequence, a splice acceptor, and one or more exonic sequences, wherein the snRNA and/or snoRNA sequence has about 90%, 95%, 98%, 99%, or 100% to a snRNA and/or snoRNA sequence identified in Table 6. In some embodiments, a composition, system, and/or the one or more nucleic acids comprising one or more nucleotide sequences described herein comprises from 5′ to 3′ one or more exonic sequences, a splice donor, an intronic sequence, a snRNA and/or snoRNA sequence, and one or more RNA binding domains described herein, wherein the snRNA and/or snoRNA sequence has about 90%, 95%, 98%, 99%, or 100% to a snRNA and/or snoRNA sequence identified in Table 6. In some embodiments, the intronic sequence has about 90%, 95%, 98%, 99%, or 100% to an intronic sequence identified in Table 6.

Compositions, Systems, and/or Nucleic Acids for Targeted Trans-Splicing

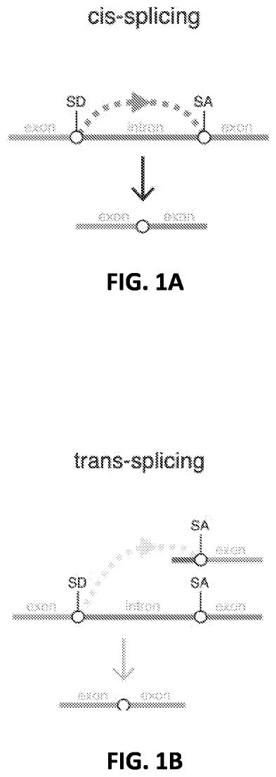

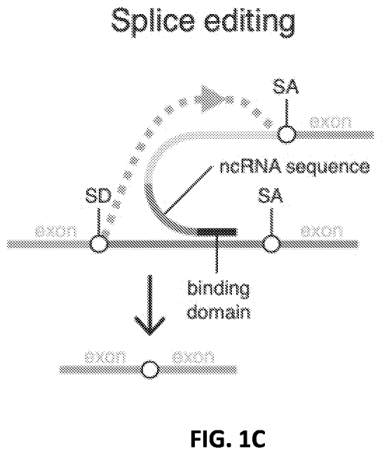

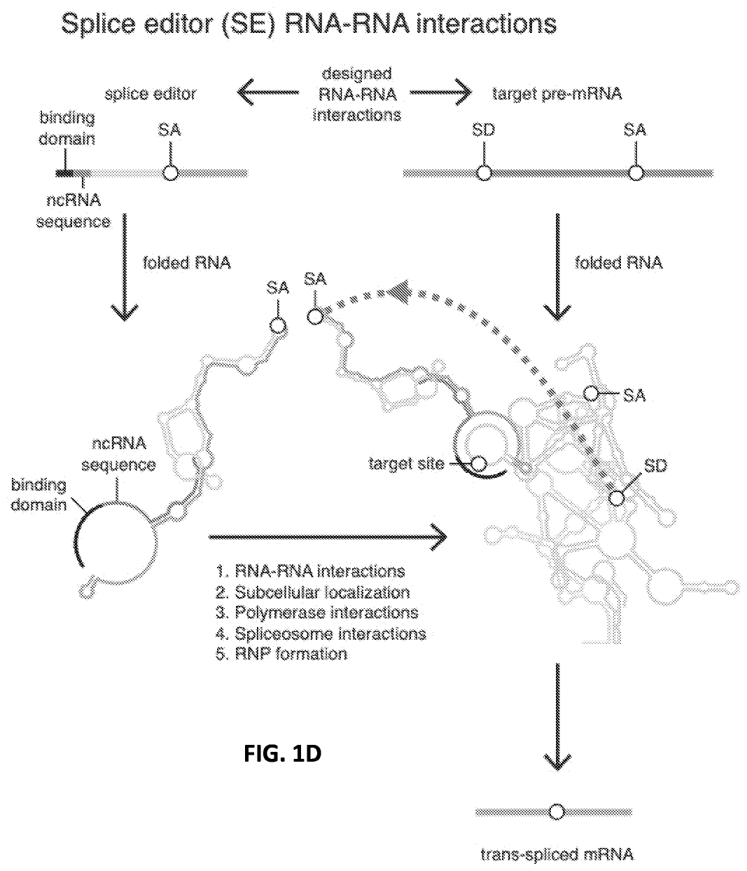

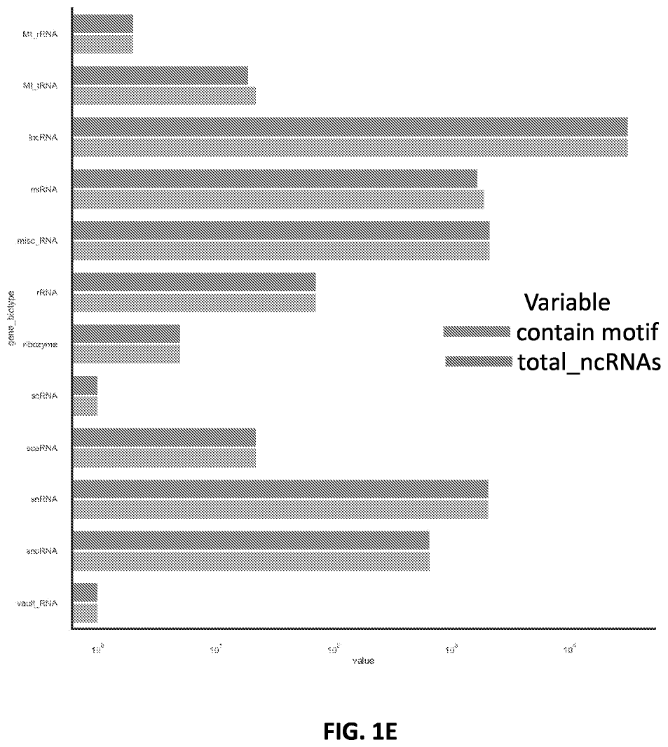

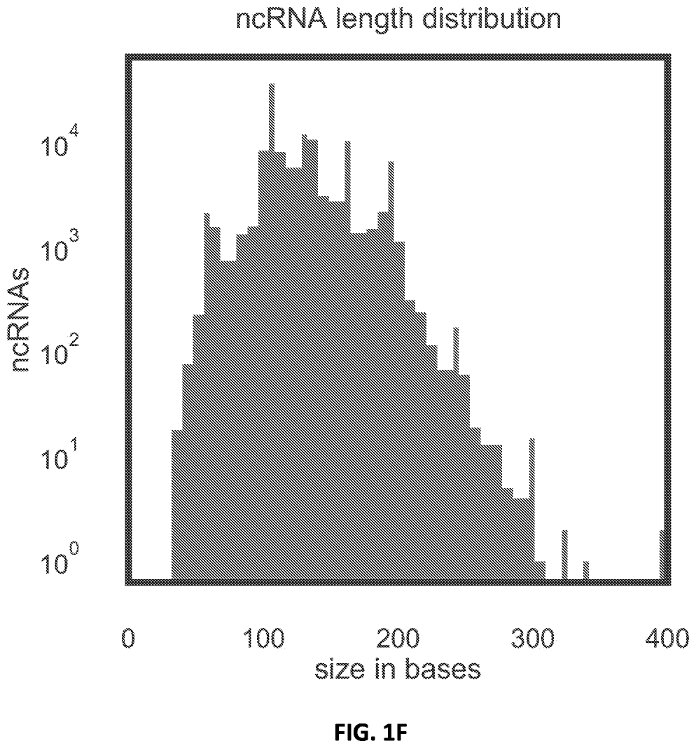

Accurate pre-mRNA splicing is critical for proper protein expression. Nuclear pre-mRNA splicing is catalyzed by the spliceosome. Vertebrate gene architecture often consists of relatively long introns and short internal exons. The exon-intron boundaries are defined by a splice donor (the 5′ splice site or splice site at the 3′end of an exon) and a splice acceptor (the 3′ splice site or splice site at the 5′ end of an exon). In addition to recognizing splice sites, the spliceosome relies on various splicing signals to mediate a splicing event, including a branch point sequence and a polypyrimidine tract. Typically, the branch point sequence comprises an adenosine situated within a consensus sequence and is situated about 18-40 nucleotides upstream of the 3′ splice site. The polypyrimidine tract comprises a repetitive sequence of uracils and is proximal the 3′ splice site. Alternative signals can enhance or decrease splicing activity, including exonic splicing enhancers (ESEs), exonic splicing silencers (ESSs), intronic splicing enhancers (ISEs), and intronic splicing silencers (ISSs). Splicing in cis (“cis-splicing”) occurs when the 2′ OH group of the branch adenosine of the intron carries out a nucleophilic attack on the 5′ splice site (splice donor). This results in cleavage at this site and ligation of the 5′ end of the intron to the branch adenosine, forming a lariat structure. The 3′ splice site (splice acceptor) is attacked by the 3′ OH of the 5′ exon, resulting in ligation of the 5′ and 3′ exons to form the mRNA and release of the intron lariat (see, e.g., FIG. 1A).

In contrast, splicing in trans (“trans-splicing”) occurs between two different RNA molecules, wherein the 3′ splice site (splice acceptor) of a second RNA is attacked by the 3′ OH of the 5′ exon of a first RNA, resulting in ligation of the 5′ exon of the first RNA and the 3′ exon of the second RNA, thereby forming a chimeric RNA (see, e.g., FIG. 1B).

The present disclosure provides composition or system suitable for targeting trans-splicing of a pre-mRNA in a cell comprising one or more nucleic acids comprising one or more nucleotide sequences comprising: (a) at least one intronic sequence comprising: (i) one or more binding domain sequences of about 4 to about 300 nucleotides each with complementarity to a pre-mRNA target sequence; and (ii) a small nuclear RNA (snRNA) or a small nucleolar RNA (snoRNA) sequence of about 7 to about 300 nucleotides in length which forms a secondary structure and/or comprises a sequence motif to direct the one or more binding domains to the pre-mRNA target sequence; (b) a splice acceptor and/or splice donor sequence; and (c) at least one exonic sequence.

In some embodiments, the composition, system, and/or the one or more nucleic acids comprising one or more nucleotide sequences is designed to bind to a specific region of a target RNA (e.g., pre-mRNA), thereby enabling splicing between the one or more splice sites of the composition, system, and/or the one or more nucleic acids comprising one or more nucleotide sequences and one or more splice sites of the target RNA (e.g., pre-mRNA). In some embodiments, the trans-splicing results in a chimeric mRNA comprising the at least one exonic sequence of the composition, system, and/or the one or more nucleic acids comprising one or more nucleotide sequences and one or more exons of the target RNA (e.g., pre-mRNA).

In humans, exon definition is determined by splice sites paired across an exon (e.g., a splice acceptor (3′ splice site) at the 5′ end of the exon and a splice donor (5′ splice site) at the 3′end of the exon). Other splicing signals (e.g., branch point sequences, polypyrimidine tracts, exonic (or intronic) splicing enhancers and silencers) contribute to proper splicing together of exons to form a mature mRNA. During pre-mRNA splicing, the spliceosome searches for a pair of closely spaced splice sites. Without being bound by theory, the composition, system, and/or the one or more nucleic acids comprising one or more nucleotide sequences described herein mediate efficient trans-splicing by bringing a splice site of the target RNA (e.g., pre-mRNA, e.g., a splice acceptor or splice donor of the target pre-mRNA) into close proximity with a splice site of the composition, system, and/or the one or more nucleic acids comprising one or more nucleotide sequences (e.g., a splice acceptor or splice donor of the composition, system, and/or the one or more nucleic acids comprising one or more nucleotide sequences), such that the spliceosome mediates splicing between the splice site of the target pre-mRNA and the splice site of the composition, system, and/or the one or more nucleic acids comprising one or more nucleotide sequences.

In some embodiments, the composition, system, and/or the one or more nucleic acids comprising one or more nucleotide sequences comprise a nucleotide sequence comprising from 5′ to 3′ (i) at least one intronic sequence comprising an RNA-guided domain; (ii) a splice acceptor; and (iii) at least one exonic sequence.

In some embodiments, the composition, system, and/or the one or more nucleic acids comprising one or more nucleotide sequences comprise a nucleotide sequence comprising from 5′ to 3′ (i) at least one exonic sequence; (ii) a splice donor; and (iii) at least one intronic sequence comprising an RNA-guided domain.

In some embodiments, the at least one intronic sequences comprises one or more splicing signals (e.g., a branch point sequence, a polypyrimidine tract, an ISE, and/or an ISS). In some embodiments, the at least one exonic sequences comprises one or more splicing signals (e.g., an ESE and/or an ESS).

In aspects, disclosed herein is a composition or system for targeting trans-splicing of a pre-mRNA in a cell, comprising one or more nucleic acids comprising one or more nucleotide sequences comprising from 5′ to 3′: (a) a splice donor and/or a splice acceptor; and (b) at least one intronic sequence comprising a snRNA or snoRNA sequence comprising an H/ACA box or a C/D box and one or more binding domain sequences of about 4 to about 300 nucleotides each with complementarity to a pre-mRNA target sequence.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the binding domain sequence is between about 1 nucleotide, or about 5 nucleotides, or about 10 nucleotides, or about 50 nucleotides, or about 100 nucleotides, or about 1000 nucleotides near a H/ACA box or C/D box sequence.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is a H/ACA snoRNA (SNORA), C/D snoRNA (SNORD), or a small cajal RNA (scaRNA).

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is SNORA101B, SNORA48, SNORA54, SNORA66, SNORA73A, or SNORA8, or the snoRNA is SNORA101B, SNORA48, SNORA54, SNORA66, SNORA73A, or SNORA8 with at least one or more mutations.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is in cis with respect to the repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is in trans with respect to the repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the repRNA comprises a splice donor and/or a splice acceptor.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the repRNA comprises a splice donor and/or a splice acceptor, and wherein the snoRNA and snRNA are in trans with respect to the repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein a mutation in H and/or ACA motifs substantially decreases the trans-splicing efficiency.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 3′ end of a 5′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the addition of a SNORA modification on a transcript stabilizes the 3′ end of the transcript after ribozyme cleavage and polyA removal.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 5′ end of a 3′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 3′ end of a 3′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the snoRNA is added to the 5′ end of a 5′ repRNA.

In the embodiments, disclosed herein is a composition or system of any one of the embodiments disclosed herein, the viral vector of any one of the embodiments disclosed herein, or the lipid nanoparticle of any one of the embodiments disclosed herein, or the pharmaceutical composition of any one of the embodiments disclosed herein, or the method of any one of the embodiments disclosed herein, or the kit of any one of the embodiments disclosed herein, wherein the SNORA sequence has at least about 80%, or at least about 85%, or at least about 90%, or at least about 95%, or at least about 96%, or at least about 97%, or at least about 98%, or at least about 99% identity to any one of SEQ ID NOs: 590-657 or 732-793.

### RNA Guided Domain

In some embodiments, the RNA guided domain comprises a nucleotide sequence comprising (i) one or more binding domains, each having complementarity to a target sequence in the target RNA (e.g., pre-mRNA); and (ii) a snRNA and/or snoRNA. In some embodiments, the one or more binding domains mediate binding of the trans-splicing nucleic acid molecules to a target RNA (e.g., pre-mRNA) in a cell. In some embodiments, the snRNA and/or snoRNA mediates assembly into an RNP.

In some embodiments, the RNA guided domain comprises a nucleotide sequence having from 5′ to 3′: (i) one or more binding domains, each having complementarity to a target sequence in the target RNA (e.g., pre-mRNA); and (ii) a snRNA and/or snoRNA.

In some embodiments, the RNA guided domain comprises a nucleotide sequence having from 5′ to 3′: (i) a snRNA and/or snoRNA; and (ii) one or more binding domains, each having complementarity to a target sequence in the target RNA (e.g., pre-mRNA).

In some embodiments, the RNA guided domain comprises a nucleotide sequence having a snRNA and/or snoRNA, wherein the one or more binding domains are inserted into the snRNA and/or snoRNA or exchanged for contiguous nucleotides of the snRNA and/or snoRNA.

### Target Sequence

In some embodiments, the one or more binding domains of the RNA guided domain are each complementary to a target sequence in a target RNA (e.g., pre-mRNA) targeted for trans-splicing. As used herein, the term “target sequence” refers to a sequence of contiguous nucleotides present in a target RNA (e.g., pre-mRNA) targeted for trans-splicing. As used herein, the term “contiguous nucleotides” refers to a string of nucleotides that are covalently linked and immediately adjacent to one another. In some embodiments, the target sequence is at least about 4, 5, 6, 7, 8, 9, 10, 11, 12, 13, 14, 15, 16, 17, 18, 19, or 20 nucleotides in length. In some embodiments, the target sequence is less than about 300, 250, 200, 100, 150, or 50 nucleotides in length. In some embodiments, the target sequence is about 5-10, about 5-15, about 5-20, about 10-20, about 10-30, about 10-40, about 10-50, about 10-60, about 10-70, about 10-80, about 10-90, about 10-100, about 50-100, about 50-150, about 50-200, about 50-250, about 50-300, about 100-200, about 100-300, or about 200-300 nucleotides in length.

In some embodiments, the target sequence is in a region comprising a splice site in the target RNA (e.g., pre-mRNA) targeted for trans-splicing. As used herein, “a splice site in the target RNA (e.g., pre-mRNA) targeted for trans-splicing” refers to a splice site in the target RNA (e.g., pre-mRNA) selected for trans-splicing, wherein upon introducing a composition, system, and/or the one or more nucleic acids comprising one or more nucleotide sequences described herein to a cell comprising the target RNA (e.g., pre-mRNA), a trans-splicing event mediates ligation between the splice site of the target RNA (e.g., pre-mRNA) and a splice site of the composition, system, and/or the one or more nucleic acids comprising one or more nucleotide sequences described herein. In some embodiments, the target sequence is upstream the splice site in the target RNA (e.g., pre-mRNA) targeted for trans-splicing. In some embodiments, the target sequence is downstream the splice site in the target RNA (e.g., pre-mRNA) targeted for trans-splicing. In some embodiments, the target sequence is in a region comprising the splice site in the target RNA (e.g., pre-mRNA) targeted for trans-splicing, wherein the region spans at least about 50, about 100, about 150, about 200, about 300, about 400, about 500, about 1,000, about 2,000, about 3,000, about 4,000, about 5,000 nucleotides.

In some embodiments, the target sequence is proximal to the splice site in the target RNA (e.g., pre-mRNA) targeted for trans-splicing. As used herein, the term “proximal to the splice site” refers to a region of less than about 500 nucleotides extending upstream and/or downstream of the splice site in the target RNA (e.g., pre-mRNA) targeted for trans-splicing.

In some embodiments, the target sequence is proximal to a splice acceptor targeted for trans-splicing. In some embodiments, the target sequence is upstream a splice acceptor targeted for trans-splicing. In some embodiments, the target sequence is downstream of a splice acceptor targeted for trans-splicing. In some embodiments, the target sequence overlaps a splice acceptor targeted for trans-splicing.

In some embodiments, the target sequence is proximal to a splice donor targeted for trans-splicing. In some embodiments, the target sequence is upstream a splice donor targeted for trans-splicing. In some embodiments, the target sequence is downstream of a splice donor targeted for trans-splicing. In some embodiments, the target sequence overlaps a splice donor targeted for trans-splicing.

In some embodiments, the target sequence is in a region of the target RNA (e.g., pre-mRNA) comprising an exon targeted for trans-splicing. As used herein, an “exon targeted for trans-splicing” refers to an exon in the target RNA that is selected for removal following trans-splicing between the target RNA and a composition, system, and/or the one or more nucleic acids comprising one or more nucleotide sequences described herein, wherein the trans-splicing results in ligation between one or more exons of the target RNA (e.g., pre-mRNA) and the at least one exonic sequence of the composition, system, and/or the one or more nucleic acids comprising one or more nucleotide sequences described herein to form a chimeric RNA molecule, and wherein the exon targeted for trans-splicing is present in the target RNA, but absent in the chimeric RNA molecule formed by the trans-splicing event.

In some embodiments, the target sequence is upstream the exon targeted for trans-splicing. In some embodiments, the target sequence is downstream the exon targeted for trans-splicing. In some embodiments, the target sequence is within the exon targeted for trans-splicing.

In some embodiments, the target sequence is proximal to a splice acceptor of the exon targeted for trans-splicing. In some embodiments, the target sequence is upstream the splice acceptor of the exon targeted for trans-splicing. In some embodiments, the target sequence is downstream the splice acceptor of the exon targeted for trans-splicing. In some embodiments, the target sequence overlaps the splice acceptor of the exon targeted for trans-splicing.

In some embodiments, the target sequence is proximal to a splice donor of the exon targeted for trans-splicing. In some embodiments, the target sequence is upstream the splice donor of the exon targeted for trans-splicing. In some embodiments, the target sequence is downstream the splice donor of the exon targeted for trans-splicing. In some embodiments, the target sequence overlaps the splice donor of the exon targeted for trans-splicing.

### RNA Binding Domain

In some embodiments, the binding domain complementary to a target sequence in the target RNA (e.g., pre-mRNA) is at least 4 nucleotides in length. In some embodiments, the binding domain is less than about 300 nucleotides in length. In some embodiments, the binding domain is at least about 5, about 6, about 7, about 8, about 9, about 10, about 11, about 12, about 13, about 14, about 15, about 16, about 17, about 18, about 19 or about 20 nucleotides in length. In some embodiments, the binding domain about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, 250, 200, 150, 100, or 50 nucleotides in length.

In some embodiments, the binding domain is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some embodiments, the binding domain is 10-50 nucleotides in length, e.g., 10-45, 10-40, 10-35, 10-30, 10-20, 11-45, 11-40, 11-35, 11-30, 11-20, 12-45, 12-40, 12-35, 12-30, 12-25, 12-20, 13-45, 13-40, 13-35, 13-30, 13-25, 13-20, 14-45, 14-40, 14-35, 14-30, 14-25, 14-20, 15-45, 15-40, 15-35, 15-30, 15-25, 15-20, 16-45, 16-40, 16-35, 16-30, 16-25, 16-20, 17-45, 17-40, 17-35, 17-30, 17-25, 17-20, 18-45, 18-40, 18-35, 18-30, 18-25, 18-20, 19-45, 19-40, 19-35, 19-30, 19-25, 19-20, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, the RNA-guided domain comprises one binding domain. In some embodiments, the RNA-guided domain comprises more than one binding domain. In some embodiments, the RNA-guided domain comprises 2, 3, 4, 5, 6, 7, 8, 9, or 10 binding domains. In some embodiments, the more than one binding domains are immediately adjacent to one another. In some embodiments, the more than one binding domains are linked by an intervening nucleotide spacer sequence.

In some embodiments, the one or more binding domains each comprise a sequence that is sufficiently complementary to its target sequence. In embodiments, the one or more binding domains comprising a sequence that is sufficiently complementary to its target sequence enables the composition, system, and/or the one or more nucleic acids comprising one or more nucleotide sequences described herein to specifically bind to the target sequence by forming base pairs. As used herein, the term “base pair” refers to two nucleobases on opposite complementary nucleic acid strands that interact by formation of specific hydrogen bonding (e.g., Watson-Crick, Hoogsteen, or reversed Hoogsteen hydrogen bonding). In some embodiments, the base pair is formed by Watson-Crick base pairing. As understood by the skilled artisan, Watson-Crick base pairing refers to the set of base pairing rules wherein a purine nucleobase binds to a pyrimidine nucleobase to form a complementary base pair. The nature of the hydrogen bonding depends upon the particular base pair. For example, a guanosine-cytosine base pair is formed by three hydrogen bonds and the adenine-thymine or adenine-uracil base pair is formed by two hydrogen bonds. It is understood that analogs or derivatives of canonical nucleobases will form base pair interactions via Watson Crick base pairing or non-canonical base pairing.

A binding domain that “specifically binds to” a target sequence in a target RNA (e.g., pre-mRNA) refers to one that will not appreciably bind to a reference sequence, e.g., a nucleic acid lacking the target sequence. For example, a composition, system, and/or the one or more nucleic acids comprising one or more nucleotide sequences described herein comprising a binding domain that specifically binds a target sequence will exhibit substantially higher binding affinity for a target RNA (e.g., pre-mRNA) comprising a nucleotide sequence comprising the target sequence compared to a target RNA (e.g., pre-mRNA) lacking the target sequence. As is understood by the skilled artisan, the binding affinity between a first nucleic acid strand and a second nucleic acid strand is measured as the melting temperature©), which is the temperature at which half the first nucleic acid strand is duplexed to the second nucleic acid strand.

In some embodiments, a binding domain is complementary to a target sequence in the target RNA (e.g., pre-mRNA) if it base-pairs to the target sequence under conditions suitable for modulating trans-splicing. Such conditions can be stringent conditions, e.g., combination of the target RNA (e.g., pre-mRNA) and composition, system, and/or the one or more nucleic acids comprising one or more nucleotide sequences described herein in buffer comprising 400 mM NaCl, 40 mM PIPES pH 6.4, 1 mM EDTA at a temperature of 50° C.-70° C. for 12-16 hours, followed by washing (see, e.g., “Molecular Cloning: A Laboratory Manual, Sambrook, et al. (1989) Cold Spring Harbor Laboratory Press). Other conditions include physiologically relevant conditions as can be encountered inside an organism. The skilled person will be able to determine the set of conditions most appropriate for a test of complementarity of two sequences in accordance with the ultimate application of the hybridized nucleotides.

snRNAs and/or snoRNAs

As used herein, a “snRNA” refers to a class of small RNA molecules that are found within the splicing speckles and Cajal bodies of the cell nucleus in eukaryotic cells. The length of an average snRNA is about 150 nucleotides. They are transcribed by either RNA polymerase II or RNA polymerase III. Their primary function is in the processing of pre-messenger RNA (hnRNA) in the nucleus. They have also been shown to aid in the regulation of transcription factors (7SK RNA) or RNA polymerase II (B2 RNA), and maintaining the telomeres. snRNA are typically associated with a set of specific proteins, and the complexes are referred to as small nuclear ribonucleoproteins (snRNP, often pronou“ced “s”urps”). Each snRNP particle is composed of a snRNA component and several snRNP-specific proteins (including Sm proteins, a family of nuclear proteins). Prior to the present disclosure, the most common human snRNA components of these complexes are known, respectively, as: U1 spliceosomal RNA, U2 spliceosomal RNA, U4 spliceosomal RNA, U5 spliceosomal RNA, and U6 spliceosomal RNA. Their nomenclature derives from their high uridine content.

In various embodiments, the present disclosure provides identifying a snRNA and/or snoRNA sequence from a database. Databases listing snRNA and/or snoRNA sequences are known in the art. For example, in some embodiments, the database is RNAcentral (see, e.g., Nucleic Acids Res 45: D128 (2017). RNAcentral is a searchable database that provides snRNA and/or snoRNA sequences annotated with unique identifiers and information regarding the one or more species in which the RNA sequence has been observed.

In some embodiments the present disclosure provides identifying a snRNA and/or snoRNA expressed by a cell or organism. Methods to identify snRNA and/or snoRNAs are known in the art. In some embodiments, cellular RNA is extracted from a cell or organism, separated by PAGE and elution from the gel, and snRNAs and/or snoRNAs are identified by sequence analysis (e.g., via 2D RNA fingerprinting or enzymatic or chemical RNA sequencing). In some embodiments, a cDNA library is generated by reverse transcription of snRNAs and/or snoRNAs obtained from a cell or organism through a selection process based on size or antibody-binding that is then subjected to sequence analysis. In some embodiments, total RNA is harvested from a cell or organism and microarray hybridization is used to detect snRNAs and/or snoRNAs. In some embodiments, genomic SELEX is used to identify snRNAs and/or snoRNAs obtained from a cell or organism. In some embodiments, the snRNA and/or snoRNA sequence is identified from any known organism. In some embodiments, the organism is a bacteria. In some embodiments, the organism is a archaebacteria. In some embodiments, the organism is a metazoan. In some embodiments, the organism is a vertebrate. In some embodiments, the organism is a mammal, amphibian, reptile, fish, or bird. In some embodiments, the organism is a human.

In some embodiments, snRNA and/or snoRNA functions to modify, alter, inhibit, or promote RNP formation and/or canonical processing. In some embodiments, the snRNA and/or snoRNA assembles into an RNP. In some embodiments, the RNP interacts with an RNA secondary structure of the composition, system, and/or the one or more nucleic acids comprising one or more nucleotide sequences described herein. In embodiments, the RNP stabilizes the RNA secondary structure of the composition, system, and/or the one or more nucleic acids comprising one or more nucleotide sequences described herein. In embodiments, the one or more RNA secondary structures comprises a single-stranded RNA sequence, a double-stranded RNA sequence, or a combination thereof. In some embodiments, the one or more RNA secondary structure comprises a duplex structure, a stem-loop, a pseudoknot, an internal loop, a multi-branch loop, a bulge loop, an external loop, or a combination thereof. In some embodiments, the RNP functions stabilize RNA-RNA interactions within the composition, system, and/or the one or more nucleic acids comprising one or more nucleotide sequences described herein and/or with a target RNA (e.g., pre-mRNA). In some embodiments, the RNP functions in to protect the composition, system, and/or the one or more nucleic acids comprising one or more nucleotide sequences described herein from degradation. In some embodiments, the RNP functions in to localize the composition, system, and/or the one or more nucleic acids comprising one or more nucleotide sequences described herein to a subcellular compartment comprising a target pre-mRNA. Methods to measure assembly of one or more nucleic acids (e.g., RNA or DNA) and one or more proteins to form an RNP are known in the art. Such methods include, but are not limited to, electrophoretic mobility shift assay (EMSA), DNA or RNA pull-down assays, oligonucleotide-targeted RNase H protection assays, fluorescent in situ hybridization co-localization, co-immunoprecipitation assays, and RNA sequencing and cross-linking methods such as high throughput sequencing crosslinking immunoprecipitation (HITS-CLIP).

In embodiments, the repRNA comprises at least one or more snRNA or snoRNA sequences. In embodiments, the at least one or more snRNA or snoRNA sequences stabilize the repRNA. In embodiments, the repRNA comprises an artificial smU7 system. In embodiments, the artificial smU7 system stabilizes the repRNA. In embodiments, the least one or more snRNA or snoRNA sequences comprise a pseudoknot at the 5′ end of the snRNA or snoRNA. In embodiments, the pseudoknot at the 5′ end of the snRNA or snoRNA stabilizes the repRNA. In embodiments, the least one or more snRNA or snoRNA sequences comprise a pseudoknot at the 3′ end of the snRNA or snoRNA. In embodiments, the pseudoknot at the 3′ end of the snRNA or snoRNA stabilizes the repRNA.

In some embodiments, a snRNA and/or snoRNA sequence identified according to a method described herein is incorporated into a composition, system, and/or the one or more nucleic acids comprising one or more nucleotide sequences described herein. In some embodiments, the entire snRNA and/or snoRNA sequence is incorporated into the composition, system, and/or the one or more nucleic acids comprising one or more nucleotide sequences described herein. In some embodiments, a portion of the snRNA and/or snoRNA sequence is incorporated into the composition, system, and/or the one or more nucleic acids comprising one or more nucleotide sequences described herein.

In some embodiments, a composition, system, and/or the one or more nucleic acids comprising one or more nucleotide sequences described herein comprises an snRNA and/or snoRNA sequence or portion thereof.

In some embodiments, the snRNA and/or snoRNA sequence or portion thereof is less than about 500 nucleotides in length. In some embodiments, the snRNA and/or snoRNA sequence or portion thereof is less than about 400 nucleotides in length. In some embodiments, the snRNA and/or snoRNA sequence or portion thereof is less than about 300 nucleotides in length. In some embodiments, the snRNA and/or snoRNA sequence or portion thereof is about 250 to about 300, about 200 to about 300, about 150 to about 300, about 100 to about 300, about 50 to about 300, about 100 to about 250, about 100 to about 200, about 100 to about 150, about 50 to about 250, about 50 to about 200, about 50 to about 150, about 50 to about 100, or about 300, 250, 200, 150, 100, or 50 nucleotides in length.

In some embodiments, the snRNA and/or snoRNA sequence or portion thereof is at least about 5, about 6, about 7, about 8, about 9, about 10, about 11, about 12, about 13, about 14, about 15, about 16, about 17, about 18, about 19 or about 20 nucleotides in length.

In some embodiments, the snRNA and/or snoRNA sequence or portion thereof is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length.

In some embodiments, the snRNA and/or snoRNA sequence or portion thereof comprises one or more RNA secondary structures that assembles into an RNP. In some embodiments, the one or more RNA secondary structures comprises a single-stranded RNA sequence, a double-stranded RNA sequence, or a combination thereof. In some embodiments, the one or more RNA secondary structure comprises a duplex structure, a stem-loop, a pseudoknot, an internal loop, a multi-branch loop, a bulge loop, an external loop, or a combination thereof. Methods to determine the secondary structure formed by an RNA sequence are known in the art. In some embodiments, the method comprises an experimental assay, e.g., nuclear magnetic resonance, cryo-electron microscopy, or X-ray crystal structure analysis. In some embodiments, the method comprises a computational prediction, e.g., based on a thermodynamic model such as Turner's nearest-neighbor model (Schroeder, et al, Methods Enzymol. 468, 371-387 (2009); Turner, et al Nucleic Acids Res. 38, D280-2 (2010)) or the Zuker algorithm (Zuker, et al Nucleic Acids Res. 9, 133-148 (1981); Zuker, et al Nucleic Acids Res. 31, 3406-3415 (2003); Markham, et al Methods Mol. Biol. 453, 3-31 (2008); Hofacker, et al Nucleic Acids Res. 31, 3429-3431 (2003); Lorenz, Algorithms Mol. Biol. 6, 26 (2011); Matthews, et al Molecular Modeling of Nucleic Acids. Vol. 682 of ACS Symposium Series. 246-257; Reuther, et al BMC Bioinform. 11, 129 (2010)); a machine learning technique such as CONTRAfold (Do, et al Bioinformatics 22, e90-8 (2006); Foo, et al Advances in Neural Information Processing Systems 20, 377-384), ContextFold (Zakov, et al J. Comput. Biol. 18, 1525-1542 (2011)); a probabilistic generative model such as stochastic context-free grammars (Rivas, et al RNA 18, 193-212 (2012)); a hybrid model such as SimFold (Andronescu, et al Bioinformatics 23, 119-28 (2007); Andronescu et al RNA 16, 2304-2318 (2010)) or MXfold (Akiyama, et al J. Bioinform. Comput. Biol. 16, 1840025 (2018)); a deep learning approach such as SPOT-RNA (Singh, et al Nat. Commun. 10, 5407 (2019)) or E2Efold (Chen et al Proceedings of the 8th International Conference on Learning Representations; arXiv: 2002.05810 (2020)).

In some embodiments, the one or more RNA secondary structures comprises a single-stranded RNA sequence, a double-stranded RNA sequence, or a combination thereof. In some embodiments, the one or more RNA secondary structure comprises a duplex structure, a stem-loop, a pseudoknot, an internal loop, a multi-branch loop, a bulge loop, an external loop, or a combination thereof. In some embodiments, the snRNA and/or snoRNA sequence or portion thereof comprises a sequence motif that assembles into an RNP. In some embodiments, the sequence motif comprises a single-stranded RNA sequence that assembles into an RNP. In some embodiments, the snRNA and/or snoRNA sequence or portion thereof comprises a sequence motif and one or more RNA secondary structures that assemble into an RNP. In some embodiments, the secondary structure and/or a sequence motif assembles with one or more proteins in a human cell to form an RNP.

In embodiments, the composition or system further comprises In embodiments, the composition or system further comprises a protein that forms an RNP or is within an RNP, with the snRNA or snoRNA, or a nucleic acid encoding the protein that forms, or is within, the RNP. In embodiments, the snRNA, snoRNA, protein that forms an RNP, a protein within the RNP, and/or a nucleic acid encoding the protein that forms or is within the RNP comprises a modification or mutation that attenuates, weakens, reduces, decreases, or ablates RNP activity, and/or leads to attenuation of RNA modifying activity as compared to an unmodified form, the RNP activity optionally being selected from cleavage, nucleic acid processing, pseudouridylation, and/or methylation. In embodiments, the snRNA, snoRNA, protein that forms an RNP, and/or a nucleic acid encoding the protein that forms the RNP or is within RNP comprises a modification or mutation that increases, stimulates, or enhances RNP activity, or enhances RNA modifying activity, the RNP activity optionally being selected from cleavage, nucleic acid processing, pseudouridylation, and/or methylation as compared to an unmodified form. In embodiments, the repRNA comprises at least one or more pseudouridylation sites. In embodiments, the repRNA comprises no pseudouridylation sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more pseudouridylation sites than the number of pseudouridylation sites in (i) an unmodified state of the snRNA or snoRNA or (ii) an exonic sequence.

In some embodiments, the snRNA and/or snoRNA sequence or portion thereof comprises one or more RNA secondary structures. In some embodiments, the snRNA and/or snoRNA sequence or portion thereof comprises one or more sequence motifs. In some embodiments, the snRNA and/or snoRNA sequence or portion thereof comprises one or more RNA secondary structures and one or more sequence motifs. In some embodiments, the sequence motif comprises a sequence selected from Table 1. In some embodiments, the sequence motif comprises an H consensus sequence comprising or consisting of a sequence set forth in Table 1. In some embodiments, the H consensus sequence comprises or consists of SEQ ID NO: 1. In some embodiments, the sequence motif comprises an ACA consensus sequence comprising or consisting of a sequence set forth in Table 1. In some embodiments, the ACA consensus sequence comprises or consists of SEQ ID NO: 2. In some embodiments, the sequence motif comprises an H/ACA box, wherein the H/ACA box comprises a sequence comprising an H consensus sequence and an ACA consensus sequence, each comprising a sequence set forth in Table 1. In some embodiments, the H/ACA box comprises a sequence comprising SEQ ID NO: 1 and SEQ ID NO: 2. In some embodiments, the sequence motif comprises a C consensus sequence comprising or consisting of a sequence set forth in Table 1. In some embodiments, the C consensus sequence comprises or consists of SEQ ID NO: 5. In some embodiments, the sequence motif comprises a D consensus sequence comprising or consisting of a sequence set forth in Table 1. In some embodiments, the D consensus sequence comprises or consists of SEQ ID NO: 6. In some embodiments, the sequence motif comprises a C/D box, wherein the C/D box comprises a C consensus sequence and a D consensus sequence, each comprising a sequence set forth in Table 1. In some embodiments, the C/D box comprises SEQ ID NO: 5 and SEQ ID NO: 6. In some embodiments, the sequence motif comprises an Sm motif comprising a sequence set forth in Table 1. In some embodiments, the Sm motif comprises SEQ ID NO: 3. In some embodiments, the Sm motif comprises SEQ ID NO: 4.

TABLE 1
snRNA and/or snoRNA sequence Motifs

| Name/Identifier | RNA Sequence | SEQ ID NO |
|---|---|---|
| H box | ANANNA | SEQ ID NO: 1 |
| | N = A, C, G, or U | |
| ACA box | ACA | SEQ ID NO: 2 |
| Sm motif | AAUUUUUGG | SEQ ID NO: 3 |
| Sm U7 motif | AAUUUGUCU | SEQ ID NO: 4 |
| C box | RUGAUGA | SEQ ID NO: 5 |
| | R = A or G | |
| D box | CUGA | SEQ ID NO: 6 |
| C′ box | RUGAUGA | SEQ ID NO: 7 |
| D′ box | CUGA | SEQ ID NO: 8 |

In some embodiments, a composition, system, and/or the one or more nucleic acids comprising one or more nucleotide sequences described herein comprises a snRNA and/or snoRNA sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 and 732-793, or portion thereof. In some embodiments, a composition, system, and/or the one or more nucleic acids comprising one or more nucleotide sequences described herein comprises a snRNA and/or snoRNA sequence selected from SEQ ID NOs: 9-657 and 732-793, or a portion thereof.

In some embodiments, the snRNA and/or snoRNA sequence or portion thereof comprises a contiguous nucleotide sequence of at least about 5, about 6, about 7, about 8, about 9, about 10, about 11, about 12, about 13, about 14, about 15, about 16, about 17, about 18, about 19 or about 20 nucleotides in length, wherein the contiguous nucleotide sequence comprises a Sm sequence motif (e.g., an Sm sequence motif set forth in Table 1).

In some embodiments, the snRNA and/or snoRNA sequence or portion thereof comprises a contiguous nucleotide sequence of about 5 to about 20, about 5 to about 30, about 5 to about 40, about 5 to about 50, about 10 to about 50, about 20 to about 50, about 30 to about 50, or about 40 to about 50 nucleotides in length, wherein the contiguous nucleotide sequence comprises a Sm sequence motif (e.g., an Sm sequence motif set forth in Table 1).

In some embodiments, the snRNA and/or snoRNA sequence or portion thereof comprises a contiguous nucleotide sequence of about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the contiguous nucleotide sequence comprises a Sm sequence motif (e.g., an Sm sequence motif set forth in Table 1).

In some embodiments, the snRNA and/or snoRNA sequence or portion thereof comprises a contiguous nucleotide sequence of about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the contiguous nucleotide sequence comprises a (i) H consensus sequence (e.g., a H consensus sequence set forth in Table 1); (ii) ACA consensus sequence (e.g., an ACA consensus sequence set forth in Table 1); or (iii) combination of (i)-(ii).

In some embodiments, the snRNA and/or snoRNA sequence or portion thereof comprises a contiguous nucleotide sequence of about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the contiguous nucleotide sequence comprises a (i)C-box motif described herein (e.g., a C-box motif set forth in Table 1), (ii) C′-box motif described herein (e.g., a C′-box motif set forth in Table 1), (iii) D-box motif described herein (e.g., a D-box motif set forth in Table 1), (iv) a D′-box motif described herein (e.g., a D′-box motif set forth in Table 1), or (v) a combination of (i)-(iv).

In some embodiments, the snRNA and/or snoRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 and 732-793, wherein the nucleotide sequence comprises a region of at least about 5, about 6, about 7, about 8, about 9, about 10, about 11, about 12, about 13, about 14, about 15, about 16, about 17, about 18, about 19 or about 20 nucleotides in length, wherein the region comprises one or more Sm sequence motifs (e.g., one or more Sm sequence motifs set forth in Table 1).

In some embodiments, the snRNA and/or snoRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 and 732-793, wherein the nucleotide sequence comprises a region of at least about 5 to about 20, about 5 to about 30, about 5 to about 40, about 5 to about 50, about 10 to about 50, about 20 to about 50, about 30 to about 50, or about 40 to about 50 nucleotides in length, wherein the region comprises one or more Sm sequence motifs (e.g., one or more Sm sequence motifs set forth in Table 1).

In some embodiments, the snRNA and/or snoRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 and 732-793, wherein the nucleotide sequence comprises a region of at least about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the region comprises one or more Sm sequence motifs (e.g., one or more Sm sequence motifs set forth in Table 1).

In some embodiments, the snRNA and/or snoRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 and 732-793, wherein the nucleotide sequence comprises a region of at least about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, wherein the region comprises (i) an H consensus sequence (e.g., an H consensus sequence set forth in Table 1); (ii) an ACA consensus sequence (e.g., an ACA consensus sequence set forth in Table 1); or (iii) a combination of (i)-(ii).

In some embodiments, the snRNA and/or snoRNA sequence or portion thereof comprises a nucleotide sequence having at least about 60%, about 65%, about 70%, about 75%, about 80%, about 85%, about 90%, about 91%, about 92%, about 93%, about 94%, about 95%, about 96%, about 97%, about 98%, about 99% identity to a sequence selected from SEQ ID NOs: 9-657 and 732-793, wherein the nucleotide sequence comprises a region of at least about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, (i) a C-box motif described herein (e.g., a C-box motif set forth in Table 1), (ii) a C′-box motif described herein (e.g., a C′-box motif set forth in Table 1), (iii) a D-box motif described herein (e.g., a D-box motif set forth in Table 1), (iv) a D′-box motif described herein (e.g., a D′-box motif set forth in Table 1), or (v) a combination of (i)-(iv).

In some embodiments, a composition, system, and/or the one or more nucleic acids comprising one or more nucleotide sequences described herein comprises a full-length snRNA or portion thereof comprising a nucleotide sequence comprising one or more sequence motifs described herein (e.g., one or more sequence motifs set forth in Table 1). In some embodiments, an snRNA sequence described herein or identified according to a method described herein is incorporated into a composition, system, and/or the one or more nucleic acids comprising one or more nucleotide sequences described herein. In some embodiments, a full-length snRNA sequence described herein or identified according to a method described herein is incorporated into a composition, system, and/or the one or more nucleic acids comprising one or more nucleotide sequences described herein. In some embodiments, a portion of a snRNA sequence described herein or identified according to a method described herein (e.g., a region of contiguous nucleotides in the snRNA) is incorporated into a composition, system, and/or the one or more nucleic acids comprising one or more nucleotide sequences described herein. In some embodiments, the full-length snRNA or portion of the snRNA assembles into a small nuclear RNP (snRNP). In some embodiments, the full-length snRNA or the portion of the snRNA comprises one or more secondary RNA structures that assembles to form an snRNP. In some embodiments, the full-length snRNA or the portion of the snRNA comprises one or more sequence motifs that assembles to form an snRNP. In some embodiments, the full-length snRNA or the portion of the snRNA comprises (i) one or more one or more secondary RNA structures, and (ii) one or more sequence motifs, wherein (i), (ii), or both assemble to form an snRNP.

Exemplary metazoan snRNA systems include U1 and U11 snRNAs, which are snRNAs that guide spliceosome RNPs to splice sites (Black, et al (1985) Cell 42:737-750; Kolossova, et al (1997) RNA 3:227). Other snRNAs include U2, U4, U4atac, U5, U6, U6atac, and U12, which also form RNPs in the major and minor spliceosome (Turunen, et al (2013) RNA 4:61-76; Nguyen, et al (2015), Nature 523:47-52; Charenton, et al (2019) Science 364:362-367). U7 RNAs arc responsible for histone pre-mRNA cleavage and polyadenylation (Strub, et al (1984) EMBO journal 3:2801-2807; Soldati, et al (1988), Molecular and Cellular Biology 8:1518-1524; Cotton, et al (1988) The EMBO Journal 7:801-808).

In some embodiments, a composition, system, and/or the one or more nucleic acids comprising one or more nucleotide sequences described herein comprises a full-length snRNA or portion thereof comprising a nucleotide sequence comprising one or more sequence motifs described herein (e.g., one or more sequence motifs set forth in Table 1), and wherein the snRNA is selected from RNU1-1, RNU1-100P, RNU1-101P, RNU1-103P, RNU1-104P, RNU1-105P, RNU1-107P, RNU1-108P, RNU1-109P, RNU1-112P, RNU1-114P, RNU1-115P, RNU1-116P, RNU1-117P, RNU1-119P, RNU1-11P, RNU1-123P, RNU1-124P, RNU1-125P, RNU1-128P, RNU1-129P, RNU1-130P, RNU1-131P, RNU1-132P, RNU1-133P, RNU1-134P, RNU1-136P, RNU1-138P, RNU1-139P, RNU1-140P, RNU1-141P, RNU1-142P, RNU1-143P, RNU1-146P, RNU1-148P, RNU1-149P, RNU1-14P, RNU1-150P, RNU1-151P, RNU1-153P, RNU1-154P, RNU1-155P, RNU1-15P, RNU1-16P, RNU1-17P, RNU1-18P, RNU1-19P, RNU1-2, RNU1-20P, RNU1-21P, RNU1-22P, RNU1-23P, RNU1-24P, RNU1-27P, RNU1-28P, RNU1-29P, RNU1-3, RNU1-30P, RNU1-31P, RNU1-32P, RNU1-33P, RNU1-34P, RNU1-35P, RNU1-36P, RNU1-38P, RNU1-39P, RNU1-4, RNU1-40P, RNU1-41P, RNU1-42P, RNU1-43P, RNU1-44P, RNU1-45P, RNU1-46P, RNU1-47P, RNU1-48P, RNU1-49P, RNU1-51P, RNU1-52P, RNU1-54P, RNU1-55P, RNU1-56P, RNU1-57P, RNU1-58P, RNU1-5P, RNU1-61P, RNU1-62P, RNU1-63P, RNU1-64P, RNU1-65P, RNU1-67P, RNU1-68P, RNU1-6P, RNU1-70P, RNU1-72P, RNU1-73P, RNU1-74P, RNU1-75P, RNU1-76P, RNU1-77P, RNU1-78P, RNU1-79P, RNU1-7P, RNU1-80P, RNU1-82P, RNU1-83P, RNU1-84P, RNU1-86P, RNU1-88P, RNU1-89P, RNU1-8P, RNU1-91P, RNU1-93P, RNU1-94P, RNU1-95P, RNU1-96P, RNU1-97P, RNU1-98P, RNU11, RNU11-2P, RNU11-3P, RNU11-4P, RNU11-5P, RNU11-6P, RNU12, RNU12-2P, RNU2-12P, RNU2-13P, RNU2-16P, RNU2-18P, RNU2-19P, RNU2-24P, RNU2-27P, RNU2-30P, RNU2-31P, RNU2-34P, RNU2-35P, RNU2-37P, RNU2-38P, RNU2-41P, RNU2-42P, RNU2-46P, RNU2-50P, RNU2-53P, RNU2-55P, RNU2-60P, RNU2-66P, RNU2-69P, RNU2-70P, RNU2-72P, RNU2-7P, RNU2-9P, RNU4-1, RNU4-10P, RNU4-11P, RNU4-12P, RNU4-13P, RNU4-14P, RNU4-15P, RNU4-16P, RNU4-17P, RNU4-18P, RNU4-2, RNU4-20P, RNU4-21P, RNU4-22P, RNU4-23P, RNU4-24P, RNU4-26P, RNU4-27P, RNU4-28P, RNU4-29P, RNU4-30P, RNU4-31P, RNU4-32P, RNU4-33P, RNU4-34P, RNU4-35P, RNU4-36P, RNU4-37P, RNU4-38P, RNU4-39P, RNU4-40P, RNU4-41P, RNU4-42P, RNU4-43P, RNU4-44P, RNU4-45P, RNU4-46P, RNU4-47P, RNU4-49P, RNU4-4P, RNU4-50P, RNU4-51P, RNU4-52P, RNU4-53P, RNU4-54P, RNU4-55P, RNU4-56P, RNU4-57P, RNU4-58P, RNU4-59P, RNU4-5P, RNU4-60P, RNU4-61P, RNU4-62P, RNU4-63P, RNU4-64P, RNU4-65P, RNU4-66P, RNU4-67P, RNU4-68P, RNU4-69P, RNU4-6P, RNU4-70P, RNU4-71P, RNU4-72P, RNU4-73P, RNU4-74P, RNU4-75P, RNU4-76P, RNU4-77P, RNU4-78P, RNU4-79P, RNU4-7P, RNU4-80P, RNU4-81P, RNU4-82P, RNU4-83P, RNU4-84P, RNU4-85P, RNU4-87P, RNU4-88P, RNU4-89P, RNU4-8P, RNU4-90P, RNU4-91P, RNU4-92P, RNU4-9P, RNU4ATAC, RNU4ATAC10P, RNU4ATAC11P, RNU4ATAC12P, RNU4ATAC13P, RNU4ATAC14P, RNU4ATAC15P, RNU4ATAC16P, RNU4ATAC17P, RNU4ATAC18P, RNU4ATAC2P, RNU4ATAC3P, RNU4ATAC4P, RNU4ATAC5P, RNU4ATAC6P, RNU4ATAC7P, RNU4ATAC8P, RNU4ATAC9P, RNUSA-1, RNUSA-2P, RNUSA-3P, RNUSA-4P, RNUSA-5P, RNUSA-6P, RNUSA-7P, RNUSA-8P, RNU5B-1, RNU5B-2P, RNUSB-3P, RNUSB-4P, RNUSB-6P, RNUSD-1, RNU5D-2P, RNUSE-1, RNUSE-10P, RNUSE-3P, RNUSE-4P, RNUSE-5P, RNUSE-6P, RNUSE-7P, RNUSE-8P, RNUSE-9P, RNU5F-1, RNU5F-2P, RNU5F-3P, RNU5F-4P, RNU5F-6P, RNU5F-7P, RNU5F-8P, RNU6-1, RNU6-1000P, RNU6-1001P, RNU6-1003P, RNU6-1004P, RNU6-1005P, RNU6-1006P, RNU6-1007P, RNU6-1008P, RNU6-1009P, RNU6-100P, RNU6-1010P, RNU6-1011P, RNU6-1012P, RNU6-1013P, RNU6-1014P, RNU6-1015P, RNU6-1016P, RNU6-1017P, RNU6-1018P, RNU6-1019P, RNU6-101P, RNU6-1020P, RNU6-1021P, RNU6-1022P, RNU6-1023P, RNU6-1024P, RNU6-1025P, RNU6-1026P, RNU6-1027P, RNU6-1028P, RNU6-1029P, RNU6-102P, RNU6-1031P, RNU6-1032P, RNU6-1034P, RNU6-1035P, RNU6-1036P, RNU6-1037P, RNU6-1038P, RNU6-1039P, RNU6-103P, RNU6-1040P, RNU6-1041P, RNU6-1042P, RNU6-1043P, RNU6-1044P, RNU6-1045P, RNU6-1046P, RNU6-1047P, RNU6-1048P, RNU6-1049P, RNU6-104P, RNU6-1050P, RNU6-1051P, RNU6-1052P, RNU6-1053P, RNU6-1054P, RNU6-1055P, RNU6-1056P, RNU6-1057P, RNU6-1059P, RNU6-105P, RNU6-1060P, RNU6-1061P, RNU6-1062P, RNU6-1064P, RNU6-1065P, RNU6-1066P, RNU6-1067P, RNU6-1068P, RNU6-1069P, RNU6-106P, RNU6-1071P, RNU6-1072P, RNU6-1073P, RNU6-1074P, RNU6-1075P, RNU6-1076P, RNU6-1077P, RNU6-1078P, RNU6-1079P, RNU6-107P, RNU6-1080P, RNU6-1081P, RNU6-1082P, RNU6-1083P, RNU6-1084P, RNU6-1085P, RNU6-1086P, RNU6-1087P, RNU6-1088P, RNU6-1089P, RNU6-108P, RNU6-1090P, RNU6-1091P, RNU6-1092P, RNU6-1093P, RNU6-1094P, RNU6-1095P, RNU6-1096P, RNU6-1097P, RNU6-1098P, RNU6-1099P, RNU6-109P, RNU6-10P, RNU6-1100P, RNU6-1101P, RNU6-1102P, RNU6-1103P, RNU6-1104P, RNU6-1105P, RNU6-1106P, RNU6-1107P, RNU6-1108P, RNU6-1109P, RNU6-110P, RNU6-1110P, RNU6-1111P, RNU6-1112P, RNU6-1113P, RNU6-1114P, RNU6-1115P, RNU6-1116P, RNU6-1117P, RNU6-1118P, RNU6-1119P, RNU6-111P, RNU6-1120P, RNU6-1121P, RNU6-1122P, RNU6-1123P, RNU6-1124P, RNU6-1125P, RNU6-1126P, RNU6-1127P, RNU6-1128P, RNU6-1129P, RNU6-112P, RNU6-1130P, RNU6-1131P, RNU6-1132P, RNU6-1133P, RNU6-1134P, RNU6-1135P, RNU6-1136P, RNU6-1137P, RNU6-1138P, RNU6-113P, RNU6-1140P, RNU6-1141P, RNU6-1143P, RNU6-1144P, RNU6-1145P, RNU6-1146P, RNU6-1147P, RNU6-1148P, RNU6-1149P, RNU6-114P, RNU6-1150P, RNU6-1151P, RNU6-1152P, RNU6-1153P, RNU6-1154P, RNU6-1155P, RNU6-1156P, RNU6-1157P, RNU6-1158P, RNU6-1159P, RNU6-115P, RNU6-1160P, RNU6-1161P, RNU6-1162P, RNU6-1163P, RNU6-1164P, RNU6-1165P, RNU6-1167P, RNU6-1168P, RNU6-1169P, RNU6-116P, RNU6-1170P, RNU6-1171P, RNU6-1172P, RNU6-1174P, RNU6-1175P, RNU6-1176P, RNU6-1177P, RNU6-1178P, RNU6-1179P, RNU6-117P, RNU6-1180P, RNU6-1181P, RNU6-1183P, RNU6-1184P, RNU6-1186P, RNU6-1187P, RNU6-1188P, RNU6-1189P, RNU6-118P, RNU6-1190P, RNU6-1191P, RNU6-1192P, RNU6-1193P, RNU6-1194P, RNU6-1195P, RNU6-1196P, RNU6-1197P, RNU6-1198P, RNU6-1199P, RNU6-119P, RNU6-11P, RNU6-1200P, RNU6-1201P, RNU6-1203P, RNU6-1204P, RNU6-1205P, RNU6-1206P, RNU6-1207P, RNU6-1208P, RNU6-1209P, RNU6-120P, RNU6-1210P, RNU6-1211P, RNU6-1212P, RNU6-1213P, RNU6-1214P, RNU6-1215P, RNU6-1216P, RNU6-1217P, RNU6-1218P, RNU6-1219P, RNU6-121P, RNU6-1220P, RNU6-1222P, RNU6-1223P, RNU6-1224P, RNU6-1225P, RNU6-1226P, RNU6-1227P, RNU6-1228P, RNU6-1229P, RNU6-122P, RNU6-1230P, RNU6-1231P, RNU6-1232P, RNU6-1233P, RNU6-1234P, RNU6-1235P, RNU6-1236P, RNU6-1237P, RNU6-1238P, RNU6-1239P, RNU6-123P, RNU6-1240P, RNU6-1241P, RNU6-1242P, RNU6-1243P, RNU6-1244P, RNU6-1245P, RNU6-1246P, RNU6-1247P, RNU6-1248P, RNU6-1249P, RNU6-1250P, RNU6-1251P, RNU6-1252P, RNU6-1254P, RNU6-1255P, RNU6-1256P, RNU6-1257P, RNU6-1258P, RNU6-125P, RNU6-1260P, RNU6-1261P, RNU6-1262P, RNU6-1263P, RNU6-1264P, RNU6-1265P, RNU6-1266P, RNU6-1267P, RNU6-1268P, RNU6-1269P, RNU6-126P, RNU6-1270P, RNU6-1271P, RNU6-1272P, RNU6-1273P, RNU6-1274P, RNU6-1275P, RNU6-1276P, RNU6-1277P, RNU6-1278P, RNU6-1279P, RNU6-127P, RNU6-1280P, RNU6-1281P, RNU6-1282P, RNU6-1283P, RNU6-1284P, RNU6-1285P, RNU6-1286P, RNU6-1287P, RNU6-1288P, RNU6-1289P, RNU6-128P, RNU6-1290P, RNU6-1291P, RNU6-1292P, RNU6-1293P, RNU6-1294P, RNU6-1296P, RNU6-1297P, RNU6-1298P, RNU6-1299P, RNU6-129P, RNU6-12P, RNU6-1300P, RNU6-1301P, RNU6-1303P, RNU6-1304P, RNU6-1305P, RNU6-1306P, RNU6-1307P, RNU6-1308P, RNU6-1309P, RNU6-130P, RNU6-1310P, RNU6-1311P, RNU6-1312P, RNU6-1313P, RNU6-1314P, RNU6-1315P, RNU6-1316P, RNU6-1317P, RNU6-1318P, RNU6-1319P, RNU6-131P, RNU6-1320P, RNU6-1321P, RNU6-1322P, RNU6-1323P, RNU6-1324P, RNU6-1325P, RNU6-1326P, RNU6-1327P, RNU6-1328P, RNU6-1329P, RNU6-132P, RNU6-1330P, RNU6-1331P, RNU6-1332P, RNU6-1333P, RNU6-1334P, RNU6-1335P, RNU6-1336P, RNU6-1337P, RNU6-1338P, RNU6-1339P, RNU6-133P, RNU6-1340P, RNU6-135P, RNU6-136P, RNU6-137P, RNU6-138P, RNU6-139P, RNU6-13P, RNU6-140P, RNU6-141P, RNU6-142P, RNU6-143P, RNU6-144P, RNU6-145P, RNU6-146P, RNU6-147P, RNU6-148P, RNU6-14P, RNU6-150P, RNU6-151P, RNU6-152P, RNU6-153P, RNU6-154P, RNU6-155P, RNU6-156P, RNU6-157P, RNU6-158P, RNU6-159P, RNU6-15P, RNU6-160P, RNU6-161P, RNU6-162P, RNU6-163P, RNU6-164P, RNU6-165P, RNU6-166P, RNU6-167P, RNU6-168P, RNU6-169P, RNU6-16P, RNU6-170P, RNU6-171P, RNU6-172P, RNU6-173P, RNU6-174P, RNU6-175P, RNU6-176P, RNU6-177P, RNU6-178P, RNU6-179P, RNU6-17P, RNU6-180P, RNU6-181P, RNU6-182P, RNU6-183P, RNU6-184P, RNU6-185P, RNU6-187P, RNU6-188P, RNU6-189P, RNU6-18P, RNU6-190P, RNU6-191P, RNU6-192P, RNU6-193P, RNU6-194P, RNU6-195P, RNU6-196P, RNU6-197P, RNU6-198P, RNU6-199P, RNU6-19P, RNU6-2, RNU6-200P, RNU6-201P, RNU6-202P, RNU6-203P, RNU6-204P, RNU6-205P, RNU6-206P, RNU6-207P, RNU6-208P, RNU6-209P, RNU6-20P, RNU6-210P, RNU6-211P, RNU6-212P, RNU6-213P, RNU6-214P, RNU6-215P, RNU6-216P, RNU6-217P, RNU6-218P, RNU6-219P, RNU6-21P, RNU6-220P, RNU6-221P, RNU6-222P, RNU6-223P, RNU6-224P, RNU6-225P, RNU6-226P, RNU6-227P, RNU6-228P, RNU6-229P, RNU6-22P, RNU6-230P, RNU6-231P, RNU6-232P, RNU6-233P, RNU6-234P, RNU6-235P, RNU6-236P, RNU6-237P, RNU6-238P, RNU6-239P, RNU6-23P, RNU6-240P, RNU6-241P, RNU6-242P, RNU6-243P, RNU6-244P, RNU6-245P, RNU6-246P, RNU6-247P, RNU6-248P, RNU6-249P, RNU6-24P, RNU6-250P, RNU6-251P, RNU6-252P, RNU6-253P, RNU6-254P, RNU6-255P, RNU6-256P, RNU6-257P, RNU6-258P, RNU6-259P, RNU6-25P, RNU6-260P, RNU6-261P, RNU6-262P, RNU6-263P, RNU6-264P, RNU6-266P, RNU6-267P, RNU6-268P, RNU6-269P, RNU6-26P, RNU6-270P, RNU6-271P, RNU6-272P, RNU6-273P, RNU6-274P, RNU6-275P, RNU6-276P, RNU6-277P, RNU6-278P, RNU6-279P, RNU6-27P, RNU6-280P, RNU6-281P, RNU6-282P, RNU6-283P, RNU6-284P, RNU6-285P, RNU6-286P, RNU6-287P, RNU6-288P, RNU6-289P, RNU6-28P, RNU6-290P, RNU6-291P, RNU6-293P, RNU6-294P, RNU6-295P, RNU6-296P, RNU6-297P, RNU6-298P, RNU6-299P, RNU6-29P, RNU6-300P, RNU6-301P, RNU6-302P, RNU6-303P, RNU6-304P, RNU6-306P, RNU6-307P, RNU6-308P, RNU6-309P, RNU6-30P, RNU6-310P, RNU6-311P, RNU6-312P, RNU6-313P, RNU6-314P, RNU6-315P, RNU6-316P, RNU6-317P, RNU6-318P, RNU6-319P, RNU6-31P, RNU6-320P, RNU6-321P, RNU6-322P, RNU6-323P, RNU6-324P, RNU6-325P, RNU6-326P, RNU6-327P, RNU6-328P, RNU6-329P, RNU6-32P, RNU6-330P, RNU6-331P, RNU6-332P, RNU6-333P, RNU6-334P, RNU6-335P, RNU6-336P, RNU6-337P, RNU6-338P, RNU6-339P, RNU6-33P, RNU6-340P, RNU6-341P, RNU6-342P, RNU6-343P, RNU6-344P, RNU6-345P, RNU6-346P, RNU6-347P, RNU6-348P, RNU6-349P, RNU6-34P, RNU6-351P, RNU6-352P, RNU6-353P, RNU6-354P, RNU6-355P, RNU6-356P, RNU6-358P, RNU6-359P, RNU6-35P, RNU6-360P, RNU6-361P, RNU6-362P, RNU6-363P, RNU6-364P, RNU6-365P, RNU6-366P, RNU6-367P, RNU6-368P, RNU6-369P, RNU6-36P, RNU6-370P, RNU6-371P, RNU6-373P, RNU6-374P, RNU6-375P, RNU6-376P, RNU6-377P, RNU6-378P, RNU6-379P, RNU6-37P, RNU6-380P, RNU6-381P, RNU6-382P, RNU6-383P, RNU6-384P, RNU6-386P, RNU6-387P, RNU6-388P, RNU6-389P, RNU6-38P, RNU6-390P, RNU6-391P, RNU6-392P, RNU6-393P, RNU6-394P, RNU6-395P, RNU6-396P, RNU6-397P, RNU6-398P, RNU6-399P, RNU6-39P, RNU6-3P, RNU6-400P, RNU6-401P, RNU6-402P, RNU6-403P, RNU6-405P, RNU6-406P, RNU6-407P, RNU6-408P, RNU6-409P, RNU6-40P, RNU6-410P, RNU6-411P, RNU6-412P, RNU6-413P, RNU6-414P, RNU6-415P, RNU6-416P, RNU6-417P, RNU6-418P, RNU6-419P, RNU6-41P, RNU6-420P, RNU6-421P, RNU6-422P, RNU6-424P, RNU6-425P, RNU6-426P, RNU6-428P, RNU6-429P, RNU6-42P, RNU6-430P, RNU6-431P, RNU6-432P, RNU6-433P, RNU6-434P, RNU6-435P, RNU6-436P, RNU6-437P, RNU6-438P, RNU6-439P, RNU6-43P, RNU6-440P, RNU6-441P, RNU6-442P, RNU6-444P, RNU6-445P, RNU6-446P, RNU6-447P, RNU6-448P, RNU6-449P, RNU6-44P, RNU6-450P, RNU6-451P, RNU6-452P, RNU6-453P, RNU6-454P, RNU6-455P, RNU6-456P, RNU6-457P, RNU6-458P, RNU6-45P, RNU6-460P, RNU6-461P, RNU6-462P, RNU6-463P, RNU6-464P, RNU6-465P, RNU6-466P, RNU6-467P, RNU6-468P, RNU6-469P, RNU6-46P, RNU6-470P, RNU6-471P, RNU6-472P, RNU6-473P, RNU6-474P, RNU6-475P, RNU6-476P, RNU6-477P, RNU6-478P, RNU6-479P, RNU6-47P, RNU6-480P, RNU6-481P, RNU6-482P, RNU6-483P, RNU6-484P, RNU6-485P, RNU6-486P, RNU6-487P, RNU6-488P, RNU6-489P, RNU6-48P, RNU6-490P, RNU6-491P, RNU6-492P, RNU6-493P, RNU6-494P, RNU6-495P, RNU6-496P, RNU6-497P, RNU6-498P, RNU6-499P, RNU6-49P, RNU6-4P, RNU6-500P, RNU6-501P, RNU6-502P, RNU6-503P, RNU6-504P, RNU6-505P, RNU6-506P, RNU6-507P, RNU6-508P, RNU6-509P, RNU6-50P, RNU6-510P, RNU6-511P, RNU6-512P, RNU6-513P, RNU6-514P, RNU6-516P, RNU6-517P, RNU6-518P, RNU6-519P, RNU6-520P, RNU6-521P, RNU6-522P, RNU6-523P, RNU6-524P, RNU6-525P, RNU6-526P, RNU6-527P, RNU6-528P, RNU6-529P, RNU6-530P, RNU6-531P, RNU6-532P, RNU6-533P, RNU6-534P, RNU6-535P, RNU6-536P, RNU6-537P, RNU6-538P, RNU6-539P, RNU6-53P, RNU6-540P, RNU6-541P, RNU6-542P, RNU6-543P, RNU6-544P, RNU6-545P, RNU6-546P, RNU6-547P, RNU6-548P, RNU6-549P, RNU6-54P, RNU6-550P, RNU6-551P, RNU6-552P, RNU6-553P, RNU6-554P, RNU6-555P, RNU6-556P, RNU6-557P, RNU6-558P, RNU6-559P, RNU6-55P, RNU6-560P, RNU6-561P, RNU6-562P, RNU6-563P, RNU6-564P, RNU6-565P, RNU6-566P, RNU6-567P, RNU6-56P, RNU6-570P, RNU6-571P, RNU6-572P, RNU6-573P, RNU6-574P, RNU6-575P, RNU6-576P, RNU6-577P, RNU6-578P, RNU6-579P, RNU6-57P, RNU6-580P, RNU6-581P, RNU6-582P, RNU6-583P, RNU6-584P, RNU6-586P, RNU6-587P, RNU6-588P, RNU6-589P, RNU6-58P, RNU6-590P, RNU6-591P, RNU6-592P, RNU6-593P, RNU6-595P, RNU6-596P, RNU6-597P, RNU6-598P, RNU6-599P, RNU6-59P, RNU6-5P, RNU6-600P, RNU6-601P, RNU6-602P, RNU6-603P, RNU6-604P, RNU6-605P, RNU6-606P, RNU6-607P, RNU6-608P, RNU6-609P, RNU6-60P, RNU6-610P, RNU6-611P, RNU6-612P, RNU6-613P, RNU6-614P, RNU6-615P, RNU6-616P, RNU6-617P, RNU6-618P, RNU6-619P, RNU6-61P, RNU6-620P, RNU6-621P, RNU6-622P, RNU6-623P, RNU6-624P, RNU6-625P, RNU6-626P, RNU6-627P, RNU6-628P, RNU6-629P, RNU6-62P, RNU6-630P, RNU6-631P, RNU6-632P, RNU6-633P, RNU6-634P, RNU6-635P, RNU6-636P, RNU6-637P, RNU6-638P, RNU6-639P, RNU6-63P, RNU6-640P, RNU6-641P, RNU6-642P, RNU6-643P, RNU6-644P, RNU6-645P, RNU6-646P, RNU6-647P, RNU6-648P, RNU6-649P, RNU6-64P, RNU6-650P, RNU6-651P, RNU6-652P, RNU6-653P, RNU6-654P, RNU6-655P, RNU6-656P, RNU6-657P, RNU6-658P, RNU6-659P, RNU6-65P, RNU6-660P, RNU6-661P, RNU6-662P, RNU6-663P, RNU6-664P, RNU6-665P, RNU6-666P, RNU6-667P, RNU6-668P, RNU6-669P, RNU6-66P, RNU6-670P, RNU6-672P, RNU6-673P, RNU6-674P, RNU6-675P, RNU6-677P, RNU6-678P, RNU6-679P, RNU6-67P, RNU6-680P, RNU6-681P, RNU6-682P, RNU6-684P, RNU6-685P, RNU6-686P, RNU6-687P, RNU6-689P, RNU6-68P, RNU6-690P, RNU6-692P, RNU6-693P, RNU6-694P, RNU6-695P, RNU6-696P, RNU6-697P, RNU6-698P, RNU6-699P, RNU6-6P, RNU6-7, RNU6-700P, RNU6-701P, RNU6-702P, RNU6-703P, RNU6-704P, RNU6-705P, RNU6-706P, RNU6-707P, RNU6-708P, RNU6-709P, RNU6-70P, RNU6-710P, RNU6-711P, RNU6-712P, RNU6-713P, RNU6-714P, RNU6-715P, RNU6-716P, RNU6-717P, RNU6-718P, RNU6-719P, RNU6-71P, RNU6-720P, RNU6-721P, RNU6-722P, RNU6-723P, RNU6-724P, RNU6-725P, RNU6-726P, RNU6-727P, RNU6-728P, RNU6-729P, RNU6-72P, RNU6-730P, RNU6-731P, RNU6-732P, RNU6-733P, RNU6-735P, RNU6-737P, RNU6-738P, RNU6-739P, RNU6-73P, RNU6-740P, RNU6-741P, RNU6-742P, RNU6-743P, RNU6-744P, RNU6-745P, RNU6-746P, RNU6-747P, RNU6-748P, RNU6-749P, RNU6-74P, RNU6-750P, RNU6-751P, RNU6-752P, RNU6-753P, RNU6-754P, RNU6-755P, RNU6-756P, RNU6-757P, RNU6-758P, RNU6-759P, RNU6-75P, RNU6-760P, RNU6-761P, RNU6-762P, RNU6-763P, RNU6-764P, RNU6-765P, RNU6-766P, RNU6-767P, RNU6-768P, RNU6-769P, RNU6-76P, RNU6-770P, RNU6-771P, RNU6-772P, RNU6-774P, RNU6-775P, RNU6-776P, RNU6-777P, RNU6-778P, RNU6-77P, RNU6-780P, RNU6-781P, RNU6-782P, RNU6-783P, RNU6-784P, RNU6-785P, RNU6-786P, RNU6-787P, RNU6-788P, RNU6-789P, RNU6-78P, RNU6-790P, RNU6-791P, RNU6-792P, RNU6-793P, RNU6-794P, RNU6-795P, RNU6-796P, RNU6-797P, RNU6-798P, RNU6-799P, RNU6-79P, RNU6-8, RNU6-800P, RNU6-801P, RNU6-803P, RNU6-804P, RNU6-805P, RNU6-806P, RNU6-807P, RNU6-808P, RNU6-809P, RNU6-80P, RNU6-810P, RNU6-811P, RNU6-812P, RNU6-813P, RNU6-815P, RNU6-816P, RNU6-817P, RNU6-818P, RNU6-819P, RNU6-81P, RNU6-820P, RNU6-821P, RNU6-822P, RNU6-823P, RNU6-824P, RNU6-826P, RNU6-827P, RNU6-828P, RNU6-829P, RNU6-82P, RNU6-830P, RNU6-831P, RNU6-832P, RNU6-833P, RNU6-834P, RNU6-835P, RNU6-836P, RNU6-837P, RNU6-838P, RNU6-839P, RNU6-83P, RNU6-840P, RNU6-841P, RNU6-842P, RNU6-843P, RNU6-844P, RNU6-845P, RNU6-847P, RNU6-848P, RNU6-849P, RNU6-84P, RNU6-850P, RNU6-851P, RNU6-853P, RNU6-854P, RNU6-855P, RNU6-856P, RNU6-857P, RNU6-858P, RNU6-859P, RNU6-85P, RNU6-860P, RNU6-861P, RNU6-862P, RNU6-863P, RNU6-864P, RNU6-865P, RNU6-866P, RNU6-867P, RNU6-869P, RNU6-86P, RNU6-871P, RNU6-873P, RNU6-874P, RNU6-875P, RNU6-876P, RNU6-877P, RNU6-878P, RNU6-879P, RNU6-87P, RNU6-880P, RNU6-881P, RNU6-882P, RNU6-883P, RNU6-884P, RNU6-885P, RNU6-886P, RNU6-887P, RNU6-888P, RNU6-889P, RNU6-88P, RNU6-890P, RNU6-891P, RNU6-892P, RNU6-893P, RNU6-894P, RNU6-895P, RNU6-896P, RNU6-897P, RNU6-898P, RNU6-899P, RNU6-89P, RNU6-9, RNU6-900P, RNU6-901P, RNU6-902P, RNU6-903P, RNU6-904P, RNU6-905P, RNU6-906P, RNU6-907P, RNU6-908P, RNU6-909P, RNU6-90P, RNU6-910P, RNU6-911P, RNU6-912P, RNU6-913P, RNU6-914P, RNU6-915P, RNU6-916P, RNU6-917P, RNU6-918P, RNU6-919P, RNU6-91P, RNU6-920P, RNU6-921P, RNU6-922P, RNU6-923P, RNU6-924P, RNU6-925P, RNU6-926P, RNU6-927P, RNU6-928P, RNU6-929P, RNU6-92P, RNU6-930P, RNU6-931P, RNU6-932P, RNU6-933P, RNU6-934P, RNU6-935P, RNU6-936P, RNU6-937P, RNU6-938P, RNU6-939P, RNU6-940P, RNU6-941P, RNU6-942P, RNU6-943P, RNU6-944P, RNU6-945P, RNU6-946P, RNU6-947P, RNU6-948P, RNU6-949P, RNU6-94P, RNU6-950P, RNU6-951P, RNU6-952P, RNU6-953P, RNU6-954P, RNU6-955P, RNU6-956P, RNU6-957P, RNU6-958P, RNU6-959P, RNU6-95P, RNU6-960P, RNU6-961P, RNU6-964P, RNU6-965P, RNU6-966P, RNU6-967P, RNU6-968P, RNU6-969P, RNU6-970P, RNU6-971P, RNU6-972P, RNU6-973P, RNU6-974P, RNU6-975P, RNU6-976P, RNU6-977P, RNU6-978P, RNU6-979P, RNU6-97P, RNU6-980P, RNU6-982P, RNU6-983P, RNU6-984P, RNU6-985P, RNU6-986P, RNU6-987P, RNU6-988P, RNU6-989P, RNU6-98P, RNU6-990P, RNU6-991P, RNU6-992P, RNU6-993P, RNU6-994P, RNU6-995P, RNU6-996P, RNU6-997P, RNU6-998P, RNU6-999P, RNU6-99P, RNU6ATAC, RNU6ATAC10P, RNU6ATAC11P, RNU6ATAC12P, RNU6ATAC13P, RNU6ATAC14P, RNU6ATAC15P, RNU6ATAC16P, RNU6ATAC17P, RNU6ATAC18P, RNU6ATAC19P, RNU6ATAC20P, RNU6ATAC21P, RNU6ATAC22P, RNU6ATAC23P, RNU6ATAC24P, RNU6ATAC25P, RNU6ATAC26P, RNU6ATAC27P, RNU6ATAC28P, RNU6ATAC29P, RNU6ATAC2P, RNU6ATAC30P, RNU6ATAC31P, RNU6ATAC32P, RNU6ATAC33P, R RNU6ATAC34P, RNU6ATAC36P, RNU6ATAC37P, RNU6ATAC38P, RNU6ATAC39P, RNU6ATAC3P, RNU6ATAC40P, RNU6ATAC41P, RNU6ATAC42P, RNU6ATAC4P, RNU6ATAC5P, RNU6ATAC6P, RNU6ATAC7P, RNU6ATAC8P, RNU6ATAC9P, RNU6V, RNU7-1, RNU7-102P, RNU7-103P, RNU7-104P, RNU7-105P, RNU7-106P, RNU7-107P, RNU7-10P, RNU7-110P, RNU7-111P, RNU7-113P, RNU7-115P, RNU7-116P, RNU7-119P, RNU7-11P, RNU7-120P, RNU7-121P, RNU7-123P, RNU7-124P, RNU7-125P, RNU7-126P, RNU7-127P, RNU7-128P, RNU7-129P, RNU7-12P, RNU7-130P, RNU7-133P, RNU7-134P, RNU7-136P, RNU7-137P, RNU7-138P, RNU7-13P, RNU7-140P, RNU7-141P, RNU7-143P, RNU7-144P, RNU7-147P, RNU7-148P, RNU7-149P, RNU7-14P, RNU7-151P, RNU7-152P, RNU7-153P, RNU7-154P, RNU7-155P, RNU7-156P, RNU7-157P, RNU7-159P, RNU7-160P, RNU7-161P, RNU7-164P, RNU7-165P, RNU7-167P, RNU7-169P, RNU7-170P, RNU7-171P, RNU7-172P, RNU7-173P, RNU7-174P, RNU7-175P, RNU7-176P, RNU7-179P, RNU7-180P, RNU7-181P, RNU7-182P, RNU7-183P, RNU7-185P, RNU7-186P, RNU7-187P, RNU7-188P, RNU7-18P, RNU7-190P, RNU7-192P, RNU7-193P, RNU7-194P, RNU7-195P, RNU7-196P, RNU7-197P, RNU7-19P, RNU7-200P, RNU7-20P, RNU7-21P, RNU7-22P, RNU7-23P, RNU7-24P, RNU7-25P, RNU7-26P, RNU7-27P, RNU7-28P, RNU7-29P, RNU7-2P, RNU7-30P, RNU7-34P, RNU7-35P, RNU7-37P, RNU7-38P, RNU7-3P, RNU7-40P, RNU7-41P, RNU7-43P, RNU7-45P, RNU7-46P, RNU7-47P, RNU7-48P, RNU7-49P, RNU7-4P, RNU7-50P, RNU7-51P, RNU7-52P, RNU7-53P, RNU7-54P, RNU7-55P, RNU7-56P, RNU7-57P, RNU7-59P, RNU7-60P, RNU7-61P, RNU7-62P, RNU7-63P, RNU7-65P, RNU7-66P, RNU7-67P, RNU7-69P, RNU7-6P, RNU7-70P, RNU7-71P, RNU7-73P, RNU7-74P, RNU7-75P, RNU7-77P, RNU7-79P, RNU7-7P, RNU7-80P, RNU7-81P, RNU7-82P, RNU7-84P, RNU7-85P, RNU7-87P, RNU7-88P, RNU7-8P, RNU7-90P, RNU7-92P, RNU7-93P, RNU7-94P, RNU7-95P, RNU7-96P, RNU7-97P, RNU7-99P, RNU7-9P, RNVU1-1, RNVU1-14, RNVU1-15, RNVU1-17, RNVU1-18, RNVU1-19, RNVU1-2, RNVU1-21, RNVU1-22, RNVU1-23, RNVU1-24, RNVU1-25, RNVU1-26, RNVU1-27, RNVU1-28, RNVU1-29, RNVU1-2A, RNVU1-3, RNVU1-30, RNVU1-31, RNVU1-32, RNVU1-33, RNVU1-34, RNVU1-4, RNVU1-6, RNVU1-7, RNVU1-8, U1, U2, U4, U6, U7.

In some embodiments, a composition, system, and/or the one or more nucleic acids comprising one or more nucleotide sequences described herein comprises a full-length snRNA or portion thereof comprising a nucleotide sequence comprising one or more sequence motifs described herein (e.g., one or more sequence motifs set forth in Table 1), and wherein the snRNA is selected from a U1 snRNA, a U2 snRNA, a U4 snRNA, a U4atac snRNA, a U5 snRNA, a U6 snRNA, a U6atac snRNA, a U11 snRNA, a U12 snRNA, and a U7 snRNA.

In embodiments, the snRNA comprises a M6A modification. In embodiments, the snRNA comprises a M6A modification when the present methods are undertaken in cis or trans, as described herein. In embodiments, the snRNA or snoRNA is modified to comprise at least one or more M6A sites. In embodiments, the snRNA or snoRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A modifications in (i) an unmodified state of the snRNA or snoRNA or (ii) an exonic sequence. In embodiments, the snRNA or snoRNA is modified to not comprise M6A sites. In embodiments, the repRNA comprises at least one or more M6A sites. In embodiments, the repRNA is modified to comprise at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30 or more M6A sites than the number of M6A sites in (i) an unmodified state of the repRNA or (ii) an exonic sequence. In embodiments, the repRNA comprises no M6A sites.

In embodiments, the repRNA further comprises a ribozyme site. In embodiments, the ribozyme site is a hairpin, hammerhead, hepatitis delta virus (HDV), Varkud satellite (VS), or glmS ribozyme site, or a variant thereof. In embodiments, the ribozyme site is a HDV ribozyme site. In embodiments, the ribozyme site is a twister ribozyme site. In embodiments, the ribozyme site is upstream of the one or more exons and/or introns of the repRNA. In embodiments, the ribozyme cleaves the target. In embodiments, the ribozyme is a trans-cleaving ribozyme.

In embodiments, the repRNA comprises at least one intronic spacer sequence comprising at least one ISE and ESS sequences. In embodiments, the at least one intronic spacer sequence comprising at least one ISE and ESS sequences increases trans-splicing efficiency of a target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences decreases trans-splicing efficiency of a target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences increases trans-splicing efficiency of a off-target RNA as compared to an unmodified form. In embodiments, the repRNA comprises at least one intronic spacer sequence comprising at least one ISE and ESS sequences. In embodiments, the at least one intronic spacer sequence comprising at least one ISE and ESS sequences decreases trans-splicing efficiency of a off-target RNA as compared to an unmodified form.

In embodiments, the repRNA comprises a ESS, ESE, ISS, and/or ISE sequence. In embodiments, the repRNA targets one or more of ESS, ESE, ISS, and/or ISE. In embodiments, an interaction, modulation and/or binding to one or more of ESS, ESE, ISS, and/or ISE reduces or ablates interaction, modulation and/or binding of the one or more of the ESS, ESE, ISS, and/or ISE with a target. In embodiments, the repRNA comprises exon sequences with ESE and ESS sequences. In embodiments, the exon sequences with ESE and ESS sequences increase or decrease trans-splicing efficiency to an RNA target as compared to an unmodified form. In embodiments, the repRNA comprises exon sequences with ESE and ESS sequences. In embodiments, the repRNA comprises exon sequences with ESE and ESS sequences increase or decrease trans-splicing efficiency to an RNA off-target as compared to an unmodified form. In embodiments, the repRNA comprises at least one or more G4 structures. In embodiments, the repRNA comprises at least one or more G4 structures sequester SD/SA motifs. In embodiments, the G4 structure is unwound, such as by DHX36 or CNBP, and remains trapped in the unwound state in the presence of a complementary sequence (e.g., endogenous target or exogenously delivered trigger RNA). In embodiments, the G4 structure decreases off-targets as compared to an unmodified form.

In embodiments, the repRNA comprises a modification comprising at least one or more scaffolding sequences. In embodiments, the at least one or more scaffolding sequences mediates (e.g., recruits) phase condensate-like formation and/or improves local concentrations of repRNAs and other targeted proteins and/or RNA as compared to an unmodified form. In embodiments, the repRNA comprises a modification comprising at least one or more sequences to target the repRNA to the promoter of the target gene of interest, or to proximal condensates that may contain the promoter. In embodiments, the one or more sequences comprises an enhancer RNA, snRNA and/or snoRNA sequences.

In embodiments, the repRNA comprises a modification. In embodiments, the repRNA modification improves interaction and localization to a DNA sequence of the non-template strand of the target gene as compared to an unmodified form. In embodiments, the DNA sequence of the non-template strand of the target gene is the promoter, intron, exon, or enhancer. In embodiments, the modification improves interaction and localization to the DNA sequence of the non-template strand of the target gene through protein-directed (e.g. transcription factor, dCas, ZNF, or other RBP) or nucleotide-directed (e.g., R-loop) methods as compared to an unmodified form.

In embodiments, the repRNA comprises a modification comprising additional RNA elements. In embodiments, the modification comprising additional RNA elements improves subnuclear localization to nuclear speckles for enhanced trans-splicing efficiency as compared to an unmodified form. In embodiments, the additional RNA element comprise NEAT1 and/or MALAT1, or a fragment thereof. In embodiments, the additional RNA element comprises a nucleotide sequence of SEQ ID NO: 712, or a fragment or variant thereof, optionally having at least about 90%, or at least about 95%, or at least about 97%, or at least about 98%, or at least about 99% identity thereto and/or or having about 1 to about 20 (e.g. about 1, or about 2, or about 3, or about 4, or about 5) nucleic acid modifications, optionally selected from substitutions, additions, or deletions. In embodiments, the repRNA comprises a modification. In embodiments, the repRNA modification enables targeting to site of transcription of target RNAs. In embodiments, the repRNA comprises a modification comprising 5′ UTR or 3′ UTR modifications. In embodiments, the modification alters intracellular or intranuclear localization based on interactions with endogenous or exogenously supplied molecules (e.g., RNA G4 interactions with transcription factors or other proteins that are localized to specific cellular compartments).

In embodiments, the repRNA comprises a modification in the 5′ UTR of the repRNA. In embodiments, the modification in the 5′ UTR of the repRNA increases stability as compared to an unmodified form. In embodiments, the modification in the 5′ UTR of the repRNA decreases stability as compared to an unmodified form. In embodiments, the modification in the 5′ UTR of the repRNA increases or decreases translation efficiency as compared to an unmodified form. In embodiments, the repRNA comprises a modification in the 3′ UTR of the repRNA. In embodiments, the modification in the 3′ UTR of the repRNA increases stability as compared to an unmodified form. In embodiments, the modification in the 3′ UTR of the repRNA decreases stability as compared to an unmodified form. In embodiments, the modification in the 3′ UTR of the repRNA increases or decreases translation efficiency as compared to an unmodified form.

In embodiments, the repRNA comprises a modification comprising modifying the repRNA to comprise a G4 structure that mediates recruitment of splicing-associated RBPs.

In embodiments, the repRNA comprises a modification comprising at least one or more toehold switches in the repRNA. In embodiments, the at least one or more toehold switches in the repRNA conditionally activate or deactivate (e.g., SD/SA occlusion, binding motif occlusion, or RBP occlusion) upon detection of an endogenous or exogenously supplied target RNA.

In embodiments, the repRNA comprises a modification comprising at least one or more complementary riboregulators in repRNAs (in cis). In embodiments, the at least one or more complementary riboregulators in repRNAs (in cis) occlude splice donor (SD) site and reduce off-target trans-splicing.

In embodiments, the repRNA comprises a modification comprising at least one or more self-complementary riboregulators in repRNAs (in cis). In embodiments, the at least one or more self-complementary riboregulators in repRNAs (in cis) occlude splice acceptor (SA) site and reduce off-target trans-splicing.

In embodiments, the repRNA comprises a modification comprising at least one or more self-complementary riboregulators in repRNAs (in trans). In embodiments, the at least one or more self-complementary riboregulators in repRNAs (in trans) occlude splice donor (SD) site and reduce off-target trans-splicing.

In embodiments, the repRNA comprises a modification comprising at least one or more self-complementary riboregulators in repRNAs (in trans). In embodiments, the at least one or more self-complementary riboregulators in repRNAs (in trans) occlude splice acceptor (SA) site and reduce off-target trans-splicing.

In embodiments, the repRNA comprises a modification comprising at least one or more binding motifs. In embodiments, the at least one or more binding motifs increase trans-splicing efficiency, target specificity, and target site occlusion (SA, SD, ISS, ISE, ESE, and ESS) as compared to an unmodified form.

In embodiments, the repRNA comprises a modification. In embodiments, the repRNA modification enables induction of trans-splicing in response to a stimulus as compared to an unmodified form. In embodiments, the repRNA comprises a modification to turn off or decrease trans-splicing in response to a stimulus as compared to an unmodified form.

In embodiments, the repRNA comprises a modification. In embodiments, the repRNA modification enables small molecule induction of trans-splicing as compared to an unmodified form. In embodiments, the repRNA comprises a modification. In embodiments, the repRNA modification represses small molecule induction of trans-splicing as compared to an unmodified form.

In embodiments, the repRNA comprises a modification. In embodiments, the repRNA modification enables light induction of trans-splicing.

In embodiments, the repRNA comprises a modification comprising at least one or more motifs that are bound and regulated by light-sensitive proteins.

In embodiments, the repRNA comprises a ribozyme site that cleaves at the 5′ end of the repRNA. In embodiments, the repRNA comprises a ribozyme site that cleaves at the 3′ end of the repRNA. In embodiments, the repRNA comprises a ribozyme site that cleaves the snRNA or snoRNA at the 5′ end of the repRNA. In embodiments, the repRNA comprises a ribozyme site that cleaves the snRNA or snoRNA at the 3′ end of the repRNA.

In embodiments, the composition or system further comprises at least one pre-rRNA stemloop to remove either the 5′cap or 3′ polyA tail.

In some embodiments, the snRNA is a U1 snRNA. In some embodiments, the U1 snRNA assembles into a U1 RNP. In some embodiments, the snRNA is a U2 snRNA. In some embodiments, the U2 snRNA assembles into a U2 RNP. In some embodiments, the snRNA is a U4 snRNA. In some embodiments, the U1 snRNA assembles into a U4 RNP. In some embodiments, the snRNA is a U4atac snRNA. In some embodiments, the U1 snRNA assembles into a U4atac RNP. In some embodiments, the snRNA is a U5 snRNA. In some embodiments, the U1 snRNA assembles into a U5 RNP. In some embodiments, the snRNA is a U6 snRNA. In some embodiments, the U1 snRNA assembles into a U6 RNP. In some embodiments, the snRNA is a U6atac snRNA. In some embodiments, the U1 snRNA assembles into a U6atac RNP. In some embodiments, the snRNA is a U7 snRNA. In some embodiments, the U1 snRNA assembles into a U7 RNP. In some embodiments, the snRNA is a U11 snRNA. On some embodiments, the U1 snRNA assembles into a U11 RNP. In some embodiments, the snRNA is a U12 snRNA. In some embodiments, the U1 snRNA assembles into a U12 RNP.

In some embodiments, the snRNA and/or snoRNA comprises a Sm sequence motif. In some embodiments, the Sm sequence motif assembles with an Sm protein to form an RNP. In some embodiments, the Sm protein is B/B′, D3, D2, D1, E, F, and G Sm proteins.

In some embodiments, a composition, system, and/or the one or more nucleic acids comprising one or more nucleotide sequences described herein comprises a full-length snoRNA or portion thereof comprising a nucleotide sequence comprising one or more sequence motifs described herein (e.g., one or more sequence motifs set forth in Table 1). In some embodiments, a snoRNA sequence described herein or identified according to a method described herein is incorporated into a composition, system, and/or the one or more nucleic acids comprising one or more nucleotide sequences described herein. In some embodiments, a full-length snoRNA sequence described herein or identified according to a method described herein is incorporated into a composition, system, and/or the one or more nucleic acids comprising one or more nucleotide sequences described herein. In some embodiments, a portion of a snoRNA sequence described herein or identified according to a method described herein (e.g., a region of contiguous nucleotides in the snRNA) is incorporated into a composition, system, and/or the one or more nucleic acids comprising one or more nucleotide sequences described herein.

In some embodiments, the full-length snoRNA or portion thereof assembles into a small nucleolar RNP (snoRNP). snoRNAs are responsible for RNA methylation and RNA pseudouridylation (Bachellerie 2002, Kiss 2004). There are two classes of snoRNAs, namely (i) H/ACA box snoRNAs which are responsible for pseudouridylation and (ii) C/D box snoRNAs which are responsible ‘or “—O-ribose methylation (Jorjani 2016, Kufel 2019). snoRNAs can also form RNPs termed snoRNPs (Khanna 2006) and hybridize to their RNA targets via Watson-Crick base pairing (Jin 2007).

In some embodiments, the full-length snoRNA or portion thereof comprises an H/ACA box. In some embodiments, the H/ACA box comprises a nucleotide sequence comprising from 5’ to 3′ an H consensus sequence (e.g., an H consensus sequence comprising the sequence set forth in SEQ ID NO: 1) and an ACA consensus sequence (e.g., an ACA consensus sequence comprising the sequence set forth in SEQ ID NO: 2). In some embodiments, the H/ACA box snoRNA assembles to form an H/ACA snoRNP. In some embodiments, the full-length snoRNA or portion thereof comprises a C/D box. In some embodiment, the C/D box comprises a nucleotide sequence comprising from 5′ to 3′ a C consensus sequence (e.g., a C consensus sequence comprising the sequence set forth in SEQ ID NO: 5), a D′ consensus sequence (e.g., a D′ consensus sequence comprising the sequence set forth in SEQ ID NO: 8), a C′ consensus sequence (e.g., a C′ consensus sequence comprising the sequence set forth in SEQ ID NO: 7), and a D consensus sequence (e.g., a D consensus sequence comprising the sequence set forth in SEQ ID NO: 6). In some embodiments, the C/D box snoRNP assembles to form a C/D snoRNP.

In some embodiments, a composition, system, and/or the one or more nucleic acids comprising one or more nucleotide sequences described herein comprises a full-length snoRNA or portion thereof comprising a nucleotide sequence comprising one or more sequence motifs described herein (e.g., one or more sequence motifs set forth in Table 1), and wherein the snoRNA is selected from SCARNA18, SCARNA18B, SNORA1, SNORA10, SNORA108, SNORA10B, SNORA1I, SNORA11B, SNORA11C, SNORA1ID, SNORA1IE, SNORA1IF, SNORA1IG, SNORA12, SNORA13, SNORA14A, SNORA14B, SNORA15, SNORA15B-1, SNORA15B-2, SNORA16A, SNORA16B, SNORA17A, SNORA17B, SNORA18, SNORA19, SNORA1B, SNORA20, SNORA20B, SNORA21, SNORA21B, SNORA22, SNORA22B, SNORA22C, SNORA24, SNORA24B, SNORA25, SNORA25B, SNORA26, SNORA27, SNORA28, SNORA29, SNORA2A, SNORA2B, SNORA2C, SNORA30, SNORA30B, SNORA31, SNORA31B, SNORA32, SNORA33, SNORA35, SNORA35B, SNORA36A, SNORA36B, SNORA36C, SNORA37, SNORA38, SNORA38B, SNORA3A, SNORA3B, SNORA3C, SNORA4, SNORA40, SNORA40B, SNORA40C, SNORA41, SNORA41B, SNORA44, SNORA46, SNORA47, SNORA48, SNORA48B, SNORA49, SNORA50A, SNORA50B, SNORA50C, SNORA50D, SNORA51, SNORA52, SNORA54, SNORA55, SNORA56, SNORA57, SNORA58, SNORA58B, SNORA59A, SNORA5A, SNORA5B, SNORA5C, SNORA6, SNORA60, SNORA61, SNORA62, SNORA63, SNORA63B, SNORA63C, SNORA63D, SNORA63E, SNORA64, SNORA65, SNORA66, SNORA67, SNORA68, SNORA68B, SNORA69, SNORA70, SNORA70B, SNORA70C, SNORA70D, SNORA70E, SNORA7OF, SNORA70G, SNORA70H, SNORA70I, SNORA70J, SNORA71, SNORA7IA, SNORA7IC, SNORA7ID, SNORA7IE, SNORA72, SNORA73, SNORA74, SNORA74D, SNORA75, SNORA75B, SNORA77, SNORA77B, SNORA78, SNORA79, SNORA79B, SNORA7A, SNORA7B, SNORA8, SNORA80A, SNORA80B, SNORA80C, SNORA80D, SNORA80E, SNORA81, SNORA84, SNORA9, SNORA9B, SNORD10, SNORD100, SNORD101, SNORD102, SNORD104, SNORD105, SNORD105B, SNORD107, SNORD108, SNORD109A, SNORD109B, SNORD11, SNORD110, SNORD111, SNORD111B, SNORD112, SNORD113-1, SNORD113-2, SNORD113-3, SNORD113-4, SNORD113-5, SNORD113-6, SNORD113-7, SNORD113-8, SNORD113-9, SNORD114-1, SNORD114-10, SNORD114-11, SNORD114-12, SNORD114-13, SNORD114-14, SNORD114-15, SNORD114-16, SNORD114-17, SNORD114-18, SNORD114-19, SNORD114-2, SNORD114-20, SNORD114-21, SNORD114-22, SNORD114-23, SNORD114-24, SNORD114-25, SNORD114-26, SNORD114-27, SNORD114-28, SNORD114-29, SNORD114-3, SNORD114-30, SNORD114-31, SNORD114-4, SNORD114-5, SNORD114-6, SNORD114-7, SNORD114-9, SNORD115, SNORD115-1, SNORD115-10, SNORD115-11, SNORD115-12, SNORD115-13, SNORD115-14, SNORD115-15, SNORD115-16, SNORD115-17, SNORD115-18, SNORD115-19, SNORD115-2, SNORD115-20, SNORD115-21, SNORD115-22, SNORD115-23, SNORD115-24, SNORD115-25, SNORD115-26, SNORD115-27, SNORD115-28, SNORD115-29, SNORD115-3, SNORD115-30, SNORD115-31, SNORD115-32, SNORD115-33, SNORD115-34, SNORD115-35, SNORD115-36, SNORD115-37, SNORD115-38, SNORD115-39, SNORD115-4, SNORD115-40, SNORD115-41, SNORD115-42, SNORD115-43, SNORD115-44, SNORD115-45, SNORD115-46, SNORD115-47, SNORD115-48, SNORD115-5, SNORD115-6, SNORD115-7, SNORD115-8, SNORD115-9, SNORD116, SNORD116-1, SNORD116-10, SNORD116-11, SNORD116-12, SNORD116-13, SNORD116-14, SNORD116-15, SNORD116-16, SNORD116-17, SNORD116-18, SNORD116-19, SNORD116-2, SNORD116-20, SNORD116-21, SNORD116-22, SNORD116-23, SNORD116-24, SNORD116-25, SNORD116-26, SNORD116-27, SNORD116-28, SNORD116-29, SNORD116-3, SNORD116-30, SNORD116-4, SNORD116-5, SNORD116-6, SNORD116-7, SNORD116-8, SNORD116-9, SNORD117, SNORD118, SNORD11B, SNORD12, SNORD121A, SNORD121B, SNORD123, SNORD124, SNORD125, SNORD126, SNORD127, SNORD12B, SNORD12C, SNORD13, SNORD13D, SNORD13E, SNORD13P1, SNORD13P3, SNORD14, SNORD14A, SNORD14B, SNORD14C, SNORD14D, SNORD14E, SNORD15A, SNORD15B, SNORD16, SNORD18, SNORD18A, SNORD18B, SNORD18C, SNORD19, SNORD19B, SNORD19C, SNORD1A, SNORD1B, SNORD1C, SNORD2, SNORD20, SNORD21, SNORD22, SNORD23, SNORD24, SNORD25, SNORD26, SNORD27, SNORD28, SNORD28B, SNORD29, SNORD30, SNORD31B, SNORD32A, SNORD32B, SNORD33, SNORD34, SNORD35A, SNORD35B, SNORD36, SNORD36A, SNORD36B, SNORD36C, SNORD37, SNORD38A, SNORD38B, SNORD38C, SNORD38D, SNORD39, SNORD41, SNORD42, SNORD42A, SNORD42B, SNORD43, SNORD45A, SNORD45B, SNORD45C, SNORD46, SNORD48, SNORD49A, SNORD49B, SNORD4A, SNORD4B, SNORD5, SNORD50B, SNORD51, SNORD52, SNORD53, SNORD53B, SNORD54, SNORD55, SNORD56, SNORD56B, SNORD57, SNORD58, SNORD58A, SNORD58B, SNORD58C, SNORD59A, SNORD6, SNORD60, SNORD61, SNORD62, SNORD62A, SNORD62B, SNORD63, SNORD63B, SNORD64, SNORD65, SNORD65B, SNORD65C, SNORD66, SNORD67, SNORD68, SNORD69, SNORD7, SNORD70, SNORD70B, SNORD71, SNORD72, SNORD73A, SNORD73B, SNORD74B, SNORD77B, SNORD79, SNORD8, SNORD81, SNORD82, SNORD83, SNORD83A, SNORD83B, SNORD84, SNORD86, SNORD87, SNORD88A, SNORD88B, SNORD88C, SNORD89, SNORD9, SNORD90, SNORD92, SNORD93, SNORD94, SNORD95, SNORD96A, SNORD96B, SNORD97, SNORD98, SNORD99, U8, snoZ196.

### Methods to Engineer a Composition, System, or Nucleic Acid of the Disclosure

The present disclosure provides methods to engineer a composition, system, and/or the one or more nucleic acids comprising one or more nucleotide sequences described herein. In some embodiments, the method comprises (A) identifying one or more candidate snRNAs and/or snoRNAs; (B) obtaining a snRNA and/or snoRNA sequence f; and (C) producing a composition, system, and/or the one or more nucleic acids comprising one or more nucleotide sequences described herein comprising a nucleotide sequence comprising (i) an intronic sequence comprising (a) the snRNA and/or snoRNA sequence, and (b) one or more binding domains described herein; (ii) a splice acceptor and/or splice donor; and (iii) one or more exonic sequences, thereby providing a composition, system, and/or the one or more nucleic acids comprising one or more nucleotide sequences described herein for targeting trans-splicing of the target RNA (e.g., target pre-mRNA). In some embodiments, the method comprises introducing the composition, system, and/or the one or more nucleic acids comprising one or more nucleotide sequences described herein to a cell or population of cells and determining the efficiency of trans-splicing of the target RNA (e.g., target pre-mRNA) according to a method described herein. In some embodiments, the efficiency of trans-splicing of the composition, system, and/or the one or more nucleic acids comprising one or more nucleotide sequences described herein is compared to that of a control nucleic acid. In some embodiments, the control nucleic acid comprises (i), (ii), and (iii) and lacks the snRNA and/or snoRNA sequence.

Methods of Identifying Candidate snRNA and/or snoRNA Sequences

In some embodiments, identifying one or more candidate snRNA and/or snoRNA sequences comprises (i) obtaining one or more snRNA and/or snoRNA sequences from a database and/or by experimental analysis of RNA expressed by a cell or organism as described herein; (ii) predicting the secondary structure formed by the one or more snRNA and/or snoRNA sequences according to a method described herein; (iii) comparing the predicted secondary structure of (ii) to a reference secondary structure (e.g., a secondary structure present in a snRNA and/or snoRNA known in the art); and (iv) selecting one or more candidate snRNA and/or snoRNA sequences with a predicted secondary structure having substantial similarity to the reference secondary structure. Computational methods for predicting the secondary structures formed by a snRNA and/or snoRNA sequence are known in the art (see, e.g., Lorenz, et al. ViennaRNA Package 2.0 Algorithms for Molecular Biology, 6:1 26, 2011). Methods for performing RNA sequence analysis by comparing predicted secondary structures to a reference secondary structure are also known in the art (see, e.g., Eddy, et al (1994) Nucleic Acids Res 22:2079).

In some embodiments, identifying one or more candidate snRNA and/or snoRNA sequences comprises (i) obtaining one or more snRNA and/or snoRNA sequences from a database and/or by experimental analysis of RNA expressed by a cell or organism as described herein; (ii) predicting the secondary structure formed by the one or more snRNA and/or snoRNA sequences according to a method described herein; (iii) comparing the predicted secondary structure of (ii) to a reference secondary structure (e.g., a secondary structure present in a snRNA and/or snoRNA known in the art); and (iv) selecting one or more candidate snRNA and/or snoRNA sequences with a predicted secondary structure having substantial similarity to the reference secondary structure and comprising a sequence motif described herein (e.g., a sequence motif comprises one or more sequences set forth in Table 1).

In some embodiments, the one or more candidate snRNA and/or snoRNA sequences are selected from any one or any combination of sequences set forth in SEQ ID NOs: 9-657 and 732-793.

Method of Obtaining a snRNA and/or snoRNA Sequence

In some embodiments, obtaining a snRNA and/or snoRNA sequence for inclusion in a composition, system, and/or the one or more nucleic acids comprising one or more nucleotide sequences described herein comprises (i) identifying one or more candidate snRNA and/or snoRNA sequences as described herein; and (ii) selecting a snRNA and/or snoRNA sequence from the one or more candidate snRNA and/or snoRNA sequences.

In some embodiments, obtaining the snRNA and/or snoRNA sequence comprises selecting a snRNA and/or snoRNA sequence from the one or more candidate snRNA and/or snoRNA sequences (e.g., one or more candidate snRNA and/or snoRNA sequences selected from any one or any combination of sequences set forth in SEQ ID NOs: 9-657 and 732-793), wherein the snRNA and/or snoRNA sequence is at least about 7, about 8, about 9, about 10, about 11, about 12, about 13, about 14, or about 15 nucleotides in length and comprises a Sm motif described herein (e.g., a Sm motif set forth in Table 1). In some embodiments, the snRNA and/or snoRNA sequence is about 7 nucleotides in length and comprises a Sm motif described herein (e.g., a Sm motif set forth in Table 1). In some embodiments, the snRNA and/or snoRNA sequence is about 8 nucleotides in length and comprises a Sm motif described herein (e.g., a Sm motif set forth in Table 1). In some embodiments, the snRNA and/or snoRNA sequence is about 9 nucleotides in length and comprises a Sm motif described herein (e.g., a Sm motif set f©rth in Table 1). In some embodiments, the snRNA and/or snoRNA sequence is about 10 nucleo©ides in length and comprises a Sm motif described herein (e.g., a Sm motif set forth in Table 1). In some embodiments, the snRNA and/or snoRNA sequence is about 11 nucleotides in length and comprises a Sm motif described herein (e.g., a Sm motif set forth in Table 1). In some embodiments, the snRNA and/or snoRNA sequence is about 12 nucleotides in length and comprises a Sm motif described herein (e.g., a Sm motif set forth in Table 1).

In some embodiments, obtaining the snRNA and/or snoRNA sequence comprises selecting a snRNA and/or snoRNA sequence from the one or more candidate snRNA and/or snoRNA sequences (e.g., one or more candidate snRNA and/or snoRNA sequences selected from any one or any combination of sequences set forth in SEQ ID NOs: 9-657 and 732-793), wherein the snRNA and/or snoRNA sequence is at least about 40, about 45, about 50, about 55, about 60, about 65, about 70, about 75, about 80, about 85, about 90, about 95, or about 100 nucleotides in length and comprises a Sm motif described herein (e.g., a Sm motif set forth in Table 1).

In some embodiments, obtaining the snRNA and/or snoRNA sequence comprises selecting a snRNA and/or snoRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate snRNA and/or snoRNA sequences or a portion thereof (e.g., one or more candidate snRNA and/or snoRNA sequences selected from any one or any combination of sequences set forth in SEQ ID NOs: 9-657 and 732-793), wherein the snRNA and/or snoRNA se©uence is at least about 40, about 45, about 50, about 55, about 60, about 65, about 70, about 75, about 80, about 85, about 90, about 95, or about 100 nucleotides in length and comprises a Sm motif described herein (e.g., a Sm motif set forth in Table 1).

In some embodiments, obtaining the snRNA and/or snoRNA sequence comprises selecting a snRNA and/or snoRNA sequence from the one or more candidate snRNA and/or snoRNA sequences (e.g., one or more candidate snRNA and/or snoRNA sequences selected from any one or any combination of sequences set forth in SEQ ID NOs: 9-657 and 732-793), wherein the snRNA and/or snoRNA sequence is at least about 40, about 45, about 50, about 55, about 60, about 65, about 70, about 75, about 80, about 85, about 90, about 95, or about 100 nucleotides in length and comprises a H-box motif described herein (e.g., a H-box motif set forth in Table 1).

In some embodiments, obtaining the snRNA and/or snoRNA sequence comprises selecting a snRNA and/or snoRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate snRNA and/or snoRNA sequences or a portion thereof (e.g., one or more candidate snRNA and/or snoRNA sequences selected from any one or any combination of sequences set forth in SEQ ID NOs: 9-657 and 732-793, or a portion thereof), wherein the snRNA and/or snoRNA sequence is at least about 40, about 45, about 50, about 55, about 60, about 65, about 70, about 75, about 80, about 85, about 90, about 95, or about 100 nucleotides in length and comprises a H-box motif described herein (e.g., a H-box motif set forth in Table 1).

In some embodiments, obtaining the snRNA and/or snoRNA sequence comprises selecting a snRNA and/or snoRNA sequence from the one or more candidate snRNA and/or snoRNA sequences (e.g., one or more candidate snRNA and/or snoRNA sequences selected from any one or any combination of sequences set forth in SEQ ID NOs: 9-657 and 732-793), wherein the snRNA and/or snoRNA sequence is at least about 40, about 45, about 50, about 55, about 60, about 65, about 70, about 75, about 80, about 85, about 90, about 95, or about 100 nucleotides in length and comprises an ACA-box motif described herein (e.g., an ACA-box motif set forth in Table 1).

In some embodiments, obtaining the snRNA and/or snoRNA sequence comprises selecting a snRNA and/or snoRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate snRNA and/or snoRNA sequences or a portion thereof (e.g., one or more candidate snRNA and/or snoRNA sequences selected from any one or any combination of sequences set forth in SEQ ID NOs: 9-657 and 732-793, or a portion thereof), wherein the snRNA and/or snoRNA sequence is at least about 40, about 45, about 50, about 55, about 60, about 65, about 70, about 75, about 80, about 85, about 90, about 95, or about 100 nucleotides in length and comprises a ACA-box motif described herein (e.g., a ACA-box motif set forth in Table 1).

In some embodiments, obtaining the snRNA and/or snoRNA sequence comprises selecting a snRNA and/or snoRNA sequence from the one or more candidate snRNA and/or snoRNA sequences (e.g., one or more candidate snRNA and/or snoRNA sequences selected from any one or any combination of sequences set forth in SEQ ID NOs: 9-657 and 732-793), wherein the snRNA and/or snoRNA sequence is at least about 40, about 45, about 50, about 55, about 60, about 65, about 70, about 75, about 80, about 85, about 90, about 95, or about 100 nucleotides in length and comprises an H-box and an ACA-box motif described herein (e.g., a H-box and a ACA-box motif set forth in Table 1).

In some embodiments, obtaining the snRNA and/or snoRNA sequence comprises selecting a snRNA and/or snoRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate snRNA and/or snoRNA sequences or a portion thereof (e.g., one or more candidate snRNA and/or snoRNA sequences selected from any one or any combination of sequences set forth in SEQ ID NOs: 9-657 and 732-793 or a portion thereof), wherein the snRNA and/or snoRNA sequence is at least about 40, about 45, about 50, about 55, about 60, about 65, about 70, about 75, about 80, about 85, about 90, about 95, or about 100 nucleotides in length and comprises an H-box and an ACA-box motif described herein (e.g., a H-box and a ACA-box motif set forth in Table 1).

In some embodiments, obtaining the snRNA and/or snoRNA sequence comprises selecting a snRNA and/or snoRNA sequence from the one or more candidate snRNA and/or snoRNA sequences (e.g., one or more candidate snRNA and/or snoRNA sequences selected from any one or any combination of sequences set forth in SEQ ID NOs: 9-657 and 732-793), wherein the snRNA and/or snoRNA sequence is at least about 40, about 45, about 50, about 55, about 60, about 65, about 70, about 75, about 80, about 85, about 90, about 95, or about 100 nucleotides in length and comprises a (i)C-box motif described herein (e.g., a C-box motif set forth in Table 1), (ii) C′-box motif described herein (e.g., a C′-box motif set forth in Table 1), (iii) D-box motif described herein (e.g., a D-box motif set forth in Table 1), (iv) a D′-box motif described herein (e.g., a D′-box motif set forth in Table 1), or (v) a combination of (i)-(iv).

In some embodiments, obtaining the snRNA and/or snoRNA sequence comprises selecting a snRNA and/or snoRNA sequence having at least about 60%, about 65%, about 70%, about 75%, about 80%, about 85%, about 90%, about 91%, about 92%, about 93%, about 94%, about 95%, about 96%, about 97%, about 98%, about 99% to one or more candidate snRNA and/or snoRNA sequences or a portion thereof (e.g., one or more candidate snRNA and/or snoRNA sequences selected from any one or any combination of sequences set forth in SEQ ID NOs: 9-657 and 732-793, or a portion thereof), wherein the sequence is at least about 40, about 45, about 50, about 55, about 60, about 65, about 70, about 75, about 80, about 85, about 90, about 95, or about 100 nucleotides in length and comprises a (i)C-box motif described herein (e.g., a C-box motif set forth in Table 1), (ii) C′-box motif described herein (e.g., a C′-box motif set forth in Table 1), (iii) D-box motif described herein (e.g., a D-box motif set forth in Table 1), (iv) a D′-box motif described herein (e.g., a D′-box motif set forth in Table 1), or (v) a combination of (i)-(iv).

Methods of Producing the Composition, System, and/or the Nucleotide Sequences

The composition, system, and/or the one or more nucleic acids comprising one or more nucleotide sequences described herein are produced by suitable nucleic acid synthesis method or means known in the art. In some embodiments, the composition, system, and/or the one or more nucleic acids comprising one or more nucleotide sequences described herein is produced as an RNA. In some embodiments, the composition, system, and/or the one or more nucleic acids comprising one or more nucleotide sequences described herein is produced as a DNA. The present disclosure further provides delivery systems comprising the composition, system, and/or the one or more nucleic acids comprising one or more nucleotide sequences described herein, e.g., a vector comprising the composition, system, and/or the one or more nucleic acids comprising one or more nucleotide sequences described herein, a lipid particle comprising the composition, system, and/or the one or more nucleic acids comprising one or more nucleotide sequences described herein. Methods of producing the composition, system, and/or the one or more nucleic acids comprising one or more nucleotide sequences described herein include, but are not limited to, in vitro transcription (IVT), synthetic and/or chemical synthesis methods, or a combination thereof. In some embodiments, enzymatic (e.g., IVT), solid-phase, liquid-phase, combined synthetic methods, small region synthesis, and ligation methods are utilized.

In embodiments, the disclosure provides compositions, systems, and/or the one or more nucleic acids comprising one or more nucleotide sequences described herein are produced using IVT enzymatic synthesis methods. Methods of making polynucleotides by IVT are known in the art and are described in International Application PCT/US2013/30062.

In embodiments, the disclosure provides composition, system, and/or the one or more nucleic acids comprising one or more nucleotide sequences described herein chemically synthesized by any means described in the art. In some embodiments, the composition, system, and/or the one or more nucleic acids comprising one or more nucleotide sequences described herein are produced by oligonucleotide synthesis. Oligonucleotide synthesis is the chemical synthesis of relatively short fragments or strands of single-stranded nucleic acids with a defined chemical structure (sequence). Methods of oligonucleotide synthesis are known in the art (see e.g., Reese (2005) Organic & Biomolecular Chemistry 3 (21): 3851). While chemical synthetic procedures are continually expanding, purifications of such nucleic acids by procedures such as high performance liquid chromatography (HPLC, which avoids the use of gels such as PAGE) tends to become more challenging as polynucleotide lengths increase significantly beyond a hundred or so nucleotides. One approach used for generating nucleic acids of greater length is to produce two or more molecules that are ligated together.

### Methods of Determining Tran Splicing Efficiency

In embodiments, the disclosure provides methods to determine the efficiency of trans-splicing of a target RNA (e.g., pre-mRNA) using a composition, system, and/or the one or more nucleic acids comprising one or more nucleotide sequences described herein.

In embodiments, the method comprises use of a fluorescence-based splicing reporter assay. In embodiments, the assay comprises contacting a reporter cell or population of cells with the composition, system, and/or the one or more nucleic acids comprising one or more nucleotide sequences described herein according to a method described herein (e.g., via transfection with a viral vector encoding the composition, system, and/or the one or more nucleic acids comprising one or more nucleotide sequences described herein), wherein the composition, system, and/or the one or more nucleic acids comprising one or more nucleotide sequences described herein comprises at least one exon encoding a reporter molecule, wherein a trans-splicing event is indicated by the presence of a fluorescent signal from the reporter molecule that is detected using a method known in the art. For example, in some embodiments, the reporter molecule is a fluorescent protein detected using fluorescence-activated cell sorting (FACS). For example, in some embodiments, the composition, system, and/or the one or more nucleic acids comprising one or more nucleotide sequences described herein comprises a nucleotide sequence encoding a first portion of a fluorescent protein and the target RNA comprises a nucleotide sequence encoding a second portion of a fluorescent protein, wherein the trans-splicing generates an RNA comprising a nucleotide sequence encoding the full-length fluorescent protein, and wherein the trans-splicing event is detected using a method of fluorescent measurement (e.g., FACS).

In embodiments, the composition, system, and/or the one or more nucleic acids comprising one or more nucleotide sequences described herein is introduced to a cell using a method described herein (e.g., via a viral or non-viral vector) for a duration, whereupon RNA from the cell is extracted and trans-splicing products are detected. For example, an mRNA spliced from a target RNA (e.g., a target pre-mRNA) is analyzed by a suitable method known in the art (e.g., end-point or quantitative RT-PCR or RNA sequencing). In some embodiments, a cell or population of cells is contacted with the composition, system, and/or the one or more nucleic acids comprising one or more nucleotide sequences described herein, wherein next-generating sequencing (NGS) techniques are used to determine the extent of trans-splicing. For example, in some embodiments, mRNA extracted from cells treated or contacted with a composition, system, and/or the one or more nucleic acids comprising one or more nucleotide sequences described herein is enzymatically converted into cDNA, which is further by analyzed by NGS analysis to determine the extent of mRNA molecule comprising exonic sequence incorporated from the composition, system, and/or the one or more nucleic acids comprising one or more nucleotide sequences described herein.

In embodiments, trans-splicing is determined by protein sequence analysis of a polypeptide translated from an mRNA spliced from the pre-mRNA. In some embodiments, an RNA-guided molecule corrects a mutation by the incorporation of a corrected exon, wherein translation of the mRNA resulting from trans-splicing of the pre-mRNA and the composition, system, and/or the one or more nucleic acids comprising one or more nucleotide sequences described herein generates a polypeptide comprising an amino acid sequence encoded by the corrected exon. The protein sequence analysis is performed using techniques including, but not limited to, Sanger sequencing, mass spectrometry, functional assays that measure an enzymatic activity of the polypeptide, or immunoblotting using an antibody reactive to the corrected amino acid sequence.

In embodiments, trans-splicing is determined by measuring the activity of a protein translated from an mRNA spliced from the pre-mRNA. For example, in some embodiments, the protein is an enzyme and the method of measuring trans-splicing comprises measuring enzymatic activity using a functional ELISA.

In embodiments, a method for measuring the efficiency of trans-splicing using a composition, system, and/or the one or more nucleic acids comprising one or more nucleotide sequences described herein is described in U.S. application Ser. No. 16/994,230, incorporated herein by reference.

In embodiments, a method for measuring the efficiency of trans-splicing using a composition, system, and/or the one or more nucleic acids comprising one or more nucleotide sequences described herein is any one described in Chen, et al (2009) Gene Ther 16:211; Rindt, et al (2012) Cell Mol Life Sci 69:4191; Monjaret, et al (2014) Mol Ther 22: 1176; Berger, et al (2015) Mol Ther 23: 918.

In embodiments, a method described herein is used to measure the efficiency of trans-splicing of a pre-mRNA using a composition, system, and/or the one or more nucleic acids comprising one or more nucleotide sequences described herein.

In embodiments, the introduction of the composition or system to the cell results in an efficiency of trans-splicing that is greater than a composition or system lacking the snRNA or snoRNA sequence.

In embodiments, the efficiency of trans-splicing is greater than about 10%, about 20%, about 30%, about 40%, about 50%, about 60%, about 70%, about 80%, about 90%, or about 99%.

In embodiments, a composition, system, and/or the one or more nucleic acids comprising one or more nucleotide sequences described herein comprising a nucleotide sequence comprising (i) at least one intronic sequence comprising one or more binding domains each having complementarity to a target sequence in the pre-mRNA and a snRNA and/or snoRNA; (ii) one or more splice sites (e.g., a splice acceptor and/or splice donor); and (iii) at least one exonic sequence results in an efficiency of trans-splicing that is greater than the composition, system, and/or the one or more nucleic acids comprising one or more nucleotide sequences described herein without the snRNA and/or snoRNA, as measured using a method described herein. In some embodiments, the composition, system, and/or the one or more nucleic acids comprising one or more nucleotide sequences described herein results in an efficiency of trans-splicing that is increased by at least about 1.5-fold, about 2-fold, about 3-fold, about 4-fold, about 5-fold, about 10-fold, or about 20-fold compared to the efficiency of trans-splicing of a composition, system, and/or the one or more nucleic acids comprising one or more nucleotide sequences described herein without the snRNA and/or snoRNA.

### Vectors

In embodiments, the disclosure provides a vector comprising the composition, system, and/or the one or more nucleic acids comprising one or more nucleotide sequences described herein. As used herein, t″e term″″vecto″″ refers to a nucleic acid molecule capable of transporting another nucleic acid to which it has been linked. In some embodiments, the vector is a DNA vector. In some embodiments, the vector is circular. In some embodiments, the vector is linear. Non-limiting exemplary vectors include plasmids, phagemids, cosmids, artificial chromosomes, minichromosomes, transposons, viral vectors, and expression vectors.

In embodiments, the vector is an expression vector, wherein the expression vector is capable of directing the expression of nucleic acids to which it is operably linked. As used herein, an “expression vector” or “recombinant expression vector” refers to a replicon, such as plasmid, phage, virus, or cosmid, to which another DNA segment, ″.e. an″″inser″″, is attached so as to bring about the replication of the attached segment in a cell.

In embodiments, the vector or expression vector is a plasmid. As used he″ein, a″″plasmi″″ refers to a circular double-stranded DNA loop into which additional nucleic acid segments are ligated. In embodiments, the vector or expression vector is a viral vector, wherein additional nucleic acid segments are ligated into the viral genome. Non-limiting exemplary viral vectors include viral vectors based on vaccinia virus; poliovirus; adenovirus; adeno-associated virus; SV40; herpes simplex virus; human immunodeficiency virus; picornaviruses. Non-limiting exemplary viral vectors also include viral vectors based on a retrovirus such as a Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus. In some embodiments, the vectors is for use in eukaryotic target cells and includes, but is not limited to, pXT1, pSG5, pSVK3, pBPV, pMSG, and pSVLSV40 (Pharmacia).

In embodiments, the vector comprises one or more transcription and/or translation control elements. In embodiments, the more transcription and/or translation control elements used depends on the target cell population and the vector system. In embodiments, any number of suitable transcription and translation control elements, including constitutive and inducible promoters, transcription enhancer elements, transcription terminators, etc. are used in the expression vector, such as those further described below.

In embodiments, a vector comprising a composition, system, and/or the one or more nucleic acids comprising one or more nucleotide sequences described herein is operably linked to a control element, e.g., a transcriptional control element, such as a promoter. In embodiments, the transcriptional control element is functional in a eukaryotic cell, e.g., a mammalian cell, e.g., a human cell. In embodiments, the composition, system, and/or the one or more nucleic acids comprising one or more nucleotide sequences described herein is operably linked to one or more control elements that enable expression in eukaryotic cells, e.g., mammalian cells, e.g., human cells.

In embodiments, the expression vector comprises a promoter that is an inducible promoter (e.g., a heat shock promoter, tetracycline-regulated promoter, steroid-regulated promoter, metal-regulated promoter, estrogen receptor-regulated promoter, etc.). Examples of inducible promoters include, but are not limited to, T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), steroid-regulated promoter, metal-regulated promoter, estrogen receptor-regulated promoter, etc. In some embodiments, an inducible promoters is regulated by molecules including, but not limited to, doxycycline; RNA polymerase, e.g., T7 RNA polymerase; an estrogen receptor; an estrogen receptor fusion; etc.

In embodiments, the promoter is a constitutive promoter (e.g., CMV promoter, UBC promoter).

In embodiments, the promoter is a spatially restricted and/or temporally restricted promoter (e.g., a tissue specific promoter, a cell type specific promoter, etc.). Spatially restricted promoters can also be referred to as enhancers, transcriptional control elements, control sequences, etc. Any convenient spatially restricted promoter is suitable for use in the present disclosure, and the choice of a suitable promoter (e.g., a liver-specific promoter, a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. For example, various spatially restricted promoters are known for plants, flies, worms, mammals, mice, etc. Thus, a spatially restricted promoter can be used to regulate the expression of a composition, system, and/or the one or more nucleic acids comprising one or more nucleotide sequences described herein in a wide variety of different tissues and cell types, depending on the organism. Some spatially restricted promoters are also temporally restricted such that the promoter is in the “ON” state or “OFF’ state during specific stages of embryonic development or during specific stages of a biological process. For illustration purposes, examples of spatially restricted promoters include, but are not limited to, liver-specific promoters, neuron-specific promoters, adipocyte-specific promoters, cardiomyocyte-specific promoters, smooth muscle-specific promoters, photoreceptor-specific promoters, etc.

Suitable promoters for use in the present disclosure include those derived from viruses and are referred to herein as viral promoters, or they include those derived from an organism, including prokaryotic or eukaryotic organisms. In embodiments, a suitable promoter for use in the present disclosure include any promoter that drives expression by an RNA polymerase (e.g., pol I, pol II, pol III).

Exemplary promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497--500 (2002)), an enhanced U6 promoter (e.g., Xia et al., Nucleic Acids Res. 2003 Sep. 1; 31(17)), a human Hl promoter (H1), and the like.

Exemplary eukaryotic promoters (i.e., promoters functional in a eukaryotic cell) include, but are not limited to, those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

In embodiments, the disclosure provides a vector comprising a composition, system, and/or the one or more nucleic acids comprising one or more nucleotide sequences described herein and an RNA polymerase III promoter (e.g., U6 and H1). Descriptions of and parameters for enhancing the use of such promoters are known in art, and additional information and approaches are regularly being described; see, e.g., Ma, H. et al., Molecular Therapy—Nucleic Acids 3, e161 (2014).

In embodiments, the expression vector comprises a ribosome binding site for translation initiation and a transcription terminator. In some embodiments, the expression vector comprises appropriate sequences for amplifying expression. In some embodiments, the expression vector comprises nucleotide sequences encoding non-native tags (e.g., histidine tag, hemagglutinin tag, green fluorescent protein, etc.), for example, that are operably-linked to the composition, system, and/or the one or more nucleic acids comprising one or more nucleotide sequences described herein.

Methods of introducing a nucleic acid to a host cell or a population of host cells are known in the art, and any known method can be used to introduce a nucleic acid (e.g., an expression construct) into a cell. In some embodiments, a composition, system, and/or the one or more nucleic acids comprising one or more nucleotide sequences described herein or vector comprising the composition, system, and/or the one or more nucleic acids comprising one or more nucleotide sequences described herein are provided to a population of cells using well-developed transfection techniques; see, e.g. Angel and Yanik (2010) pLOS ONE 5 (7): e 11756, and the commercially available TransMessenger® reagents from Qiagen, Stemfect™ RNA Transfection Kit from Stemgent, and TransIT®-mRNA Transfection Kit from Mims Bio LLC (See, also Beumer et al. (2008). PNAS 105 (50): 19821-19826).

In embodiments, the composition, system, and/or the one or more nucleic acids comprising one or more nucleotide sequences described herein is introduced to the cell or a population of cells as an RNA. In some embodiments, the RNA has chemistries suitable for delivery, tolerability, and stability within cells, e.g., following in vivo or in vitro administration. In some embodiments, the RNA is modified, e.g., comprises a modified sugar moiety, a modified internucleoside linkage, a modified nucleoside, a modified nucleotide and/or combinations thereof. In some embodiments, the modified RNA exhibits one or more of the following properties: is not immune stimu©atory; is nuclease resistant; has improved cell uptake; has increased half-life; has increased translation efficiency; and/or is not toxic to cells or mammals, e.g., following contact with cells in vivo or ex vivo or in vitro.

### Delivery Agents

In embodiments, delivery of a composition, system, and/or the one or more nucleic acids comprising one or more nucleotide sequences described herein is performed by one or more methods described herein. In embodiments, the composition, system, and/or the one or more nucleic acids comprising one or more nucleotide sequences described herein is delivered by viral vectors, lipid nonaparticles (LNPs), synthetic polymers, or a combination thereof. In some embodiments, the methods of delivery described herein are suitable for administering a composition, system, and/or the one or more nucleic acids comprising one or more nucleotide sequences described herein nucleic acid of the disclosure to a target cell population or target tissue for the purpose of cellular, ex vivo, or in vivo targeting of a pre-mRNA in the target cell or target tissue for trans-splicing.

In embodiments, the delivery comprises administering the composition, system, and/or the one or more nucleic acids comprising one or more nucleotide sequences described herein as RNA or DNA. In embodiments, the delivery comprises administering the composition, system, and/or the one or more nucleic acids comprising one or more nucleotide sequences described herein as a DNA formulated as an LNP or a polymeric nanoparticle. In some embodiments, the delivery comprises administering composition, system, and/or the one or more nucleic acids comprising one or more nucleotide sequences described herein as an RNA formulated as an LNP or a polymeric nanoparticle.

In embodiments, the delivery comprises administering a recombinant expression vector comprising the composition, system, and/or the one or more nucleic acids comprising one or more nucleotide sequences described herein (e.g., plasmid, viral vector). In some embodiments, the recombinant expression vector is a non-viral vector (e.g., a plasmid). In some embodiments, the recombinant expression vector is a viral vector (e.g., an AAV). In some embodiments, the recombinant expression vector is a circular RNA (cirRNA). In some embodiments, the delivery comprises formulation of the one or more recombinant expression vectors using LNPs or polymeric nanoparticles. In some embodiments, a combination of a viral vector and a non-viral delivery vehicle are used.

In embodiments, the composition, system, and/or the one or more nucleic acids comprising one or more nucleotide sequences described herein are delivered by non-viral delivery vehicles including, but not limited to, nanoparticles, liposomes, ribonucleoproteins, positively charged peptides, small molecule-RNA conjugates, aptamer-RNA chimeras, and RNA-fusion protein complexes. Non-limiting exemplary non-viral delivery vehicles include those described in Peer and Lieberman, Gene Therapy, 18:1127-1133 (2011) (which focuses on non-viral delivery vehicles for siRNA that are also useful for delivery of other polynucleotides).

In embodiments, e.g., for trans-delivery, multiple delivery agents are used to deliver the elements. For example, in embodiments, multiple AAVs are used to deliver the elements. In embodiments, e.g., for trans-delivery, multiple different delivery agents are used to deliver the elements. In embodiments, e.g., for trans-delivery, multiple different delivery agents are used to deliver the elements, e.g., an AAV and a different delivery agent, e.g., an LNP.

### Viral Delivery

In some embodiments, the composition or system is introduced to the cell by a single viral vector, such as AAV. In some embodiments, the viral vector (e.g., AAV vector) comprises one or more composition, system, and/or the one or more nucleic acids comprising one or more nucleotide sequences described herein. In some embodiments, the cloning capacity of the viral vector is sufficient to deliver the composition, system, and/or the one or more nucleic acids comprising one or more nucleotide sequences described herein.

In some embodiments, a recombinant adeno-associated virus (rAAV) vector is used for delivery. Techniques to produce rAAV particles, in which an AAV genome to be packaged that includes the polynucleotide to be delivered (e.g., nucleic acid encoding one or more gRNAs and/or a site-directed endonuclease), rep and cap genes, and helper virus functions are provided to a cell are standard in the art. Production of rAAV typically requires that the following components are present within a single cell (denoted herein as a packaging cell): a rAAV genome, AAV rep and cap genes separate from (i.e., not in) the rAAV genome, and helper virus functions. The AAV rep and cap genes can be from any AAV serotype for which recombinant virus can be derived, and can be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13 AAV rh.74 and tropism modified AAV vectors. Production of pseudotyped rAAV is disclosed in, for example, U.S. Pat. No. 7,056,602.

In some embodiments, a method of generating a packaging cell involves creating a cell line that stably expresses all of the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line can then be infected with a helper virus, such as adenovirus. The advantages of this method are that the cells are selectable and are suitable for large-scale production of rAAV. Other examples of suitable methods employ adenovirus or baculovirus, rather than plasmids, to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial. And Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595.

In addition to adeno-associated viral vectors, other viral vectors can be used. Such viral vectors include, but are not limited to, adenovirus, lentivirus, alphavirus, enterovirus, pestivirus, baculovirus, herpesvirus, Epstein Barr virus, papovavirus, poxvirus, vaccinia virus, and herpes simplex virus.

### Nanoparticle Compositions

In some embodiments, the composition or system for targeting trans-splicing of a pre-mRNA in a cell comprising one or more nucleic acids comprising one or more nucleotide sequences is delivered to a host cell (e.g., ex vivo) or a subject by a nanoparticle (e.g., a lipid nanoparticle). In some embodiments, the nucleic acid or expression vector is formulated in nanoparticles or other delivery vehicles, (e.g., polymeric nanoparticles) to facilitate cellular uptake and/or to protect them from degradation when delivered to a subject.

In embodiments, the composition or system is formulated as a lipid nanoparticle. Lipid nanoparticles include, but are not limited to, liposomes and micelles. Any number of lipids may be present, including cationic and/or ionizable lipids, anionic lipids, neutral lipids, amphipathic lipids, conjugated lipids (e.g., PEGylated lipids), and/or structural lipids. Such lipids can be used alone or in combination.

Nanoparticles are ultrafine particles typically ranging between 1 and 100 to 500 nanometers (nm) in size with a surrounding interfacial layer and often exhibiting a size-related or size-dependent property. Nanoparticle compositions are myriad and encompass lipid nanoparticles (LNPs), liposomes (e.g., lipid vesicles), and lipoplexes. For example, a nanoparticle composition can be a liposome having a lipid bilayer with a diameter of 500 nm or less. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In embodiments, the nanoparticle composition comprises a composition, system, and/or the one or more nucleic acids comprising one or more nucleotide sequences described herein and/or a recombinant expression vector comprising the composition, system, and/or the one or more nucleic acids comprising one or more nucleotide sequences described herein.

In embodiments, the disclosure provides LNP compositions comprising: (a) a composition, system, and/or the one or more nucleic acids comprising one or more nucleotide sequences described herein or an expression vector comprising the composition, system, and/or the one or more nucleic acids comprising one or more nucleotide sequences described herein; and (b) one or more lipid moieties selected from the group consisting of amino lipids, helper lipids, structural lipids, phospholipids, ionizable lipids, PEG lipids, lipoid, and cholesterol or cholesterol derivatives. In some embodiments, the disclosure provides LNP compositions comprising: (a) a composition, system, and/or the one or more nucleic acids comprising one or more nucleotide sequences described herein or an expression vector comprising the composition, system, and/or the one or more nucleic acids comprising one or more nucleotide sequences described herein; and (b) one or more lipid moieties selected from the group consisting of ionizable lipids, amino lipids, anionic lipids, neutral lipids, amphipathic lipids, helper lipids, structural lipids, PEG lipids, and lipoids, and optionally (c) targeting moieties.

In some embodiments, the LNP composition comprise one or more lipid moieties promote or enhances cellular uptake by the apolipoprotein E (apoE)-low density lipoprotein receptor (LDLR) pathway. For example, certain ionizable lipids are known in the art for increasing cellular uptake of LNPs by the apoE-LDLR pathway (see, e.g., Semple, et al (2010) NAT BIOTECH 28:172). In some embodiments, the LNP composition comprises one or more lipid moieties that promote or enhances cellular uptake by an apoE-LDLR independent pathway.

In some embodiments, the LNPs of the present disclosure are formed by any method known in the art including, but not limited to, a continuous mixing method, a direct dilution process, and an in-line dilution process. Additional techniques and methods suitable for the preparation of the LNPs described herein include coacervation, microemulsions, supercritical fluid technologies, phase-inversion temperature (PIT) techniques.

### Pharmaceutical Compositions

In embodiments, the disclosure provides pharmaceutical compositions comprising the composition or system of any one of the embodiments described herein, the viral vector of any one of the embodiments described herein, or the lipid nanoparticle of any one of the embodiments described herein, and a pharmaceutically acceptable carrier.

In embodiments, the pharmaceutical composition comprises (1) an expression vector comprising one or more nucleic acids comprising one or more nucleotide sequences described herein, and (2) a pharmaceutically acceptable carrier or diluent. In some embodiments, the pharmaceutical composition comprises one or more nucleic acids comprising one or more nucleotide sequences or recombinant expression vector (e.g., AAV) comprising the one or more nucleic acids comprising one or more nucleotide sequences formulated as a lipid composition (e.g., LNP), and (2) a pharmaceutically acceptable carrier or diluent. In some embodiments, the pharmaceutical composition comprises a therapeutically effective amount of the one or more nucleic acids comprising one or more nucleotide sequences or recombinant expression vectors.

Exemplary pharmaceutically acceptable excipients such as carriers, solvents, stabilizers, adjuvants, diluents, etc., depending upon the particular mode of administration and dosage form. Contemplated pharmaceutical compositions can be generally formulated to achieve a physiologically compatible pH, depending on the formulation and route of administration. In some embodiments, the compositions comprise a therapeutically effective amount of one or more nucleic acids comprising one or more nucleotide sequences or recombinant expression vectors, together with one or more pharmaceutically acceptable excipients.

Suitable excipients can include, for example, carrier molecules that include large, slowly metabolized macromolecules. Other exemplary excipients can include antioxidants, chelating agents, carbohydrates, stearic acid, liquids such as oils, water, saline, glycerol and ethanol, wetting or emulsifying agents, pH buffering substances, and the like.

Pharmaceutical compositions can be formulated into preparations in solutions, suppositories, injections. In some embodiments, the pharmaceutical composition is formulated to result in systemic administration of the composition, system, and/or the one or more nucleic acids comprising one or more nucleotide sequences described herein or recombinant expression vectors, for example, following enteral or parenteral administration. In some embodiments, the pharmaceutical composition is formulated to result in localized administration of the composition, system, and/or the one or more nucleic acids comprising one or more nucleotide sequences described herein, and/or the viral vectors, for example, following regional administration or implantation. In some embodiments, the pharmaceutical composition is formulated for immediate activity or for sustained release of the composition, system, and/or the one or more nucleic acids comprising one or more nucleotide sequences described herein, and/or the viral vectors or recombinant expression vectors.

Typically, an effective amount the composition, system, and/or the one or more nucleic acids comprising one or more nucleotide sequences described herein, and/or viral vector described herein, can be provided, for example, for use in a method of treating a subject having a disease or disorder. Methods of calculating the effective amount or effective dose are within the skill of one of ordinary skill in the art. The final amount to be administered is dependent upon the route of administration and upon the nature of the disorder that is to be treated. A competent clinician will be able to determine an effective amount of the composition, system, and/or the one or more nucleic acids comprising one or more nucleotide sequences described herein, recombinant expression vectors, or delivery system described herein to administer to the patient to halt or reverse the progression of the disorder.

In some embodiments, based on animal data, and other information available for the trans-splicing system, a clinician can determine the maximum safe dose for an individual, depending on the route of administration. For instance, an intravenously administered dose can be more than an intrathecally administered dose, given the greater body of fluid into which the therapeutic composition is being administered. Similarly, compositions which are rapidly cleared from the body can be administered at higher doses, or in repeated doses, in order to maintain a therapeutic concentration. Utilizing ordinary skill, the competent clinician will be able to optimize the dosage of a particular therapeutic in the course of routine clinical trials.

For inclusion in a medicament, the composition, system, and/or the one or more nucleic acids comprising one or more nucleotide sequences described herein, including the viral vector described herein, can be obtained from a suitable commercial source. In some embodiments, therapies based on the composition, system, and/or the one or more nucleic acids comprising one or more nucleotide sequences described herein, and/or the viral vector, recombinant expression vectors, or delivery system described herein to be used for therapeutic administration, must be sterile. Therapeutic compositions can be generally placed into a container having a sterile access port, for example, an intravenous solution bag or vial having a stopper pierceable by a hypodermic injection needle. In some embodiments, the therapeutic components are stored in unit or multi-dose containers, for example, sealed ampules or vials, as an aqueous solution or as a lyophilized formulation for reconstitution.

### Methods and Use

In embodiments, the disclosure provides cellular, ex vivo, in vitro, and/or in vivo methods comprising use of the compositions, systems, and/or the one or more nucleic acids comprising one or more nucleotide sequences described herein to target trans-splicing of a target RNA (e.g., pre-mRNA) in a cell. In some embodiments, the methods comprise use of the composition, system, and/or the one or more nucleic acids comprising one or more nucleotide sequences described herein to correct a mutation in a target RNA (e.g., pre-mRNA). In some embodiments, the disclosure provides methods of treating a patient with a disease or disorder, comprising administering a composition, system, and/or the one or more nucleic acids comprising one or more nucleotide sequences described herein, the viral vector, or pharmaceutical composition described herein to target trans-splicing of a target RNA (e.g., pre-mRNA) in a target cell population and/or target tissue, thereby treating the disease or disorder.

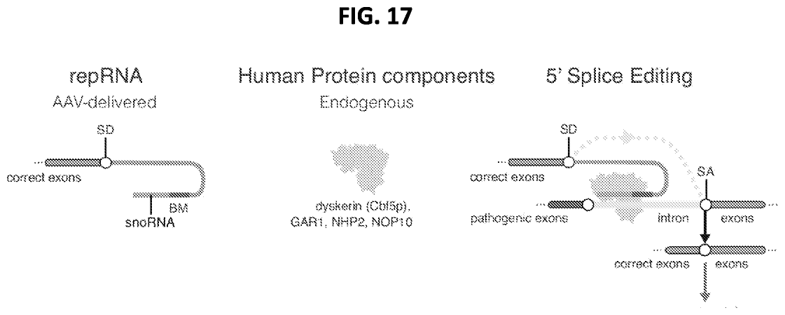

Without wishing to be bound by theory, in embodiments, trans-splicing methods with snoRNA exhibit higher activity and/or efficiency than methods of trans-splicing using snRNA, or other RNA-based methods. Traditionally, without wishing to be bound by theory, snoRNAs have not been incorporated into long ncRNA for splicing because they form secondary and tertiary structures (e.g., lariat) that are generally processed into smaller RNAs, which limits their use. In embodiments, the snoRNA sequence binds a ribonucleoprotein (RNP), such as dyskerin (Cbf5p), GAR1, NHP2, NOP10, etc., to direct trans-splicing of the pre-mRNA target sequence (e.g., as shown in FIG. 17).

In embodiments, the method for trans-splicing of a pre-mRNA target sequence comprises introducing into a cell one or more trans-splicing RNA molecules comprising one or more small nucleolar RNA (snoRNA) sequence of about 7 to about 300 nucleotides in length, at least one splice acceptor site or splice donor site (e.g., at a boundary of one or more intron/exon), one or more binding domains comprising a nucleic acid sequence that binds to one or more pre-mRNA target sequences, and replacing at least a portion of the pre-mRNA target sequence. In embodiments, at least a portion of the one or more trans-splicing RNA molecules recognizes one or more pre-mRNA target sequences. In embodiments, the method comprises splicing at least one exonic sequence into the pre-mRNA target sequence.

In embodiments, the RNA trans-splicing molecule of methods herein is oriented 3′ to 5′ or 5′ to 3′, e.g., with a snoRNA sequence at the 5′ end or 3′ end of the molecule. In embodiments, the RNA trans-splicing molecule is snoRNA-based and uses a H/ACA for 5′ editing, which is placed on 3′ end of one or more intronic sequence. In embodiments, the RNA trans-splicing molecule is snoRNA-based and uses a C/D box for 3′ editing, which is placed on 5′ end of one or more intronic sequence.

In embodiments, the RNA trans-splicing molecule is snoRNA-based and comprises one or more target binding domain (e.g., anti-sense region (ASR)), located within the snoRNA region and/or outside the snoRNA region.

In embodiments, the snoRNA comprises H/ACA box consensus sequences, where the H consensus sequence is oriented 5′ (upstream) of an ACA consensus sequence. In embodiments, the snoRNA sequence comprises at least one binding domain sequence positioned: (i) upstream the H consensus sequence; (ii) downstream the ACA consensus sequence; (iii) between the H consensus sequence and the ACA consensus sequence; or (iv) a combination of (i)-(iii).

In embodiments, the snoRNA comprises C/D box consensus sequences. In embodiments, the C/D box consensus sequence, oriented 5′ to 3′, comprises a C consensus sequence, a D′ consensus sequence, a C′ consensus sequence, and a D consensus sequence. In embodiments, the snoRNA sequence comprises the snoRNA sequence comprises at least one binding domain positioned (i) upstream the C consensus sequence; (ii) between the C consensus sequence and the D′ consensus sequence; (iii) between the D′ consensus sequence and the C′ consensus sequence; (iv) between the C′ consensus sequence and the D consensus sequence; (v) downstream the D consensus sequence; or (vi) a combination of (i)-(v).

In embodiments, the snoRNA sequence binds a ribonucleoprotein (RNP) to direct trans-splicing of the pre-mRNA target sequence.

In embodiments, the RNA trans-splicing comprises a snoRNA and one or more additional intronic RNA sequences. In embodiments, the one or more additional intronic RNA sequences comprises one or more snRNA. In embodiments, the snRNA is selected from a U7 snRNA, a U1 snRNA, a U2 snRNA, a U4 snRNA, a U4atac snRNA, a U5 snRNA, a U6 snRNA, a U6atac snRNA, a U11 snRNA, and a U12 snRNA. In embodiments, the snRNA is a U7 snRNA. In embodiments, the snRNA and/or snoRNA sequence assembles into an RNP. In embodiments, the snRNA and/or snoRNA sequence comprises a sequence motif that assembles into an RNP.

Persons skilled in the art, with the benefit of this disclosure in its entirety, will be aware of the methods to identify various sequence motifs (snoRNA, snRNA, or otherwise) which may bind RNPs and/or act as splice signals (e.g., splice acceptor, splice donor, etc.), for example using tools such as SPLICEAI, as described in Jaganathan, et al. “Predicting Splicing from Primary Sequence with Deep Learning,” (2019) Cell, Vol. 176: pp. 535-548, the entire contents of which are incorporated by reference, and/or using PANGOLIN, as described in Zeng & Li, “Predicting RNA splicing from DNA sequence using Pangolin,” (2022), Genome Biology, Vol. 23, Article 103, the entire contents of which are incorporated by reference.

In embodiments, the RNA trans-splicing methods, compositions, and/or systems further comprises one or more proteins that forms, is associated with, or is within, an RNP. In embodiments, the RNA trans-splicing methods, compositions, and/or systems further comprises one or more nucleic acid sequences encoding one or more proteins that are associated with, or are within, the RNP. In embodiments, the RNP is associated with an snRNA and/or snoRNA. In embodiments, the methods comprise introduction into the cell a plasmid, viral vector, non-viral vector, or circular RNA (cirRNA) which encodes one or more proteins for RNP formation.

In embodiments, the trans-splicing RNA comprises one or more binding domains, each comprising about 4 nucleotides to about 300 nucleotides. In embodiments, the one or more binding domains are located outside the snoRNA sequence. In embodiments, the one or more binding domains are located within the snoRNA sequence (e.g., between one or more H/ACA box consensus sequences, or between one or more C/D box consensus sequences). In embodiments, the one or more binding domains are located both within and outside of the snoRNA sequence. In embodiments, the snoRNA comprises 2 binding domain sequences, or comprises more than 2 binding domains (e.g., comprises 3 binding domains, 4 binding domains, 5 binding domains, etc.). In embodiments, the one or more binding domain sequences is about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, including size ranges therebetween, each binding domain having at least 80% complementarity to a pre-mRNA target sequence.

In embodiments, the one or more binding domain sequences is a reverse complementary sequence (e.g., relative to the pre-mRNA target sequence) that comprises about or at least about 80% complementarity to one or more pre-mRNA target sequence, about or at least about 85% complementarity to one or more pre-mRNA target sequence, about or at least about 90% complementarity to one or more pre-mRNA target sequence, about or at least about 95% complementarity to one or more pre-mRNA target sequence, about or at least about 96% complementarity to one or more pre-mRNA target sequence, about or at least about 97% complementarity to one or more pre-mRNA target sequence, about or at least about 98% complementarity to one or more pre-mRNA target sequence, or about or at least about 99% complementarity to one or more pre-mRNA target sequence, including percent identity therebetween.

In embodiments, the one or more reverse complimentary sequences between the one or more binding domains and one or more pre-mRNA target sequences is about 4 nucleotides to about 20,000 nucleotides or more in length (e.g., about or at least about 100 nucleotides, 500 nucleotides, 1000 nucleotides, 2000 nucleotides, 3000 nucleotides, 4000 nucleotides, 5000 nucleotides, 6000 nucleotides, 7000 nucleotides, 8000 nucleotides, 9000 nucleotides, 10000 nucleotides, 15000 nucleotides, 20000 nucleotides, etc., including size ranges therebetween).

In embodiments, the pre-mRNA target sequence comprises an USH2A pre-mRNA sequence, or wherein the target comprises intron 12 and/or exon 13 of the USH2A pre-mRNA.

In embodiments, the snoRNA comprises one or more M6A sites. In embodiments, the snoRNA comprises at least 1, 2, 3 4, 5, 6, 7, 8, 9, 10, 11, 12, 13, 14, 15, 16, 17, 18, 19, 20, 21, 22, 23, 24, 25, 26, 27, 28, 29, 30, 40, or 50 or more M6A sites.

In embodiments, the snoRNA comprises a nucleic acid sequence of SNORA101B, SNORA48, SNORA54, SNORA66, SNORA73A, or SNORA8; or one of SNORA101B, SNORA48, SNORA54, SNORA66, SNORA73A, or SNORA5 comprising one or more nucleic acid substitutions, deletions, or extensions relative to a wild-type sequence.

In embodiments, the snoRNA nucleic acid sequence has at least about 90%, about 95%, about 98%, or about 99% identity to a nucleic acid selected from any one of SEQ ID NOs: 590-657 and 732-793 (e.g., Table 6). In embodiments, the snoRNA nucleic acid sequence comprises a nucleic acid sequence selected from any one of SEQ ID NOs: 590-657 and 732-793.

In embodiments, the at least one intronic sequence comprises one or more splicing signals, e.g., as described herein. In embodiments, the one or more splicing signals comprise one or more of an exonic splicing enhancer (ESE), an intronic splicing enhancer (ISE), an exonic splicing silencer (ESS), intronic splicing silencer (ISS), a polypyrimidine tract, a branch point, and a combination thereof. In embodiments, the one or more splicing signals includes splice acceptors and/or splice donors. In embodiments, the one or more splicing signals includes cryptic splice sites (css). In embodiments, the one or more splicing signals includes a boundary sequence between an intron and an exon. In embodiments, the one or more splicing signals includes one or more splice sites, including sequence regions where splicing factors (e.g., like U1, etc.) and/or complex formation with splicing factors, and/or splicing factor recruitment occurs, whether or not splicing takes place at such sites. Persons skilled in the art, with the benefit of this disclosure in its entirety, will be aware of the methods to identify various splicing signals (ESE, ISE, ESS, ISS, polypyrimidine tract, branch point, css, SA, SD, etc.) and various sequences thereof, including sequences which may recruit or form RNPs and/or act as splice signals, for example using tools such as SPLICEAI (as described in Jaganathan, et al. 2019) and/or PANGOLIN (as described in Zeng & Li, 2022).

In embodiments, the trans-splicing RNA molecule comprises one or more polypyrimidine tracts (PPT), branch points (BP), and splice acceptors (SA), optionally comprising one or more splice donor (SD), and is suitable for 3′ editing of the pre-mRNA target sequence.

In embodiments, the trans-splicing RNA molecule comprises one or more splice donors (SD), and optionally comprises one or more polypyrimidine tracts, branch points, and splice acceptors (SA), and is suitable for 5′ editing of the pre-mRNA target sequence.

In embodiments, the trans-splicing RNA molecule further comprises one or more ribozyme sites, e.g., as described herein. In embodiments, the one or more ribozyme sites comprises a hairpin, hammerhead, hepatitis delta virus (HDV), twister ribozyme site, Varkud satellite (VS), or glmS ribozyme site, or a variant thereof. In embodiments, the ribozyme removes the 3′ UTR of the trans-splicing molecule.

In embodiments, the trans-splicing molecule comprises a sequence of MALAT1 and/or NEAT1 (e.g., SEQ ID NO: 712).

In embodiments, the trans-splicing RNA molecule further comprises one or more of a 5′ cap, 5′ UTR, 3′ untranslated region (UTR), and 3′ polyA signal/tail.

In embodiments, the method comprises introducing the one or more trans-splicing RNA molecules into one or more cells. In embodiments, the introduction further comprises contacting the cell with a plasmid, viral vector, non-viral vector, or circular RNA (cirRNA) encoding the one or more trans-splicing RNA molecules. In embodiments, the introduction further comprises contacting the cell with multiple plasmids, viral vectors, non-viral vectors, or circular RNAs (cirRNAs), each encoding distinct trans-splicing RNA molecules. In embodiments, the contacting the cell comprises using a delivery vehicle comprising a lipid nanoparticle (LNP) or polymeric nanoparticles (e.g., wherein the nucleic acid payload is encapsulated in the delivery vehicle components). In embodiments, the viral vector is an adenoviral vector (AAV).

In aspects, described herein are AAV-based vectors for use in performing methods herein as a delivery vehicle to target cells for trans-splicing. In embodiments, the AAV is a cell-specific trans-serotypes for cell-specific delivery. In embodiments, method herein comprise using AAVs encoding one or more trans-splicing nucleic acids, e.g., as described herein.

### Cellular RNA Editing

In embodiments, the method comprises introducing a composition, system, and/or the one or more nucleic acids comprising one or more nucleotide sequences described herein, the viral vector, or pharmaceutical composition described herein to a cell or cell population. In some embodiments, the method comprises contacting the cell with a composition, system, and/or the one or more nucleic acids comprising one or more nucleotide sequences described herein, the viral vector, or pharmaceutical composition described herein. In some embodiments, the cell is a eukaryotic cell. In some embodiments, the eukaryotic cell is a mammalian cell. In some embodiments, the eukaryotic cell is a rodent cell. In some embodiments, the eukaryotic cell is a human cell. In some embodiments, the cell is a patient-derived cell.

The composition, system, and/or the one or more nucleic acids comprising one or more nucleotide sequences described herein, the viral vector, or pharmaceutical composition described herein may be introduced into the cell via any methods known in the art, such as, e.g., viral or bacteriophage infection, transfection, conjugation, protoplast fusion, lipofection, electroporation, calcium phosphate precipitation, polyethyleneimine (PEI)-mediated transfection, DEAE-dextran-mediated transfection, liposome-mediated transfection, particle gun technology, calcium phosphate precipitation, shear-driven cell permeation, fusion to a cell-penetrating peptide followed by cell contact, microinjection, and nanoparticle-mediated delivery. In some embodiments, the vector system may be introduced into the cell via viral infection.

In embodiments, the disclosure provides a method for targeting trans-splicing of a target RNA (e.g., pre-mRNA) in a cell, the method comprising contacting the cell with a composition, system, and/or the one or more nucleic acids comprising one or more nucleotide sequences described herein, the viral vector, or pharmaceutical composition described herein, wherein when the cell is contacted with the composition, system, and/or the one or more nucleic acids comprising one or more nucleotide sequences described herein, the viral vector, or pharmaceutical composition described herein, the one or more binding domains of the one or more nucleic acids comprising one or more nucleotide sequences described herein binds to the target RNA (e.g., pre-mRNA) and trans-splicing results in ligation of one or more exons of the target RNA (e.g., pre-mRNA) to one or more exons of the one or more nucleic acids comprising one or more nucleotide sequences described herein.

In embodiments, the disclosure provides a method for targeting trans-splicing of a pre-mRNA in a cell or a population of cells comprising a disease-causing mutation, the method comprising contacting the cell or population of cells with a composition, system, and/or the one or more nucleic acids comprising one or more nucleotide sequences described herein, the viral vector, or pharmaceutical composition described herein, wherein when the cell is contacted with the one or more nucleic acids comprising one or more nucleotide sequences described herein, the one or more binding domains of the one or more nucleic acids comprising one or more nucleotide sequences described herein binds to the pre-mRNA and trans-splicing results in ligation of one or more exons of the pre-mRNA to one or more exons of the one or more nucleic acids comprising one or more nucleotide sequences described herein, thereby resulting in a mRNA lacking the disease-causing mutation.

In embodiments, the disclosure provides a method for targeting trans-splicing of a pre-mRNA in a cell or a population of cells derived from a patient having a disease or disorder, the method comprising contacting the cell or population of cells with a one or more nucleic acids comprising one or more nucleotide sequences described herein, the viral vector, or pharmaceutical composition described herein, wherein when the cell or population of cells is contacted with the composition, system, and/or the one or more nucleic acids comprising one or more nucleotide sequences described herein, the viral vector, or pharmaceutical composition described herein, the one or more binding domains of the one or more nucleic acids comprising one or more nucleotide sequences described herein binds to the target RNA (e.g., pre-mRNA) and trans-splicing results in ligation of one or more exons of the target RNA (e.g., pre-mRNA) to one or more exons of the one or more nucleic acids comprising one or more nucleotide sequences described herein, wherein the cell or population of cells is reintroduced to the patient, thereby treating or ameliorating the disease or disorder.

### In Vivo RNA Editing

The present disclosure provides methods for treating a patient having a disease or disorder using the composition, system, and/or the one or more nucleic acids comprising one or more nucleotide sequences described herein, the viral vector, or pharmaceutical composition described herein. In some embodiments, the disease or disorder is associated with one or more mutations in a target RNA, wherein the method targets trans-splicing of the target RNA to remove the one or more mutations.

In embodiments, the disclosure provides a method of treating a patient having a disease or disorder, comprising administering to the patient a composition, system, and/or the one or more nucleic acids comprising one or more nucleotide sequences described herein, the viral vector, or pharmaceutical composition described herein.

In embodiments, the disclosure provides a method of treating a patient having a disease or disorder by targeting trans-splicing of a target RNA (e.g. pre-mRNA) in a target tissue or cell population, the method comprising administering to the patient a composition, system, and/or the one or more nucleic acids comprising one or more nucleotide sequences described herein, the viral vector, or pharmaceutical composition described herein, wherein when the composition, system, and/or the one or more nucleic acids comprising one or more nucleotide sequences described herein, the viral vector, or pharmaceutical composition described herein is administered the one or more nucleic acids comprising one or more nucleotide sequences described herein binds to a target RNA (e.g., pre-mRNA) and trans-splicing results in ligation of one or more exons of the target RNA (e.g., pre-mRNA) to one or more exons of the composition, system, and/or the one or more nucleic acids comprising one or more nucleotide sequences described herein, thereby treating or ameliorating the disease or disorder.

In embodiments, the disclosure provides a method of treating a patient having a disease or disorder associated with one or more mutations in a pre-mRNA in a target tissue or cell population, the method comprising administering to the patient a composition, system, and/or the one or more nucleic acids comprising one or more nucleotide sequences described herein, the viral vector, or pharmaceutical composition described herein, wherein when the composition, system, and/or the one or more nucleic acids comprising one or more nucleotide sequences described herein is administered, the one or more nucleic acids comprising one or more nucleotide sequences described herein binds to a pre-mRNA and trans-splicing results in ligation of one or more exons of the pre-mRNA to one or more exons of the one or more nucleic acids comprising one or more nucleotide sequences described herein, wherein the trans-splicing results in an mRNA lacking the disease-causing mutation, thereby treating or ameliorating the disease or disorder.

In embodiments, the route of administration is any sufficient for delivery of the composition, system, and/or the one or more nucleic acids comprising one or more nucleotide sequences described herein, the viral vector, or pharmaceutical composition described herein to the target tissue or cell population ascertained by one of skill in the art.

In embodiments, administration of the composition, system, and/or the one or more nucleic acids comprising one or more nucleotide sequences described herein, the viral vector, or pharmaceutical composition described herein results in correction of a mutation in a pre-mRNA in a target tissue or cell population in the patient.

The term “treatment” refers to the application of one or more methods described herein for the amelioration of a disease. In some embodiments, the specific procedure is the administration of a composition, system, and/or the one or more nucleic acids comprising one or more nucleotide sequences described herein, the viral vector, or pharmaceutical composition described herein.

“Treatment” of an individual (e.g. a mammal, such as a human) or a cell is any type of intervention used in an attempt to alter the natural course of the individual or cell. Treatment includes, but is not limited to, administration of a composition, system, and/or the one or more nucleic acids comprising one or more nucleotide sequences described herein, the viral vector, or pharmaceutical composition described herein, and may be performed either prophylactically or subsequent to the initiation of a pathologic event or contact with an etiologic agent. Treatment includes any desirable effect on the symptoms or pathology of a disease or condition, and may include, for example, minimal changes or improvements in one or more measurable markers of the disease or condition, and may include, for example, minimal changes or improvements in one or more measurable markers of the disease or condition being treated.

The terms “patient,” “subject,” “individual,” and the like are used interchangeably herein, and refer to any animal amenable to the methods described herein. In some embodiments, the patient, subject, or individual is a human.

### Kits

The present disclosure provides kits for carrying out the methods described herein. In some embodiments, the kit comprises a composition, system, and/or the one or more nucleic acids comprising one or more nucleotide sequences described herein, the viral vector, or pharmaceutical composition described herein.

In some embodiments, the kit comprises a composition, system, and/or the one or more nucleic acids comprising one or more nucleotide sequences described herein, the viral vector, or pharmaceutical composition described herein and a reagent for reconstitution and/or dilution of the composition, system, and/or the one or more nucleic acids comprising one or more nucleotide sequences described herein, the viral vector, or pharmaceutical composition described herein.

In some embodiments, the kit comprise one or more additional reagents, where such additional reagents are selected from a buffer, a buffer for introducing the composition, system, and/or the one or more nucleic acids comprising one or more nucleotide sequences described herein, recombinant expression vector, delivery system into a cell, a wash buffer, a control reagent, a control vector, a control polynucleotide, a reagent for in vitro production of the recombinant expression vector or delivery system, adaptors for sequencing and the like. A buffer can be a stabilization buffer, a reconstituting buffer, a diluting buffer, or the like. A kit can also comprise one or more components that can be used to facilitate or enhance the on-target binding or the trans-splicing of the composition, system, and/or the one or more nucleic acids comprising one or more nucleotide sequences described herein.

In addition to the above-mentioned components, a kit can further comprise instructions for using the components of the kit to practice the methods. The instructions for practicing the methods can be recorded on a suitable recording medium. For example, the instructions can be printed on a substrate, such as paper or plastic, etc. The instructions can be present in the kits as a package insert, in the labeling of the container of the kit or components thereof (i.e., associated with the packaging or subpackaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g. via the Internet), can be provided. An example of this case is a kit that comprises a web address where the instructions can be viewed and/or from which the instructions can be downloaded. As with the instructions, this means for obtaining the instructions can be recorded on a suitable substrate.

In some embodiments, the kit comprises a container comprising the composition, system, and/or the one or more nucleic acids comprising one or more nucleotide sequences described herein, the viral vector, or pharmaceutical composition described herein described herein, and instructions for use targeting trans-splicing of a target RNA (e.g., pre-mRNA) in a cell or a population of cells.

In some embodiments, the kit comprises a container comprising the composition, system, and/or the one or more nucleic acids comprising one or more nucleotide sequences described herein, the viral vector, or pharmaceutical composition described herein, and instructions for administering the one or more nucleic acids comprising one or more nucleotide sequences, the recombinant expression vector, the delivery system, or the pharmaceutical composition to a patient in need thereof to target trans-splicing of a target RNA (e.g., pre-mRNA) in a cell or a population of cells of the patient.

In various embodiments, the “subject” refers to any animal (e.g., a mammal), including, but not limited to, humans, and non-human animals (including, but not limited to, non-human primates, dogs, cats, rodents, horses, cows, pigs, mice, rats, hamsters, rabbits, and the like (e.g., which is to be the recipient of a particular treatment, or from whom cells are harvested)). In embodiments, the subject is a human.

It will also be understood that, although the terms first, second, etc., may be used herein to describe various elements, these elements should not be limited by these terms. These terms are only used to distinguish one element from another. For example, a first subject could be termed a second subject, and, similarly, a second subject could be termed a first subject, without departing from the scope of the present disclosure. The first subject and the second subject are both subjects, but they are not the same subject. Furthermore, the terms “subject,” “user,” and “patient” are used interchangeably herein.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Selection of ncRNAs

The experiments of this example describe the methods used to identify ncRNAs for inclusion in composition, system, and/or the one or more nucleic acids comprising one or more nucleotide sequences described herein capable of targeting a pre-mRNA and producing a trans-splicing event. As shown in FIG. 1E, it was determined that nearly all ncRNA sequences mined from public databases contain a sequence motif identified in Table 1. RNAlib-2.5.1 software was used to predict the secondary structure of the ncRNA sequences identified in the public domain, including metazoan U1 snRNA sequences, U11 snRNA sequences, U7 snRNA sequences, Sm sequences, and H/ACA snoRNA sequences. The predicted secondary structures were compared to known secondary structures using an RNA covariance model (see, e.g., Eddy, et al (1994) Nucleic Acids Research 22:2079-2088). ncRNA sequences having a predicted secondary structure with similarity to known secondary structures were selected for further evaluation. This approach provided more than 120,000 candidate ncRNA sequences. As shown in FIG. 1F, the candidate ncRNA sequences ranged from approximately 7 nucleotides to more than 300 nucleotides in length. Exemplary candidate ncRNA sequences that were identified by this computational analysis are set forth in SEQ ID NOs: 9-657.

### Example 2: Design and Testing of Compositions, Systems and Nucleic Acids for Trans-Splicing

The experiments of this example show the design and testing of composition, system, and/or the one or more nucleic acids comprising one or more nucleotide sequences described herein for targeted trans-splicing. The nucleic acid molecules comprise a nucleotide sequence with (a) an intronic sequence having (i) at least one binding domain sequence complementary to a target sequence in a pre-mRNA, and (ii) a ncRNA sequence; (b) a splice site; and (c) at least one exonic sequence. The ncRNA sequences were selected from the candidate ncRNAs identified as described in Example 1. The composition, system, and/or the one or more nucleic acids comprising one or more nucleotide sequences described herein were engineered to incorporate the entire candidate ncRNA sequence or a portion thereof comprising a secondary structure and/or sequence motif identified in Table 1.

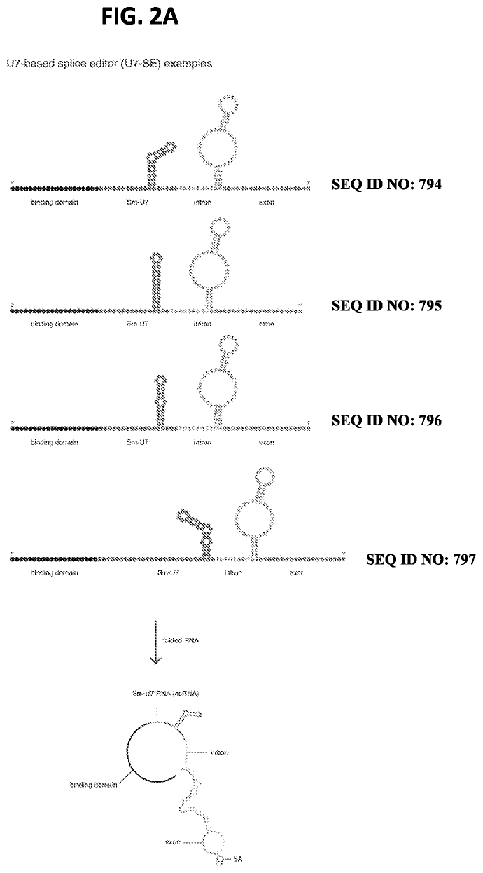

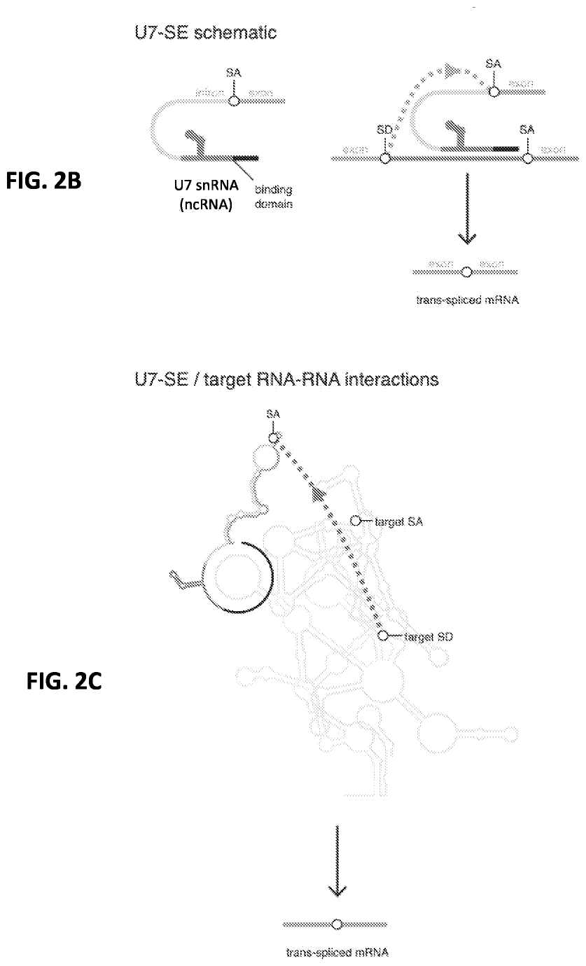

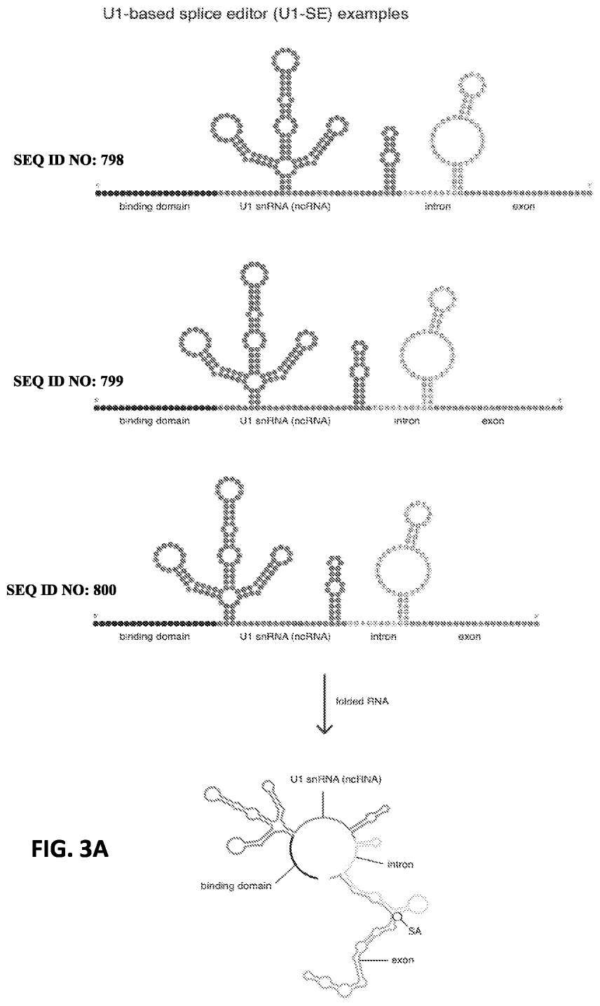

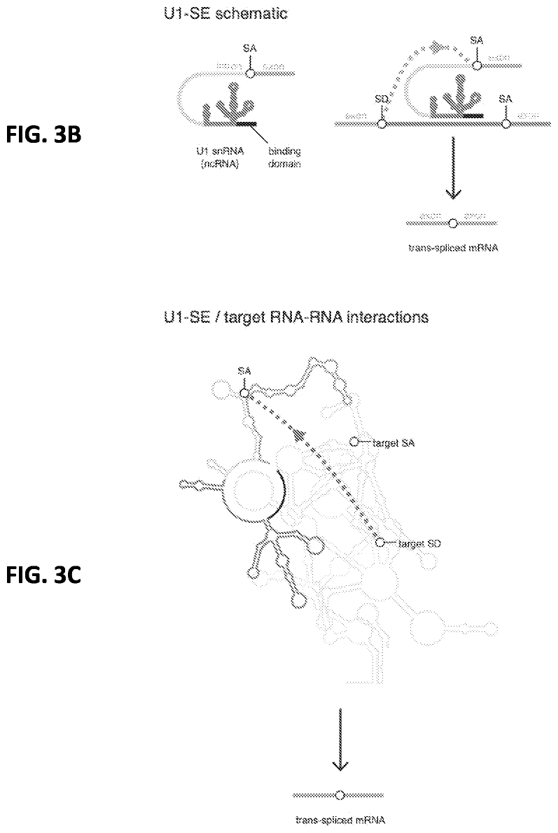

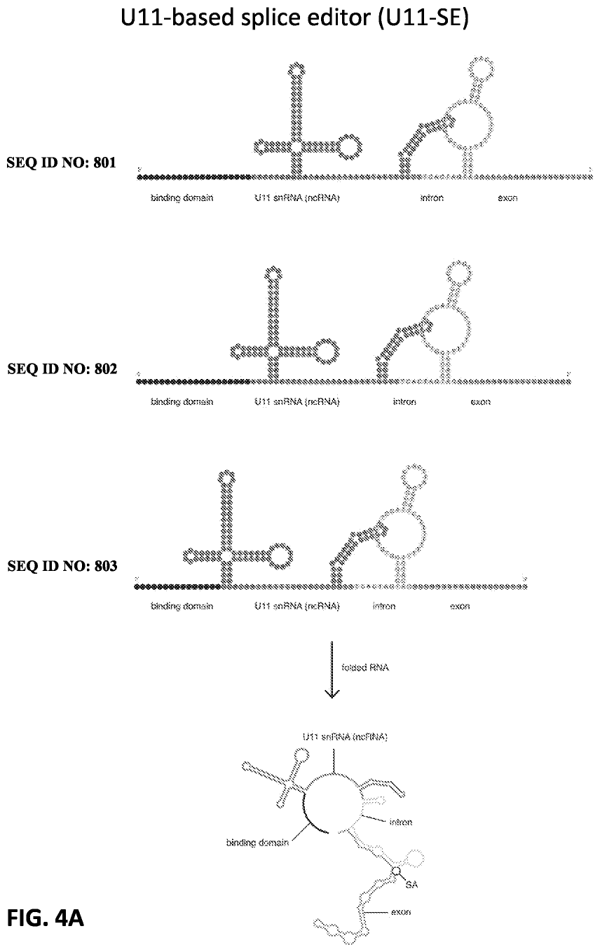

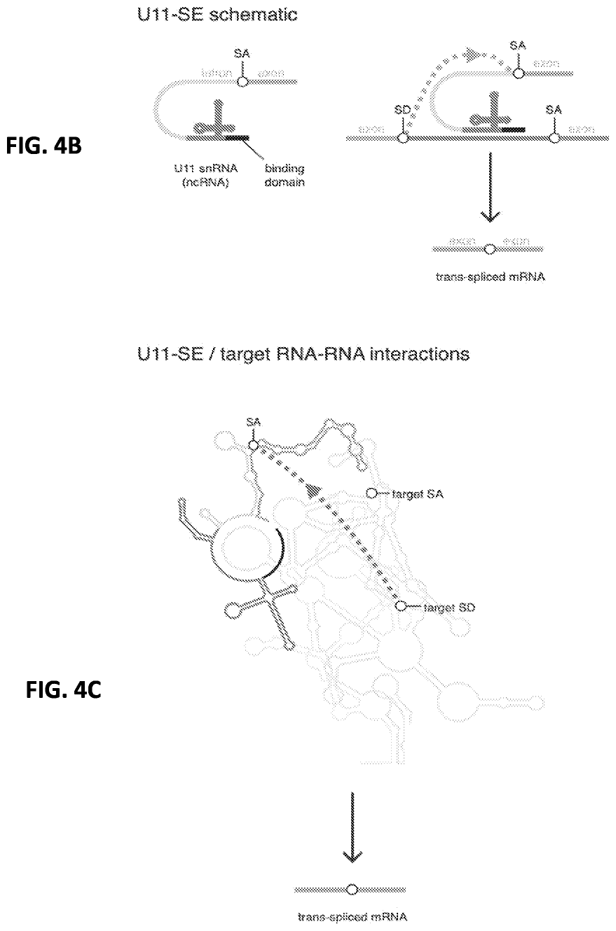

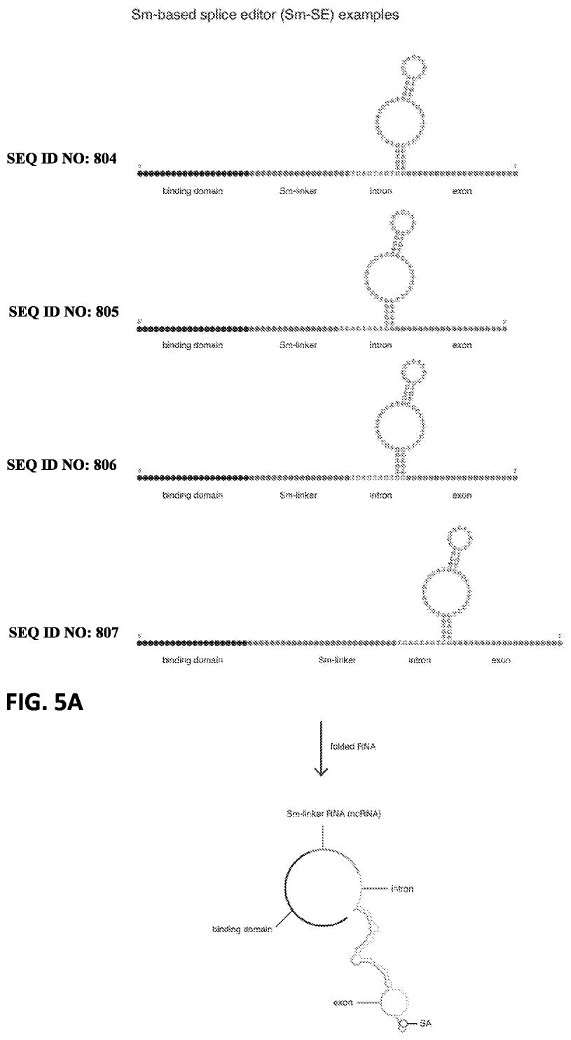

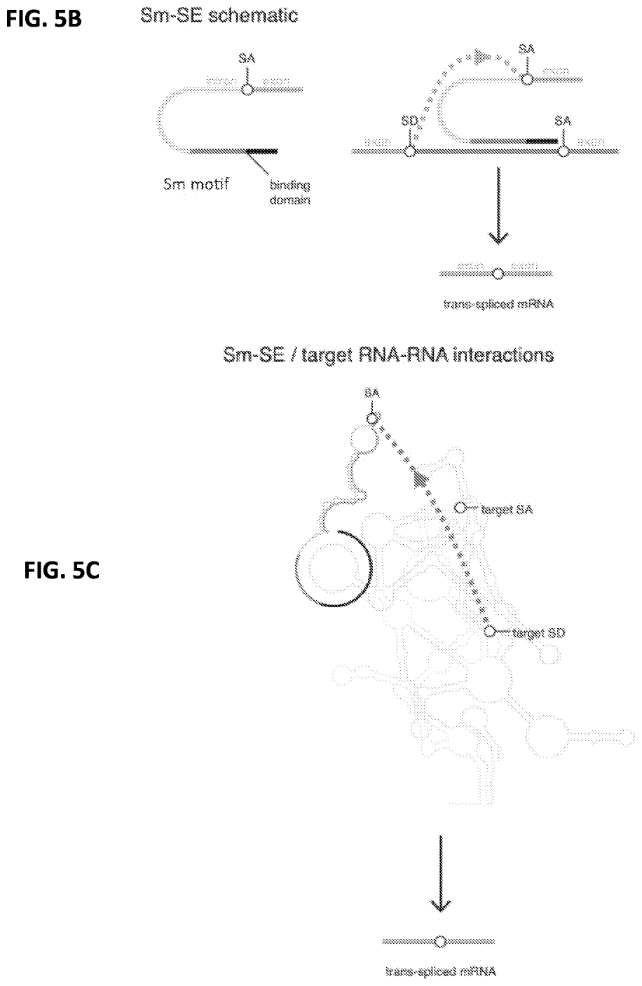

A first set of nucleic acid molecules were designed to have a ncRNA sequence derived from a snRNA and to undergo trans-splicing at a splice donor in a pre-mRNA (for correction of a mutation at the 5′end of an exon). The nucleic acid molecules had a nucleotide sequence arranged 5′ to 3′ (a) an intron having (i) a binding domain sequence, (ii) a snRNA sequence (a U1 snRNA; U11 snRNA; a Sm sequence motif and U7 snRNA; or a Sm sequence motif), (iii) a branch point, and (iv) a polypyrimidine tract; (b) a splice acceptor; and (c) an exon. Schematics of exemplary U1-based composition, system, and/or the one or more nucleic acids comprising one or more nucleotide sequences described herein composition, system, and/or the one or more nucleic acids comprising one or more nucleotide sequences described herein are shown in FIG. 2A, FIG. 2B, and FIG. 2C and sequences are provided in the table of Table 6 (in rows of Table 6 in which the column titled “Region 2” is assigned a label of “U1_X_Y” in which X and Y are integers). Schematics of exemplary U11-based composition, system, and/or the one or more nucleic acids comprising one or more nucleotide sequences described herein are shown in FIG. 3A, FIG. 3B, and FIG. 3C and sequences are provided in the table of Table 6 (in rows of Table 6 in which the column titled “Region 2” is assigned a label of “U11_X_Y” in which X and Y are integers). Schematics of exemplary U7-based composition, system, and/or the one or more nucleic acids comprising one or more nucleotide sequences described herein are shown in FIG. 4A, FIG. 4B, and FIG. 4C and sequences are provided in Table 6 (in rows of Table 6 in which the column titled “Region 2” is assigned a label beginning with “Sm_Z” in which Z is an integer and the column titled “Region 3” is assigned a label beginning with “U7_X” in which X is an integer). Schematics of exemplary Sm-based composition, system, and/or the one or more nucleic acids comprising one or more nucleotide sequences described herein are shown in FIG. 5A, FIG. 5B, and FIG. 5C and sequences are provided in Table 6 (in rows of Table 6 in which the column titled “Region 2” is assigned a label beginning with “Sm_Z” in which Z is an integer and the column titled “Region 3” is assigned a label “adeno_intron”).

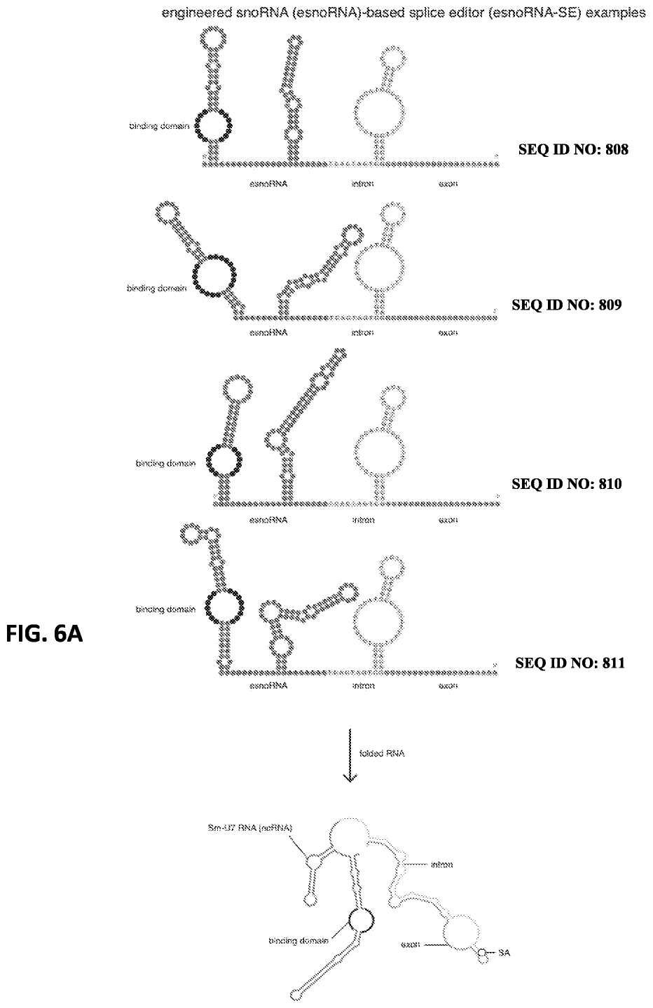

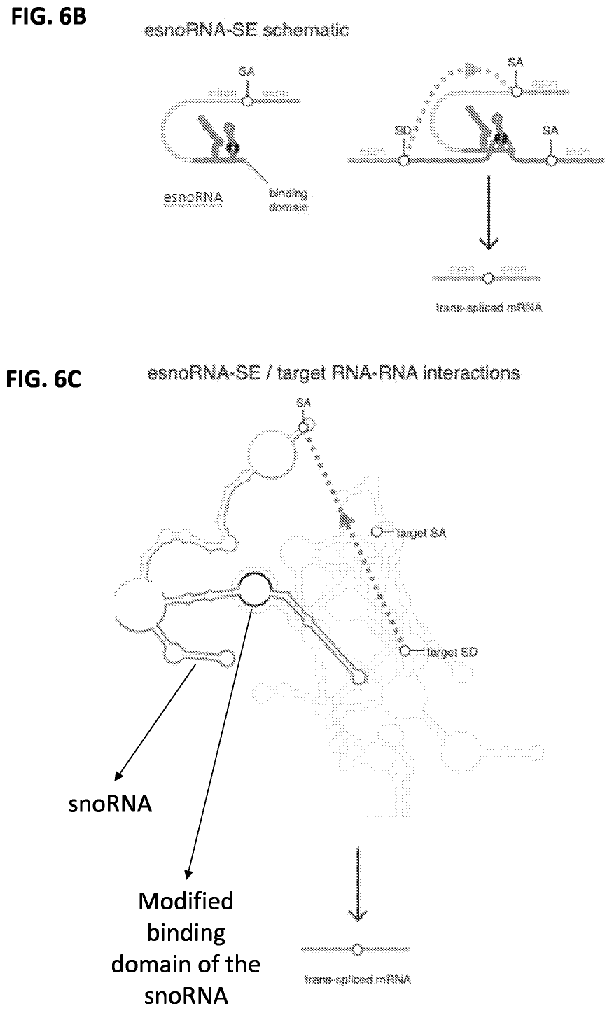

A second set of nucleic acid molecule were designed to have a ncRNA sequence derived from a H/ACA snoRNA and to undergo trans-splicing at a splice donor in a pre-mRNA (for correction of a mutation at the 5′end of an exon). The nucleic acid molecules had a nucleotide sequence arranged 5′ to 3′ (a) an intron having (i) a first and second binding domain inserted into an H/ACA box snoRNA sequence, (ii) a branch point, and (iii) a polypyrimidine tract; (b) a splice acceptor; and (c) an exon. Schematics of exemplary snoRNA-based composition, system, and/or the one or more nucleic acids comprising one or more nucleotide sequences described herein are shown in FIG. 6A, FIG. 6B, and FIG. 6C and sequences are provide in Table 6 (in rows of Table 6 in which the column titled “Region 1” is assigned a label beginning with “sno” “SNO” or “SCARNA”).

The nucleic acid molecules are evaluated for trans-splicing by using reporter cells, where correct RNA edits generate a mRNA that produces a fluorescent protein. The composition, system, and/or the one or more nucleic acids comprising one or more nucleotide sequences described herein are be introduced to the reporter cells via viral or non-viral methods. Viral methods include but are not limited to lentivirus, AAV, and adenovirus. Non-viral methods include but are not limited to transfection or electroporation. The cells are first transfected with a splice donor reporter construct encoding a pre-mRNA under control of a CMV promoter, the pre-mRNA comprising a blue fluorescent protein (BFP), a self-cleaving p2A linker, a truncated GFP (5′GFP), and a splice donor. The BFP is used to confirm stable expression of the reporter construct. The reporter construct has a matrix metallopeptidase 9 (MMP9) intron 1 and exon 2 downstream the splice donor to ensure splicing an event can occur, followed by a bovine growth hormone polyadenylation signal (bGHpA) to allow for stable expression of the construct. The composition, system, and/or the one or more nucleic acids comprising one or more nucleotide sequences described herein have an exon that is the second half of the truncated GFP (3′GFP) and trans-splicing results in expression of full-length GFP. Reporter cells with correct edits generate signal via a fluorescent reporter and are sorted via FACS. Sorted cells are sequenced to identify active compositions, systems, and/or the one or more nucleic acids comprising one or more nucleotide sequences described herein.

### Example 3: U7 gRNAs Enable and Enhance Trans-Splicing

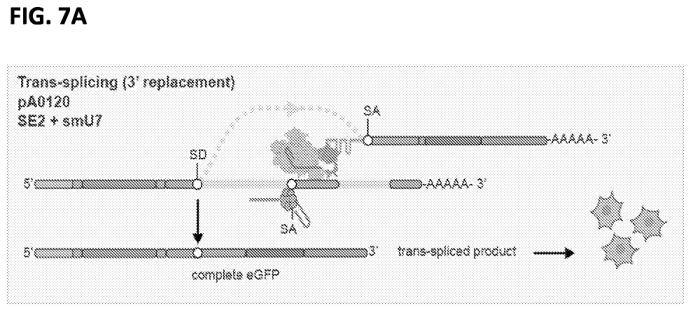

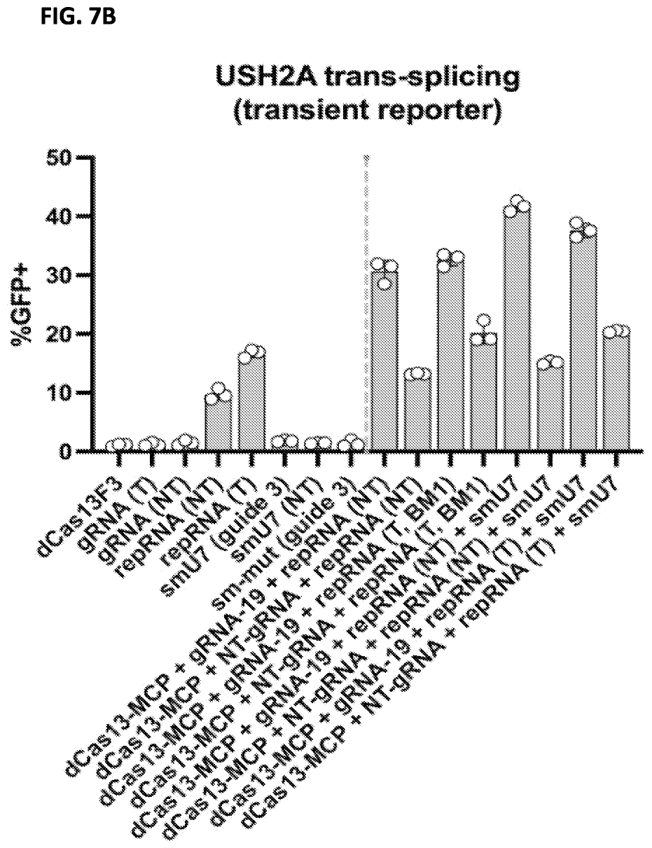

The experiments of this example show how U7 gRNAs enable and enhance trans-splicing in a variety of experimental conditions. In FIG. 7A and FIG. 7B, WT HEK293FT cells or cells with a PiggyBac integrated reporter were seeded 16 hours prior to transfection. At a confluence of 60-80%, cells were transfected with 25 ng reporter plasmid, 25 ng smU7 guide plasmid, 25 ng dCas13F3-MCP plasmid, 25 ng Cas13 gRNA plasmid, and 25 ng repRNA plasmid. For various conditions lacking one or more of these elements, pUC19 plasmid was substituted at equivalent inputs to normalize transfection efficiency. All cells were transfected using Lipofectamine 2000. Approximately 48 hours after transfection, cells were trypsinized using TrypLE and assayed via flow cytometry. Editing efficiency was determined as the percentage of all GFP+ cells after gating for BFP+ singlets. FIG. 7A provides a schematic, without wishing to be bound by theory, depicting a U7 gRNA enhancing Cas13-based trans-splicing. FIG. 7B is a graph showing data of the USH2A trans-splicing system and the combination U7 gRNAs with an RNA-targeting CRISPR-Cas (protein and CRISPR gRNA) and a repair RNA enables, compared to conditions where the U7 gRNA are not provided (RNA-targeting CRISPR-Cas system and repair RNA alone), or conditions where the U7 gRNA sequence is mutated, or conditions where the U7 gRNA is targeting a different sequence (non-targeting guide). These experiments in FIG. 7A and FIG. 7B demonstrate how U7 gRNAs enable and enhance Cas13-based trans-splicing.

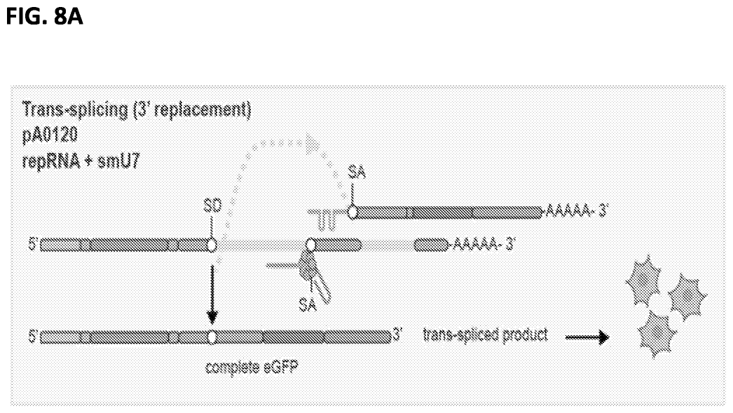

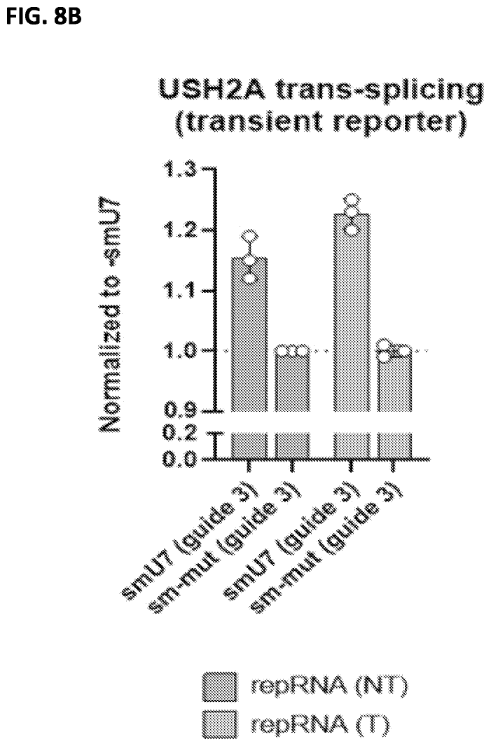

The experiment in FIG. 8A and FIG. 8B show experimental data demonstrating the combination of U7 gRNAs with a repRNA in trans, which enables exon replacement. In these experiments, WT HEK293FT cells or cells with a PiggyBac integrated reporter were seeded 16 hours prior to transfection. At a confluence of 60-80%, cells were transfected with 25 ng reporter plasmid, 25 ng smU7 guide plasmid, and 25 ng repRNA plasmid. For various conditions lacking one or more of these elements, pUC19 plasmid was substituted at equivalent inputs to normalize transfection efficiency. Antisense region (ASR) sequences used in these experiments are shown in Table 3 and Table 4. All cells were transfected using Lipofectamine 2000. Approximately 48 hours after transfection cells, were trypsinized using TrypLE and assayed via flow cytometry. Editing efficiency was determined as the percentage of all GFP+ cells after gating for BFP+ singlets. FIG. 8A provides a schematic, without wishing to be bound by theory, depicting a U7 gRNA enhancing Cas13-based trans-splicing. The experiments in FIG. 8B show data of the USH2A trans-splicing system and the combination U7 gRNAs with a repair RNA in trans, which enables exon replacement, compared to conditions where the U7 gRNA is not provided (repRNA alone), or conditions where the gRNA sequence is mutated, or conditions where the U7 gRNA is targeting a different sequence (non-targeting guide).

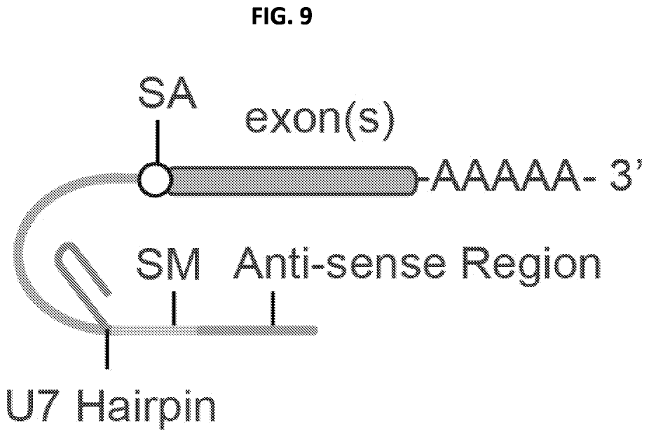

FIG. 9 provides a schematic, without wishing to be bound by theory, depicting U7 grepRNA design for 3′ replacement. The U7 grepRNA design shown in FIG. 9 can include an additional anti-sense binding motif elsewhere in the grepRNA to increase hybridization to the target RNA. In these experiments, the length of the U7 ASR sequence can be variable and maintain function, such as at least or about 10 nucleotides to at least or about 80 nucleotides.

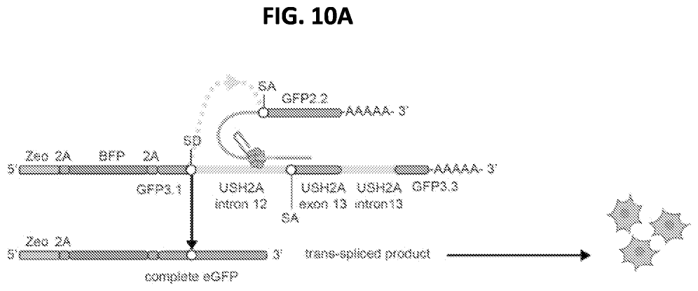

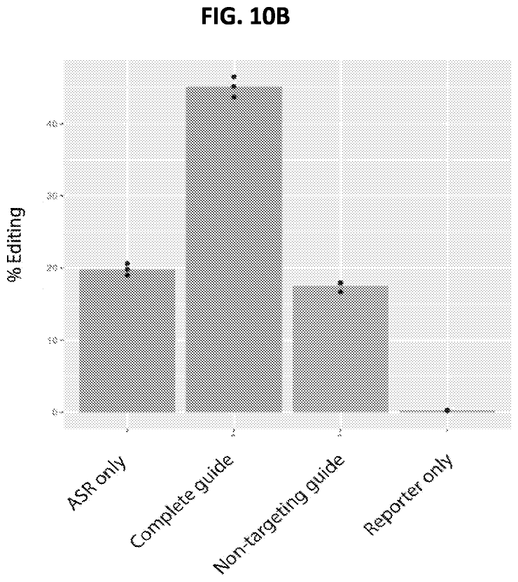

The experiments in FIG. 10A and FIG. 10B show experimental data demonstrating 3′ trans-splicing using a U7 grepRNA. In these experiments, WT HEK293 cells were transfected with 25 ng pA0120 reporter, 50 ng (g) repRNA and 25 ng pUC19 using Lipofectamine 3000. The cells were incubated for 48 hours before being trypsinized and analyzed on flow cytometry. FIG. 10A shows the design of an experiment of 3′ trans-splicing and exon replacement using a U7 grepRNA, compared to a non-targeting ASR sequence, or a grepRNA lacking the U7 hairpin. (Table 4) FIG. 10B is a graph showing U7-guided trans-splicing on an USH2A target (3′ RNA replacement) with transient transfection leading to 45% trans-splicing efficiency.

Accordingly, the results of the experiments of this example demonstrate U7 gRNAs enable and enhance trans-splicing.

### Example 4: snoRNAs Enable and Enhance Trans-Splicing

The experiments in this example demonstrate how snoRNAs enable and enhance trans-splicing activity and efficiency in a variety of conditions.

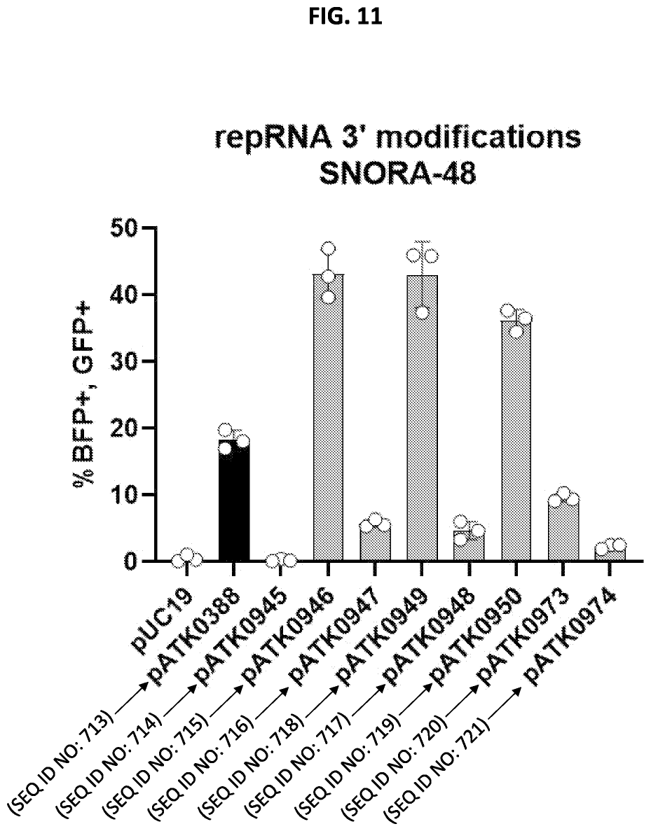

The experiments in FIG. 11 show how the addition of SNORA48-like sequences to the 3′ end of repRNAs designed to edit the 5′ end of the target significantly increased trans-splicing efficiency. FIG. 11 shows pATK0388 (SEQ ID NO: 713), pATK0945 ((SEQ ID NO: 714), pATK0946 (SEQ ID NO: 715), pATK0947 (SEQ ID NO: 716), pATK0948 (SEQ ID NO: 717), pATK0949 (SEQ ID NO: 718), pATK0950 (SEQ ID NO: 719), pATK0973 (SEQ ID NO: 720), and pATK0974 (SEQ ID NO: 721). The experiments in FIG. 11 show how mutations in the H and ACA motifs substantially decrease the trans-splicing efficiency because they cannot form RNPs (ribonuclear proteins), which confirms, without wishing to be bound by theory, the role of H and ACA motifs in the formation of RNPs, and also without wishing to be bound by theory, how the RNP is important for the observed increase in activity. In these experiments, repRNAs were designed with or without (pATK388 control) SNORA modifications. pATK388 lacks a snoRNA. 50 ng of each repRNA plasmid was co-transfected with pUC19 (filler DNA) into a reporter cell line (HEK293FT cells) using Lipofectamine 3000. Cells were collected and analyzed by flow cytometry 48-hrs post-transfection. The y-axis shows the percentage of GFP positive cells of BFP+/GFP+ singlets. These experiments demonstrate the addition of SNORA48 significantly improved trans-splicing with repRNAs.

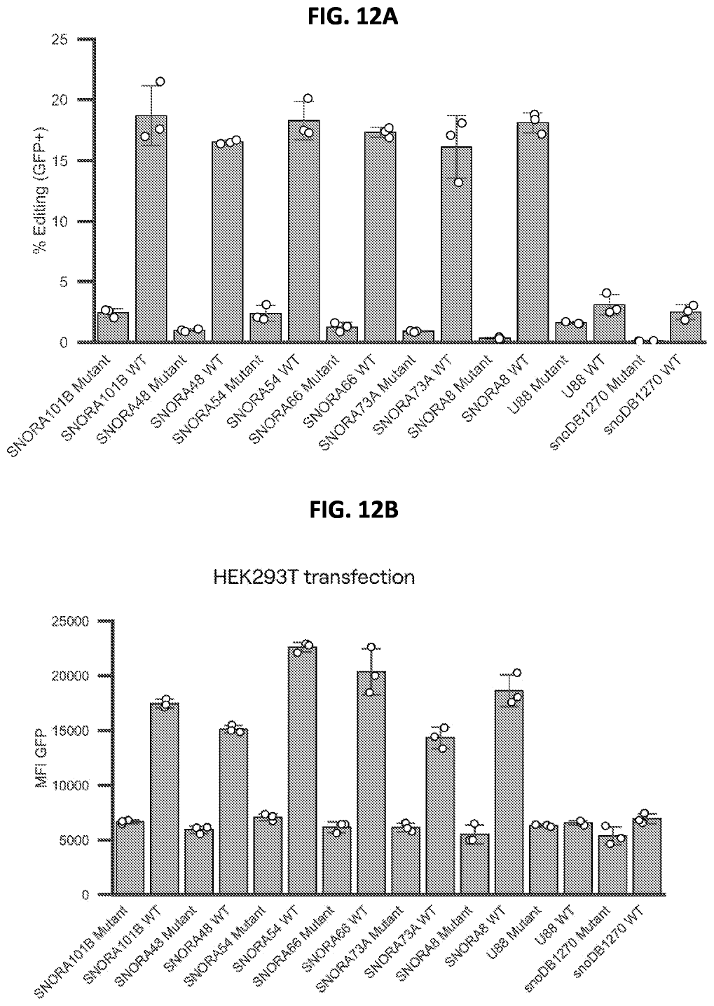

The experiments in FIGS. 12A-12B show how snoRNAs broadly increase trans-splicing efficiency. In these experiments, repRNAs were designed with wildtype or mutant SNORA modifications. 50 ng of each repRNA plasmid was co-transfected with pUC19 (filler DNA) into a reporter cell line (HEK293FT cells) using Lipofectamine 3000. Cells were collected and analyzed by flow cytometry 48-hrs post-transfection. The y-axis shows the percentage of GFP positive cells of BFP+/GFP+ singlets. Mutant conditions impair RNP formation, thus resulting in lower trans-splicing. Overall, the experiments of this example demonstrate the RNA can be stabilized with RNP formation, and the experiments show that snoRNA sequences led to increased trans-splicing efficiency.

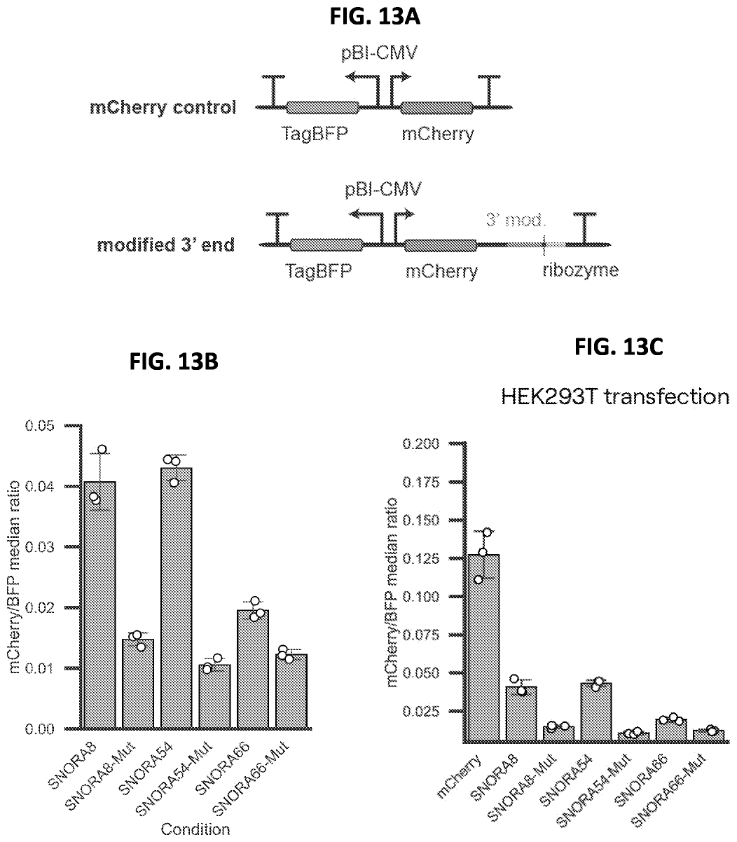

The experiments in FIG. 13A, FIG. 13B, and FIG. 13C show how SNORA modifications on transcripts stabilize the 3′ ends of transcripts after ribozyme cleavage and polyA removal. In these experiments, plasmids were designed with bidirectional promoters constitutively expressing TagBFP (reference control) and mCherry, with or without a 3′ end modification followed by a self-cleaving ribozyme. Transcripts for mCherry were hypothesized to fail to produce mCherry protein after ribozyme cleavage and polyA removal unless stabilized by a 3′ end modification. The WT MALAT1 3′ UTR (contains RNase P cleavage site) has previously been shown to enable “leaky” translation. Cells were collected and analyzed by flow cytometry 48-hrs post-transfection. The y-axis shows ratio of mCherry and BFP fluorescent medians (MFI).

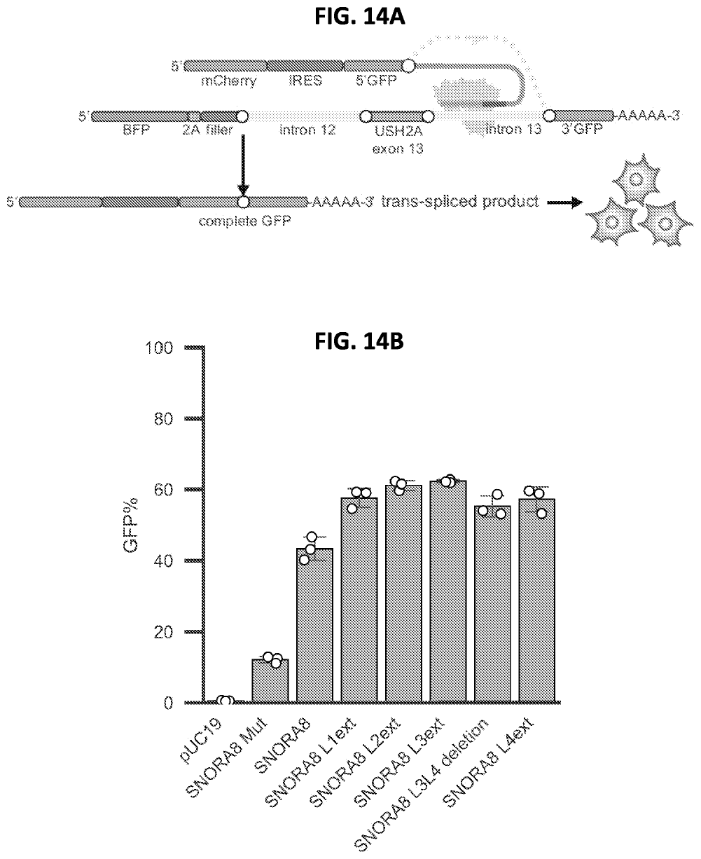

The experiments in FIGS. 14A-14B show how SNORA8 is permissive of extensions and deletions in the targeting regions. In these experiments, repRNAs were designed with wildtype or mutant SNORA modifications. In these experiments, the pATK388 repRNA lacks any SNORA modification and serves as a reference control for trans-splicing. 50 ng of each repRNA plasmid was co-transfected with pUC19 (filler DNA) into a reporter cell line (HEK293FT cells) using Lipofectamine 3000. Cells were collected and analyzed by flow cytometry 48-hrs post-transfection. The y-axis shows the percentage of GFP positive cells of BFP+/GFP+ singlets. This data demonstrates the snoRNA is permissive to modifications that can modulate and/or increase trans-splicing efficiency and activity.

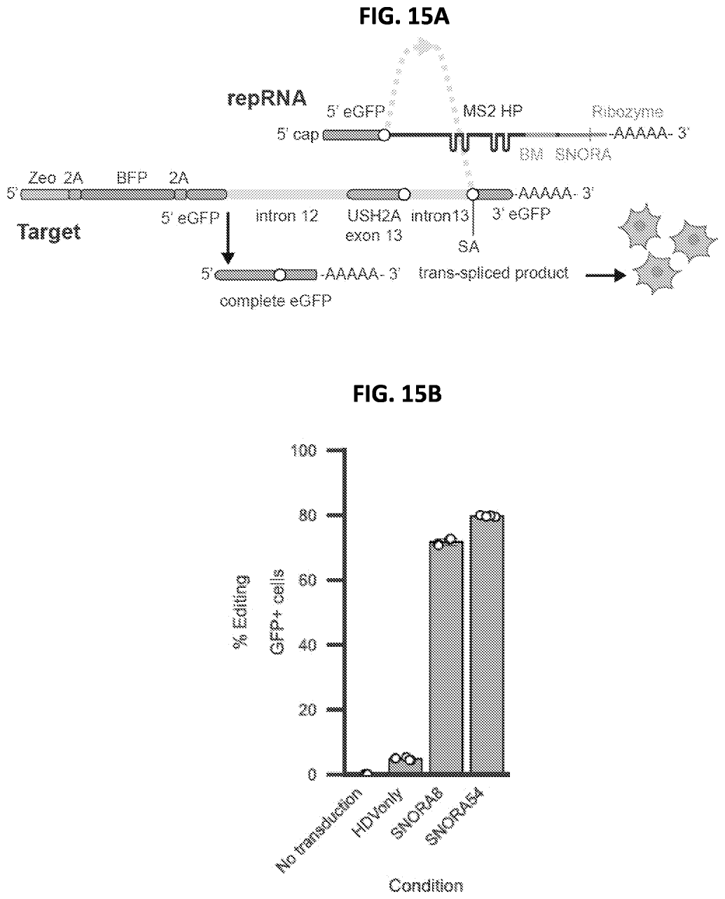

The experiments in FIG. 15A and FIG. 15B show how repRNAs with SNORA modifications are delivered to cells via AAV to edit endogenous reporter RNAs. The experiments tested whether or not SNORA modifications stabilize the 3′ end of a repRNA. In these experiments, AAV2 virus with repRNA cargos were transduced into a reporter cell line (HEK293FT cells) at an MOI of 1E5. Cells were collected and analyzed by flow cytometry 48-hours post-transduction. The y-axis shows the percentage of GFP positive cells of BFP+/GFP+ singlets. SE3 vector is the repRNA without any 3′ end modification. These experiments show how cells transduced with repRNAs containing SNORA modifications showed greatly improved trans-splicing efficiency relative to the SE3 control, which does not include snoRNA sequences.

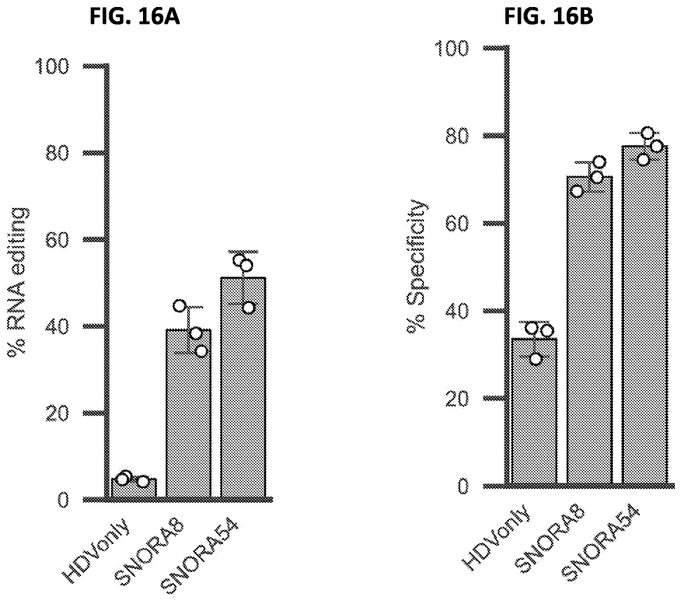

The experiments in FIG. 16A and FIG. 16B are show how repRNAs with SNORA modifications are delivered to cells via AAV to edit endogenous reporter RNAs. In these experiments, an AAV2 virus with repRNA cargos was transduced into a reporter cell line (HEK293FT cells) at an MOI of 1E5. Cells were collected and RNA was analyzed by nanopore sequencing. SE3 vector is a repRNA without any 3′ end modification. These experiments demonstrate that cells transduced with repRNAs containing SNORA modifications showed a significant improvement in trans-splicing efficiency relative to the SE3 control (HDV only), which does not include snoRNA sequences. SNORA-based repRNAs led to a higher activity for trans-splicing, as well as increased specificity.

Accordingly, the results of the experiments of this example demonstrate how snoRNAs enable and enhance trans-splicing activity and efficiency in a variety of conditions.

### Example 5: Mutability and Functional Screening of snoRNAs for Trans-Splicing

The experiments in this example demonstrates modification of snoRNAs to modulate trans-splicing activity in vitro.

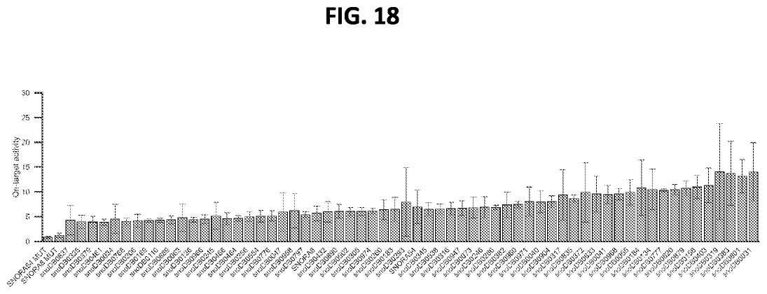

As shown in FIG. 18, various trans-splicing molecules were modified to include snoRNA sequences. All snoRNA sequences containing trans-splicing repair RNAs demonstrated higher trans-splicing activity than trans-splicing repair RNAs which did not form human RNPs (e.g., SNORA54 MUT, SNORA8 MUT). HEK293FT cells were transfected with trans-splicing molecules using LIPOFECTAMINE 2000, per the manufacturer's instructions. RNA was extracted from cells using QIAGEN RNEASY mini extraction kit, per the manufacturer's protocol. RNA-seq libraries were constructed and sequenced on an ILLUMINA platform to determine the trans-splicing rates. Various snoRNA sequences had a >10-fold effect on trans-splicing efficiency. All snoRNA sequences had a >4-fold effect on trans-splicing efficiency.

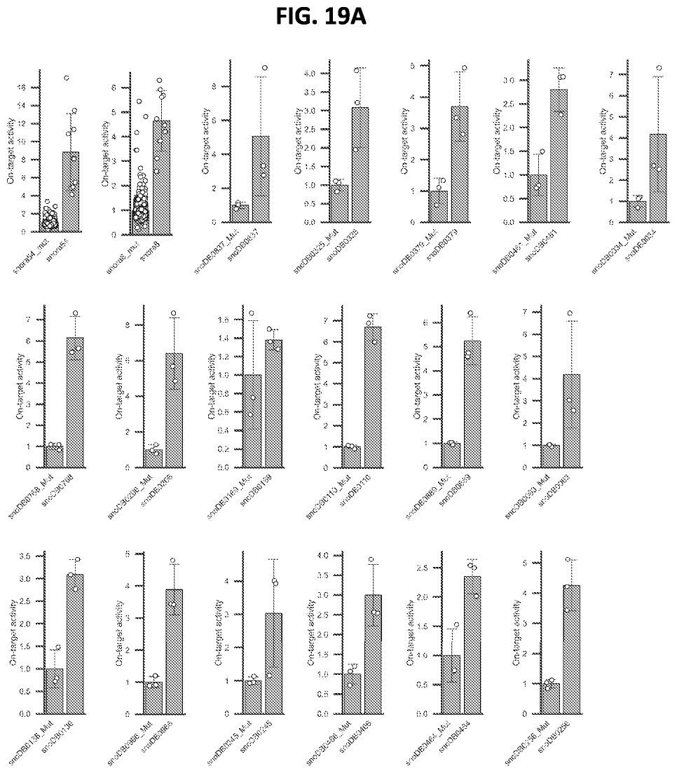

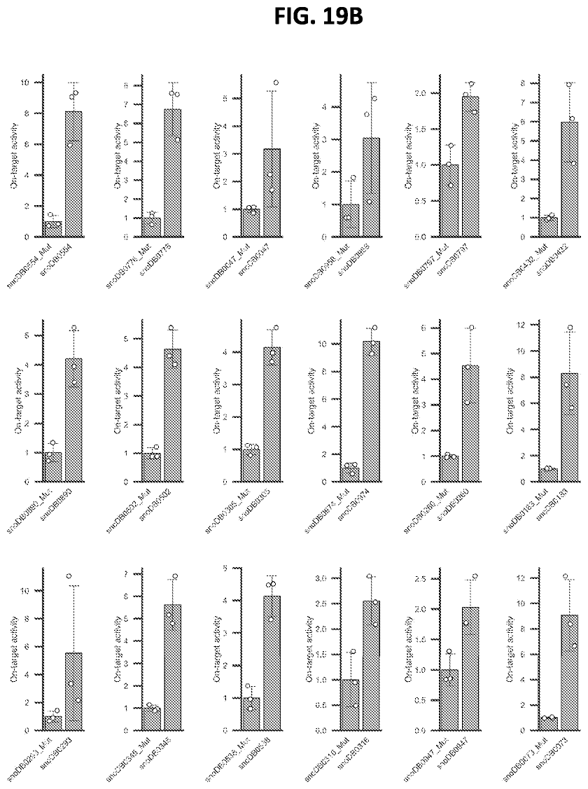

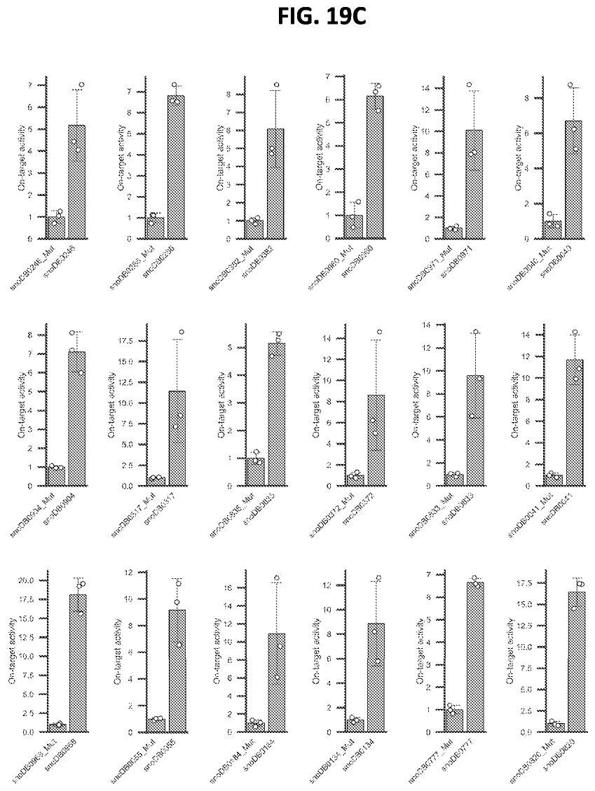

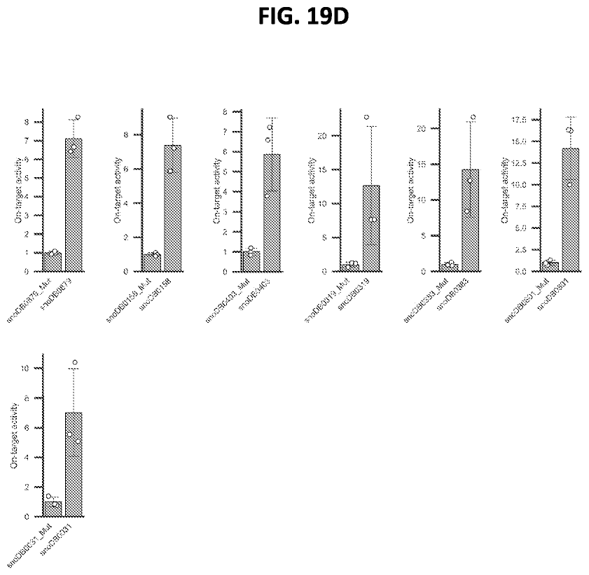

As shown in FIG. 19, a repair RNA (repRNA) modified with snoRNAs depicted in Table 6 (SEQ ID NOs: 732-793) shows significantly higher trans-splicing activity than compared to a repRNA that has a mutated snoRNA sequence that does not form a human RNP (“_Mut” versions of each as shown in FIG. 19). snoRNA sequences which were mutated to no longer form human RNP complexes still showed some degree of trans-splicing, albeit several fold lower in efficiency. HEK293FT cells were transfected with trans-splicing repRNA molecules using LIPOFECTAMINE 2000, per the manufacturer's instructions. RNA was extracted from cells using QIAGEN RNEASY mini extraction kit, per the manufacturer's protocol. RNA-seq libraries were constructed and sequenced on an ILLUMINA platform to determine trans-splicing rates.

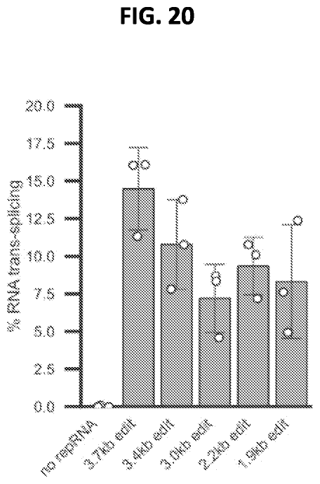

As shown in FIG. 20, repRNAs with SNORA54 (as an illustrative example) were able to efficiently trans-splice independent of the size of the edit. Larger edits did not result in lower efficiency. HEK293FT cells were transfected with trans-splicing repRNA molecules using LIPOFECTAMINE 2000, per the manufacturer's instructions. RNA was extracted from cells using QIAGEN RNEASY mini extraction kit, per the manufacturer's protocol. RNA was sequenced with NANOPORE sequencing to determine trans-splicing efficiency.

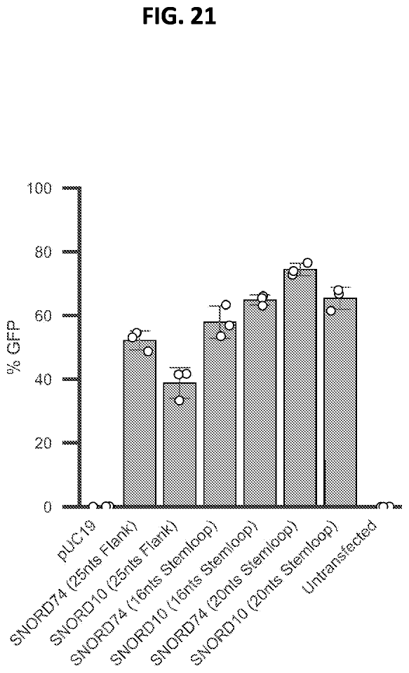

As shown in FIG. 21, repRNAs with C/D-box snoRNA sequences enabled high trans-splicing rates. HEK293FT cells were transfected with trans-splicing repRNA molecules using LIPOFECTAMINE 2000, per the manufacturer's instructions. Cells were analyzed for GFP correction using a BECKMAN COULTER CYTOFLEX system.

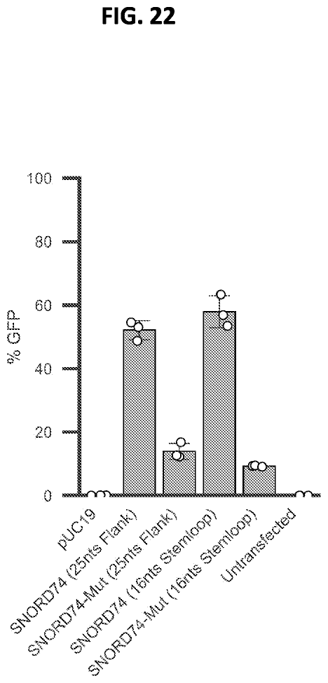

As shown in FIG. 22, repRNAs with C/D-box snoRNA sequences enabled high trans-splicing rates. Mutants that prevented snoRNP formation edit at baseline trans-splicing rates (e.g., “-Mut” forms as shown in FIG. 22) demonstrated that RNP formation dramatically improves trans-splicing rates. HEK293FT cells were transfected with trans-splicing repRNA molecules using lipofectamine 2000, per the manufacturer's instructions. Cells were analyzed for GFP correction using a BECKMAN COULTER CYTOFLEX system.

TABLE 2

| Category | description | element_seq |
|---|---|---|
| SD targeting | Sm_consensus-U7- | (SEQ ID NO: 658) ACACAGGCACTGGCCACTGA-AATTTTTGGAG- |
| | hg_USH2A_int13_SD_20 bp_1- | (SEQ ID NO: 659) taggctttctggcttttcaccggaaagcccct_ |
| | filler | AAATGATTAAATTAA |
| SD targeting | Sm_consensus-U7- | (SEQ ID NO: 660) GATTAGGCACACACAGGCAC-AATTTTTGGAG- |
| | hg_USH2A_int13_SD_20 bp_3- | (SEQ ID NO: 661) taggctttctggcttttcaccggaaagcccct_ |
| | filler | AAATGATTAAATTAA |
| SD targeting | Sm_consensus-U7- | (SEQ ID NO: 662) CTTCCTTGACGATTAGGCAC-AATTTTTGGAG- |
| | hg_USH2A_int13_SD_20 bp_5- | (SEQ ID NO: 663) taggctttctggcttttcaccggaaagcccct_ |
| | filler | AAATGATTAAATTAA |
| SD targeting | Sm_consensus-U7- | (SEQ ID NO: 664) ACCTTCTTCCTTGACGATTA-AATTTTTGGAG- |
| | hg_USH2A_int13_SD_20 bp_6- | (SEQ ID NO: 665) taggctttctggcttttcaccggaaagcccct_ |
| | filler | AAATGATTAAATTAA |
| Random U7 | Sm.rand-U7- | (SEQ ID NO: 666) ACACAGGCACTGGCCACTGA-TGCGTGTCATT- |
| control | hg_USH2A_int13_SD_20 bp_1- | (SEQ ID NO: 667) taggctttctggcttttcaccggaaagcccct_ |
| | filler | AAATGATTAAATTAA |
| Random U7 | Sm.rand-U7- | (SEQ ID NO: 668) GGCACACACAGGCACTGGCC-TGCGTGTCATT- |
| control | hg_USH2A_int13_SD_20 bp_2- | (SEQ ID NO: 669) taggctttctggcttttcaccggaaagcccct_ |
| | filler | AAATGATTAAATTAA |
| Random Sm | Sm_consensus-U7-NT_BM_2- | (SEQ ID NO: 670) ACGAGCTCAGCCTATGCGAG-AATTTTTGGAG- |
| control | filler | (SEQ ID NO: 671) taggctttctggcttttcaccggaaagcccct_ |
| | | AAATGATTAAATTAA |
| Random Sm | Sm_consensus-U7-NT_BM_3- | (SEQ ID NO: 672) CCACCTACCCTATCGTGCGG-AATTTTTGGAG- |
| control | filler | (SEQ ID NO: 673) taggctttctggcttttcaccggaaagcccct_ |
| | | AAATGATTAAATTAA |
| | | Format for the above sequences: |
| | | {Binding Motif}-{Sm consensus/Sm rand}-{U7/U7 |
| | | rand}_{filler} |

TABLE 3

| ASR ID | ASR se′ue′ce (″-″) (SEQ ID NO.) | Length (nt) | Sm sequence (SEQ ID NO.) |
|---|---|---|---|
| smU7 guide 3 | TAAAATATATTTAAAAGGTG (SEQ ID | 20 | AATTTTTGGAG (SEQ ID NO: |
| | NO: 702) | | 701) |
| sm-mut | TAAAATATATTTAAAAGGTG | 20 | AACGCGTCATG (SEQ ID NO: |
| | (SEQ ID NO: 702) | | 703) |
| NT | GTTCCGCGTTACATAACTTA | 20 | AATTTTTGGAG (SEQ ID NO: |
| | (SEQ ID NO: 707) | | 701) |

TABLE 4

| | ASR se′ue′ce (″-″) | |
|---|---|---|
| ASR ID | (SEQ ID NO.) | Length (nt) |
| 7 | TCACACCTAAGCCCTAAAGA | 20 |
| | (SEQ ID NO: 706) | |
| NT | GTTCCGCGTTACATAACTTA | 20 |
| | (SEQ ID NO: 707) | |

TABLE 5
grepRNA sequences:
GGCTTTCTGGCTTTTTACCGGAAAGCC U7 hairpin
AATTTTTGGAG smOPT (SEQ ID NO: 701)
AACGCGTCATG sm-mut (SEQ ID NO: 703)
TCACACCTAAGCCCTAAAGA Anti-sense Region (SEQ ID NO: 706)

| | | |
|---|---|---|
| pATK1014 | TCACACCTAAGCCCTAAAGAGCCTGTACTTGTTAACACCGAATGTGATGCCCTGTAGGCCTTCCTGA | g7 ASR only |
| | CTATGGCGGCCTACTTATCCTGTCCCTTTTTTTTCCACAGGAGCGCACCATCTTCTTCAAGGACGACG | |
| | GCAACTACAAGACCCGCGCCGAGGTGAAGTTCGAGGGCGACACCCTGGTGAACCGCATCGAGCTG | |
| | AAGGGCATCGACTTCAAGGAGGACGGCAACATCCTGGGGCACAAGCTGGAGTACAACTACAACAG | |
| | CCACAACGTCTATATCATGGCCGACAAGCAGAAGAACGGCATCAAGGTGAACTTCAAGATCCGCCA | |
| | CAACATCGAGGACGGCAGCGTGCAGCTCGCCGACCACTACCAGCAGAACACCCCCATCGGCGACG | |
| | GCCCCGTGCTGCTGCCCGACAACCACTACCTGAGCACCCAGTCCGCCCTGAGCAAAGACCCCAACG | |
| | AGAAGCGCGATCACATGGTCCTGCTGGAGTTCGTGACCGCCGCCGGGATCACTCTCGGCATGGACG | |
| | AGCTGTACAAGTAATGGCTTGTTTATTGCAGCTTATAATGGTTACAAATAA (SEQ ID NO: 708) | |
| pATK1016 | TCACACCTAAGCCCTAAAGAAATTTTTGGATAGGCTTTCTGGCTTTTTACCGGAAAGCCCCTGCC | g7 full U7 |
| | TGTACTTGTTAACACCGAATGTGATGCCCTGTAGGCCTTCCTGACTATGGCGGCCTACTTATCCTGTC | gRNA |
| | CCTTTTTTTTCCACAGGAGCGCACCATCTTCTTCAAGGACGACGGCAACTACAAGACCCGCGCCGAG | |
| | GTGAAGTTCGAGGGCGACACCCTGGTGAACCGCATCGAGCTGAAGGGCATCGACTTCAAGGAGGA | |
| | CGGCAACATCCTGGGGCACAAGCTGGAGTACAACTACAACAGCCACAACGTCTATATCATGGCCGA | |
| | CAAGCAGAAGAACGGCATCAAGGTGAACTTCAAGATCCGCCACAACATCGAGGACGGCAGCGTGC | |
| | AGCTCGCCGACCACTACCAGCAGAACACCCCCATCGGCGACGGCCCCGTGCTGCTGCCCGACAACC | |
| | ACTACCTGAGCACCCAGTCCGCCCTGAGCAAAGACCCCAACGAGAAGCGCGATCACATGGTCCTGC | |
| | TGGAGTTCGTGACCGCCGCCGGGATCACTCTCGGCATGGACGAGCTGTACAAGTAATGGCTTGTTT | |
| | ATTGCAGCTTATAATGGTTACAAATAAA (SEQ ID NO: 709) | |
| pATK1019 | GTTCCGCGTTACATAACTTAAATTTTTGGATAGGCTTTCTGGCTTTTTACCGGAAAGCCCCTGCCT | NT full U7 |
| | GTACTTGTTAACACCGAATGTGATGCCCTGTAGGCCTTCCTGACTATGGCGGCCTACTTATCCTGTCC | gRNA |
| | CTTTTTTTTCCACAGGAGCGCACCATCTTCTTCAAGGACGACGGCAACTACAAGACCCGCGCCGAGG | |
| | TGAAGTTCGAGGGCGACACCCTGGTGAACCGCATCGAGCTGAAGGGCATCGACTTCAAGGAGGAC | |
| | GGCAACATCCTGGGGCACAAGCTGGAGTACAACTACAACAGCCACAACGTCTATATCATGGCCGAC | |
| | AAGCAGAAGAACGGCATCAAGGTGAACTTCAAGATCCGCCACAACATCGAGGACGGCAGCGTGCA | |
| | GCTCGCCGACCACTACCAGCAGAACACCCCCATCGGCGACGGCCCCGTGCTGCTGCCCGACAACCA | |
| | CTACCTGAGCACCCAGTCCGCCCTGAGCAAAGACCCCAACGAGAAGCGCGATCACATGGTCCTGCT | |
| | GGAGTTCGTGACCGCCGCCGGGATCACTCTCGGCATGGACGAGCTGTACAAGTAATGGCTTGTTTA | |
| | TTGCAGCTTATAATGGTTACAAATAA (SEQ ID NO: 710) | |
| b_g7_ | GTCGGTCTCGGATCTCACACCTAAGCCCTAAAGAAACGCGTCATGTAGGCTTTCTGGCTTTTTACCG | guide 7 BM |
| smMut_ | GAAAGCCCCTGCCTGTACTTGTTAACACCGAATGTGATGCCCTGTAGGCCTTCCTGACTATGGCGGC | smU7 Mut- |
| 3repRNA | CTACTTATCCTGTCCCTTTTTTTTCCACAGGAGCGCACCATCTTCTTCAAGGACGACGGCAACTACAA | intronic |
| | GACCCGCGCCGAGGTGAAGTTCGAGGGCGACACCCTGGTGAACCGCATCGAGCTGAAGGGCATCG | spacer |
| | ACTTCAAGGAGGACGGCAACATCCTGGGGCACAAGCTGGAGTACAACTACAACAGCCACAACGTCT | (50 nts)- |
| | ATATCATGGCCGACAAGCAGAAGAACGGCATCAAGGTGAACTTCAAGATCCGCCACAACATCGAG | Splice |
| | GACGGCAGCGTGCAGCTCGCCGACCACTACCAGCAGAACACCCCCATCGGCGACGGCCCCGTGCTG | acceptor- |
| | CTGCCCGACAACCACTACCTGAGCACCCAGTCCGCCCTGAGCAAAGACCCCAACGAGAAGCGCGAT | GFP 2.2 |
| | CACATGGTCCTGCTGGAGTTCGTGACCGCCGCCGGGATCACTCTCGGCATGGACGAGCTGTACAAG | |
| | TAATGGCCGAGACC (SEQ ID NO: 711) | |

TABLE 6
SEQUENCE LISTING

| SEQ | | |
|---|---|---|
| ID | Name/ | |
| NO | ID | Sequence |
| 1 | H box | ANANNA N = A, C, G, OR U |
| 2 | ACA box | ACA |
| 3 | Sm | AAUUUUUGG |
| | consensus | |
| | motf | |
| 4 | Sm U7 | AAUUUGUCU |
| | motif | |
| 5 | C box | RUGAUGA R = A OR G |
| 6 | D box | ′UGA |
| 7 | ′′ box | RUG′UGA |
| 8 | ′′ box | CUGA |
| 9 | U1_1_0 | AUACUUACGUAACAGGAGAAAAUACGGCCAUGAAGUUGGUGUUUCUCGGGGGCGAUUUCUCCAUUGUACUCAGUAUG |
| | | UGCUGACUGACUCCUGUUACUUCCACAUGUGGGGAAACUGGACUGUAAUUUGUGGUGGUGGGGAAUUGCGUUCGCGC |
| 10 | U1_1_1 | UACUUACGUAACAGGAGAAAAUACGGCCAUGAAGUUGGUGUUUCUCGGGGGCGAUUUCUCCAUUGUACUCAGUAUGU |
| | | GCUGACUGACUCCUGUUACUUCCACAUGUGGGGAAACUGGACUGUAAUUUGUGGUGGUGGGGAAUUGCGUUCGCGCU |
| 11 | U1_1_2 | ACUUACGUAACAGGAGAAAAUACGGCCAUGAAGUUGGUGUUUCUCGGGGGCGAUUUCUCCAUUGUACUCAGUAUGUG |
| | | CUGACUGACUCCUGUUACUUCCACAUGUGGGGAAACUGGACUGUAAUUUGUGGUGGUGGGGAAUUGCGUUCGCGCUU |
| 12 | U1_1_3 | CUUACGUAACAGGAGAAAAUACGGCCAUGAAGUUGGUGUUUCUCGGGGGCGAUUUCUCCAUUGUACUCAGUAUGUGC |
| | | UGACUGACUCCUGUUACUUCCACAUGUGGGGAAACUGGACUGUAAUUUGUGGUGGUGGGGAAUUGCGUUCGCGCUUU |
| 13 | U1_1_4 | UUACGUAACAGGAGAAAAUACGGCCAUGAAGUUGGUGUUUCUCGGGGGCGAUUUCUCCAUUGUACUCAGUAUGUGCU |
| | | GACUGACUCCUGUUACUUCCACAUGUGGGGAAACUGGACUGUAAUUUGUGGUGGUGGGGAAUUGCGUUCGCGCUUUC |
| 14 | U1_1_5 | UACGUAACAGGAGAAAAUACGGCCAUGAAGUUGGUGUUUCUCGGGGGCGAUUUCUCCAUUGUACUCAGUAUGUGCUG |
| | | ACUGACUCCUGUUACUUCCACAUGUGGGGAAACUGGACUGUAAUUUGUGGUGGUGGGGAAUUGCGUUCGCGCUUUCU |
| 15 | U1_1_6 | ACGUAACAGGAGAAAAUACGGCCAUGAAGUUGGUGUUUCUCGGGGGCGAUUUCUCCAUUGUACUCAGUAUGUGCUGA |
| | | CUGACUCCUGUUACUUCCACAUGUGGGGAAACUGGACUGUAAUUUGUGGUGGUGGGGAAUUGCGUUCGCGCUUUCUU |
| 16 | U1_1_7 | CGUAACAGGAGAAAAUACGGCCAUGAAGUUGGUGUUUCUCGGGGGCGAUUUCUCCAUUGUACUCAGUAUGUGCUGAC |
| | | UGACUCCUGUUACUUCCACAUGUGGGGAAACUGGACUGUAAUUUGUGGUGGUGGGGAAUUGCGUUCGCGCUUUCUUC |
| 17 | U1_1_8 | GUAACAGGAGAAAAUACGGCCAUGAAGUUGGUGUUUCUCGGGGGCGAUUUCUCCAUUGUACUCAGUAUGUGCUGACU |
| | | GACUCCUGUUACUUCCACAUGUGGGGAAACUGGACUGUAAUUUGUGGUGGUGGGGAAUUGCGUUCGCGCUUUCUUCU |
| 18 | U1_1_9 | UAACAGGAGAAAAUACGGCCAUGAAGUUGGUGUUUCUCGGGGGCGAUUUCUCCAUUGUACUCAGUAUGUGCUGACUG |
| | | ACUCCUGUUACUUCCACAUGUGGGGAAACUGGACUGUAAUUUGUGGUGGUGGGGAAUUGCGUUCGCGCUUUCUUCU |
| 19 | U1_2_0 | AUAUUUACUUGGCAGGGGAGAUAACGUGACCACGAAGGUGGUUUUCCCAGGGCUGAGGCUUAUUCAUUGUACUCCGG |
| | | AUGUGCUGACCCCUGCGAUUUCCCCAAAUGUGGGAAACUCGACUGCAUAAUUUGUGGUAGUGGGGGGCUGUGUCCGU |
| 20 | U1_2_1 | UAUUUACUUGGCAGGGGAGAUAACGUGACCACGAAGGUGGUUUUCCCAGGGCUGAGGCUUAUUCAUUGUACUCCGGA |
| | | UGUGCUGACCCCUGCGAUUUCCCCAAAUGUGGGAAACUCGACUGCAUAAUUUGUGGUAGUGGGGGGCUGUGUCCGUG |
| 21 | U1_2_2 | AUUUACUUGGCAGGGGAGAUAACGUGACCACGAAGGUGGUUUUCCCAGGGCUGAGGCUUAUUCAUUGUACUCCGGAU |
| | | GUGCUGACCCCUGCGAUUUCCCCAAAUGUGGGAAACUCGACUGCAUAAUUUGUGGUAGUGGGGGGCUGUGUCCGUGC |
| 22 | U1_2_3 | UUUACUUGGCAGGGGAGAUAACGUGACCACGAAGGUGGUUUUCCCAGGGCUGAGGCUUAUUCAUUGUACUCCGGAUG |
| | | UGCUGACCCCUGCGAUUUCCCCAAAUGUGGGAAACUCGACUGCAUAAUUUGUGGUAGUGGGGGGCUGUGUCCGUGCU |
| 23 | U1_2_4 | UUACUUGGCAGGGGAGAUAACGUGACCACGAAGGUGGUUUUCCCAGGGCUGAGGCUUAUUCAUUGUACUCCGGAUGU |
| | | GCUGACCCCUGCGAUUUCCCCAAAUGUGGGAAACUCGACUGCAUAAUUUGUGGUAGUGGGGGGCUGUGUCCGUGCUU |
| 24 | U1_2_5 | UACUUGGCAGGGGAGAUAACGUGACCACGAAGGUGGUUUUCCCAGGGCUGAGGCUUAUUCAUUGUACUCCGGAUGUG |
| | | CUGACCCCUGCGAUUUCCCCAAAUGUGGGAAACUCGACUGCAUAAUUUGUGGUAGUGGGGGGCUGUGUCCGUGCUUU |
| 25 | U1_2_6 | ACUUGGCAGGGGAGAUAACGUGACCACGAAGGUGGUUUUCCCAGGGCUGAGGCUUAUUCAUUGUACUCCGGAUGUGC |
| | | UGACCCCUGCGAUUUCCCCAAAUGUGGGAAACUCGACUGCAUAAUUUGUGGUAGUGGGGGGCUGUGUCCGUGCUUUU |
| 26 | U1_2_7 | CUUGGCAGGGGAGAUAACGUGACCACGAAGGUGGUUUUCCCAGGGCUGAGGCUUAUUCAUUGUACUCCGGAUGUGCU |
| | | GACCCCUGCGAUUUCCCCAAAUGUGGGAAACUCGACUGCAUAAUUUGUGGUAGUGGGGGGCUGUGUCCGUGCUUUUC |
| 27 | U1_2_8 | UUGGCAGGGGAGAUAACGUGACCACGAAGGUGGUUUUCCCAGGGCUGAGGCUUAUUCAUUGUACUCCGGAUGUGCUG |
| | | ACCCCUGCGAUUUCCCCAAAUGUGGGAAACUCGACUGCAUAAUUUGUGGUAGUGGGGGGCUGUGUCCGUGCUUUUCC |
| 28 | U1_2_9 | UGGCAGGGGAGAUAACGUGACCACGAAGGUGGUUUUCCCAGGGCUGAGGCUUAUUCAUUGUACUCCGGAUGUGCUGA |
| | | CCCCUGCGAUUUCCCCAAAUGUGGGAAACUCGACUGCAUAAUUUGUGGUAGUGGGGGGCUGUGUCCGUGCUUUUCCC |
| 29 | U1_3_0 | AUACUUACCUGGCAGGGCAGAUACCAUGAUCUUAAAGGCAGUUUUCCCAGGGCAAGGCUUAUCCAUUCCACUCUGGA |
| | | UCCAUUAUAGGGGCAUGCUGAUCCCUGGAAUUGCCCCAAAUGUGGGAAGCUCUACUGCAAAAUUUUUGGUAGUGAGC |
| 30 | U1_3_1 | UACUUACCUGGCAGGGCAGAUACCAUGAUCUUAAAGGCAGUUUUCCCAGGGCAAGGCUUAUCCAUUCCACUCUGGAU |
| | | CCAUUAUAGGGGCAUGCUGAUCCCUGGAAUUGCCCCAAAUGUGGGAAGCUCUACUGCAAAAUUUUUGGUAGUGAGCG |
| 31 | U1_3_2 | ACUUACCUGGCAGGGCAGAUACCAUGAUCUUAAAGGCAGUUUUCCCAGGGCAAGGCUUAUCCAUUCCACUCUGGAUC |
| | | CAUUAUAGGGGCAUGCUGAUCCCUGGAAUUGCCCCAAAUGUGGGAAGCUCUACUGCAAAAUUUUUGGUAGUGAGCGA |
| 32 | U1_3_3 | CUUACCUGGCAGGGCAGAUACCAUGAUCUUAAAGGCAGUUUUCCCAGGGCAAGGCUUAUCCAUUCCACUCUGGAUCC |
| | | AUUAUAGGGGCAUGCUGAUCCCUGGAAUUGCCCCAAAUGUGGGAAGCUCUACUGCAAAAUUUUUGGUAGUGAGCGAU |
| 33 | U1_3_4 | UUACCUGGCAGGGCAGAUACCAUGAUCUUAAAGGCAGUUUUCCCAGGGCAAGGCUUAUCCAUUCCACUCUGGAUCCA |
| | | UUAUAGGGGCAUGCUGAUCCCUGGAAUUGCCCCAAAUGUGGGAAGCUCUACUGCAAAAUUUUUGGUAGUGAGCGAUG |
| 34 | U1_3_5 | UACCUGGCAGGGCAGAUACCAUGAUCUUAAAGGCAGUUUUCCCAGGGCAAGGCUUAUCCAUUCCACUCUGGAUCCAU |
| | | UAUAGGGGCAUGCUGAUCCCUGGAAUUGCCCCAAAUGUGGGAAGCUCUACUGCAAAAUUUUUGGUAGUGAGCGAUGG |
| 35 | U1_3_6 | ACCUGGCAGGGCAGAUACCAUGAUCUUAAAGGCAGUUUUCCCAGGGCAAGGCUUAUCCAUUCCACUCUGGAUCCAUU |
| | | AUAGGGGCAUGCUGAUCCCUGGAAUUGCCCCAAAUGUGGGAAGCUCUACUGCAAAAUUUUUGGUAGUGAGCGAUGGC |
| 36 | U1_3_7 | CCUGGCAGGGCAGAUACCAUGAUCUUAAAGGCAGUUUUCCCAGGGCAAGGCUUAUCCAUUCCACUCUGGAUCCAUUA |
| | | UAGGGGCAUGCUGAUCCCUGGAAUUGCCCCAAAUGUGGGAAGCUCUACUGCAAAAUUUUUGGUAGUGAGCGAUGGCA |
| 37 | U1_3_8 | CUGGCAGGGCAGAUACCAUGAUCUUAAAGGCAGUUUUCCCAGGGCAAGGCUUAUCCAUUCCACUCUGGAUCCAUUAU |
| | | AGGGGCAUGCUGAUCCCUGGAAUUGCCCCAAAUGUGGGAAGCUCUACUGCAAAAUUUUUGGUAGUGAGCGAUGGCAU |
| 38 | U1_3_9 | UGGCAGGGCAGAUACCAUGAUCUUAAAGGCAGUUUUCCCAGGGCAAGGCUUAUCCAUUCCACUCUGGAUCCAUUAUA |
| | | GGGGCAUGCUGAUCCCUGGAAUUGCCCCAAAUGUGGGAAGCUCUACUGCAAAAUUUUUGGUAGUGAGCGAUGGCAUU |
| 39 | U1_4_0 | AUACUUACCUGGCAGGGGAGAUACCAUGAUCACGAAGGUGGUUUUCCCAGGGCGAGGCUUAUCCAUUGCACUCCGGA |
| | | UGUGCUGACCCCUGCGAUUUCCCCAAAUGUGGGAAACUCGACUGCAUAAUUUGUGGUAGUGGGGGACUGCGUUCGCG |
| 40 | U1_4_1 | UACUUACCUGGCAGGGGAGAUACCAUGAUCACGAAGGUGGUUUUCCCAGGGCGAGGCUUAUCCAUUGCACUCCGGAU |
| | | GUGCUGACCCCUGCGAUUUCCCCAAAUGUGGGAAACUCGACUGCAUAAUUUGUGGUAGUGGGGGACUGCGUUCGCGC |
| 41 | U1_4_2 | ACUUACCUGGCAGGGGAGAUACCAUGAUCACGAAGGUGGUUUUCCCAGGGCGAGGCUUAUCCAUUGCACUCCGGAUG |
| | | UGCUGACCCCUGCGAUUUCCCCAAAUGUGGGAAACUCGACUGCAUAAUUUGUGGUAGUGGGGGACUGCGUUCGCGCU |
| 42 | U1_4_3 | CUUACCUGGCAGGGGAGAUACCAUGAUCACGAAGGUGGUUUUCCCAGGGCGAGGCUUAUCCAUUGCACUCCGGAUGU |
| | | GCUGACCCCUGCGAUUUCCCCAAAUGUGGGAAACUCGACUGCAUAAUUUGUGGUAGUGGGGGACUGCGUUCGCGCUU |
| 43 | U1_4_4 | UUACCUGGCAGGGGAGAUACCAUGAUCACGAAGGUGGUUUUCCCAGGGCGAGGCUUAUCCAUUGCACUCCGGAUGUG |
| | | CUGACCCCUGCGAUUUCCCCAAAUGUGGGAAACUCGACUGCAUAAUUUGUGGUAGUGGGGGACUGCGUUCGCGCUUU |
| 44 | U1_4_5 | UACCUGGCAGGGGAGAUACCAUGAUCACGAAGGUGGUUUUCCCAGGGCGAGGCUUAUCCAUUGCACUCCGGAUGUGC |
| | | UGACCCCUGCGAUUUCCCCAAAUGUGGGAAACUCGACUGCAUAAUUUGUGGUAGUGGGGGACUGCGUUCGCGCUUUC |
| 45 | U1_4_6 | ACCUGGCAGGGGAGAUACCAUGAUCACGAAGGUGGUUUUCCCAGGGCGAGGCUUAUCCAUUGCACUCCGGAUGUGCU |
| | | GACCCCUGCGAUUUCCCCAAAUGUGGGAAACUCGACUGCAUAAUUUGUGGUAGUGGGGGACUGCGUUCGCGCUUUCC |
| 46 | U1_4_7 | CCUGGCAGGGGAGAUACCAUGAUCACGAAGGUGGUUUUCCCAGGGCGAGGCUUAUCCAUUGCACUCCGGAUGUGCUG |
| | | ACCCCUGCGAUUUCCCCAAAUGUGGGAAACUCGACUGCAUAAUUUGUGGUAGUGGGGGACUGCGUUCGCGCUUUCCC |
| 47 | U1_4_8 | CUGGCAGGGGAGAUACCAUGAUCACGAAGGUGGUUUUCCCAGGGCGAGGCUUAUCCAUUGCACUCCGGAUGUGCUGA |
| | | CCCCUGCGAUUUCCCCAAAUGUGGGAAACUCGACUGCAUAAUUUGUGGUAGUGGGGGACUGCGUUCGCGCUUUCCCC |
| 48 | U1_4_9 | UGGCAGGGGAGAUACCAUGAUCACGAAGGUGGUUUUCCCAGGGCGAGGCUUAUCCAUUGCACUCCGGAUGUGCUGAC |
| | | CCCUGCGAUUUCCCCAAAUGUGGGAAACUCGACUGCAUAAUUUGUGGUAGUGGGGGACUGCGUUCGCGCUUUCCCCU |
| 49 | U1_5_0 | AUACUUCCCUGACAGGGGAGAUACCUAGGAAUCCAACUUCCCAGGGCAAGACUGAUCUAUUGCACUAUGGAUGUGCC |
| | | GACCCCUGAGAUUUACAAAAUUGUGGGAAACUCAACUGCAUAAUUUAUGGAAAUGAAGGACUGUGUUUGCGCUUUCA |
| 50 | U1_5_1 | UACUUCCCUGACAGGGGAGAUACCUAGGAAUCCAACUUCCCAGGGCAAGACUGAUCUAUUGCACUAUGGAUGUGCCG |
| | | ACCCCUGAGAUUUACAAAAUUGUGGGAAACUCAACUGCAUAAUUUAUGGAAAUGAAGGACUGUGUUUGCGCUUUCA |
| 51 | U1_5_2 | ACUUCCCUGACAGGGGAGAUACCUAGGAAUCCAACUUCCCAGGGCAAGACUGAUCUAUUGCACUAUGGAUGUGCCGA |
| | | CCCCUGAGAUUUACAAAAUUGUGGGAAACUCAACUGCAUAAUUUAUGGAAAUGAAGGACUGUGUUUGCGCUUUCA |
| 52 | U1_5_3 | CUUCCCUGACAGGGGAGAUACCUAGGAAUCCAACUUCCCAGGGCAAGACUGAUCUAUUGCACUAUGGAUGUGCCGAC |
| | | CCCUGAGAUUUACAAAAUUGUGGGAAACUCAACUGCAUAAUUUAUGGAAAUGAAGGACUGUGUUUGCGCUUUCA |
| 53 | U1_5_4 | UUCCCUGACAGGGGAGAUACCUAGGAAUCCAACUUCCCAGGGCAAGACUGAUCUAUUGCACUAUGGAUGUGCCGACC |
| | | CCUGAGAUUUACAAAAUUGUGGGAAACUCAACUGCAUAAUUUAUGGAAAUGAAGGACUGUGUUUGCGCUUUCA |
| 54 | U1_5_5 | UCCCUGACAGGGGAGAUACCUAGGAAUCCAACUUCCCAGGGCAAGACUGAUCUAUUGCACUAUGGAUGUGCCGACCC |
| | | CUGAGAUUUACAAAAUUGUGGGAAACUCAACUGCAUAAUUUAUGGAAAUGAAGGACUGUGUUUGCGCUUUCA |
| 55 | U1_5_6 | CCCUGACAGGGGAGAUACCUAGGAAUCCAACUUCCCAGGGCAAGACUGAUCUAUUGCACUAUGGAUGUGCCGACCCC |
| | | UGAGAUUUACAAAAUUGUGGGAAACUCAACUGCAUAAUUUAUGGAAAUGAAGGACUGUGUUUGCGCUUUCA |
| 56 | U1_5_7 | CCUGACAGGGGAGAUACCUAGGAAUCCAACUUCCCAGGGCAAGACUGAUCUAUUGCACUAUGGAUGUGCCGACCCCU |
| | | GAGAUUUACAAAAUUGUGGGAAACUCAACUGCAUAAUUUAUGGAAAUGAAGGACUGUGUUUGCGCUUUCA |
| 57 | U1_5_8 | CUGACAGGGGAGAUACCUAGGAAUCCAACUUCCCAGGGCAAGACUGAUCUAUUGCACUAUGGAUGUGCCGACCCCUG |
| | | AGAUUUACAAAAUUGUGGGAAACUCAACUGCAUAAUUUAUGGAAAUGAAGGACUGUGUUUGCGCUUUCA |
| 58 | U1_5_9 | UGACAGGGGAGAUACCUAGGAAUCCAACUUCCCAGGGCAAGACUGAUCUAUUGCACUAUGGAUGUGCCGACCCCUGA |
| | | GAUUUACAAAAUUGUGGGAAACUCAACUGCAUAAUUUAUGGAAAUGAAGGACUGUGUUUGCGCUUUCA |
| 59 | U1_6_0 | AAACUUCUCUGCCAGGGGAGAUACCAUGAUCACAAAGGUGGCUUUCUCAGGGAAAGGCUGAUUUAUUGCACUCCAGA |
| | | UGUGCUGACCCCUGAGAUUUCUCCAAAUGUGGGAAACUCAGCUGCAUAAUUUGUGGAAGCGAAGGACUGUGUUUGUG |
| 60 | U1_6_1 | AACUUCUCUGCCAGGGGAGAUACCAUGAUCACAAAGGUGGCUUUCUCAGGGAAAGGCUGAUUUAUUGCACUCCAGAU |
| | | GUGCUGACCCCUGAGAUUUCUCCAAAUGUGGGAAACUCAGCUGCAUAAUUUGUGGAAGCGAAGGACUGUGUUUGUGC |
| 61 | U1_6_2 | ACUUCUCUGCCAGGGGAGAUACCAUGAUCACAAAGGUGGCUUUCUCAGGGAAAGGCUGAUUUAUUGCACUCCAGAUG |
| | | UGCUGACCCCUGAGAUUUCUCCAAAUGUGGGAAACUCAGCUGCAUAAUUUGUGGAAGCGAAGGACUGUGUUUGUGCU |
| 62 | U1_6_3 | CUUCUCUGCCAGGGGAGAUACCAUGAUCACAAAGGUGGCUUUCUCAGGGAAAGGCUGAUUUAUUGCACUCCAGAUGU |
| | | GCUGACCCCUGAGAUUUCUCCAAAUGUGGGAAACUCAGCUGCAUAAUUUGUGGAAGCGAAGGACUGUGUUUGUGCUU |
| 63 | U1_6_4 | UUCUCUGCCAGGGGAGAUACCAUGAUCACAAAGGUGGCUUUCUCAGGGAAAGGCUGAUUUAUUGCACUCCAGAUGUG |
| | | CUGACCCCUGAGAUUUCUCCAAAUGUGGGAAACUCAGCUGCAUAAUUUGUGGAAGCGAAGGACUGUGUUUGUGCUUU |
| 64 | U1_6_5 | UCUCUGCCAGGGGAGAUACCAUGAUCACAAAGGUGGCUUUCUCAGGGAAAGGCUGAUUUAUUGCACUCCAGAUGUGC |
| | | UGACCCCUGAGAUUUCUCCAAAUGUGGGAAACUCAGCUGCAUAAUUUGUGGAAGCGAAGGACUGUGUUUGUGCUUUC |
| 65 | U1_6_6 | CUCUGCCAGGGGAGAUACCAUGAUCACAAAGGUGGCUUUCUCAGGGAAAGGCUGAUUUAUUGCACUCCAGAUGUGCU |
| | | GACCCCUGAGAUUUCUCCAAAUGUGGGAAACUCAGCUGCAUAAUUUGUGGAAGCGAAGGACUGUGUUUGUGCUUUCA |
| 66 | U1_6_7 | UCUGCCAGGGGAGAUACCAUGAUCACAAAGGUGGCUUUCUCAGGGAAAGGCUGAUUUAUUGCACUCCAGAUGUGCUG |
| | | ACCCCUGAGAUUUCUCCAAAUGUGGGAAACUCAGCUGCAUAAUUUGUGGAAGCGAAGGACUGUGUUUGUGCUUUCAU |
| 67 | U1_6_8 | CUGCCAGGGGAGAUACCAUGAUCACAAAGGUGGCUUUCUCAGGGAAAGGCUGAUUUAUUGCACUCCAGAUGUGCUGA |
| | | CCCCUGAGAUUUCUCCAAAUGUGGGAAACUCAGCUGCAUAAUUUGUGGAAGCGAAGGACUGUGUUUGUGCUUUCAUG |
| 68 | U1_6_9 | UGCCAGGGGAGAUACCAUGAUCACAAAGGUGGCUUUCUCAGGGAAAGGCUGAUUUAUUGCACUCCAGAUGUGCUGAC |
| | | CCCUGAGAUUUCUCCAAAUGUGGGAAACUCAGCUGCAUAAUUUGUGGAAGCGAAGGACUGUGUUUGUGCUUUCAUGU |
| 69 | U1_7_0 | AUACUUCUCUGCCAGGGGAGAUACUGUGAUCAUGAAGGUGGCUUUCCCAGGGCAAGGCUGAUCUAUUGCAUUCUGGA |
| | | UGUGCUGACUCCUACGAUUUCCCCAAGUGUGGGAAACUCAACUGCAUAAUUUGUGGAAGUAAUGACUGUGUUUGCAC |
| 70 | U1_7_1 | UACUUCUCUGCCAGGGGAGAUACUGUGAUCAUGAAGGUGGCUUUCCCAGGGCAAGGCUGAUCUAUUGCAUUCUGGAU |
| | | GUGCUGACUCCUACGAUUUCCCCAAGUGUGGGAAACUCAACUGCAUAAUUUGUGGAAGUAAUGACUGUGUUUGCACU |
| 71 | U1_7_2 | ACUUCUCUGCCAGGGGAGAUACUGUGAUCAUGAAGGUGGCUUUCCCAGGGCAAGGCUGAUCUAUUGCAUUCUGGAUG |
| | | UGCUGACUCCUACGAUUUCCCCAAGUGUGGGAAACUCAACUGCAUAAUUUGUGGAAGUAAUGACUGUGUUUGCACUU |
| 72 | U1_7_3 | CUUCUCUGCCAGGGGAGAUACUGUGAUCAUGAAGGUGGCUUUCCCAGGGCAAGGCUGAUCUAUUGCAUUCUGGAUGU |
| | | GCUGACUCCUACGAUUUCCCCAAGUGUGGGAAACUCAACUGCAUAAUUUGUGGAAGUAAUGACUGUGUUUGCACUUU |
| 73 | U1_7_4 | UUCUCUGCCAGGGGAGAUACUGUGAUCAUGAAGGUGGCUUUCCCAGGGCAAGGCUGAUCUAUUGCAUUCUGGAUGUG |
| | | CUGACUCCUACGAUUUCCCCAAGUGUGGGAAACUCAACUGCAUAAUUUGUGGAAGUAAUGACUGUGUUUGCACUUUC |
| 74 | U1_7_5 | UCUCUGCCAGGGGAGAUACUGUGAUCAUGAAGGUGGCUUUCCCAGGGCAAGGCUGAUCUAUUGCAUUCUGGAUGUGC |
| | | UGACUCCUACGAUUUCCCCAAGUGUGGGAAACUCAACUGCAUAAUUUGUGGAAGUAAUGACUGUGUUUGCACUUUCA |
| 75 | U1_7_6 | CUCUGCCAGGGGAGAUACUGUGAUCAUGAAGGUGGCUUUCCCAGGGCAAGGCUGAUCUAUUGCAUUCUGGAUGUGCU |
| | | GACUCCUACGAUUUCCCCAAGUGUGGGAAACUCAACUGCAUAAUUUGUGGAAGUAAUGACUGUGUUUGCACUUUCAC |
| 76 | U1_7_7 | UCUGCCAGGGGAGAUACUGUGAUCAUGAAGGUGGCUUUCCCAGGGCAAGGCUGAUCUAUUGCAUUCUGGAUGUGCUG |
| | | ACUCCUACGAUUUCCCCAAGUGUGGGAAACUCAACUGCAUAAUUUGUGGAAGUAAUGACUGUGUUUGCACUUUCACG |
| 77 | U1_7_8 | CUGCCAGGGGAGAUACUGUGAUCAUGAAGGUGGCUUUCCCAGGGCAAGGCUGAUCUAUUGCAUUCUGGAUGUGCUGA |
| | | CUCCUACGAUUUCCCCAAGUGUGGGAAACUCAACUGCAUAAUUUGUGGAAGUAAUGACUGUGUUUGCACUUUCACGU |
| 78 | U1_7_9 | UGCCAGGGGAGAUACUGUGAUCAUGAAGGUGGCUUUCCCAGGGCAAGGCUGAUCUAUUGCAUUCUGGAUGUGCUGAC |
| | | UCCUACGAUUUCCCCAAGUGUGGGAAACUCAACUGCAUAAUUUGUGGAAGUAAUGACUGUGUUUGCACUUUCACGU |
| 79 | U1_8_0 | AUACUUCCCUGCCAGGGGAGAUACCGUGAUCAUGAAGGUGGCUUUCCCAGGGAAAGGCCGAUAUGUUGCACUCUAGA |
| | | UGUGCUGACCCGUGAGAUUUCCCCAAAUGUGGGACACUCAAAUGCAUAAUUGGUGGAAGUGAAGGACUGUGUUUGUG |
| 80 | U1_8_1 | UACUUCCCUGCCAGGGGAGAUACCGUGAUCAUGAAGGUGGCUUUCCCAGGGAAAGGCCGAUAUGUUGCACUCUAGAU |
| | | GUGCUGACCCGUGAGAUUUCCCCAAAUGUGGGACACUCAAAUGCAUAAUUGGUGGAAGUGAAGGACUGUGUUUGUGC |
| 81 | U1_8_2 | ACUUCCCUGCCAGGGGAGAUACCGUGAUCAUGAAGGUGGCUUUCCCAGGGAAAGGCCGAUAUGUUGCACUCUAGAUG |
| | | UGCUGACCCGUGAGAUUUCCCCAAAUGUGGGACACUCAAAUGCAUAAUUGGUGGAAGUGAAGGACUGUGUUUGUGCU |
| 82 | U1_8_3 | CUUCCCUGCCAGGGGAGAUACCGUGAUCAUGAAGGUGGCUUUCCCAGGGAAAGGCCGAUAUGUUGCACUCUAGAUGU |
| | | GCUGACCCGUGAGAUUUCCCCAAAUGUGGGACACUCAAAUGCAUAAUUGGUGGAAGUGAAGGACUGUGUUUGUGCUU |
| 83 | U1_8_4 | UUCCCUGCCAGGGGAGAUACCGUGAUCAUGAAGGUGGCUUUCCCAGGGAAAGGCCGAUAUGUUGCACUCUAGAUGUG |
| | | CUGACCCGUGAGAUUUCCCCAAAUGUGGGACACUCAAAUGCAUAAUUGGUGGAAGUGAAGGACUGUGUUUGUGCUUU |
| 84 | U1_8_5 | UCCCUGCCAGGGGAGAUACCGUGAUCAUGAAGGUGGCUUUCCCAGGGAAAGGCCGAUAUGUUGCACUCUAGAUGUGC |
| | | UGACCCGUGAGAUUUCCCCAAAUGUGGGACACUCAAAUGCAUAAUUGGUGGAAGUGAAGGACUGUGUUUGUGCUUUC |
| 85 | U1_8_6 | CCCUGCCAGGGGAGAUACCGUGAUCAUGAAGGUGGCUUUCCCAGGGAAAGGCCGAUAUGUUGCACUCUAGAUGUGCU |
| | | GACCCGUGAGAUUUCCCCAAAUGUGGGACACUCAAAUGCAUAAUUGGUGGAAGUGAAGGACUGUGUUUGUGCUUUCA |
| 86 | U1_8_7 | CCUGCCAGGGGAGAUACCGUGAUCAUGAAGGUGGCUUUCCCAGGGAAAGGCCGAUAUGUUGCACUCUAGAUGUGCUG |
| | | ACCCGUGAGAUUUCCCCAAAUGUGGGACACUCAAAUGCAUAAUUGGUGGAAGUGAAGGACUGUGUUUGUGCUUUCA |
| 87 | U1_8_8 | CUGCCAGGGGAGAUACCGUGAUCAUGAAGGUGGCUUUCCCAGGGAAAGGCCGAUAUGUUGCACUCUAGAUGUGCUGA |
| | | CCCGUGAGAUUUCCCCAAAUGUGGGACACUCAAAUGCAUAAUUGGUGGAAGUGAAGGACUGUGUUUGUGCUUUCA |
| 88 | U1_8_9 | UGCCAGGGGAGAUACCGUGAUCAUGAAGGUGGCUUUCCCAGGGAAAGGCCGAUAUGUUGCACUCUAGAUGUGCUGAC |
| | | CCGUGAGAUUUCCCCAAAUGUGGGACACUCAAAUGCAUAAUUGGUGGAAGUGAAGGACUGUGUUUGUGCUUUCA |
| 89 | U1_9_0 | UUAACUACCUGACAGAGGAGAUACUGUGAUCAUGAAAGUGGUUUUCCUAGGGCAAGACUUAUCCGUUGCACUCCAGA |
| | | UGUGCUGACUCAUGCAAUUUCCCCAAAUGUGGGAAACUCGACUACAUAAUUUCUGGUGGUAGGGGACUGCGUUCAUG |
| 90 | U1_9_1 | UAACUACCUGACAGAGGAGAUACUGUGAUCAUGAAAGUGGUUUUCCUAGGGCAAGACUUAUCCGUUGCACUCCAGAU |
| | | GUGCUGACUCAUGCAAUUUCCCCAAAUGUGGGAAACUCGACUACAUAAUUUCUGGUGGUAGGGGACUGCGUUCAUGU |
| 91 | U1_9_2 | AACUACCUGACAGAGGAGAUACUGUGAUCAUGAAAGUGGUUUUCCUAGGGCAAGACUUAUCCGUUGCACUCCAGAUG |
| | | UGCUGACUCAUGCAAUUUCCCCAAAUGUGGGAAACUCGACUACAUAAUUUCUGGUGGUAGGGGACUGCGUUCAUGUU |
| 92 | U1_9_3 | ACUACCUGACAGAGGAGAUACUGUGAUCAUGAAAGUGGUUUUCCUAGGGCAAGACUUAUCCGUUGCACUCCAGAUGU |
| | | GCUGACUCAUGCAAUUUCCCCAAAUGUGGGAAACUCGACUACAUAAUUUCUGGUGGUAGGGGACUGCGUUCAUGUUC |
| 93 | U1_9_4 | CUACCUGACAGAGGAGAUACUGUGAUCAUGAAAGUGGUUUUCCUAGGGCAAGACUUAUCCGUUGCACUCCAGAUGUG |
| | | CUGACUCAUGCAAUUUCCCCAAAUGUGGGAAACUCGACUACAUAAUUUCUGGUGGUAGGGGACUGCGUUCAUGUUCU |
| 94 | U1_9_5 | UACCUGACAGAGGAGAUACUGUGAUCAUGAAAGUGGUUUUCCUAGGGCAAGACUUAUCCGUUGCACUCCAGAUGUGC |
| | | UGACUCAUGCAAUUUCCCCAAAUGUGGGAAACUCGACUACAUAAUUUCUGGUGGUAGGGGACUGCGUUCAUGUUCUC |
| 95 | U1_9_6 | ACCUGACAGAGGAGAUACUGUGAUCAUGAAAGUGGUUUUCCUAGGGCAAGACUUAUCCGUUGCACUCCAGAUGUGCU |
| | | GACUCAUGCAAUUUCCCCAAAUGUGGGAAACUCGACUACAUAAUUUCUGGUGGUAGGGGACUGCGUUCAUGUUCUCC |
| 96 | U1_9_7 | CCUGACAGAGGAGAUACUGUGAUCAUGAAAGUGGUUUUCCUAGGGCAAGACUUAUCCGUUGCACUCCAGAUGUGCUG |
| | | ACUCAUGCAAUUUCCCCAAAUGUGGGAAACUCGACUACAUAAUUUCUGGUGGUAGGGGACUGCGUUCAUGUUCUCCC |
| 97 | U1_9_8 | CUGACAGAGGAGAUACUGUGAUCAUGAAAGUGGUUUUCCUAGGGCAAGACUUAUCCGUUGCACUCCAGAUGUGCUGA |
| | | CUCAUGCAAUUUCCCCAAAUGUGGGAAACUCGACUACAUAAUUUCUGGUGGUAGGGGACUGCGUUCAUGUUCUCCCC |
| 98 | U1_9_9 | UGACAGAGGAGAUACUGUGAUCAUGAAAGUGGUUUUCCUAGGGCAAGACUUAUCCGUUGCACUCCAGAUGUGCUGAC |
| | | UCAUGCAAUUUCCCCAAAUGUGGGAAACUCGACUACAUAAUUUCUGGUGGUAGGGGACUGCGUUCAUGUUCUCCCCU |
| 99 | U1_10_0 | AGACUUAUCUGGCAGGGGAGACACCAUGAACAUGAGGAUAGUUUUCCCAAGGCAAGUUUCAACCCUUGCACUCUAGA |
| | | UGAUGAGAUUACUUAAUGGGUACAAUGCAUGUGAUUUGGGUAAUGGAUACUGUAGAAACACUGACGUCAC |
| 100 | U1_10_1 | GACUUAUCUGGCAGGGGAGACACCAUGAACAUGAGGAUAGUUUUCCCAAGGCAAGUUUCAACCCUUGCACUCUAGAU |
| | | GAUGAGAUUACUUAAUGGGUACAAUGCAUGUGAUUUGGGUAAUGGAUACUGUAGAAACACUGACGUCAC |
| 101 | U1_10_2 | ACUUAUCUGGCAGGGGAGACACCAUGAACAUGAGGAUAGUUUUCCCAAGGCAAGUUUCAACCCUUGCACUCUAGAUG |
| | | AUGAGAUUACUUAAUGGGUACAAUGCAUGUGAUUUGGGUAAUGGAUACUGUAGAAACACUGACGUCAC |
| 102 | U1_10_3 | CUUAUCUGGCAGGGGAGACACCAUGAACAUGAGGAUAGUUUUCCCAAGGCAAGUUUCAACCCUUGCACUCUAGAUGA |
| | | UGAGAUUACUUAAUGGGUACAAUGCAUGUGAUUUGGGUAAUGGAUACUGUAGAAACACUGACGUCAC |
| 103 | U1_10_4 | UUAUCUGGCAGGGGAGACACCAUGAACAUGAGGAUAGUUUUCCCAAGGCAAGUUUCAACCCUUGCACUCUAGAUGAU |
| | | GAGAUUACUUAAUGGGUACAAUGCAUGUGAUUUGGGUAAUGGAUACUGUAGAAACACUGACGUCAC |
| 104 | U1_10_5 | UAUCUGGCAGGGGAGACACCAUGAACAUGAGGAUAGUUUUCCCAAGGCAAGUUUCAACCCUUGCACUCUAGAUGAUG |
| | | AGAUUACUUAAUGGGUACAAUGCAUGUGAUUUGGGUAAUGGAUACUGUAGAAACACUGACGUCAC |
| 105 | U1_10_6 | AUCUGGCAGGGGAGACACCAUGAACAUGAGGAUAGUUUUCCCAAGGCAAGUUUCAACCCUUGCACUCUAGAUGAUGA |
| | | GAUUACUUAAUGGGUACAAUGCAUGUGAUUUGGGUAAUGGAUACUGUAGAAACACUGACGUCAC |
| 106 | U1_10_7 | UCUGGCAGGGGAGACACCAUGAACAUGAGGAUAGUUUUCCCAAGGCAAGUUUCAACCCUUGCACUCUAGAUGAUGAG |
| | | AUUACUUAAUGGGUACAAUGCAUGUGAUUUGGGUAAUGGAUACUGUAGAAACACUGACGUCAC |
| 107 | U1_10_8 | CUGGCAGGGGAGACACCAUGAACAUGAGGAUAGUUUUCCCAAGGCAAGUUUCAACCCUUGCACUCUAGAUGAUGAGA |
| | | UUACUUAAUGGGUACAAUGCAUGUGAUUUGGGUAAUGGAUACUGUAGAAACACUGACGUCAC |
| 108 | U1_10_9 | UGGCAGGGGAGACACCAUGAACAUGAGGAUAGUUUUCCCAAGGCAAGUUUCAACCCUUGCACUCUAGAUGAUGAGAU |
| | | UACUUAAUGGGUACAAUGCAUGUGAUUUGGGUAAUGGAUACUGUAGAAACACUGACGUCAC |
| 109 | U1_11_0 | AGACUCAUCUGGCAGCGGAGAUACCAUGAACAUCACGGUAGUUUUCCCAAAGCAAGUUUCCACCCUUGCACUCCGGA |
| | | UGAUGAGACAUUACUUAAUGGAUACAAUGCAUGUGAUUUGAGUGAUGGAUACUGUGGAAACACCGACUUCAC |
| 110 | U1_11_1 | GACUCAUCUGGCAGCGGAGAUACCAUGAACAUCACGGUAGUUUUCCCAAAGCAAGUUUCCACCCUUGCACUCCGGAU |
| | | GAUGAGACAUUACUUAAUGGAUACAAUGCAUGUGAUUUGAGUGAUGGAUACUGUGGAAACACCGACUUCAC |
| 111 | U1_11_2 | ACUCAUCUGGCAGCGGAGAUACCAUGAACAUCACGGUAGUUUUCCCAAAGCAAGUUUCCACCCUUGCACUCCGGAUG |
| | | AUGAGACAUUACUUAAUGGAUACAAUGCAUGUGAUUUGAGUGAUGGAUACUGUGGAAACACCGACUUCAC |
| 112 | U1_11_3 | CUCAUCUGGCAGCGGAGAUACCAUGAACAUCACGGUAGUUUUCCCAAAGCAAGUUUCCACCCUUGCACUCCGGAUGA |
| | | UGAGACAUUACUUAAUGGAUACAAUGCAUGUGAUUUGAGUGAUGGAUACUGUGGAAACACCGACUUCAC |
| 113 | U1_11_4 | UCAUCUGGCAGCGGAGAUACCAUGAACAUCACGGUAGUUUUCCCAAAGCAAGUUUCCACCCUUGCACUCCGGAUGAU |
| | | GAGACAUUACUUAAUGGAUACAAUGCAUGUGAUUUGAGUGAUGGAUACUGUGGAAACACCGACUUCAC |
| 114 | U1_11_5 | CAUCUGGCAGCGGAGAUACCAUGAACAUCACGGUAGUUUUCCCAAAGCAAGUUUCCACCCUUGCACUCCGGAUGAUG |
| | | AGACAUUACUUAAUGGAUACAAUGCAUGUGAUUUGAGUGAUGGAUACUGUGGAAACACCGACUUCAC |
| 115 | U1_11_6 | AUCUGGCAGCGGAGAUACCAUGAACAUCACGGUAGUUUUCCCAAAGCAAGUUUCCACCCUUGCACUCCGGAUGAUGA |
| | | GACAUUACUUAAUGGAUACAAUGCAUGUGAUUUGAGUGAUGGAUACUGUGGAAACACCGACUUCAC |
| 116 | U1_11_7 | UCUGGCAGCGGAGAUACCAUGAACAUCACGGUAGUUUUCCCAAAGCAAGUUUCCACCCUUGCACUCCGGAUGAUGAG |
| | | ACAUUACUUAAUGGAUACAAUGCAUGUGAUUUGAGUGAUGGAUACUGUGGAAACACCGACUUCAC |
| 117 | U1_11_8 | CUGGCAGCGGAGAUACCAUGAACAUCACGGUAGUUUUCCCAAAGCAAGUUUCCACCCUUGCACUCCGGAUGAUGAGA |
| | | CAUUACUUAAUGGAUACAAUGCAUGUGAUUUGAGUGAUGGAUACUGUGGAAACACCGACUUCAC |
| 118 | U1_11_9 | UGGCAGCGGAGAUACCAUGAACAUCACGGUAGUUUUCCCAAAGCAAGUUUCCACCCUUGCACUCCGGAUGAUGAGAC |
| | | AUUACUUAAUGGAUACAAUGCAUGUGAUUUGAGUGAUGGAUACUGUGGAAACACCGACUUCAC |
| 119 | U1_12_0 | AGACUUGGCAGGGGAGAUAGCAUGAUCACGAAGGUGGUUUUCCCAAGGCAAGAUUUAUUCACUGCACUCUGGAUGUG |
| | | CUGACCCCUACGAUUUCCGCCCAAUGGGGAAGCUUGACUGCUUAGUUUGUUGUGGCAGGGGACUGUGUUCACACUUU |
| 120 | U1_12_1 | GACUUGGCAGGGGAGAUAGCAUGAUCACGAAGGUGGUUUUCCCAAGGCAAGAUUUAUUCACUGCACUCUGGAUGUGC |
| | | UGACCCCUACGAUUUCCGCCCAAUGGGGAAGCUUGACUGCUUAGUUUGUUGUGGCAGGGGACUGUGUUCACACUUUU |
| 121 | U1_12_2 | ACUUGGCAGGGGAGAUAGCAUGAUCACGAAGGUGGUUUUCCCAAGGCAAGAUUUAUUCACUGCACUCUGGAUGUGCU |
| | | GACCCCUACGAUUUCCGCCCAAUGGGGAAGCUUGACUGCUUAGUUUGUUGUGGCAGGGGACUGUGUUCACACUUUUC |
| 122 | U1_12_3 | CUUGGCAGGGGAGAUAGCAUGAUCACGAAGGUGGUUUUCCCAAGGCAAGAUUUAUUCACUGCACUCUGGAUGUGCUG |
| | | ACCCCUACGAUUUCCGCCCAAUGGGGAAGCUUGACUGCUUAGUUUGUUGUGGCAGGGGACUGUGUUCACACUUUUCC |
| 123 | U1_12_4 | UUGGCAGGGGAGAUAGCAUGAUCACGAAGGUGGUUUUCCCAAGGCAAGAUUUAUUCACUGCACUCUGGAUGUGCUGA |
| | | CCCCUACGAUUUCCGCCCAAUGGGGAAGCUUGACUGCUUAGUUUGUUGUGGCAGGGGACUGUGUUCACACUUUUCCC |
| 124 | U1_12_5 | UGGCAGGGGAGAUAGCAUGAUCACGAAGGUGGUUUUCCCAAGGCAAGAUUUAUUCACUGCACUCUGGAUGUGCUGAC |
| | | CCCUACGAUUUCCGCCCAAUGGGGAAGCUUGACUGCUUAGUUUGUUGUGGCAGGGGACUGUGUUCACACUUUUCCCG |
| 125 | U1_12_6 | GGCAGGGGAGAUAGCAUGAUCACGAAGGUGGUUUUCCCAAGGCAAGAUUUAUUCACUGCACUCUGGAUGUGCUGACC |
| | | CCUACGAUUUCCGCCCAAUGGGGAAGCUUGACUGCUUAGUUUGUUGUGGCAGGGGACUGUGUUCACACUUUUCCCGG |
| 126 | U1_12_7 | GCAGGGGAGAUAGCAUGAUCACGAAGGUGGUUUUCCCAAGGCAAGAUUUAUUCACUGCACUCUGGAUGUGCUGACCC |
| | | CUACGAUUUCCGCCCAAUGGGGAAGCUUGACUGCUUAGUUUGUUGUGGCAGGGGACUGUGUUCACACUUUUCCCGG |
| 127 | U1_12_8 | CAGGGGAGAUAGCAUGAUCACGAAGGUGGUUUUCCCAAGGCAAGAUUUAUUCACUGCACUCUGGAUGUGCUGACCCC |
| | | UACGAUUUCCGCCCAAUGGGGAAGCUUGACUGCUUAGUUUGUUGUGGCAGGGGACUGUGUUCACACUUUUCCCGG |
| 128 | U1_12_9 | AGGGGAGAUAGCAUGAUCACGAAGGUGGUUUUCCCAAGGCAAGAUUUAUUCACUGCACUCUGGAUGUGCUGACCCCU |
| | | ACGAUUUCCGCCCAAUGGGGAAGCUUGACUGCUUAGUUUGUUGUGGCAGGGGACUGUGUUCACACUUUUCCCGG |
| 129 | U1_13_0 | AUACUUAACAUGGCAGAGGAGAUAUCAUAAUCACAAAGGUAGUUUUCCCAGGGCAAGCCUUAUCCACUGCAUUCCAG |
| | | AUGUGCUCACCUCUGUGGUUUCCCCAAAUGUGGAAAACUGGACUGCAUAAUUUGUGGUAGCGGGGGACUGCAUUAAU |
| 130 | U1_13_1 | UACUUAACAUGGCAGAGGAGAUAUCAUAAUCACAAAGGUAGUUUUCCCAGGGCAAGCCUUAUCCACUGCAUUCCAGA |
| | | UGUGCUCACCUCUGUGGUUUCCCCAAAUGUGGAAAACUGGACUGCAUAAUUUGUGGUAGCGGGGGACUGCAUUAAUA |
| 131 | U1_13_2 | ACUUAACAUGGCAGAGGAGAUAUCAUAAUCACAAAGGUAGUUUUCCCAGGGCAAGCCUUAUCCACUGCAUUCCAGAU |
| | | GUGCUCACCUCUGUGGUUUCCCCAAAUGUGGAAAACUGGACUGCAUAAUUUGUGGUAGCGGGGGACUGCAUUAAUAC |
| 132 | U1_13_3 | CUUAACAUGGCAGAGGAGAUAUCAUAAUCACAAAGGUAGUUUUCCCAGGGCAAGCCUUAUCCACUGCAUUCCAGAUG |
| | | UGCUCACCUCUGUGGUUUCCCCAAAUGUGGAAAACUGGACUGCAUAAUUUGUGGUAGCGGGGGACUGCAUUAAUACC |
| 133 | U1_13_4 | UUAACAUGGCAGAGGAGAUAUCAUAAUCACAAAGGUAGUUUUCCCAGGGCAAGCCUUAUCCACUGCAUUCCAGAUGU |
| | | GCUCACCUCUGUGGUUUCCCCAAAUGUGGAAAACUGGACUGCAUAAUUUGUGGUAGCGGGGGACUGCAUUAAUACCU |
| 134 | U1_13_5 | UAACAUGGCAGAGGAGAUAUCAUAAUCACAAAGGUAGUUUUCCCAGGGCAAGCCUUAUCCACUGCAUUCCAGAUGUG |
| | | CUCACCUCUGUGGUUUCCCCAAAUGUGGAAAACUGGACUGCAUAAUUUGUGGUAGCGGGGGACUGCAUUAAUACCUU |
| 135 | U1_13_6 | AACAUGGCAGAGGAGAUAUCAUAAUCACAAAGGUAGUUUUCCCAGGGCAAGCCUUAUCCACUGCAUUCCAGAUGUGC |
| | | UCACCUCUGUGGUUUCCCCAAAUGUGGAAAACUGGACUGCAUAAUUUGUGGUAGCGGGGGACUGCAUUAAUACCUUC |
| 136 | U1_13_7 | ACAUGGCAGAGGAGAUAUCAUAAUCACAAAGGUAGUUUUCCCAGGGCAAGCCUUAUCCACUGCAUUCCAGAUGUGCU |
| | | CACCUCUGUGGUUUCCCCAAAUGUGGAAAACUGGACUGCAUAAUUUGUGGUAGCGGGGGACUGCAUUAAUACCUUCC |
| 137 | U1_13_8 | CAUGGCAGAGGAGAUAUCAUAAUCACAAAGGUAGUUUUCCCAGGGCAAGCCUUAUCCACUGCAUUCCAGAUGUGCUC |
| | | ACCUCUGUGGUUUCCCCAAAUGUGGAAAACUGGACUGCAUAAUUUGUGGUAGCGGGGGACUGCAUUAAUACCUUCCU |
| 138 | U1_13_9 | AUGGCAGAGGAGAUAUCAUAAUCACAAAGGUAGUUUUCCCAGGGCAAGCCUUAUCCACUGCAUUCCAGAUGUGCUCA |
| | | CCUCUGUGGUUUCCCCAAAUGUGGAAAACUGGACUGCAUAAUUUGUGGUAGCGGGGGACUGCAUUAAUACCUUCCUC |
| 139 | U11_1_0 | AAAAAGGGCUUCUGCUGUGAGUGGCACACAUAGGGCAUCGUUUGCUCUUGGUGCCAGAAUCAACAUCAAGAGAUUUC |
| | | AGAAGCAUAAUUUUUUGGUACUUGGGCAGCUGGUGAUCAUUGGUCCUGUAGCCCUU |
| 140 | U11_1_1 | AAAAGGGCUUCUGCUGUGAGUGGCACACAUAGGGCAUCGUUUGCUCUUGGUGCCAGAAUCAACAUCAAGAGAUUUCA |
| | | GAAGCAUAAUUUUUUGGUACUUGGGCAGCUGGUGAUCAUUGGUCCUGUAGCCCUU |
| 141 | U11_1_2 | AAAGGGCUUCUGCUGUGAGUGGCACACAUAGGGCAUCGUUUGCUCUUGGUGCCAGAAUCAACAUCAAGAGAUUUCAG |
| | | AAGCAUAAUUUUUUGGUACUUGGGCAGCUGGUGAUCAUUGGUCCUGUAGCCCUU |
| 142 | U11_1_3 | AAGGGCUUCUGCUGUGAGUGGCACACAUAGGGCAUCGUUUGCUCUUGGUGCCAGAAUCAACAUCAAGAGAUUUCAGA |
| | | AGCAUAAUUUUUUGGUACUUGGGCAGCUGGUGAUCAUUGGUCCUGUAGCCCUU |
| 143 | U11_1_4 | AGGGCUUCUGCUGUGAGUGGCACACAUAGGGCAUCGUUUGCUCUUGGUGCCAGAAUCAACAUCAAGAGAUUUCAGAA |
| | | GCAUAAUUUUUUGGUACUUGGGCAGCUGGUGAUCAUUGGUCCUGUAGCCCUU |
| 144 | U11_1_5 | GGGCUUCUGCUGUGAGUGGCACACAUAGGGCAUCGUUUGCUCUUGGUGCCAGAAUCAACAUCAAGAGAUUUCAGAAG |
| | | CAUAAUUUUUUGGUACUUGGGCAGCUGGUGAUCAUUGGUCCUGUAGCCCUU |
| 145 | U11_1_6 | GGCUUCUGCUGUGAGUGGCACACAUAGGGCAUCGUUUGCUCUUGGUGCCAGAAUCAACAUCAAGAGAUUUCAGAAGC |
| | | AUAAUUUUUUGGUACUUGGGCAGCUGGUGAUCAUUGGUCCUGUAGCCCUU |
| 146 | U11_1_7 | GCUUCUGCUGUGAGUGGCACACAUAGGGCAUCGUUUGCUCUUGGUGCCAGAAUCAACAUCAAGAGAUUUCAGAAGCA |
| | | UAAUUUUUUGGUACUUGGGCAGCUGGUGAUCAUUGGUCCUGUAGCCCUU |
| 147 | U11_1_8 | CUUCUGCUGUGAGUGGCACACAUAGGGCAUCGUUUGCUCUUGGUGCCAGAAUCAACAUCAAGAGAUUUCAGAAGCAU |
| | | AAUUUUUUGGUACUUGGGCAGCUGGUGAUCAUUGGUCCUGUAGCCCUU |
| 148 | U11_1_9 | UUCUGCUGUGAGUGGCACACAUAGGGCAUCGUUUGCUCUUGGUGCCAGAAUCAACAUCAAGAGAUUUCAGAAGCAUA |
| | | AUUUUUUGGUACUUGGGCAGCUGGUGAUCAUUGGUCCUGUAGCCCUU |
| 149 | U11_2_0 | AAAAAAAGCUGCUGUUGUGAGUGAUACAUGCAGGGCAACUUGAUUGCUCUUAGUGCAGAAUUGACAUCAAGGAAUUU |
| | | UGGAAGUAUAAUUUUUUGGCAGGUGGAUAGCUGGUUGUAUUAGUCCAUUCUC |
| 150 | U11_2_1 | AAAAAAGCUGCUGUUGUGAGUGAUACAUGCAGGGCAACUUGAUUGCUCUUAGUGCAGAAUUGACAUCAAGGAAUUUU |
| | | GGAAGUAUAAUUUUUUGGCAGGUGGAUAGCUGGUUGUAUUAGUCCAUUCUC |
| 151 | U11_2_2 | AAAAAGCUGCUGUUGUGAGUGAUACAUGCAGGGCAACUUGAUUGCUCUUAGUGCAGAAUUGACAUCAAGGAAUUUUG |
| | | GAAGUAUAAUUUUUUGGCAGGUGGAUAGCUGGUUGUAUUAGUCCAUUCUC |
| 152 | U11_2_3 | AAAAGCUGCUGUUGUGAGUGAUACAUGCAGGGCAACUUGAUUGCUCUUAGUGCAGAAUUGACAUCAAGGAAUUUUGG |
| | | AAGUAUAAUUUUUUGGCAGGUGGAUAGCUGGUUGUAUUAGUCCAUUCUC |
| 153 | U11_2_4 | AAAGCUGCUGUUGUGAGUGAUACAUGCAGGGCAACUUGAUUGCUCUUAGUGCAGAAUUGACAUCAAGGAAUUUUGGA |
| | | AGUAUAAUUUUUUGGCAGGUGGAUAGCUGGUUGUAUUAGUCCAUUCUC |
| 154 | U11_2_5 | AAGCUGCUGUUGUGAGUGAUACAUGCAGGGCAACUUGAUUGCUCUUAGUGCAGAAUUGACAUCAAGGAAUUUUGGAA |
| | | GUAUAAUUUUUUGGCAGGUGGAUAGCUGGUUGUAUUAGUCCAUUCUC |
| 155 | U11_2_6 | AGCUGCUGUUGUGAGUGAUACAUGCAGGGCAACUUGAUUGCUCUUAGUGCAGAAUUGACAUCAAGGAAUUUUGGAAG |
| | | UAUAAUUUUUUGGCAGGUGGAUAGCUGGUUGUAUUAGUCCAUUCUC |
| 156 | U11_2_7 | GCUGCUGUUGUGAGUGAUACAUGCAGGGCAACUUGAUUGCUCUUAGUGCAGAAUUGACAUCAAGGAAUUUUGGAAGU |
| | | AUAAUUUUUUGGCAGGUGGAUAGCUGGUUGUAUUAGUCCAUUCUC |
| 157 | U11_2_8 | CUGCUGUUGUGAGUGAUACAUGCAGGGCAACUUGAUUGCUCUUAGUGCAGAAUUGACAUCAAGGAAUUUUGGAAGUA |
| | | UAAUUUUUUGGCAGGUGGAUAGCUGGUUGUAUUAGUCCAUUCUC |
| 158 | U11_2_9 | UGCUGUUGUGAGUGAUACAUGCAGGGCAACUUGAUUGCUCUUAGUGCAGAAUUGACAUCAAGGAAUUUUGGAAGUAU |
| | | AAUUUUUUGGCAGGUGGAUAGCUGGUUGUAUUAGUCCAUUCUC |
| 159 | U11_3_0 | AAAAAGGGCUUCUGUCAUGAGUGGCACACAUAGGACAACUCAAUUUCUCUUCAUGCAGAAUAAACAUCAAGAGAUUU |
| | | UGGAAGCGUAAUUUUUGGUAGUUGGGCAGCUGGUGAUCACUGGUGCCAGCACCCUU |
| 160 | U11_3_1 | AAAAGGGCUUCUGUCAUGAGUGGCACACAUAGGACAACUCAAUUUCUCUUCAUGCAGAAUAAACAUCAAGAGAUUUU |
| | | GGAAGCGUAAUUUUUGGUAGUUGGGCAGCUGGUGAUCACUGGUGCCAGCACCCUU |
| 161 | U11_3_2 | AAAGGGCUUCUGUCAUGAGUGGCACACAUAGGACAACUCAAUUUCUCUUCAUGCAGAAUAAACAUCAAGAGAUUUUG |
| | | GAAGCGUAAUUUUUGGUAGUUGGGCAGCUGGUGAUCACUGGUGCCAGCACCCUU |
| 162 | U11_3_3 | AAGGGCUUCUGUCAUGAGUGGCACACAUAGGACAACUCAAUUUCUCUUCAUGCAGAAUAAACAUCAAGAGAUUUUGG |
| | | AAGCGUAAUUUUUGGUAGUUGGGCAGCUGGUGAUCACUGGUGCCAGCACCCUU |
| 163 | U11_3_4 | AGGGCUUCUGUCAUGAGUGGCACACAUAGGACAACUCAAUUUCUCUUCAUGCAGAAUAAACAUCAAGAGAUUUUGGA |
| | | AGCGUAAUUUUUGGUAGUUGGGCAGCUGGUGAUCACUGGUGCCAGCACCCUU |
| 164 | U11_3_5 | GGGCUUCUGUCAUGAGUGGCACACAUAGGACAACUCAAUUUCUCUUCAUGCAGAAUAAACAUCAAGAGAUUUUGGAA |
| | | GCGUAAUUUUUGGUAGUUGGGCAGCUGGUGAUCACUGGUGCCAGCACCCUU |
| 165 | U11_3_6 | GGCUUCUGUCAUGAGUGGCACACAUAGGACAACUCAAUUUCUCUUCAUGCAGAAUAAACAUCAAGAGAUUUUGGAAG |
| | | CGUAAUUUUUGGUAGUUGGGCAGCUGGUGAUCACUGGUGCCAGCACCCUU |
| 166 | U11_3_7 | GCUUCUGUCAUGAGUGGCACACAUAGGACAACUCAAUUUCUCUUCAUGCAGAAUAAACAUCAAGAGAUUUUGGAAGC |
| | | GUAAUUUUUGGUAGUUGGGCAGCUGGUGAUCACUGGUGCCAGCACCCUU |
| 167 | U11_3_8 | CUUCUGUCAUGAGUGGCACACAUAGGACAACUCAAUUUCUCUUCAUGCAGAAUAAACAUCAAGAGAUUUUGGAAGCG |
| | | UAAUUUUUGGUAGUUGGGCAGCUGGUGAUCACUGGUGCCAGCACCCUU |
| 168 | U11_3_9 | UUCUGUCAUGAGUGGCACACAUAGGACAACUCAAUUUCUCUUCAUGCAGAAUAAACAUCAAGAGAUUUUGGAAGCGU |
| | | AAUUUUUGGUAGUUGGGCAGCUGGUGAUCACUGGUGCCAGCACCCUU |
| 169 | U11_4_0 | AAAAAGGGCUUCUGUCAUGAGUGGCCUAUGUAGGGCAACCAGAUUGCUCUUCAUGUGGAAUUGACAUCAAGAGCUUU |
| | | CAGAAGUGUAUUUUUUGGAAGUUGGGCAGCUGGUAAUCAUUGGUCUUGGCAUCCUU |
| 170 | U11_4_1 | AAAAGGGCUUCUGUCAUGAGUGGCCUAUGUAGGGCAACCAGAUUGCUCUUCAUGUGGAAUUGACAUCAAGAGCUUUC |
| | | AGAAGUGUAUUUUUUGGAAGUUGGGCAGCUGGUAAUCAUUGGUCUUGGCAUCCUU |
| 171 | U11_4_2 | AAAGGGCUUCUGUCAUGAGUGGCCUAUGUAGGGCAACCAGAUUGCUCUUCAUGUGGAAUUGACAUCAAGAGCUUUCA |
| | | GAAGUGUAUUUUUUGGAAGUUGGGCAGCUGGUAAUCAUUGGUCUUGGCAUCCUU |
| 172 | U11_4_3 | AAGGGCUUCUGUCAUGAGUGGCCUAUGUAGGGCAACCAGAUUGCUCUUCAUGUGGAAUUGACAUCAAGAGCUUUCAG |
| | | AAGUGUAUUUUUUGGAAGUUGGGCAGCUGGUAAUCAUUGGUCUUGGCAUCCUU |
| 173 | U11_4_4 | AGGGCUUCUGUCAUGAGUGGCCUAUGUAGGGCAACCAGAUUGCUCUUCAUGUGGAAUUGACAUCAAGAGCUUUCAGA |
| | | AGUGUAUUUUUUGGAAGUUGGGCAGCUGGUAAUCAUUGGUCUUGGCAUCCUU |
| 174 | U11_4_5 | GGGCUUCUGUCAUGAGUGGCCUAUGUAGGGCAACCAGAUUGCUCUUCAUGUGGAAUUGACAUCAAGAGCUUUCAGAA |
| | | GUGUAUUUUUUGGAAGUUGGGCAGCUGGUAAUCAUUGGUCUUGGCAUCCUU |
| 175 | U11_4_6 | GGCUUCUGUCAUGAGUGGCCUAUGUAGGGCAACCAGAUUGCUCUUCAUGUGGAAUUGACAUCAAGAGCUUUCAGAAG |
| | | UGUAUUUUUUGGAAGUUGGGCAGCUGGUAAUCAUUGGUCUUGGCAUCCUU |
| 176 | U11_4_7 | GCUUCUGUCAUGAGUGGCCUAUGUAGGGCAACCAGAUUGCUCUUCAUGUGGAAUUGACAUCAAGAGCUUUCAGAAGU |
| | | GUAUUUUUUGGAAGUUGGGCAGCUGGUAAUCAUUGGUCUUGGCAUCCUU |
| 177 | U11_4_8 | CUUCUGUCAUGAGUGGCCUAUGUAGGGCAACCAGAUUGCUCUUCAUGUGGAAUUGACAUCAAGAGCUUUCAGAAGUG |
| | | UAUUUUUUGGAAGUUGGGCAGCUGGUAAUCAUUGGUCUUGGCAUCCUU |
| 178 | U11_4_9 | UUCUGUCAUGAGUGGCCUAUGUAGGGCAACCAGAUUGCUCUUCAUGUGGAAUUGACAUCAAGAGCUUUCAGAAGUGU |
| | | AUUUUUUGGAAGUUGGGCAGCUGGUAAUCAUUGGUCUUGGCAUCCUU |
| 179 | U11_5_0 | AAAGGGGGCUUCUGUCAUGAGUGGCACACAUUGGGCAACUCAACUGCUCUUCAUGAGGAAUCAACAUCAGGAGGUUU |
| | | UGGAAGAAUGAUUUUUUUGGUAGUUGGGCAGCUUGUGAGAAAAAAAUGUUUUCAGGCAAAUCCUC |
| 180 | U11_5_1 | AAGGGGGCUUCUGUCAUGAGUGGCACACAUUGGGCAACUCAACUGCUCUUCAUGAGGAAUCAACAUCAGGAGGUUUU |
| | | GGAAGAAUGAUUUUUUUGGUAGUUGGGCAGCUUGUGAGAAAAAAAUGUUUUCAGGCAAAUCCUC |
| 181 | U11_5_2 | AGGGGGCUUCUGUCAUGAGUGGCACACAUUGGGCAACUCAACUGCUCUUCAUGAGGAAUCAACAUCAGGAGGUUUUG |
| | | GAAGAAUGAUUUUUUUGGUAGUUGGGCAGCUUGUGAGAAAAAAAUGUUUUCAGGCAAAUCCUC |
| 182 | U11_5_3 | GGGGGCUUCUGUCAUGAGUGGCACACAUUGGGCAACUCAACUGCUCUUCAUGAGGAAUCAACAUCAGGAGGUUUUGG |
| | | AAGAAUGAUUUUUUUGGUAGUUGGGCAGCUUGUGAGAAAAAAAUGUUUUCAGGCAAAUCCUC |
| 183 | U11_5_4 | GGGGCUUCUGUCAUGAGUGGCACACAUUGGGCAACUCAACUGCUCUUCAUGAGGAAUCAACAUCAGGAGGUUUUGGA |
| | | AGAAUGAUUUUUUUGGUAGUUGGGCAGCUUGUGAGAAAAAAAUGUUUUCAGGCAAAUCCUC |
| 184 | U11_5_5 | GGGCUUCUGUCAUGAGUGGCACACAUUGGGCAACUCAACUGCUCUUCAUGAGGAAUCAACAUCAGGAGGUUUUGGAA |
| | | GAAUGAUUUUUUUGGUAGUUGGGCAGCUUGUGAGAAAAAAAUGUUUUCAGGCAAAUCCUC |
| 185 | U11_5_6 | GGCUUCUGUCAUGAGUGGCACACAUUGGGCAACUCAACUGCUCUUCAUGAGGAAUCAACAUCAGGAGGUUUUGGAAG |
| | | AAUGAUUUUUUUGGUAGUUGGGCAGCUUGUGAGAAAAAAAUGUUUUCAGGCAAAUCCUC |
| 186 | U11_5_7 | GCUUCUGUCAUGAGUGGCACACAUUGGGCAACUCAACUGCUCUUCAUGAGGAAUCAACAUCAGGAGGUUUUGGAAGA |
| | | AUGAUUUUUUUGGUAGUUGGGCAGCUUGUGAGAAAAAAAUGUUUUCAGGCAAAUCCUC |
| 187 | U11_5_8 | CUUCUGUCAUGAGUGGCACACAUUGGGCAACUCAACUGCUCUUCAUGAGGAAUCAACAUCAGGAGGUUUUGGAAGAA |
| | | UGAUUUUUUUGGUAGUUGGGCAGCUUGUGAGAAAAAAAUGUUUUCAGGCAAAUCCUC |
| 188 | U11_5_9 | UUCUGUCAUGAGUGGCACACAUUGGGCAACUCAACUGCUCUUCAUGAGGAAUCAACAUCAGGAGGUUUUGGAAGAAU |
| | | GAUUUUUUUGGUAGUUGGGCAGCUUGUGAGAAAAAAAUGUUUUCAGGCAAAUCCUC |
| 189 | U11_6_0 | AAAAAGGGCUUCUGUCGUGAGUGGCACACGUAGGGCAACUCGAUUGCUCUGCGUGCGGAAUCGACAUCAAGAGAUUU |
| | | CGGAAGCAUAAUUUUUUGGUAUUUGGGCAGCUGGUGAUCGUUGGUCCCGGCGCCCUU |
| 190 | U11_6_1 | AAAAGGGCUUCUGUCGUGAGUGGCACACGUAGGGCAACUCGAUUGCUCUGCGUGCGGAAUCGACAUCAAGAGAUUUC |
| | | GGAAGCAUAAUUUUUUGGUAUUUGGGCAGCUGGUGAUCGUUGGUCCCGGCGCCCUU |
| 191 | U11_6_2 | AAAGGGCUUCUGUCGUGAGUGGCACACGUAGGGCAACUCGAUUGCUCUGCGUGCGGAAUCGACAUCAAGAGAUUUCG |
| | | GAAGCAUAAUUUUUUGGUAUUUGGGCAGCUGGUGAUCGUUGGUCCCGGCGCCCUU |
| 192 | U11_6_3 | AAGGGCUUCUGUCGUGAGUGGCACACGUAGGGCAACUCGAUUGCUCUGCGUGCGGAAUCGACAUCAAGAGAUUUCGG |
| | | AAGCAUAAUUUUUUGGUAUUUGGGCAGCUGGUGAUCGUUGGUCCCGGCGCCCUU |
| 193 | U11_6_4 | AGGGCUUCUGUCGUGAGUGGCACACGUAGGGCAACUCGAUUGCUCUGCGUGCGGAAUCGACAUCAAGAGAUUUCGGA |
| | | AGCAUAAUUUUUUGGUAUUUGGGCAGCUGGUGAUCGUUGGUCCCGGCGCCCUU |
| 194 | U11_6_5 | GGGCUUCUGUCGUGAGUGGCACACGUAGGGCAACUCGAUUGCUCUGCGUGCGGAAUCGACAUCAAGAGAUUUCGGAA |
| | | GCAUAAUUUUUUGGUAUUUGGGCAGCUGGUGAUCGUUGGUCCCGGCGCCCUU |
| 195 | U11_6_6 | GGCUUCUGUCGUGAGUGGCACACGUAGGGCAACUCGAUUGCUCUGCGUGCGGAAUCGACAUCAAGAGAUUUCGGAAG |
| | | CAUAAUUUUUUGGUAUUUGGGCAGCUGGUGAUCGUUGGUCCCGGCGCCCUU |
| 196 | U11_6_7 | GCUUCUGUCGUGAGUGGCACACGUAGGGCAACUCGAUUGCUCUGCGUGCGGAAUCGACAUCAAGAGAUUUCGGAAGC |
| | | AUAAUUUUUUGGUAUUUGGGCAGCUGGUGAUCGUUGGUCCCGGCGCCCUU |
| 197 | U11_6_8 | CUUCUGUCGUGAGUGGCACACGUAGGGCAACUCGAUUGCUCUGCGUGCGGAAUCGACAUCAAGAGAUUUCGGAAGCA |
| | | UAAUUUUUUGGUAUUUGGGCAGCUGGUGAUCGUUGGUCCCGGCGCCCUU |
| 198 | U11_6_9 | UUCUGUCGUGAGUGGCACACGUAGGGCAACUCGAUUGCUCUGCGUGCGGAAUCGACAUCAAGAGAUUUCGGAAGCAU |
| | | AAUUUUUUGGUAUUUGGGCAGCUGGUGAUCGUUGGUCCCGGCGCCCUU |
| 199 | U11_7_0 | AAAAAGGGCUUCUGUCGUGAGUGGCACACGUAGGGCAACUCGAUUGCUCUGCGUGCGGAAUCGACAUCAAGAGAUUU |
| | | CGGAAGCAUAAUUUUUUGGUAUUUGGGCAGCUGGUGAUCGUUGGUCCCGGCGCC |
| 200 | U11_7_1 | AAAAGGGCUUCUGUCGUGAGUGGCACACGUAGGGCAACUCGAUUGCUCUGCGUGCGGAAUCGACAUCAAGAGAUUUC |
| | | GGAAGCAUAAUUUUUUGGUAUUUGGGCAGCUGGUGAUCGUUGGUCCCGGCGCC |
| 201 | U11_7_2 | AAAGGGCUUCUGUCGUGAGUGGCACACGUAGGGCAACUCGAUUGCUCUGCGUGCGGAAUCGACAUCAAGAGAUUUCG |
| | | GAAGCAUAAUUUUUUGGUAUUUGGGCAGCUGGUGAUCGUUGGUCCCGGCGCC |
| 202 | U11_7_3 | AAGGGCUUCUGUCGUGAGUGGCACACGUAGGGCAACUCGAUUGCUCUGCGUGCGGAAUCGACAUCAAGAGAUUUCGG |
| | | AAGCAUAAUUUUUUGGUAUUUGGGCAGCUGGUGAUCGUUGGUCCCGGCGCC |
| 203 | U11_7_4 | AGGGCUUCUGUCGUGAGUGGCACACGUAGGGCAACUCGAUUGCUCUGCGUGCGGAAUCGACAUCAAGAGAUUUCGGA |
| | | AGCAUAAUUUUUUGGUAUUUGGGCAGCUGGUGAUCGUUGGUCCCGGCGCC |
| 204 | U11_7_5 | GGGCUUCUGUCGUGAGUGGCACACGUAGGGCAACUCGAUUGCUCUGCGUGCGGAAUCGACAUCAAGAGAUUUCGGAA |
| | | GCAUAAUUUUUUGGUAUUUGGGCAGCUGGUGAUCGUUGGUCCCGGCGCC |
| 205 | U11_7_6 | GGCUUCUGUCGUGAGUGGCACACGUAGGGCAACUCGAUUGCUCUGCGUGCGGAAUCGACAUCAAGAGAUUUCGGAAG |
| | | CAUAAUUUUUUGGUAUUUGGGCAGCUGGUGAUCGUUGGUCCCGGCGCC |
| 206 | U11_7_7 | GCUUCUGUCGUGAGUGGCACACGUAGGGCAACUCGAUUGCUCUGCGUGCGGAAUCGACAUCAAGAGAUUUCGGAAGC |
| | | AUAAUUUUUUGGUAUUUGGGCAGCUGGUGAUCGUUGGUCCCGGCGCC |
| 207 | U11_7_8 | CUUCUGUCGUGAGUGGCACACGUAGGGCAACUCGAUUGCUCUGCGUGCGGAAUCGACAUCAAGAGAUUUCGGAAGCA |
| | | UAAUUUUUUGGUAUUUGGGCAGCUGGUGAUCGUUGGUCCCGGCGCC |
| 208 | U11_7_9 | UUCUGUCGUGAGUGGCACACGUAGGGCAACUCGAUUGCUCUGCGUGCGGAAUCGACAUCAAGAGAUUUCGGAAGCAU |
| | | AAUUUUUUGGUAUUUGGGCAGCUGGUGAUCGUUGGUCCCGGCGCC |
| 209 | Sm | AAUUUUUGGAG |
| | consensus | |
| 210 | Sm_1 | AAUUUUUGGAG |
| 211 | Sm_2 | UAUUUUUGGAG |
| 212 | Sm_ | GAUUUUUGGAG |
| 213 | Sm_4 | CAUUUUUGGAG |
| 214 | Sm_5 | AUUUUUUGGAG |
| 215 | Sm_6 | AGUUUUUGGAG |
| 216 | Sm_7 | ACUUUUUGGAG |
| 217 | Sm_8 | AAAUUUUGGAG |
| 218 | Sm_9 | AAGUUUUGGAG |
| 219 | Sm_10 | AACUUUUGGAG |
| 220 | Sm_11 | AAUAUUUGGAG |
| 221 | Sm_12 | AAUGUUUGGAG |
| 222 | Sm_13 | AAUCUUUGGAG |
| 223 | Sm_14 | AAUUAUUGGAG |
| 224 | Sm_15 | AAUUGUUGGAG |
| 225 | Sm_16 | AAUUCUUGGAG |
| 226 | Sm_17 | AAUUUAUGGAG |
| 227 | Sm_18 | AAUUUGUGGAG |
| 228 | Sm_19 | AAUUUCUGGAG |
| 229 | Sm_20 | AAUUUUAGGAG |
| 230 | Sm_21 | AAUUUUGGGAG |
| 231 | Sm_22 | AAUUUUCGGAG |
| 232 | Sm_23 | AAUUUUUAGAG |
| 233 | Sm_24 | AAUUUUUUGAG |
| 234 | Sm_25 | AAUUUUUCGAG |
| 235 | Sm_26 | AAUUUUUGAAG |
| 236 | Sm_27 | AAUUUUUGUAG |
| 237 | Sm_28 | AAUUUUUGCAG |
| 238 | Sm_29 | AAUUUUUGGUG |
| 239 | Sm_30 | AAUUUUUGGGG |
| 240 | Sm_31 | AAUUUUUGGCG |
| 241 | Sm_32 | AAUUUUUGGAA |
| 242 | Sm_33 | AAUUUUUGGAU |
| 243 | Sm_34 | AAUUUUUGGAC |
| 244 | Sm_35 | AAUGUUUUGAG |
| 245 | Sm_36 | AAUUUGUGUAG |
| 246 | Sm_37 | AGUUUUUGGAA |
| 247 | Sm_38 | AUUUUGUGGAG |
| 248 | Sm_39 | AAUGUUUGCAG |
| 249 | Sm_40 | AAUGUUGGGAG |
| 250 | Sm_41 | AAUUGCUGGAG |
| 251 | Sm_42 | AAUUUUAGCAG |
| 252 | Sm_43 | AAGGUUUGGAG |
| 253 | Sm_44 | GAUUUGUGGAG |
| 254 | Sm_45 | ACUUUUUGGAA |
| 255 | Sm_46 | AAAUUAUGGAG |
| 256 | Sm_47 | AAUUUUGCGAG |
| 257 | Sm_48 | AAUUUCUGGAA |
| 258 | Sm_49 | UAUUUUUGGUG |
| 259 | Sm_50 | GAUUUUAGGAG |
| 260 | Sm_51 | AAUCUGUGGAG |
| 261 | Sm_52 | AAAUUUUAGAG |
| 262 | Sm_53 | AAUUUUUCGAC |
| 263 | Sm_54 | AAUUUUUGGGU |
| 264 | Sm_55 | AAUCUUUGGAA |
| 265 | Sm_56 | AAUUAUUGGUG |
| 266 | Sm_57 | AAUUUUAAGAG |
| 267 | Sm_58 | AAUGUUAGGAG |
| 268 | Sm_59 | AAUUUUAGAAG |
| 269 | Sm_60 | AUUUUUUGGCG |
| 270 | Sm_61 | AACUUUUGGUG |
| 271 | Sm_62 | AAUGUUUGGAU |
| 272 | Sm_63 | AAUUUUAGGAU |
| 273 | Sm_64 | AAUUUUUGAUG |
| 274 | Sm_65 | AAUUCUUGGUG |
| 275 | Sm_66 | AAUCCUUGGAG |
| 276 | Sm_67 | AAUUUUUCAAG |
| 277 | Sm_68 | CAUUUUUGCAG |
| 278 | Sm_69 | CAUUUUUCGAG |
| 279 | Sm_70 | AAUUUUGGGCG |
| 280 | Sm_71 | AGUUUUUGCAG |
| 281 | Sm_72 | AAAAUUUGGAG |
| 282 | Sm_73 | AACUCUUGGAG |
| 283 | Sm_74 | AAUUUGUGGAC |
| 284 | Sm_75 | ACUUUUUGGUG |
| 285 | Sm_76 | AUUUUUUGAAG |
| 286 | Sm_77 | AUUUUUUGGAA |
| 287 | Sm_78 | AAGUUAUGGAG |
| 288 | Sm_79 | CAUUUCUGGAG |
| 289 | Sm_80 | AGUUCUUGGAG |
| 290 | Sm_81 | AAUUUUGGAAG |
| 291 | Sm_82 | AGUAUUUGGAG |
| 292 | Sm_83 | AAUUUUUGGGC |
| 293 | Sm_84 | AAUUCUUGUAG |
| 294 | Sm_85 | AAUUUUGGGAA |
| 295 | Sm_86 | UAUUUUUUGAG |
| 296 | Sm_87 | AACUUUUGUAG |
| 297 | Sm_88 | AAUUAUUGCAG |
| 298 | Sm_89 | AAUUUUUGGUA |
| 299 | Sm_90 | AAUUUUCGAAG |
| 300 | Sm_91 | UAUCUUUGGAG |
| 301 | Sm_92 | AAUUUUUCGUG |
| 302 | Sm_93 | AAUCUUUGGCG |
| 303 | Sm_94 | GAUUUAUGGAG |
| 304 | Sm_95 | AAUUUUUCGAA |
| 305 | Sm_96 | CCUUUUUGGAG |
| 306 | Sm_97 | UAUUUUUGAAG |
| 307 | Sm_98 | AAUUUUUGUAA |
| 308 | Sm_99 | AAUUUUGAGAG |
| 309 | Sm_100 | AAGUAUUGGAG |
| 310 | Sm_101 | AAUUUUAGGAC |
| 311 | Sm_102 | AAUUUUUAAAG |
| 312 | Sm_103 | AAUUCUUGGAU |
| 313 | Sm_104 | AAUUUGUGGUG |
| 314 | Sm_105 | AAUUUUCGGGG |
| 315 | Sm_106 | ACUUUUUUGAG |
| 316 | Sm_107 | AAGUUUCGGAG |
| 317 | Sm_108 | ACUUUUGGGAG |
| 318 | Sm_109 | GACUUUUGGAG |
| 319 | Sm_110 | CUUUUUUGGAG |
| 320 | Sm_111 | CAUUGUUGGAG |
| 321 | Sm_112 | AAUAUUUUGAG |
| 322 | Sm_113 | AAGUUUUGGCG |
| 323 | Sm_114 | AAUUUUAGGAA |
| 324 | Sm_115 | AACUUGUGGAG |
| 325 | Sm_116 | AUUUUUUGGGG |
| 326 | Sm_117 | AAUUUUUAGAU |
| 327 | Sm_118 | UAUUUUGGGAG |
| 328 | Sm_119 | AAUUUGUGGGG |
| 329 | Sm_120 | AAUUUUUAGGG |
| 330 | Sm_121 | AAUUUUCGUAG |
| 331 | Sm_122 | AAUUUUCUGAG |
| 332 | Sm_123 | GAUUCUUGGAG |
| 333 | Sm_124 | AAUCUUUGGAU |
| 334 | Sm_125 | AAAUUUUGGCG |
| 335 | Sm_126 | AAUCAUUGGAG |
| 336 | Sm_127 | AACUUUUGGCG |
| 337 | Sm_128 | AAUUUUUGGGA |
| 338 | Sm_129 | AAUGUUUGGUG |
| 339 | Sm_130 | AAUUUAUGCAG |
| 340 | Sm_131 | AAUCUCUGGAG |
| 341 | Sm_132 | AGUUUGUGGAG |
| 342 | Sm_133 | AAGUUUUGGAU |
| 343 | Sm_134 | UAUUUUUGGCG |
| 344 | Sm_135 | AAUUUCUGAAG |
| 345 | Sm_136 | UAUUUCUGGAG |
| 346 | Sm_137 | AAUUUUUGCUG |
| 347 | Sm_138 | AAAUCUUGGAG |
| 348 | Sm_139 | AAAUUUUUGAG |
| 349 | Sm_140 | UAUUUUUGUAG |
| 350 | Sm_141 | GAUUAUUGGAG |
| 351 | Sm_142 | AAGUUCUGGAG |
| 352 | Sm_143 | AAUCUUUGAAG |
| 353 | Sm_144 | AAAUAUUGGAG |
| 354 | Sm_145 | AACUAUUGGAG |
| 355 | Sm_146 | AAUAUAUGGAG |
| 356 | Sm_147 | AUUCUUUGGAG |
| 357 | Sm_148 | GAUUUUUGAAG |
| 358 | Sm_149 | AAGUUUUGGUG |
| 359 | Sm_150 | AAUCUUUGCAG |
| 360 | Sm_151 | ACUUUUUGGCG |
| 361 | Sm_152 | GAUUUUGGGAG |
| 362 | Sm_153 | CAUUUUUGGAA |
| 363 | Sm_154 | AAUUUUUUGAC |
| 364 | Sm_155 | AAAUUUUGCAG |
| 365 | Sm_156 | ACUUUUUGAAG |
| 366 | Sm_157 | AGUCUUUGGAG |
| 367 | Sm_158 | AGUUUAUGGAG |
| 368 | Sm_159 | ACUUCUUGGAG |
| 369 | Sm_160 | AAUUUUUUCAG |
| 370 | Sm_161 | AAUUUUCGGAU |
| 371 | Sm_162 | AAUUUUUGUAU |
| 372 | Sm_163 | AAUGUCUGGAG |
| 373 | Sm_164 | AAUUGUUGGGG |
| 374 | Sm_165 | AAUUUAUGGAC |
| 375 | Sm_166 | AAUUUAUGAAG |
| 376 | Sm_167 | AAUUUAUGGGG |
| 377 | Sm_168 | AAUUUGUCGAG |
| 378 | Sm_169 | AAUUGUUGGAA |
| 379 | Sm_170 | UGUUUUUGGAG |
| 380 | Sm_171 | AAAUUUAGGAG |
| 381 | Sm_172 | AAUUUUUUGUG |
| 382 | Sm_173 | CAUUCUUGGAG |
| 383 | Sm_174 | AAUUCUCGGAG |
| 384 | Sm_175 | AAUUUUUAUAG |
| 385 | Sm_176 | ACUUUUUAGAG |
| 386 | Sm_177 | AAUUGUUAGAG |
| 387 | Sm_178 | CGUUUUUGGAG |
| 388 | Sm_179 | AAUUUUUGCGG |
| 389 | Sm_180 | AAGUUUUGAAG |
| 390 | Sm_181 | AAUUUUCGGAA |
| 391 | Sm_182 | AAUUUUUCCAG |
| 392 | Sm_183 | AUUUUUUGUAG |
| 393 | Sm_184 | AAUCUUUAGAG |
| 394 | Sm_185 | AAUUUUACGAG |
| 395 | Sm_186 | CAUUUUUGGCG |
| 396 | Sm_187 | AAUUUGCGGAG |
| 397 | Sm_188 | CAUCUUUGGAG |
| 398 | Sm_189 | AACUUUUGAAG |
| 399 | Sm_190 | AAUUUUUCUAG |
| 400 | U7_1 | CAGGUUUUCCAGUGUUCACUGAAAUUUGUCUCU |
| 401 | U7_2 | AAGGUUUUCUGGUCUUUAUCAGAAAGCCUCC |
| 402 | U7_3 | CAGGUUUUCUGGUUUUCACUGCAAAACCCCA |
| 403 | U7_4 | CAGACUUUACGGUGCUUACCAGAAAGCUCCC |
| 404 | U7_5 | CACGUUUUCCGGUUGUCACUCCCAGGUAGGCUGGGGAAGAGGCAU |
| 405 | U7_6 | CAGUCUUUCCAGUUUUUGCCGGAAAGCCCCU |
| 406 | U7_7 | CAGGCUUUCUGGUUUUUGCCAGAAAGCCCCC |
| 407 | U7_8 | CAGGUUUUUCAGUUUUCACCAGAAUGCCCAC |
| 408 | U7_9 | CAGGUUUUCUGGUCUUUACUGGAAAGCCCAA |
| 409 | U7_10 | CAGGCUUUCCGGUUUUUACCAGAAAGCCCCC |
| 410 | U7_11 | CAGGUUUUUCGGUUUUUACUGGAAAGCCCCA |
| 411 | U7_12 | CAGGCUUUCCAGUAAAUACAGGAAAGCCCUC |
| 412 | U7_13 | CAGGCUUUCCGGUUUUUGCUGGAAAGACUCC |
| 413 | U7_14 | AAGGUUUUCUGGUCUUUACUGAAAAGCCCCA |
| 414 | U7_15 | UGGGCUUUCUGUUUUUUACGAGAAAGUCCUC |
| 415 | U7_16 | CAGAUUUCUCAGUUUUCACUGGAAACCCUU |
| 416 | U7_17 | CAGGUUUUCCUGUCUUCACCGGAACUCCCCA |
| 417 | U7_18 | CAGGUUUUCUGGUUUGCACUAGAAAAACCAUA |
| 418 | U7_19 | CAGAUUUUCUGGUUUUUACCAGAAAAUUUAA |
| 419 | U7_20 | CAGGUCUUCUUGUUUUUACUGGAAAAUCCUC |
| 420 | U7_21 | CAGGUUUUCCGGUCUUCACCAGAAAACCCCU |
| 421 | U7_22 | CAGGUUUUCCGAUUUUUACUGGAAAGCCCUU |
| 422 | U7_23 | CAGGUUUUCUGGUUUUCCAGAAAACCUCC |
| 423 | U7_24 | CAGGUUUUCCAGUUUUCACUGGAACCUCU |
| 424 | U7_25 | CCGGCUUUCCAGUUUUGCCGGAAAGCCCCC |
| 425 | U7_26 | CAGGUUUUCUGGUUUUUACCAGAACUUAA |
| 426 | U7_27 | CACGUUUUCCGGUUGUCACUCCCAGGUGGGCUCGGGAAGAGGCAU |
| 427 | U7_28 | CAGGCUUUCCGGUCUUUACCGGAAAGCCUAU |
| 428 | U7_29 | CAGGCUUUCUGGUUUUUGCCGGAAAGCCCUC |
| 429 | U7_30 | CAGGCUUUCAGGUCUUUGCCAGAAAGCCCCA |
| 430 | U7_31 | CAGGCUUUCUUGUUUUCACUGGAAGCCUCC |
| 431 | U7_32 | CAGGUUUUUCAGUCUUCACCGGAAAGCUCCC |
| 432 | U7_33 | CAGAUUUUCUGGUUUUCACCACGAAAGAAAUGUCAUU |
| 433 | U7_34 | CAGUUCUCCUGGAUUUUACAGGAAAACCCC |
| 434 | U7_35 | CAGGCUUUCUGGCAUUUGCCAGAAAGCCCUG |
| 435 | U7_36 | CAGACUUUCCUGUUUUUAACAGAAAGCCCCC |
| 436 | U7_37 | CAGGCUUUCCGGUUUUUGCUGGAAAGCUCCC |
| 437 | U7_38 | CAGCUUUUCCAGUUUUCACUGAGGUAAA |
| 438 | U7_39 | UAGGUUUUCUGGUUUUUAUUGGGAAAUCACA |
| 439 | U7_40 | CAGGGUUUCUGGUGGUACCAGCAGACUCCACA |
| 440 | U7_41 | CAAGUUUCCCAGUUUUCACUGGAACCUCCG |
| 441 | U7_42 | CAGAUUUUCCAGUUUUUACUAGAAACCCCCC |
| 442 | U7_43 | CAGGGUUUCCAGUUUUCACUGUAUACCAUCC |
| 443 | U7_44 | CAAGUUCUCUGGUCUUCACUGGAAAACCCAU |
| 444 | U7_45 | UAGAUUUUCUGGUCUUUACCAGAAAGACUCC |
| 445 | U7_46 | GCAGGUUUUCUGGUUUUUAUUGGAAAACCUUC |
| 446 | U7_47 | CAGGUUUUCCACUUUUCACCAGAAACUGCCUCU |
| 447 | U7_48 | UAGGCUUUCCGGUUUUUGCAGGAAAGCCCCC |
| 448 | U7_49 | CAGGUUUUCCAGUCUUUACCAGAAAGCCACU |
| 449 | U7_50 | CAGGUUUUUUGGUCUUCACAGGAAACUUCCCCU |
| 450 | U7_51 | CAGGUUUUCUGCUUUUCACUGGAAGACUCCC |
| 451 | U7_52 | CAGACUUUCCAGUUUUUGCCAGAAAGCCCAC |
| 452 | U7_53 | CAGGCUUUCUGGUUUUUGCCCAAAAGCCUCU |
| 453 | U7_54 | CAGGUUUUCCAAUCUUCACUGAAAAGCUUUA |
| 454 | U7_55 | CUGGUUCCUAGUCUUCACUGGAAGCACCCUC |
| 455 | U7_56 | GGCUUUCCGCUCUUCAUUGGAAAGCCCAU |
| 456 | U7_57 | CAGGUUUUUCAGUUUUUAGCAGAAAACCUCC |
| 457 | U7_58 | CAGGUUCUCUGGUUUUUACUGAAACCAAA |
| 458 | U7_59 | CAGGUUUUCCAGUCUUCACUGGAAAGCCCUU |
| 459 | U7_60 | CAGGCUUUCUGGUUUUUGCCGGAAGGCCUCC |
| 460 | U7_61 | CAGGUUUUCUGUUUUUUAACUAGAAAACUCCC |
| 461 | U7_62 | UAGGCUUUCUGGUUUUUGCUGGAAAGCCCCC |
| 462 | U7_63 | CAGGUUUCCCGGUUUUUACCAGAAAAAUCUAA |
| 463 | U7_64 | CAGGUUUUCCAGUCUUUACCAGAAAUGCCCCC |
| 464 | U7_65 | GGUUUUCUGGUCAUCGCUGGAAACACCCUC |
| 465 | U7_66 | CAACCUUUCUGGUUUUUGCUAGAAAGUUCUC |
| 466 | U7_67 | CAGGCCUUUGGGUCUUUACUGGAAAACCCCU |
| 467 | U7_68 | UAUGUUUUCCGGUUUUCACUCCCAGGUAGGCUCGG |
| 468 | U7_69 | CAAAUUUUCUGGCUUUUCCAGGAAAAUCCCC |
| 469 | U7_70 | UAGGUUUUCCAGUGACCAGAAAAUCCUC |
| 470 | U7_71 | CAGGUCUUCGGGUUUUUAACUGGAAACCUUC |
| 471 | U7_72 | GCAGGCUUUCCAGUUUUUUCUGGAAAGCCUCA |
| 472 | U7_73 | GGCUUUCUGGUUUUUACUGGAAAGCCCCC |
| 473 | U7_74 | CAGGUUUCCCAGUAUUCACCGGAAAGCUCCA |
| 474 | U7_75 | CAGGCUUUCUAGUUUUCCCACACUAAGAAACAAAAAAACCUGU |
| 475 | U7_76 | GCAAGCUUUCUGGUUUUGGCCAAAAAGCCACC |
| 476 | U7_77 | UUCUGGUUUUCACCAGAAACCACU |
| 477 | U7_78 | GCAAGUUUUCUAGCCUGUACCUGAAAGCCUCA |
| 478 | U7_79 | AAGUUUUCUUGUUUUUACCAGAAAACUCCU |
| 479 | U7_80 | GGCUUUCCAAUUUUUGCCAGAAAGCCCCC |
| 480 | U7_81 | AGCUGGUUUUCUGGUUUUCACCGGAAGACCCAU |
| 481 | U7_82 | CAGGCCUUCUGGUUUUUGCUGGGAAGUCCCCA |
| 482 | U7_83 | CAGGUUUUCCAGUUUGUACCAGAAAACCCCU |
| 483 | U7_84 | CAGGUUUCACCCAAAAACCCAC |
| 484 | U7_85 | UAGGCUUUCUGGCUUUUUACCGGAAAGCCCCU |
| 485 | U7_86 | CAGGGUUUCUGGUUUUCACCAGGAAACAAAA |
| 486 | U7_87 | CAGGUUUUCCUGUCUUUACUAGAAACCCCUC |
| 487 | U7_88 | CAGGUUUUAUAGGUUUUACCAGAAAACUUCC |
| 488 | U7_89 | CAGGCUUCCCAAUUUUUGCUGGAAAGCCCCU |
| 489 | U7_90 | CAGGCUUUUUGGGUUAGGUCAGAAAGUCCCC |
| 490 | U7_91 | CAGGUUUUUGGUUUUUUAGCGAAACCCUC |
| 491 | U7_92 | CAGGCUUUCUGGUUUUCACCAUAAAAUGCCCCA |
| 492 | U7_93 | CAGGUCUUCGGGGUUUUACAAAAAGAAGAAGAAAUCCACCCCC |
| 493 | U7_94 | CAAGUUCUCUGGUCUCCCAACAAGAAACCCCC |
| 494 | U7_95 | CAGGAUUUCUGGUUUCUGGUGGAAAGCUCCCAU |
| 495 | U7_96 | AGGCUUUCCAGUUUUUGCUGGAAAGCCCCU |
| 496 | U7_97 | CAGGUUUUCCAGUCUUUGUCAAAUGCCUUC |
| 497 | U7_98 | CAGGCUUUCCAGUUUUUGCCAGUGUGAACCCAAA |
| 498 | U7_99 | CAGGCUUUCUGGUUUUUAUCAGAAAGCCUCC |
| 499 | U7_100 | GAGGUUUUCUGGUUUUCACCAGAACCAACCCUU |
| 500 | U7_101 | CAGAUUUUCCAGCUUUUACUGGAAGCCCCCU |
| 501 | U7_102 | CAGGUUUUCUGGUUUUCACUGGAAAAUACCUCA |
| 502 | U7_103 | CAUGUUUUUUGGUGUUAAUAAAAACCCGCACAC |
| 503 | U7_104 | AAGGCUUUCCGGUUUUUGCAGGAAAGCAACC |
| 504 | U7_105 | AGUCUUUCCGGGUUUUGUCAGAAAGGCCCU |
| 505 | U7_106 | GCAGGUUUUCUGGUUUCUACUGGAAGCUUCU |
| 506 | U7_107 | CAGGCUUUCUGGUUUUUACUGGAAAGCCCCU |
| 507 | U7_108 | CAGGCUUUCCAAUUUUUGCCAGAAAGCUCCC |
| 508 | U7_109 | CAGGUUUUCUGGUUCUCACCAGAAAACGCCA |
| 509 | U7_110 | CAGGUUUUCUGGUCUUCACUGGAAGACCACU |
| 510 | U7_111 | CAGGUGGUUUUCCGGUCUUUACCAGUAAGCCCCC |
| 511 | U7_112 | CAGGUUUUCCAGUUUCUACCAGAAACCCCUA |
| 512 | U7_113 | CAGGUUUUCUGGUCUUUACCAGAAAAGUCUCC |
| 513 | U7_114 | CCAGGCUUUCUGGUUUUUACCGGAAAGCCCCG |
| 514 | U7_115 | CAGGUUUUUCACUUUUUACUAGAAAAACCAGU |
| 515 | U7_116 | UAGGCUUUCUGGGAGGUCUUUACCAGAAAGCCUCC |
| 516 | U7_117 | UACAUUUUCCAGUUUUCACCAGGAAUCUGCCU |
| 517 | U7_118 | UAGGUUUUCCAGUUUUCACCAGAAAAUCCCG |
| 518 | U7_119 | CAGGUUUUCUGGUUUUUACGAGACUCCCCCA |
| 519 | U7_120 | CAGGCUUUCUGUUUUUUGCUAAAAUCCUCC |
| 520 | U7_121 | CUGGGCGUACUGGCUCACGCCUAUAAUCCCAA |
| 521 | U7_122 | GGCUCUCCGGUUUUUGCCAGAAUGCCCAC |
| 522 | U7_123 | UGGGUUUUCUGGUGAAAACUAGACUGCCCCC |
| 523 | U7_124 | CAGGCUUUCUGGUUUUUACAGGAAGGCCCCU |
| 524 | U7_125 | GGUUUUCUGGUUCUCACUGGAAAACCCCC |
| 525 | U7_126 | CAGGCUUUCCGGUAAAGACUGGAAAGCCCCU |
| 526 | U7_127 | CAGGUUUUCCAGUUUUUACCAGAAAACUCUC |
| 527 | U7_110_ | AAGGUUUUCUGGUCUUCACUGGAAGACCACU |
| | C1A | |
| 528 | U7_110_ | GAGGUUUUCUGGUCUUCACUGGAAGACCACU |
| | C1G | |
| 529 | U7_110_ | UAGGUUUUCUGGUCUUCACUGGAAGACCACU |
| | C1T | |
| 530 | U7_110_ | CCGGUUUUCUGGUCUUCACUGGAAGACCACU |
| | A2C | |
| 531 | U7_110_ | CGGGUUUUCUGGUCUUCACUGGAAGACCACU |
| | A2G | |
| 532 | U7_110_ | CUGGUUUUCUGGUCUUCACUGGAAGACCACU |
| | A2T | |
| 533 | U7_110_ | CAUGUUUUCUGGUCUUCACUGGAAGACAACU |
| | G3T_C28A | |
| 534 | U7_110_ | CACGUUUUCUGGUCUUCACUGGAAGACGACU |
| | G3C_C28G | |
| 535 | U7_110_ | CAAGUUUUCUGGUCUUCACUGGAAGACUACU |
| | G3A_C28T | |
| 536 | U7_110_ | CAGCUUUUCUGGUCUUCACUGGAAGAGCACU |
| | G4C_C27G | |
| 537 | U7_110_ | CAGAUUUUCUGGUCUUCACUGGAAGAUCACU |
| | G4A_C27T | |
| 538 | U7_110_ | CAGUUUUUCUGGUCUUCACUGGAAGAACACU |
| | G4T_C27A | |
| 539 | U7_110_ | CAGGGUUUCUGGUCUUCACUGGAAGCCCACU |
| | T5G_A26C | |
| 540 | U7_110_ | CAGGAUUUCUGGUCUUCACUGGAAGUCCACU |
| | T5A_A26T | |
| 541 | U7_110_ | CAGGCUUUCUGGUCUUCACUGGAAGGCCACU |
| | T5C_A26G | |
| 542 | U7_110_ | CAGGUGUUCUGGUCUUCACUGGAACACCACU |
| | T6G_G25C | |
| 543 | U7_110_ | CAGGUAUUCUGGUCUUCACUGGAAUACCACU |
| | T6A_G25T | |
| 544 | U7_110_ | CAGGUCUUCUGGUCUUCACUGGAAGACCACU |
| | T6C_G25G | |
| 545 | U7_110_ | CAGGUUUUCUGGUCUUCACUGGAAAACCACU |
| | T6T_G25A | |
| 546 | U7_110_ | CAGGUUCUCUGGUCUUCACUGGAGGACCACU |
| | T7C_A24G | |
| 547 | U7_110_ | CAGGUUGUCUGGUCUUCACUGGACGACCACU |
| | T7G_A24C | |
| 548 | U7_110_ | CAGGUUAUCUGGUCUUCACUGGAUGACCACU |
| | T7A_A24T | |
| 549 | U7_110_ | CAGGUUUACUGGUCUUCACUGGUAGACCACU |
| | T8A_A23T | |
| 550 | U7_110_ | CAGGUUUGCUGGUCUUCACUGGCAGACCACU |
| | T8G_A23C | |
| 551 | U7_110_ | CAGGUUUCCUGGUCUUCACUGGGAGACCACU |
| | T8C_A23G | |
| 552 | U7_110_ | CAGGUUUUUUGGUCUUCACUGAAAGACCACU |
| | C9T_G22A | |
| 553 | U7_110_ | CAGGUUUUGUGGUCUUCACUGCAAGACCACU |
| | C9G_G22C | |
| 554 | U7_110_ | CAGGUUUUAUGGUCUUCACUGUAAGACCACU |
| | C9A_G22T | |
| 555 | U7_110_ | CAGGUUUUCCGGUCUUCACUGGAAGACCACU |
| | T10C_G21G | |
| 556 | U7_110_ | CAGGUUUUCAGGUCUUCACUUGAAGACCACU |
| | 1T0A_G21T | |
| 557 | U7_110_ | CAGGUUUUCUGGUCUUCACUAGAAGACCACU |
| | T10T_G21A | |
| 558 | U7_110_ | CAGGUUUUCGGGUCUUCACUCGAAGACCACU |
| | T10G_G21C | |
| 559 | U7_110_ | CAGGUUUUCUUGUCUUCACAGGAAGACCACU |
| | G11T_T20A | |
| 560 | U7_110_ | CAGGUUUUCUAGUCUUCACUGGAAGACCACU |
| | G11A_T20T | |
| 561 | U7_110_ | CAGGUUUUCUGGUCUUCACCGGAAGACCACU |
| | G11G_T20C | |
| 562 | U7_110_ | CAGGUUUUCUCGUCUUCACGGGAAGACCACU |
| | G11C_T20G | |
| 563 | U7_110_ | CAGGUUUUCUGUUCUUCAAUGGAAGACCACU |
| | G12T_C19A | |
| 564 | U7_110_ | CAGGUUUUCUGAUCUUCAUUGGAAGACCACU |
| | G12A_C19T | |
| 565 | U7_110_ | CAGGUUUUCUGCUCUUCAGUGGAAGACCACU |
| | G12C_C19G | |
| 566 | U7_110_ | CAGGUUUUCUGGCCUUCGCUGGAAGACCACU |
| | T13C_A18G | |
| 567 | U7_110_ | CAGGUUUUCUGGGCUUCCCUGGAAGACCACU |
| | T13G_A18C | |
| 568 | U7_110_ | CAGGUUUUCUGGACUUCUCUGGAAGACCACU |
| | T13A_A18T | |
| 569 | U7_110_ | CAGGUUUUCUGGUUUUCACUGGAAGACCACU |
| | C14T | |
| 570 | U7_110_ | CAGGUUUUCUGGUAUUCACUGGAAGACCACU |
| | C14A | |
| 571 | U7_110_ | CAGGUUUUCUGGUGUUCACUGGAAGACCACU |
| | C14G | |
| 572 | U7_110_ | CAGGUUUUCUGGUCAUCACUGGAAGACCACU |
| | T15A | |
| 573 | U7_110_ | CAGGUUUUCUGGUCGUCACUGGAAGACCACU |
| | T15G | |
| 574 | U7_110_ | CAGGUUUUCUGGUCCUCACUGGAAGACCACU |
| | T15C | |
| 575 | U7_110_ | CAGGUUUUCUGGUCUACACUGGAAGACCACU |
| | T16A | |
| 576 | U7_110_ | CAGGUUUUCUGGUCUGCACUGGAAGACCACU |
| | T16G | |
| 577 | U7_110_ | CAGGUUUUCUGGUCUCCACUGGAAGACCACU |
| | T16C | |
| 578 | U7_110_ | CAGGUUUUCUGGUCUUAACUGGAAGACCACU |
| | C17A | |
| 579 | U7_110_ | CAGGUUUUCUGGUCUUGACUGGAAGACCACU |
| | C17G | |
| 580 | U7_110_ | CAGGUUUUCUGGUCUUUACUGGAAGACCACU |
| | C17T | |
| 581 | U7_110_ | CAGGUUUUCUGGUCUUCACUGGAAGACCGCU |
| | A29G | |
| 582 | U7_110_ | CAGGUUUUCUGGUCUUCACUGGAAGACCCCU |
| | A29C | |
| 583 | U7_110_ | CAGGUUUUCUGGUCUUCACUGGAAGACCUCU |
| | A29T | |
| 584 | U7_110_ | CAGGUUUUCUGGUCUUCACUGGAAGACCAGU |
| | C30G | |
| 585 | U7_110_ | CAGGUUUUCUGGUCUUCACUGGAAGACCAUU |
| | C30T | |
| 586 | U7_110_ | CAGGUUUUCUGGUCUUCACUGGAAGACCAAU |
| | C30A | |
| 587 | U7_110_ | CAGGUUUUCUGGUCUUCACUGGAAGACCACA |
| | T31A | |
| 588 | U7_110_ | CAGGUUUUCUGGUCUUCACUGGAAGACCACG |
| | T31G | |
| 589 | U7_110_ | CAGGUUUUCUGGUCUUCACUGGAAGACCACC |
| | T31C | |
| 590 | SNORA38 | UCCUCCUACAAAGGCGUGUCUGUGGUUCCCUGUCUUUGGACACGUAAGAAUUGGAGGAAAAUAAAUGUGGAUUUGGG |
| | | AAACUUUGAGGCCAGCUUGCUUCUUGCAGGCUCAUGAUCAACCAAUCUCACAUAA |
| 591 | SNORA24 | CUCCAUGUAUCUUUGGGACCUGUCAGCCGUGGCAGUCUCCCUUCCUAGCCAUGGAAGAGCAUAUCCUUGUUUAUUGG |
| | | CAAAGCUGUCACCAUUUAAUUGGUAUCAGAUUCUGACUUGCACAAGUAACAUUC |
| 592 | SNORA72 | CUGCGAAUAUUCUCGCUGUUCUGAUUUUGUAAUAGUCAGGACAGGCUAAACAUUCGCUAUAUUAAGACCAUGCAUGU |
| | | GUCCCCAAACCUAGUUCUUUCCCUAGGUCUGGUUUCAUAAAUGCUGGUGAUAAAC |
| 593 | SNORA15 | GCAUGGCCGAAUACUGUGUUUUUAUCAGUAGUUUACACAGCCAGACACCAUGCAAAAGCAGUCUUCCCUUUAGAAUG |
| | | ACUGAUGGUAUGCUAAGGUUUUUCAUAGCAUAUCAUUAUUAAAGGUGAAUACAAAU |
| 594 | SNORA8 | UGCACUGCAUGGUAUCUGCACUCAGCAGUUUACACCUGCUAGGGUGUUCAAAGGUCAGUGCUAUAGAAAUUCAGUAU |
| | | CUGGCAUCGUUGGUUUUCUUGGCUUUGUGCUUGUUAAACCUGGUAUUUCUACUGAUACAGUA |
| 595 | SNORA52 | UGGUCCAUCCUAAUCCCUGCCGGUCCAUCUGUGGCCUGCCAGGUUUCGCUUGUGGACCAGAGCACCCUAGAAGCCUC |
| | | ACCCGAGGAGUGAGCAGGGCUCCAGUGGGCUCACGUCAUGGGCACUUCUAGACACUC |
| 596 | SNORA60 | CACCUGCAUUCAAAAAUGAUCACGGGCUGCCUGUGCUCUGGUCAUCAAUAACGCAGGGAGAGGAAUUGCUGAAAGCC |
| | | GUUUCCCGUGUUUGGAGGGUUCACACCUGUCCCUUUCAAAUGCUGGCGCUUUCACACAC |
| 597 | SNORA14A | UGCAUUCUUAAACCCUCUUGGUGGCUUCCCUGUAAAUGCUUCCAAGAUAUGAGCGAAUGCUAUAGAAAUUGCAGGAA |
| | | AGUCCAAAGGGCUGCGCGUCUCCUGUGGCUCAGUCUUAUUUCAUACCUGCAACAUCU |
| 598 | SNORA68 | AUUGCACCUAAACCCAAGAAUCACUGUUUCUUAUAGCGGUGGUUUAAACAGAGGUGCAAACAGCAAGCGGAUCUUGU |
| | | CGCCUUUGGGGGGCUGUGGCCGUGCCCCUCAAAGUGAAUUUGGAGGUUCCACAACU |
| 599 | SNORA20 | CUUCCCAUUUAUUUGCUGCUUGUAGUCUCACAGUGAUACGAGCAGUUAUACGCAUGGGAUAAAAUAACAUUGGGCCA |
| | | CUGUAAAUUGAGAUGAAGUAACCAUUUUCAUCUCUUCUGCAGGGACUAGACAUUG |
| 600 | SNORA14B | CUGCAUUCUUAAACCCUCUUGGUAGCUUCGUUCUAAGUGCUUCCAAGAUAUGAGUGAAUGCUAUAGAAAUUGCAGGG |
| | | GAGUCCAAAGGGCUGCGCUUCUCCCGUGGCUCAGUCUUAUUUCAUACCUGCGACAUCU |
| 601 | SNORA63 | AGGCAGGAUCUAGUUACAUUGUAGCUGUGAAGUGCUGCAUUGUCUUUGCCCCCUGCUCAAAAUAAAACUGUUACCUU |
| | | UCAAGCCCUGUCUGCCAUGGUGCUGUAGCAGCAGGGAUGUUUGGUCUCAUACAU |
| 602 | SCARNA4 | ACUGGAGGACUAAGGAGGCUGGGUCUGAUGAGGCAAGAUUUUGCUGAUACAUUGCUCCUAGAAAAAAGGGUUGGCAA |
| | | GAGCAGCCCUGGAGACUCACACGGCUGACUGUUCUACCCAACACUC |
| 603 | SNORA63C | AAGCAGGAUUCAGACUACAAUAUAGCUGUUAAGUGCUGUAUUGUCAUUCCCCCUGCUCAAAUUAAAGUUGUUUCUUA |
| | | ACUAUACCCAUCUGCUAUUCUGUAGCAGCCAGGGAUGCUUGGUCACAUACAU |
| 604 | SNORA51 | GGCUUCCUAGUACUUACCAUGGUCUGUGUUCUUACGCUGACUGUAUAGAAACAGGAGGCAGAGUAAACCGACCCCAC |
| | | AUAUACCUCAGCCCAGGCCCUGUGCUGCGUCUGUAUUGUGAAUCAGGAGACAUGG |
| 605 | SCARNA21 | UGGCUCGAUUUCCUGGGGGGUGGUCUCAGCCCACUCCACCUCCCCUCAGCCGAGCCUAGAGUAGAGGGGCCAGGCAU |
| | | CCUCCCCAGGGGAGGGGCGUUGAAGCAAGGAGCCUCUCCUGGGCUGUCCUAGCCUCACAUUU |
| 606 | SNORA18 | GUUGAGGUCUAUCCCGAUAGGUCUUUUCCUGUAGCCUGCACGUUGUUGGAAAUGCCUCAUAGAGUAACUCUGUGAUU |
| | | UUACUUUACUUACAGGACUAUUGUUACAUCUGUGGGAAGGAACCACAAGACAGUU |
| 607 | SNORA68B | UUCUCACCUAAACCCAAGAAUCACUGUUUCUUAUAGCGGUGGUUUAAACAGAGGUGCAAACAGCAAGUGAAUCUCGU |
| | | CGCCUUUGCGGGGCUGUGGCCAUGCCCCUCAAAGGAAAUUUGGAGGCUCUACAGCC |
| 608 | SNORA25 | AGGUCAUUUCAAAGAGGUCUUGUGAGGCUGUGAAACCAAGAGCUCUUAACACUGCGACCAAAGAUGGAAGUUCUCUA |
| | | UAGGAUGCCAUGGCAUUUGAUGGUGCUAUGUUUUCUUGAGGAGAUAUAAGA |
| 609 | SNORA38B | CCCUCCUACAAAGGCAUGUCUAUAGUUCCUUGUCUUUGGACAUGUAAGAAUUGGAGGCAAAGAAAUGUGGACUUGGA |
| | | GAAAUCUGGGGCCAGCUUGCUCUCCGCAGGCUCAAGAUCAACCAUCCCACAUAG |
| 610 | SNORA77 | GCAGACUCACUAUGCACCUGACUGUACUUCCAGGCAGGUGCUUUUUCUGUCUGCCAGAGAAACAUUCCAGGGUGCUG |
| | | UGGCUGCCUCACCUAUCCAGGGCGAUGCAGCUCCCUGGGGACACAGGU |
| 611 | SNORA77B | GCAGACUCACUCUGCAUCUGACUAUACUUCCAGGCGGGUGCUUUUUCUGUCUGCCAGAUAAACAUUCCAGGGUGCUG |
| | | UGGCCGCCUCACGUAUCCAGAGUGAUGCAGCUCCCUGGGGACACAGGU |
| 612 | SNORA79B | UGAUGGCUGUUCCUCUCACUGCUUGAAGCCUUAGGCAGUGGGAUUUUGAUCCAUCAUAUAUCAAAAAUGGCUUAUCU |
| | | UCACUCAGGGCACCAUGAGGAUGGGCUGGCUGUCCGUUAGUGCCUUCUGAUUUUUGCGGAGUCAAACAAUU |
| 613 | SNORA70EL5 | CUGGGGAAUUCAAACUUGUGUUAAGAAAAUGUGUCCCAGUGUGCAAUGGCUGCAAACAGCAGCUUCCUUGGUAGUGU |
| | | AUGCAGCCUGUUUGUUGUACGGGUUGCUCUAAAGGGCCUUGGAGACAGUC |
| 614 | SNORA25L6 | AGGUCACUUCAAAGAGGGCUUGUGGGGCUGUGAAACCAAGAGGUCUUAACAGUAUGACCAAAAACUGAAGUUCUCUA |
| | | UAGGAUGCUGUAGCACUCAAUGGUGCUAUGUUUUCCUCAGGAGAUAUGA |
| 615 | SNORA104 | CUGUCCGUUGCUGGCUUCACAAGUACUAGUAUAAUUUUUAAAAUGUUUUAUUAUUUUGAAAAUAAUGUUGUAAUUCA |
| | | UGCCAGGGACUGACAAAAGACUUGAGACAGGAUGGUUAUUCUUGUCAGCUAAGGUCACAUUG |
| 616 | SNORA98 | UCCAAACAGACACUGAUGGCACCUUCUGCCAUUUAGGAAUUUGUUUUAAAACAGACAUUUGUCUAGAUAUUUCCUUU |
| | | GUGGCCUCCUCCCCAUCAAAAGUCAAUCAAACAUCG |
| 617 | ACA59 | GCCCUAUGUUAAAAUUUUAAUUCUGCACUUACUAACUAUCUUGGGAACCUUGGGCAAGUAACCAACCUCUUGUGCUU |
| | | UGGUUUCCUCAUUGGUAAAAUGGGGAUAACAGUACUUACCUCACAGAGUUGUUGAGAGGAACAAAU |
| 618 | SNORA63B | AUGCAGGUACUGUUACAAUACAACUGAUGUGUUUUGUUGUCGUUCCCCCUGCUUAAAGCACUUGAUGCAUAACUCUG |
| | | UCUACCUUCAUUCCGUAGUAAGACAGAGACGCUUGGCUUCAGACAUUU |
| 619 | SNORA120 | UCACUGCCCUGCUCACCCUUCCUGAGUCCGGCGGCAAGGGUAACUCUGGGAGCAUCGUAGAGGGCAGAGAAGAAGAA |
| | | ACCCUGAGGUCCCAUUAUGUCAGCCCCUUCUAUCACACGGGAGGAGACUGAGGACAGAAAGGGAACAGAG |
| 620 | SNORA116 | CUUUUCUCAGUGGUGCAAGAAGAUUAAGCCACAUUCUGGCUUUAGAGAGGCAUUUCUGAGAGAGAUGAAGGACACUU |
| | | CGUUCCCCAGCCCCAACCUAAGCAUGUGACUGUACUCACCUUGUCAGAUGCUGUUGGAACCUGGCUGACA |
| 621 | SCARNA15 | CUGGAGGACUAAGGAGGCUGGGUCUGAUGAGGCAAGAUUUUGCUGAUACAUUGCUCCUAGAAAAAAGGGUUGGCAAG |
| | | AGCAGCCCUGGAGACUCACACGGCUGACUGUUCUACCCAACACUC |
| 622 | snoU109 | UAGUGUGGAACUGUCUACUCCUCAUUCCUGUGGAAGCAGGAAUACAUUCAUAACAUGCUCCAUUAAAAAAGGAGUUC |
| | | UAGGCCAGGCAGCGUGCCUCAUGCCUGGAAUCCCAGCACUU |
| 623 | SNORA68L2 | AUACCUAAACCCAAGAAUCACUUUCUUAUAGUGAUGAUUUAAACAGAUGCAAACAGCGAGCACAUCUUGUCACCUUU |
| | | GCGGGACUGUGGCUGUGCCCCUCGCAGUAAAUUUGGAGGUUCUACAUCC |
| 624 | SNORA63E | AGGCAGGAUCUAGUUACAUUGUAGCUGUGAAGUGCUGCAUUGUCUUUGCCCCCUGCUCAAAAUAAAACUGUUACCUU |
| | | UCAAGCCCUGUCUGCCAUGGUGCUGUAGCAGCAGGGAUGUUUGGUCUCAUACAUGU |
| 625 | SNORA75 | CACUAGAAGACAGAAUUCACAGAAGUAGCAUUUCACCUUUUGCCUUUACAGAAGUAUAUUUGGCUGUUUUGUGAGAC |
| | | AUUC |
| 626 | SNORA95 | ACUUUUACAGGUAGAAUAGUAAAGCACAGUGUUGAUUGCCCAAGAUUUAUUUUACUUUGAAAAAAUUAGAAAUUUAU |
| | | UACUAUAGCAAAUGUCUAGAACUUUGGAAACAAGU |
| 627 | SNORA31B | AUGCAUCUAUUUGACAGACCUGGAGCAGUUGCUAUCUGCUGCUAUGGUUUCCACCACAGAUGCAAGAAGAACAUGUC |
| | | CUUGCGCUUUCCGUCUGUCUAAUUGUGGCAGCUGAGAUUGAAUAGAGGAAUACAGGA |
| 628 | SNORA501 | AAGCACUGCCUUUGAACCUGAUGUGUCUUGUUUGUAGCUUCACGGGCCAAGCAACAGUGCUAGAGCAUAACGACUUG |
| | | UUAUAACUGGGGCUCUUCAGCUCUCAACUGAACUGCUCUUUUAAAAACAAGGUACAUUU |
| 628 | SNORA502 | AAGCACUGCCUUUGAACCUGAUGUGUCUUGUUUGUAGCUUCACGGGCCAAGCAACAGUGCUAGAGCAUAACGACUUG |
| | | UUAUAACUGGGGCUCUUCAGCUCUCAACUGAACUGCUCUUUUAAAAACAAGGUACAUUU |
| 630 | ACA13 | AGCCUUUGUGUUGCCCAUUCACUUUGGAAACUAGUGAAUGUGGUGUCAAAAAAGGCGUAAAUUAAACGCUUUGCAGC |
| | | CUUUUCCUGCCCUUAAAUUUGAUACCUUUGGUGUAGGAGCUGCAUAAGUAACAGUU |
| 631 | ACA36_2 | UUCCAAAGUGUUGAGUUCAGUCCAGGGCAGCUUCCCUGUUCUGUUAAUUAAACUUUGGGACAUUAAAAUGGGCUAAG |
| | | GGAGAUGAUUGGGUAGAAAGUAUUAUUCUAUUCAUUUGCCUCCCAGCCUACAAAA |
| 632 | ACA28_1 | AAGCAACACUCUGUGGCAGAUGAUCAAAACUGUCUGACACAAUUUGAGCUUGCUAUAGCAAGAAAGUCUAACCUAUU |
| | | CCGGUGUUCUCUCUCCCAUGAGACAAGCCGUUAUAUAGACUUAAACAGU |
| 632 | ACA28_2 | AAGCAACACUCUGUGGCAGAUGAUCAAAACUGUCUGACACAAUUUGAGCUUGCUAUAGCAAGAAAGUCUAACCUAUU |
| | | CCGGUGUUCUCUCUCCCAUGAGACAAGCCGUUAUAUAGACUUAAACAGU |
| 634 | ACA41 | UUCCACAGCUACUGGUCUGCAGCUGUUCUUAUGGUAGCAGUUGUGGCAUUCCUCUGUGGGAAAGAAACUGUUAACAC |
| | | AAACACCUCUUUCUUAGCAAAACAGAAAGUGGGUAUAUAUGUGUGACAGACACAA |
| 635 | ACA5_2 | UGCAGCCGUGUCAAAUUCAGUACCUGUCCUAUGCAUGGUAGGCACUGGCCCAGAAGGCUGCCACAGAAACACUGUGA |
| | | CUCAUGGGCCCUGUUCCUGUGUCCCAGGCUCAGGGAUAAAUUUGGUUACAGACAUCA |
| 594 | ACA8_1 | UGCACUGCAUGGUAUCUGCACUCAGCAGUUUACACCUGCUAGGGUGUUCAAAGGUCAGUGCUAUAGAAAUUCAGUAU |
| | | CUGGCAUCGUUGGUUUUCUUGGCUUUGUGCUUGUUAAACCUGGUAUUUCUACUGAUACAGUA |
| 594 | ACA8_2 | UGCACUGCAUGGUAUCUGCACUCAGCAGUUUACACCUGCUAGGGUGUUCAAAGGUCAGUGCUAUAGAAAUUCAGUAU |
| | | CUGGCAUCGUUGGUUUUCUUGGCUUUGUGCUUGUUAAACCUGGUAUUUCUACUGAUACAGUA |
| 638 | ACA5_1 | UGCAGCCGUGUCAAUUCAGUACCUGUCCUAUGCAUGGUAGGCACUGGCCCAGAAGGCUGCCACAGAAACACUGUGAC |
| | | UCAUGGGCCCUGUUCCUGUGUCCCAGGCUCAGGGAUAAAUUUGGUUACAGACAUCA |
| 631 | ACA36_1 | UUCCAAAGUGUUGAGUUCAGUCCAGGGCAGCUUCCCUGUUCUGUUAAUUAAACUUUGGGACAUUAAAAUGGGCUAAG |
| | | GGAGAUGAUUGGGUAGAAAGUAUUAUUCUAUUCAUUUGCCUCCCAGCCUACAAAA |
| 640 | ACA42_1 | UGGUAAUGGAUUUAUGGUGGGUCCUUCUCUGUGGGCCUCUCAUAGUGUACCCAUGCCAUAGCAAAUGGCAGCCUCGA |
| | | ACCAUUGCCCAGUCCCCUUACCUGUGGGCUGUGAGCACUGAAGGGGGUUGCACAGUG |
| 640 | ACA42_2 | UGGUAAUGGAUUUAUGGUGGGUCCUUCUCUGUGGGCCUCUCAUAGUGUACCCAUGCCAUAGCAAAUGGCAGCCUCGA |
| | | ACCAUUGCCCAGUCCCCUUACCUGUGGGCUGUGAGCACUGAAGGGGGUUGCACAGUG |
| 642 | ACA44_1 | CAGCAUGUUUCCAAGGGCUGUGGCUGGUCAUAGCCAUGGGAUCUCCAACUGCAUGCAAGAGCAACCUGGAAAGACUU |
| | | UGACAGCGCAGGUCAGUACAAUACCUGCAAGCUGCCACUCAGCUUUCCUAUAAUG |
| 642 | ACA44_2 | CAGCAUGUUUCCAAGGGCUGUGGCUGGUCAUAGCCAUGGGAUCUCCAACUGCAUGCAAGAGCAACCUGGAAAGACUU |
| | | UGACAGCGCAGGUCAGUACAAUACCUGCAAGCUGCCACUCAGCUUUCCUAUAAUG |
| 644 | ACA10_2 | GGUCUCUCAGCUCCGCUUAACCACACGGGUCCAGUGUGUGCUUGGCGUGUUUUCAGGGAGGCAGAGAAAGGCUCUCC |
| | | UAAUGCACGACAGACCCGCCCAGAAUGGCCUCUCUGUUCCUAGGAGUGCGACAAUU |
| 645 | ACA46 | AGCACUAUAUUUAAACCUGUGGAUGGGAAUAUUCCCCAUUCUUGGUUACGCUGUAGUGCAAAAGAAUUCCUGGCUCU |
| | | CUGUUGCACAGCUGACUUGUGCCAUUCUGCUGUUGCUGUAUAGAGUUAAGGAACAUGG |
| 644 | ACA10_1 | GGUCUCUCAGCUCCGCUUAACCACACGGGUCCAGUGUGUGCUUGGCGUGUUUUCAGGGAGGCAGAGAAAGGCUCUCC |
| | | UAAUGCACGACAGACCCGCCCAGAAUGGCCUCUCUGUUCCUAGGAGUGCGACAAUU |
| 647 | ACA1 | UGCCUCAUUCUAGAGAAUGGGCACUGUUGAUCAUGGUGUCCAAAAAUAGUUAAUGUGGCUAAAUUGAGACAGGUUAU |
| | | GCUUCCAUCACAGUAUGCAUAUUGCAGUGGUGACAAUGAGACCUGUAACAUUU |
| 648 | ACA2A_1 | UAGGCCCUGAAUCAAGACCAAUGGUUUGCUGUAGCUGUUGGUUUCAAACAGGAGCUAAGAGUGAUGUCUUCCUUGUG |
| | | GUCUGUUGGCUAUUCAGUAUUCCAGUGCGAAUUGCCAAUUCAGUUGGAAGAAACAUAG |
| 649 | ACA2A_2 | UAGGCCCUGAAUCAAGACCAAUGGUUUGCUGUAGCUGUUGGUUUCAAACAGGAGCUAAGAGUGAUGUCUUCCUUGUG |
| | | GUCUGUUGGCUAUUCAGUAUUCCAGUGCGAAUUGCCAAUUCAGUUGGAAGAAACAUAG |
| 650 | ACA3_1 | AUCGAGGCUAGAGUCACGCUUGGGUAUCGGCUAUUGCCUGAGUGUGCUAGAGUCCUCGAAGAGUAACUGCUGACCUU |
| | | AUUCACUGGCUGUGGGCCUUAUGGCACAGUCAGUCACCAGGUUAGAGACAUGC |
| 650 | ACA3_2 | AUCGAGGCUAGAGUCACGCUUGGGUAUCGGCUAUUGCCUGAGUGUGCUAGAGUCCUCGAAGAGUAACUGCUGACCUU |
| | | AUUCACUGGCUGUGGGCCUUAUGGCACAGUCAGUCACCAGGUUAGAGACAUGC |
| 652 | ACA6 | UGCACACUAUUAAAGCUCAGGGUGGAGGCCAGUCUUGGCUCAUGAACUUCUGAGUGUCGGAAGUGUGCUAUAUCAAU |
| | | GGCAGGAUUUUCGCUAACACCAGUAGAGCUUGCCUCUAUGACUGGAGUUUGGUAGUACUCGCUGCCACAUAG |
| 653 | ACA7_1 | GACCUCCUGGGAUCGCAUCUGGAGAGUGCCUAGUAUUCUGCCAGCUUCGGAAAGGGAGGGAAAGCAAGCCUGGCAGA |
| | | GGCACCCAUUCCAUUCCCAGCUUGCUCCGUAGCUGGCGAUUGGAAGACACUCUGCGACAGUG |
| 653 | ACA7_2 | GACCUCCUGGGAUCGCAUCUGGAGAGUGCCUAGUAUUCUGCCAGCUUCGGAAAGGGAGGGAAAGCAAGCCUGGCAGA |
| | | GGCACCCAUUCCAUUCCCACUUGCUCCGUAGCUGGCGAUUGGAAGACACUCUGCGACAGUG |
| 655 | ACA9_1 | UAGCAAGCCUCCAGCGUGCUUGGGUCUGCGGUGACCCUAUGCAUUCCUUCAGUGCUUGCUAGAACAGUUUUGAAACG |
| | | GUUUGAGGCCUUGCCCUGCUCCAUCCAGAGCAAGGUUAUAGAAAUUUCAGACAAUG |
| 655 | ACA9_2 | UAGCAAGCCUCCAGCGUGCUUGGGUCUGCGGUGACCCUAUGCAUUCCUUCAGUGCUUGCUAGAACAGUUUUGAAACG |
| | | GUUUGAGGCCUUGCCCUGCUCCAUCCAGAGCAAGGUUAUAGAAAUUUCAGACAAUG |
| 657 | ACA16 | UUGGCCCUUAUCGAAGCUGCAGCUGCUUCCGCAUAGCUGCUGUGGUCAAAAAGGAGCCCAGAGUGACAGUUUUCCUU |
| | | GACGGUCGCCGUUCUGUUUGUUGUAACUGAUCUGCAACAUUUUGGGAAAAUACAGUU |
| 732 | snoDB0837 | AUCCAAGGUGAUUCCCUCUCCAAGGGGACAUCAGUGCCUCUCAGGAAAGUAGCAGCUUGGAAUAGAAUCUGGCAUGC |
| | | CUAAGGCCUUUGGGGAACUGGGAUGCUUAUUUCCUCUGCCUUCCUUGGCUGCCCACAUGG |
| 733 | snoDB0325 | UUGCACAGUGAACACCCAAGUGUGCUUUAUAGUUCCCUUGGCUUUGACCCUGUGCUAGAGCAUUGCCUGCUCUUCUC |
| | | CUCUGCAUUAAAAGGAAUAUUUAUCCUUUUAAAUGUAUUCAGAAAGCCAGCACAUUA |
| 734 | snoDB0379 | UACCUCCCAGGAUGGCAUCUGGAAGUGGAUAGUAUUCUGCCAGCUUUGGAAACUGGAUGAAAAGCAAAUCUGGCAGA |
| | | GGUACCCAUUUCAUUCCCAGCUUGCUCAGUAGCUGGUGAUUGGAAGAAACUCUGCAACAGUG |
| 735 | snoDB0461 | GGGUCAUUUCAAAGAGGGCUUAUGAGGCUGUGAAACCCAGAGCUCUUAACGCUGUGACCAAAGAUGGAAGUUCUCUA |
| | | UAGGAAGCCAUAGCACUCCUAAUGUUUGGUGCUAUGUUUUCCUGAGGAGAUAUAAAA |
| 736 | snoDB0034 | CUGCAGCCAAUUAAGCCGACUGAGUUCCUUUCCUCAUGGGGGCCCAGUGUGCAAUGGCUGUAAAUAGCAGCUUCCUU |
| | | GGUAGUGUAUGCAGCCUGUUUGUUGUAUGGGUUGCUCUAAGGGACCUUGGAGACAGUC |
| 737 | snoDB0768 | CUCCAUGUAUCUUUGGGACCUGUCAAGUGUGGCAGUCUCCCUUCCUUGCCAUGGAAGAGCAUAUUCUUGUUUACCAG |
| | | CAAAGCUGUCACCAUUUAAUUGGUAUCAGAUUCUGACUUGCACAAGUAACAUUC |
| 738 | snoDB0206 | CUCCAUGUAUCUUUGGGACCUGUCAGCCGUGGCAGUCUCCCUUCCUAGCCAUGGAAGAGCAUAUCCUUGUUUAUUGG |
| | | CAAAGCUGUCACCAUUUAAUUGGUAUCAGAUUCUGACUUGCACAAGUAACAUUC |
| 739 | snoDB0169 | AGGCAGGAUCUAGUUACAUUGUAGCUGUGAAGUGCUGCAUUGUCUUUGCCCCCUGCUCAAAAUAAAACUGUUACCUU |
| | | UCAAGCCCUGUCUGCCAUGGUGCUGUAGCAGCAGGGAUGUUUGGUCUCAUACAU |
| 740 | snoDB0110 | UUCCACAGCUACUGGUCUGCAGCUGUUCUUAUGGUAGCAGUUGUGGCAUUCCUCUGUGGGAAAGAAACUGUUAACAC |
| | | AAACACCUCUUUCUUAGCAAAACAGAAAGUGGGUAUAUAUGUGUGACAGACACAA |
| 741 | snoDB0889 | GCCUGCAUUCGUAAGUGAUCACGGGCUGCCCGUGUCCUGGUCAUUGGUAGUGCAGGCAGAGGAAAUGCGGGAAAGGU |
| | | UGCUGUGUUUGGAGGGUCCACAUCUUCACCCUCCUGUCCCAGGAGCUUUCCUACA |
| 742 | snoDB0063 | GUGCAAACUCGAUCACUAGCUCUGCGUGAUGUGGCAGAAGCGAAGGGAACCAGGUUUGCAAAAGUAACUGUGGUGAU |
| | | GGAAAUGUGUUAGCCUCAGACACUACUGAGGUGGUUCUUUCUAUCCUAGUACAGUC |
| 743 | snoDB0136 | UUCCAAAGUGUUGAGUUCAGUCCAGGGCAGCUUCCCUGUUCUGUUAAUUAAACUUUGGGACAUUGAAAUGGGCUAGG |
| | | GGAGAUGAUUGGGUAGAAAGCAUUAUUUUAUUCAUUUGCCUCCCAGCCUACAAAA |
| 744 | snoDB0966 | CCCUUGGCCCUUAUCGAAGCUGCAGCUGCUUCCGCAUAGCUGCUGUGGUCAAAAAGGAGCCCAGAGUGACAGUUUUC |
| | | CUUGACGGUCGCCGUUCUGUUUGUUGUAACUGAUCUGCAACAUUUUGGGAAAAUACAGUU |
| 745 | snoDB0245 | UGGAGGACUGAGAAGGUGAGGCAGUUUUGCCCCGUGCUGCCUUCCACCGGUUAAGACCUCCAAAAUCGAAGGGCUGC |
| | | CCAGGCAGAGGAUGUCCCCUUGCCACCCUUGGAGGGGCAGCGCUGUGCUGGCCGACAUUUG |
| 746 | snoDB0466 | GUUGAGGUCUAUCCCGAUGGGGCUUUUCCUGUAGCCUGCACAUCGUUGGAAACGCCUCAUAGAGUAACUCUGUGGUU |
| | | UUACUUUACUCACAGGACUAUUGUUAGAUCUGUGGGAAGGAAUUACAAGACAGUU |
| 747 | snoDB0464 | UGCACUGCAUGGUAUCUGCACUCAGCAGUUUACACCUGCUAGGGUGUUCAAAGGUCAGUGCUAUAGAAAUUCAGUAU |
| | | CUGGCAUCGUUGGUUUUCUUGGCUUUGUGCUUGUUAAACCUGGUAUUUCUACUGAUACAGUA |
| 748 | snoDB0256 | UCCAGCUGUAGGCAGCUGCCUAGGUUGUCUUGUACCUAGGCAAGUGUUACACUGCUGGGAGAACAGCAGCCAAUAGC |
| | | UGGUUGGCAUUCUGGCCCUGGUUCAUGCCAACUCUUGUGUUGACUACCCCAGGAUGCCAGCAUA |
| 749 | snoDB0554 | CUUCCUCAGCCUUACUCCAGGGACUUUUUGUUGCCUGUAAAGUGCUCUGGCAUUGCCUGAGGAUAGAUGAGAAAGCA |
| | | CAUAUCCCUCCCCAGUAAGACGCUGUUUUCUUUUGGGGCCUACAAGUUGAGCUGACAGU |
| 750 | snoDB0776 | GGUCUCUCAGCUCCGCUUAACCACACGGGUCCAGUGUGUGCUUGGCGUGUUUUCAGGGAGGCAGAGAAAGGCUCUCC |
| | | UAAUGCACGACAGACCCGCCCAGAAUGGCCUCUCUGUUCCUAGGAGUGCGACAAUU |
| 751 | snoDB0047 | GCAUGGUAAUGGAUUUAUGGUGGGUCCUUCUCUGUGGGCCUCUCAUAGUGUACCCAUGCCAUAGCAAAUGGCAGCCU |
| | | CGAACCAUUGCCCAGUCCCCUUACCUGUGGGCUGUGAGCACUGAAGGGGGUUGCACAGUG |
| 752 | snoDB0958 | GCGCUGUCUUUGAGCCCCCGCCGAGCUUCCUCGUGGCGCCGGGGGUCAAUCUGCAGCGCUAGAGCAUGUGCUUGCGC |
| | | AUAACUGGGGCCGCCUGGCCUCCCGCGGGCGGCCUUUUUAACCGCGAGCGACAAGA |
| 753 | snoDB0797 | GCAUGGGUUUGGAUUUAUGAUGGGCUGGAUUCCCUAGGCCUCUCAUAGUACCCCAUGCCAGAGCAAACUGUAGCCCC |
| | | AACCAUUGCCGGGCCUCUAUGCCUGUAGGCUGCUGGCACUGAAGUGGGUUGCACAGUA |
| 754 | snora8 | UGCACUGCAUGGUAUCUGCACUCAGCAGUUUAUACCUGCUAGGGUGUUCAAAGGUCAGUGCUAUAGAAAUUCAGUAU |
| | | CUGGCAUCGUUGGUUUUCUUGGCUUUGUGCUUGUUAAACCUGGUAUUUCUACUGAUACAGU |
| 755 | snoDB0432 | GCCCUGUGGUUGCUGGAUGCUGUUGUGCAUGGACAGCUCUCCAGUGGAUUCGAUGGGCCAUAGCAAUCCUGUGAUUU |
| | | AUGCAUGGAGGCUGCUUCUCCUCAGCAGCUGCCAUAGCCCGGUCGCUGGUACAUGA |
| 756 | snoDB0890 | ACCUGCAUUCAAAAAUGAUCACGGGCUGCCUGUGCUCUGGUCAUCAAUAACGCAGGGAGAGGAAUUGCUGAAAGCCG |
| | | UUUCCCGUGUUUGGAGGGUUCACACCUGUCCCUUUCAAAUGCUGGCGCUUUCACACA |
| 757 | snoDB0502 | GCCGAGACUAGAGUCACAUCCUGACACAACUCUUGUCCUGGUGUGCUAGAGUACUCGAAGAGAAUCUACUGGUCUUG |
| | | AUUCACUGGUGGGGGCAGUCGGUGCCCCCGUUAGUGCCCAGAUCAGAAACAUAC |
| 758 | snoDB0305 | GCAUGGGUUUGGAUUAUGACAGGCCCAUCCCCCUGGACCUCUCAUAGUGCCCCAUGCCAGAGCAAACUGUGGCCCCG |
| | | AACCAUUGCCUGGCUUCUGUACCCGUGGGCCACUGGCACUGAAGAGGGUUACACAGUG |
| 759 | snoDB0974 | AAGCACUGCCUUUGAACCUGAUGUGUCUUGUUUGUAGCUUCACGGGCCAAGCAACAGUGCUAGAGCAUAACGACUUG |
| | | UUAUAACUGGGGCUCUUCAGCUCUCAACUGAACUGCUCUUUUAAAAACAAGGUACAUUU |
| 760 | snoDB0260 | CUGCAGCCAAUUAAGCCGACUGAGUUCCUUUCCUCAUGGGGGCCCAGUGUGCAAUCGCUGCAAACAGCAGCUUCCUU |
| | | GGUAGUAUAUGCAGCCUGUUUAUUGUACGGGUUGCUCUACGGGACCUUGGAGACAGGC |
| 761 | snoDB0183 | AAGCAGGAUUCAGACUACAAUAUAGCUGCUAAGUGCUGUGUUGUCGUUCCCCCUGCUUAAAAUAAAGUUGUUUCUUA |
| | | ACUAUACCUGUCUGCUAUUCUCCUGUAGCAGCCAGGGACGCUUGGUCUCAUACAU |
| 762 | snoDB0293 | UCCUCCUACAAAGGCGUGUCUGUGGUUCCCUGUCUUUGGACACGUAAGAAUUGGAGGAAAAUAAAUGUGGAUUUGGG |
| | | AAACUUUGAGGCCAGCUUGCUUCUUGCAGGCUCAUGAUCAACCAAUCUCACAUAA |
| 763 | snora54 | GAGCACUGUUCGUAACCCGUUAGCCUGGCUGUAGCUAAUGGGUUCCAUUCCGGUGCAAUAGCAUUUCCAGCGACACA |
| | | UGACUGACUGACUGGUGGCUUUCAGUUUCAGGUCUUGGAGACAAAU |
| 764 | snoDB0345 | UUUCCCUGACCUGGGUAGAGUGGCAUCCAGUUGGUGGUGCCCAUCUCAUAUCAGCCAGGGACAAAGCAACCCCUUGU |
| | | UCCUCCCAGCUUGGCUUUUCAUCUGUGCCUAUGCCUGGUUCAUGCCUUGGACACAUUU |
| 765 | snoDB0538 | UUGGCCCUGAAUCAAGGCCAGCAGUUUGCUGAAGCUGUUGGUUUCAAGCAGGAGCCUAAAGAAUUGUCUUUCUAUGG |
| | | UCUGUUGGCCAUUUCAUAACUUUGGAAAUGUAAUGGUCAAUUCAUUAGAAAGAAACAUGA |
| 766 | snoDB0316 | UGCAGCCGUGUCAAAUUCAGUACCUGUCCUAUGCAUGGUAGGCACUGGCCCAGAAGGCUGCCACAGAAACACUGUGA |
| | | CUCAUGGGCCCUGUUCCUGUGUCCCAGGCUCAGGGAUAAAUUUGGUUACAGACAUCA |
| 767 | snoDB0947 | GCAUGGGUUUGGAUUUAUGAUGGGCCCGUCCCCCUGGACCUCUCAUAGUACCCCAUGCCAGAGCAAACUGUAGCCCU |
| | | GAACCAUUGCCUGGCCUCUGUUCCCGUAGGCUGCUGGCACUGAAGUGGGUUGCACAAUA |
| 768 | snoDB0073 | CAGCAUGUUUCCAAGGGCUGUGGCUGGUCAUAGCCAUGGGAUCUCCAACUGCAUGCAAGAGCAACCUGGAAAGACUU |
| | | UGACAGCGCAGGUCAGUACAAUACCUGCAAGCUGCCACUCAGCUUUCCUAUAAUG |
| 769 | snoDB0246 | AGCCUUUGUGUUGCCCAUUCACUUUGGAAACUAGUGAAUGUGGUGUCAAAAAAGGCGUAAAUUAAACGCUUUGCAGC |
| | | CUUUUCCUGCCCUUAAAUUUGAUACCUUUGGUGUAGGAGCUGCAUAAGUAACAGUU |
| 770 | snoDB0286 | CUUCCCAUUUAUUUGCUGCUUGUAGUCUCACAGUGAUACGAGCAGUUAUACGCAUGGGAUAAAAUAACAUUGGGCCA |
| | | CUGUAAAUUGAGAUGAAGUAACCAUUUUCAUCUCUUCUGCAGGGACUAGACAUUG |
| 771 | snoDB0382 | AAAGCAGGUUGCAAUUACAGUGCUUCAUUUUGUGGAAGUACUGCCAUUAUCCUGCUGAAAGAAAAGCCGUGUUAAUC |
| | | AUUUUUGAUUUUGCCUUUAUGAGGGUAAAAUCAUGACAGAUUGACAUGGACAAUU |
| 772 | snoDB0960 | GCACUGCUUUAGAGCCUCUGCGUGUCUGGCUGUGGCCUCAGGGGUCUAGCAGCAGUGCUAGAGCAGACCCAGCUUGU |
| | | CAGAUCCGGGGCUGCUUAGAGGCCACCUAAGUGAUUCCUUUGGCAGCAAGCAACAUUC |
| 773 | snoDB0971 | GCUGUCCUGGACCUGUUGGCACCACAGACAGUUGCUCUGCUGUGCCUGUGGCCUCGGGGCAAAGAGAAAGUGGCGAU |
| | | UUCUACACUCAGUGCUCGGGAACCAGUGGGCACUGAGAAUGGUUUAUGGCCUGACAUUA |
| 774 | snoDB0040 | UUCCAAAGUGUUAAGUUCAGUUCAGGGUAGCUUCCCUGCUCUGUUAAUUAAACUUUGGAACAUUGAAACUGGCUAGG |
| | | GAAAAUGAUUGGAUAGAAACUAUUAUUCUAUUCAUUUAUCCCCAGCCUACAAAA |
| 775 | snoDB0904 | AUUGCACCUAAACCCAAGAAUCACUGUUUCUUAUAGCGGUGGUUUAAACAGAGGUGCAAACAGCAAGCGGAUCUUGU |
| | | CGCCUUUGGGGGGCUGUGGCCGUGCCCCUCAAAGUGAAUUUGGAGGUUCCACAACU |
| 776 | snoDB0317 | UGCAGUCAAGUCAAAUUCAGUGCCCGUUUCUGUCAUAGCGGGGGCUGGCCCAGAUGGCUGCCACAGCAAGCUCCACA |
| | | GCUCAUGGGCCCUGGGUCACCUACCCUGGGACCUGGGGAUAAGUUUGGCUGUGGACAGUG |
| 777 | snoDB0835 | UGUCCCUGACCUGGGUAGAGUGGCAUCUGGUUGGUGAUGCCCAUCUCAUAUCAGCCAGGGACAAAGCAACUCCUUGU |
| | | UCAUCCCAGCUUGGCUUUUGAUCCGUGCCCAUGCCUGGUUCAUGCCUUGGACACAUAG |
| 778 | snoDB0372 | CCGCAGCCAAUUAAGCCGACUGAGUUCCUUUCCUCAUGGGGACCCAGUGUGCGAUGGCUGCACACAGCAGCUUCCUU |
| | | GGUAGUGUACGCAGCCUGUUGGUUGUAUGGGUUGCUCUAAGGGACCUUGGAGACAGGC |
| 779 | snoDB0833 | CCCUCCUACAAAGGCAUGUCUAUAGUUCCUUGUCUUUGGACAUGUAAGAAUUGGAGGCAAAGAAAUGUGGACUUGGA |
| | | GAAAUCUGGGGCCAGCUUGCUCUCCGCAGGCUCAAGAUCAACCAUCCCACAUAG |
| 780 | snoDB0041 | GGGCAUACCCGUAGACCUUGUCUGACUGUGCUCAUGGCCAGGCAGGGGGGACAGUGUAUGCAAGAGUAAUGUGGAGU |
| | | UUGUGCUAACUCUAGCCAGCUUAAUUAGUGACUGGAUAAAUUGCACAACUCUCACAUUC |
| 781 | snoDB0968 | ACUGCCCCUAGAGGCGUUGCAGCUGUGGCUGCCGUGUCACAUCUGUGUCAUUAGGUGGCAGAGAUUAGAGAGGCUAU |
| | | GUCUACGCUCAGCGUUCUGCCCCGUGAACGUUUGAAUGUUUGAUAGUCUCACACUC |
| 782 | snoDB0055 | GCACCUGAAUCUUUCCCAUUCCUUGCUGCCUCGUGCCGGUGUGGGGACAGAUGGUGCUACAGAAUGAGCAGAGGAAA |
| | | UCCAGACAGGUUGUUUUCCAUUUGUCUUGGGGCCUGUCUCUACAGCUCUGCCACAUUU |
| 783 | snoDB0184 | CUACCAAAAGUUAGCUUUUUGGGGGGCAGGUUUUUAAGUAACCUUUGCCAACUUGGGCUAUUUGGAAGAGUAAAAGA |
| | | CCACACUCCACAGUGGGCUAUACCACUUAGUAUAGUUCGCUACUAUUUUGUGGCCUACAUG |
| 784 | snoDB0134 | GCAUGGGUUUGGAUUUAUGACAGGCCCGUCACCCUGGGCCUGUCAUAGUACCCCAUGCCAGAGCAAACUGUGUCCCC |
| | | GAACCAUUGCCUGGCCUCUGUGCCCGUAGGCUGCUGGCACUGAAGUGGGUUGCACAGUG |
| 785 | snoDB0777 | ACUCUCUCGGCUCUGCAUAGUUGCACUUGGCUUCACCCGUGUGACUUUCGUAACGGGGAGAGAGAGAAAAGAUCUCC |
| | | UCAGGACCUCGGAUGGGCCUUACUGUGGCCUCUCUUUCCUUGAGGGGUGCAACAGGC |
| 786 | snoDB0820 | CCCCUUUUAAAAGCACUCAAUGGGCCUGUGGCUAAUGACCUAUUGAGCCGUCAAGAAAGGGGAGAGUGAAAACAUCG |
| | | CUUUUGGGUGAAGUGGCAACAUGUGUUGUUUGCUUCAAUCGGUGGUGUGACAAGG |
| 787 | snoDB0879 | GGCCUCCUGGUGCUUACCACAGGCUGUGUUCUUACACUGACUGUAUAGAAAGAGGAGGUAGAGUAAACCUACCCCAU |
| | | AUACACCUCAGCUCAGGCCCUGUGCCUGGUCUGUAUUGUGAAUGGGGGAACAUAG |
| 788 | snoDB0158 | GGGCAUACUCGUAGACCUUGCCUGACUGUGCUCAUGUCCAGGCAGGGGGGACAGUGUAUGCAAGAAUAAUUUGGAGU |
| | | UCCUGCCAGCUCUAACCAGCUUCAUCAGUGGCUGGAUAAAUUGCAGGACUCUAAACAUUU |
| 789 | snoDB0403 | CUGCGAAUAUUCUCGCUGUUCUGAUUUUGUAAUAGUCAGGACAGGCUAAACAUUCGCUAUAUUAAGACCAUGCAUGU |
| | | GUCCCCAAACCUAGUUCUUUCCCUAGGUCUGGUUUUAUAAAUGCUGGUGAUAAAC |
| 790 | snoDB0319 | UAGCAAGCCUCCAGCGUGCUUGGGUCUGCGGUGACCCUAUGCAUUCCUUCAGUGCUUGCUAGAACAGUUUUGAAACG |
| | | GUUUGAGGCCUUGCCCUGCUCCAUCCAGAGCAAGGUUAUAGAAAUUUCAGACAAUG |
| 791 | snoDB0383 | UUCCAAAGUGUUGAGUUCAGUCCAGGGCAGCUUCCCUGUUCUGUUAAUUAAACUUUGGGACAUUAAAAUGGGCUAAG |
| | | GGAGAUGAUUGGGUAGAAAGUAUUAUUCUAUUCAUUUGCCUCCCAGCCUACAAAA |
| 792 | snoDB0801 | AGCACUAUAUUUAAACCUGUGGAUGGGAAUAUUCCCCAUUCUUGGUUACGCUGUAGUGCAAAAGAAUUCCUGGCUCU |
| | | CUGUUGCACAGCUGACUUGUGCCAUUCUGCUGUUGCUGUAUAGAGUUAAGGAACAUGG |
| 793 | snoDB0031 | CUGCAUUCUUAAACCCUCUUGGUAGCUUCGUUCUAAGUGCUUCCAAGAUAUGAGUGAAUGCUAUAGAAAUUGCAGGG |
| | | GAGUCCAAAGGGCUGCGCUUCUCCCGUGGCUCAGUCUUAUUUCAUACCUGCGACAUCU |

1-379. (canceled)

380. A method for trans-splicing of pre-mRNA target sequences comprising:
(a) introducing into a eukaryotic cell a trans-splicing RNA molecule comprising:
(i) a small nucleolar RNA (snoRNA) sequence, wherein the snoRNA sequence comprises an H box RNA sequence having the polynucleotide sequence of SEQ ID NO: 1, an ACA box RNA sequence having the polynucleotide sequence of SEQ ID NO: 2, and has at least 80% to a nucleic acid selected from any one of SEQ ID NOS: 590-628 and 732-793;
(ii) one or more intronic sequences and/or exonic sequences to replace at least a portion of the one or more pre-mRNA target sequences;
(iii) at least one splice acceptor site or splice donor site located at a 5′ or 3′ boundary of the one or more intronic sequences and/or exonic sequences, wherein the at least one splice acceptor site or splice donor site and the one or more intronic sequences and/or exonic sequences are located 5′ or 3′ of the one or more snoRNA sequences; and
(iv) one or more binding domains comprising a nucleic acid sequence of about 5 to about 300 nucleotides in length that binds to the one or more pre-mRNA target sequences, wherein the one or more binding domain sequences are positioned (i) upstream of the H consensus sequence; (ii) downstream of the ACA consensus sequence; (iii) between the H consensus sequence and the ACA consensus sequence; or (iv) a combination of (i)-(iii); and
(b) replacing at least a portion of the one or more pre-mRNA target sequences with the one or more intronic sequences and/or exonic sequences.

381. (canceled)

382. The method of claim 380, wherein the H/ACA box comprises 5′ to 3′ an H consensus sequence and an ACA consensus sequence.

383. The method of claim 380, wherein the snoRNA sequence comprises at least one binding domain sequence positioned: upstream of the H consensus sequence; downstream of the ACA consensus sequence.

384-385. (canceled)

386. The method of claim 380, wherein the snoRNA sequence binds a ribonucleoprotein (RNP) to direct trans-splicing of the pre-mRNA target sequence.

387. The method of claim 380, wherein the trans-splicing RNA molecule comprises from 3′ to 5′: the snoRNA sequence and the at least one splice acceptor or splice donor site.

388. The method of claim 380, wherein the trans-splicing RNA molecule comprises from 5′ to 3′: the snoRNA sequence and the at least one splice acceptor or splice donor site.

389. The method of claim 380, wherein the one or more binding domains are located outside the snoRNA sequence.

390. The method of claim 380, wherein the one or more binding domains are located within the snoRNA sequence.

391. The method of claim 380, wherein the snoRNA sequence comprises 2 binding domains, or comprises more than 2 binding domains.

392. The method of claim 380, wherein the one or more binding domains are about 5 to about 20, about 5 to about 30, about 5 to about 40, about 5 to about 50, about 10 to about 50, about 10 to about 100, about 20 to about 100, about 30 to about 100, about 40 to about 100, about 50 to about 100, about 50 to about 150, about 50 to about 200, about 50 to about 250, about 100 to about 150, about 100 to about 200, about 100 to about 250, or about 100 to about 300 nucleotides in length, each having at least 80% complementarity to one or more pre-mRNA target sequences.

393. The method of claim 380, wherein the trans-splicing RNA molecule comprises one or more binding domains, wherein the one or more binding domains each have at least 80% complementarity to one or more pre-mRNA target sequences.

394. The method of claim 393, wherein each of the one or more binding domains have at least 95% complementarity to one or more pre-mRNA target sequences.

395. The method of claim 380, wherein the one or more pre-mRNA target sequences comprises an USH2A pre-mRNA sequence, or wherein the one or more pre-mRNA target sequences comprises intron 12 and/or exon 13 of an USH2A pre-mRNA.

396. The method of claim 380, wherein the snoRNA sequence comprises one or more M6A sites.

397. The method of claim 396, wherein the snoRNA sequence comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 M6A sites.

398. The method of claim 380, wherein the snoRNA sequence comprises a nucleic acid sequence of SNORA101B, SNORA48, SNORA54, SNORA66, SNORA73A, or SNORA8.

399. The method of claim 380, wherein the snoRNA nucleic acid sequence has at least 90%, at least 95%, at least 98%, or at least 99% identity to a nucleic acid selected from any one of SEQ ID NOs: 590-657 and 732-793.

400. The method of claim 399, wherein the snoRNA nucleic acid sequence comprises a nucleic acid sequence selected from any one of SEQ ID NOs: 590-657 and 732-793.

401. The method of claim 380, wherein the trans-splicing RNA molecule comprises one or more splicing signals.

402. The method of claim 401, wherein the one or more splicing signals are selected from an exonic splicing enhancer (ESE), an intronic splicing enhancer (ISE), an exonic splicing silencer (ESS), intronic splicing silencer (ISS), a U1 binding motif, a polypyrimidine tract, a branch point, and a combination thereof.

403. The method of claim 380, wherein the trans-splicing RNA molecule comprises one or more polypyrimidine tracts, branch points, and splice acceptors (SA) and optionally comprises a splice donor (SD) and is suitable for 3′ editing of the pre-mRNA target sequence.

404. The method of claim 380, wherein the trans-splicing RNA comprises one or more splice donors (SD) and optionally comprises one or more polypyrimidine tracts, branch points, and splice acceptors (SA) and is suitable for 5′ editing of the pre-mRNA target sequence.

405. The method of claim 380, wherein the trans-splicing RNA molecule further comprises a ribozyme site.

406. The method of claim 405, wherein the ribozyme site comprises a hairpin, hammerhead, hepatitis delta virus (HDV), twister ribozyme site, Varkud satellite (VS), or glmS ribozyme site, or a variant thereof.

407. The method of claim 380, wherein the trans-splicing RNA molecule further comprises one or more of a 5′ cap, 5′ UTR, 3′ untranslated region (UTR), and 3′ polyA signal/tail.

408. The method of claim 380, wherein the trans-splicing RNA molecule is introduced into the cell via a plasmid, viral vector, non-viral vector, or circular RNA (cirRNA), encoding the one or more trans-splicing RNA molecules; and/or wherein the trans-splicing RNA molecule is introduced into the cell via a delivery vehicle comprising a lipid nanoparticle (LNP) or a polymeric nanoparticle.

409. The method of claim 380, wherein the method further comprises introducing multiple trans-splicing RNA molecules into the cell via multiple plasmids, viral vectors, non-viral vectors, or circular RNAs (cirRNAs), wherein each of the multiple plasmids, viral vectors, non-viral vectors, or cirRNAs encodes a distinct trans-splicing RNA molecules.
